SEQUENCE LISTING (21 sequences)

SEQ ID NO: 1 - DNA, 67 bp, Homo sapiens
gctcatactacaatgtcacttactgtaagagcagatccctggacaggcaaggaatacaggtaaaaaa

SEQ ID NO: 2 - DNA, 67 bp, Homo sapiens
gctcatactacaatgtcacttactgtaagagcagatccctggacaggcgaggaatacaggtaaaaaa

SEQ ID NO: 3 - DNA, 57 bp, Artificial Sequence
  Primer for PCR
  misc_feature (34)..(45): the segment 34 to 45 contains 2′-O-Me modifications and serves as a second primer region to bind the first region of the controller oligonucleotide
  misc_feature (44)..(45): Between nucleotide 44 and 45 there is a C3 linker which serves the termination of the polymerase
  misc_feature (45)..(57): Segments 45 to 57 serve as primers
  modified_base (45)..(45): Uracil instead of thymidine
  misc_feature (45)..(46): Between nucleotide 45 and 46 there is a C3 linker which serves the termination of the polymerase
  modified_base (47)..(47): Uracil instead of thymidine
  modified_base (50)..(50): Uracil instead of thymidine
  modified_base (53)..(53): Uracil instead of thymidine
  modified_base (54)..(54): Uracil instead of thymidine
aactcagacaagatgtgatttttttacctgtatctctgatgcttctacctgtattcc

SEQ ID NO: 4 - DNA, 56 bp, Artificial Sequence
  Controller
  misc_feature (1)..(39): The segments 1 to 39 comprise 2′-O-Me nucleotide modifications
  modified_base (1)..(1): Uracil instead of thymidine
  modified_base (4)..(4): Uracil instead of thymidine
  modified_base (6)..(6): Uracil instead of thymidine
  modified_base (8)..(8): Uracil instead of thymidine
  modified_base (18)..(18): Uracil instead of thymidine
  modified_base (22)..(22): Uracil instead of thymidine
  modified_base (37)..(37): Uracil instead of thymidine
  misc_feature (56)..(56): The end of the controller contains a 3′-phosphate group for blockade possible extension by polymerase
taatctgtaagagcagatccctggacaggcaaggaatacaggtagaagcatcagag

SEQ ID NO: 5 - DNA, 70 bp, Artificial Sequence
  Primer for PCR
  misc_feature (35)..(36): Nucleotide 35 and 36 are connected via a HEG linker
ctacagaactcagacaagatgtgaactacaatgttgctcatactacaatgtcacttactgtaagagcaga

SEQ ID NO: 6 - DNA, 70 bp, Artificial Sequence; identical to SEQ ID NO: 5
  Primer for PCR
  misc_feature (35)..(36): Nucleotide 35 and 36 are linked via a HEG linker

SEQ ID NO: 7 - DNA, 60 bp, Artificial Sequence
  Mismatch controller oligonucleotide
  misc_feature (28)..(46): The middle segment of the oligonucleotide comprises 2′-O-Me nucleotide modifications
  modified_base (41)..(41): Uracil instead of thymidine
  misc_feature (60)..(60): 3′-phosphate group to block a possible extension by polymerase
gctctaatctgtaagagcagatccctggacaggcaaagaatacaggtagaagcatcagag

SEQ ID NO: 8 - DNA, 120 bp, Homo sapiens
tgacgtggacatcatgagagacatcgcctctgggctaataggactacttctaatctgtaagagcagatccctggacaggcaaggaatacaggtattttgtccttgaagtaacctttcaga

SEQ ID NO: 9 - DNA, 60 bp, Artificial Sequence
  Block oligonucleotide
  misc_feature (1)..(12): Sequence segment 1 to 12 comprises LNA modifications
ccagaggcgatgtatctcatgatgtccacaacactgtagtatggtcttgttaagcaaaaa

SEQ ID NO: 10 - DNA, 44 bp, Artificial Sequence
  Controller
  modified_base (1)..(1): Uracil instead of thymidine
  modified_base (4)..(4): Uracil instead of thymidine
  modified_base (6)..(6): Uracil instead of thymidine
  modified_base (8)..(8): Uracil instead of thymidine
  modified_base (18)..(18): Uracil instead of thymidine
  modified_base (22)..(22): Uracil instead of thymidine
  modified_base (37)..(37): Uracil instead of thymidine
taatctgtaagagcagatccctggacaggcaaggaatacaggta

SEQ ID NO: 11 - DNA, 59 bp, Homo sapiens
cttacagtaagtgacattgtagtatgagcgatccctggacaggcaaggaatacaggtaa

SEQ ID NO: 12 - DNA, 58 bp, Homo sapiens
cttacagtaagtgacattgtagtatgagcgatccctggacaggcaagaatacaggtaa

SEQ ID NO: 13 - DNA, 57 bp, Homo sapiens
cttacagtaagtgacattgtagtatgagcgatccctggacaggcaaaatacaggtaa

SEQ ID NO: 14 - DNA, 59 bp, Homo sapiens
cttacagtaagtgacattgtagtatgagcgatccctggacaggcaaggaataaaggtaa

SEQ ID NO: 15 - DNA, 59 bp, Homo sapiens
cttacagtaagtgacattgtagtatgagcgatccctggacaggcaaagaatacaggtaa

SEQ ID NO: 16 - DNA, 58 bp, Homo sapiens; identical to SEQ ID NO: 12
  modified_base (47)..(47): Iso-dC instead of cytosine

SEQ ID NO: 17 - DNA, 58 bp, Homo sapiens
  modified_base (43)..(43): Iso-dC instead of cytosine
cttacagtaagtgacattgtagtatgagcgatccctggacagcaaggaatacaggtaa

SEQ ID NO: 18 - DNA, 47 bp, Artificial Sequence
  Primer for PCR
  misc_feature (36)..(47): Segments 36 to 47 serve as primer
acgaagctcgcaagaactcagagtgtgctcgacactacctgtattcc

SEQ ID NO: 19 - DNA, 22 bp, Artificial Sequence
  Primer for PCR
  misc_feature (1)..(1): Tetramethyl-Rhodamin at the 5′-end
  misc_feature (1)..(10): Segments 1 to 10 comprise 24-O-Me modifications
  modified_base (2)..(2): Uracil instead of thymidine
  modified_base (5)..(5): Uracil instead of thymidine
  modified_base (8)..(8): Uracil instead of thymidine
  modified_base (9)..(9): Uracil instead of thymidine
  misc_feature (10)..(11): nucleotide 10 and 11 are connected via a C3 linker
ctgatgcttctacctgtattcc

SEQ ID NO: 20 - DNA, 56 bp, Artificial Sequence; identical to SEQ ID NO: 4
  Controller oligonucleotide
  misc_feature (1)..(39): The segments 1 to 39 comprise 2′-O-Me nucleotide modifications
  modified_base (1)..(1): Uracil instead of thymidine
  modified_base (4)..(4): Uracil instead of thymidine
  modified_base (6)..(6): Uracil instead of thymidine
  modified_base (8)..(8): Uracil instead of thymidine
  modified_base (18)..(18): Uracil instead of thymidine
  modified_base (22)..(22): Uracil instead of thymidine
  modified_base (37)..(37): Uracil instead of thymidine
  misc_feature (56)..(56): Phosphate group to block a possible extension by polymerase

SEQ ID NO: 21 - DNA, 48 bp, Artificial Sequence
  Fluorescence probe
  misc_feature (14)..(15): Nucleotide 14 and 15 are connected via a HEG linker
  misc_feature (22)..(23): Nucleotide 22 and 23 are linked via dT-BHQ1
tagtatgagcttttgctcatacacaatgtcacttactgtaagagcaga

(19) United States
(12) Patent Application Publication
(10) Pub. No.: US 2021/0071243 A1
(43) Pub. Date: Mar. 11, 2021

(54) METHOD FOR PRIMER EXTENSION REACTION WITH IMPROVED SPECIFICITY

(71) Applicant: AGCT GMBH, Lübeck (DE)

(72) Inventors: Dmitry CHERKASOV, Marburg (DE); Christian GRUNWALD, Gießen (DE)

(73) Assignee: AGCT GMBH, Lübeck (DE)

(21) Appl. No.: 16/975,542

(22) PCT Filed: Feb. 26, 2019
(86) PCT No.: PCT/EP2019/054755

§ 371 Date: Nov. 23, 2020

(30) Foreign Application Priority Data
Feb. 26, 2018 (EP) 18158720.5
Feb. 28, 2018 (DE) 10 2018 001 639.1
Sep. 18, 2018 (EP) 18195189.8

(51) Int. Cl. C12Q 1/6848

(52) U.S. Cl. CPC C12Q 1/6848

(57) ABSTRACT

The present invention relates to a method for the extension of an oligonucleotide primer with improved specificity using a specific oligonucleotide primer and a controller nucleotide, wherein the controller oligonucleotide enables sequence-specific strand opening of a double strand of extension product and template. The invention further relates to a respective kit for carrying out the method according to the invention.

The present invention relates to a method for the template-dependent primer extension reaction using a template-dependent polymerase. The method can generate primer extension products which can be utilized in an amplification of nucleic acids. The method can thus contribute to improving the specificity of analyses comprising a primer extension step.

The present application claims the priority of the following applications: European patent applications EP18158720.5 of 26.02.18; EP18195189.8 of 18.09.2018; German patent application 10 201 8 001639.1 of 28.02.2018; these and all other patent documents mentioned herein shall be deemed to be incorporated by reference into the present description (incorporation by reference).

The nucleic acid sequences provided herewith are shown using standard letter abbreviations for nucleotide bases as defined in 37 C.F.R. 1.822. Only one strand of each nucleic acid sequence is shown, but the complementary strand is understood as included by any reference to the displayed strand. The Sequence Listing is submitted as an ASCII text file named 95083_371_3_seqlist, created Nov. 23, 2020, about 9.1 KB, which is incorporated by reference herein.

### BACKGROUND

The primer extension reaction plays a role in many genetic analyses, e.g. in amplification methods such as PCR or in single steps of the analyses. An enzymatic primer extension is performed by means of a polymerase, wherein a synthesized strand is formed. In many methods this strand is detached from the template by various means independent of sequence and used in further process steps. Such means include temperature or denaturing agents such as alkalis or formamide. Furthermore, strands can be separated by enzymes using chemical energy sources such as dNTP or ATP. These include helicases or polymerases with strand displacing effect. Such separations are mainly sequence-unspecific.

Many reactions in genetic analyses require single-stranded or double-stranded, complementary states of nucleic acid chains or nucleic acid chain segments in order to achieve certain effects. A change between the single-stranded form and the double-stranded form is often required to switch a reaction on/off or to bring about a functionality. The binding of certain elements of the reactions (e.g. primer/template or probe/template, etc.) depends on the inducement of a permanent or temporary sequence-specific binding of components to each other. In many cases, this binding takes place by forming hydrogen bonds among complementary strands (in Watson-Crick base pairing). A strand at a certain position at a specific point in time can only form a bond with another strand. A third strand is therefore excluded from the interaction at this point in time. Thus, the availability of free complementary positions for potential interactions with other reaction components can play a role in many methods. Furthermore, the frequency and duration of the binding of reaction partners to each other via complementary positions can play a role in the speed or yield of a certain reaction step. Many reactions in the field of molecular analysis are based on the creation of more or less stable bonds between individual reaction components. The effects achieved in this way can influence the specificity of reactions.

Primer extension is used in many genetic analyses. The result of a correct priming (formation of a primer-template complex) as well as the continuation of template-dependent enzymatic synthesis by means of a polymerase plays a role. The synthesis phase is completed with the preparation of the product of a sequence-specific primer extension reaction (primer extension product). After completion of the primer extension reaction, the primer extension product can be detached from the template and used in other steps of the method.

In each of these steps, errors or deviations can occur, which can influence the specificity of the respective procedures and finally also mean certain limits of the applicability of respective procedures. Many examples are known, e.g. wrong priming, polymerase-related errors, faulty binding of probes, etc.

Genetic analysis methods often have to distinguish between several variants of a known sequence (e.g. allele-specific detection methods or mutation analysis in the presence of wild-type sequences). The presence of several sequences in the same reaction approach often leads to interferences (unintended interactions or parallel side reactions, etc.) between the sequences. Such interferences can occur at different times of a method, starting with the production of the first copies up to the detection. Generally, interference between very similar sequences can lead to misinterpretation of results. High specificity in such methods therefore plays an important role.

Especially in the case of minor genetic differences, such as single nucleotide changes (SNV) and short insertions/deletions (2-10 nucleotides), it would be advantageous if the specificity of primer extension dependent methods or method steps could be improved.

This can improve both the synthesis of the primer extension product itself and the provision of the synthesized primer extension product for further use.

The primer extension reaction is currently optimized in most cases by adjusting the primer binding (e.g. by using optimal reaction temperatures, buffer conditions and complementarity of the primers to a primer binding site). Some tools have been developed to improve primer binding. These are limited to primer binding enhancement only and do not extend beyond the primer length. By using a polymerase with a 3′ exonuclease activity (polymerases with proofreading) a further improvement of the quality of the synthesis result can be achieved. The 3′-end of the synthesized strand of exonuclease associated with the polymerase is checked.

Except for these phases of the primer extension reaction (priming and proofreading by the polymerase), the primer extension reaction is in most cases not checked for quality at all.

Particularly for trace analyses in the presence of strong interferences (e.g. detection of ctDNA in liquid biopsy methods), current methods reach their limits, which makes it difficult or impossible to increase the specificity of analyses.

It is therefore an objective of the present invention to provide means and methods for improving the specificity of genetic analysis methods which comprise or presuppose a primer extension reaction.

It is a further objective of the present invention to provide methods and components for improving specificity, especially in methods which comprise an enzymatic, template-dependent synthesis in a primer extension reaction.

It is a further objective of the present invention to provide methods and components for improving the specificity in methods which comprise the detachment or separation of a synthesized nucleic acid strand from its template.

Particularly, it is a further objective of the present invention to provide methods and components for a sequence-specific or selective separation of a synthesized nucleic acid strand from its template in a template-dependent primer extension reaction, especially in the simultaneous presence of very similar template sequences.

These objectives are performed by the subjects of the independent claims. Further advantageous embodiments of the invention are listed in the dependent claims and the following description.

### SUMMARY OF THE INVENTION

According to a first aspect, a method for the extension of an oligonucleotide primer is provided. The method for the specific extension of an oligonucleotide primer comprises the steps (FIGS. 1, 2):
a) Providing a nucleic acid chain comprising a target sequence (template strand) (M1)
b) hybridizing an oligonucleotide primer (P1.1) to a segment comprising a portion of the target sequence (primer binding site) of the nucleic acid chain (M1) provided, wherein the oligonucleotide primer (P1.1) comprises the following regions:
   a first region that can bind, essentially sequence specifically, to a complementary segment (primer binding site) comprising a portion of the target sequence within a provided nucleic acid chain (M1),
   a second region (overhang) adjacent to the 5′ end of the first region or connected by a linker, wherein the second region can be bound by a controller oligonucleotide (C1.1);
c) extension of the oligonucleotide primer (P.1 .1) by a template-dependent polymerase to obtain a primer extension product (P1.1-Ext) which comprises, besides the oligonucleotide primer (P1.1), a synthesized region which is essentially complementary to nucleic acid chain comprising a target sequence (M1), wherein the primer extension product (P1.1-Ext) and the nucleic acid chain (M1) form a double strand under reaction conditions;
d) binding of a controller oligonucleotide (C1.1) to the primer extension product (P1.1-Ext), wherein the controller oligonucleotide (C1.1) comprises the following regions:
   a first region which can bind to the second region (overhang) of the first primer extension product (P1.1-Ext),
   a second region which is complementary to the first region of the oligonucleotide primer (P1.1), and
   a third region which is essentially complementary to at least a part of the synthesized region of the primer extension product (P1.1-Ext); and
   wherein the controller oligonucleotide (C1.1) does not serve as a template for a primer extension of the oligonucleotide primer (P1.1), and the controller oligonucleotide (C1.1) can bind with its first, second and third regions to the primer extension product (P1.1-Ext) while displacing complementary segments of the nucleic acid chain (M1).

The second region of the oligonucleotide primer is particularly a polynucleotide tail.

The term “essentially sequence-specific” in the sense of the invention means particularly that the mutually complementary regions of the oligonucleotide primer and the nucleic acid to be amplified do not have more than 5, 4, 3, 2, or 1 mismatches.

The term “essentially complementary” in the sense of the invention means particularly that the mutually complementary regions of nucleic acids have no more than 5, 4, 3, 2, or 1 mismatches.

The above-mentioned steps can be carried out in one batch or in separate batches. If the procedures are to be carried out in one batch, they can be carried out under the same conditions, e.g. isothermal, or under different conditions, e.g. as thermocycling. In certain embodiments, oligonucleotide primers and controller oligonucleotide are present at the beginning of the reaction. However, sequential addition of individual reagents is possible as well.

Combinations with other amplification methods are possible as well, e.g. with PCR, wherein the PCR, for example, is first carried out over 1 to 10 cycles and then, for example, continued under isothermal conditions.

In certain embodiments of the method according to the invention, it is provided that the first region of the oligonucleotide primer can bind to the nucleic acid to be amplified completely sequence-specifically, i.e. particularly without mismatches.

In certain embodiments of the method according to the invention, it is provided that the second region (overhang) of the oligonucleotide primer remains essentially uncopied by a polymerase used for the primer extension under the selected reaction conditions.

In certain embodiments of the method according to the invention (FIG. 3, 4), it is provided that the method further comprises the following step: hybridizing a block oligonucleotide to a region of the nucleic acid chain comprising a target sequence (template strand) (M1) located in 5′-direction of the region bound by the first region (primer binding site) of the oligonucleotide primer, wherein the block oligonucleotide is designed to block the extension of the oligonucleotide primer at the bound region.

In certain embodiments of the invention it is provided that the block oligo-nucleotide is characterized by a length of about 15 nucleotides to about 60 nucleotides and optionally has modifications (e.g. PTO or LNA (locked nucleic acid, 2′O, 4′C methylene-bridged ribosyl residues in the polymer) or PNA (peptides nucleic acid, N-(2-aminoethyl) glycine backbone).

In certain embodiments of the method according to the invention, it is provided that the third region of the controller oligonucleotide is essentially complementary to that part of the synthesized region of the primer extension product which is immediately adjacent to the oligonucleotide primer portion of the primer extension product. This has the advantage of improving the sequence-specific displacement of the nucleic acid to be amplified.

In certain embodiments of the method according to the invention, the part of the synthesized region which is essentially complementary to the third region of the controller nucleotide has a length in the range from 5 nucleotides to 50 nucleotides.

In certain embodiments of the method according to the invention, the part of the synthesized region which is essentially complementary to the third region of the controller nucleotide has a length in the range of 3 nucleotides to 70 nucleotides, particularly 5 nucleotides to 50 nucleotides, particularly 5 nucleotides to 50 nucleotides, particularly 5 nucleotides to 30 nucleotides, particularly 5 nucleotides to 20 nucleotides.

In certain embodiments of the method according to the invention it is provided that the method further comprises the following step: hybridization of a probe to a region of the primer extension product which is not bound by the controller oligonucleotide.

In certain embodiments of the method according to the invention, it is provided that the method is carried out essentially isothermally.

For the purposes of the present description, the term “essentially isothermal” means particularly that the reaction temperature is carried out within a temperature range of 10 K temperature difference between the highest and the lowest temperature.

In certain embodiments of the method according to the invention, it is provided that the target sequence of a nucleic acid has a length in the range from about 20 nucleotides to about 300 nucleotides.

In certain embodiments of the method according to the invention, it is provided that the oligonucleotide primer has a length in the range from 15 nucleotides to 100 nucleotides.

In certain embodiments of the method according to the invention it is provided that the controller oligonucleotide has a length in the range from 20 nucleotides to 100 nucleotides.

In certain embodiments of the method according to the invention, it is provided that the reaction conditions chosen comprise a reaction temperature in the range from 50° C. to 70° C.

In certain embodiments of the method according to the invention, it is provided that the oligonucleotide primer comprises one or more modifications in the second region, particularly immediately following the first region of the oligonucleotide primer (FIG. 26), which stops the polymerase used at the second region. It is advantageous that only the first regions of the oligonucleotide primers are amplified, if necessary in later process steps.

This can preferably be achieved by using nucleotide modifications which, although they can undergo complementary binding with the primer extension product, are not accepted by the polymerase as templates. Examples of such nucleotide modifications are nucleotide compounds with modified phosphate-sugar-backbone-portions, e.g. 2′-O-alkyl RNA modifications (e.g. 2′-OMe), LNA modifications or morpholino modifications. The presence of such modifications in a strand prevents a DNA-dependent polymerase from reading-off such a strand. The number of such modifications can vary; generally, a few modifications (between 1 and 20) can be sufficient to prevent a polymerase from reading off such a strand. Such nucleotide modifications can, for example, be used at or around the binding site of the oligonucleotide primer to the controller oligonucleotide and/or as constituents of the second region of the oligonucleotide primer.

Due to the use of such modifications, the polymerase function is locally hindered so that certain segments of the structures used cannot be copied by the polymerase and remain predominantly single-stranded. In this single-stranded form they can bind further reaction components and thus carry out their function.

The second primer region is uncopyable in only one of several alternative embodiments. Said embodiment allows, for example, the use of identical primers in the primer extension and in any subsequent amplification.

Only when using short templates, said second primer region is uncopyable, since the 3′-end of such short templates can be located immediately before the second region and thus primes a synthesis (thus also extending the template strand, see FIGS. 16-18: the 3′-end of a template can be located immediately before the second region). To prevent the 2nd region of the primer from being copied, primers must have a “protection”. This can consist of a “first blocking unit” in front of the second region, or the total second region can consist of modifications which cannot be copied.

Said second primer region can be copied when using long templates (see FIGS. 1-3), as the 3′-end of the template is not located immediately in front of the second region and therefore synthesis cannot be primed. In said embodiment, the primer extension primer cannot be used as an amplification primer.

Furthermore, it is of relevance that the second region is not allowed to bind to the template and is therefore single-stranded for the interaction with the controller.

Folowing, several aspects of the invention are summarized:

1. The method for specifically extending an oligonucleotide primer, comprising the steps of:
a) providing a nucleic acid chain comprising a target sequence (template strand) (M1)1
b) hybridizing an oligonucleotide primer (P1.1) to a segment comprising a portion of the target sequence (primer binding site) of the nucleic acid chain (M1) provided, wherein the primer oligonucleotide (P1.1) comprises the following regions:
   a first region which can bind, essentially sequence-specifically, to a complementary segment (primer binding site) comprising a portion of the target sequence within a provided nucleic acid chain (M1),
   a second region (overhang) which is adjacent to the 5′ end of the first region or is connected via a linker, wherein the second region can be bound by a controller oligonucleotide (C1.1);
c) (c) Extension of the oligonucleotide primer (P.1 .1) by a template-dependent polymerase to obtain a primer extension product (primer extension product, P1.1-Ext) which comprises, besides the oligonucleotide primer (P1.1), a synthesized region which is essentially complementary to the nucleic acid chain comprising a target sequence (M1), wherein the primer extension product (P1.1-Ext) and the nucleic acid chain (M1) form a double strand under reaction conditions;
d) binding a controller oligonucleotide (C1.1) to the primer extension product (P1.1-Ext), wherein the controller oligonucleotide (C1.1) comprises the following regions:
   a first region which can bind to the second region (overhang) of the first primer extension product (P1.1-Ext),
   a second region which is complementary to the first region of the oligonucleotide primer (P1.1), and
   a third region which is essentially complementary to at least part of the synthesized region of the primer extension product (P1.1-Ext); and
   wherein the controller oligonucleotide (C1.1) does not serve as a template for a primer extension of the oligonucleotide primer (P1.1), and the controller oligonucleotide (C1.1) can bind with its first, second and third region to the primer extension product (P1.1-Ext) while displacing complementary segments of the nucleic acid chain (M1).

In certain embodiments, a target sequence comprises a polymorphic locus which comprises at least one nucleotide position which comprises at least two characteristic sequence variants of a target sequence.

In certain embodiments, a target sequence comprises a polymorphic locus which comprises at least two nucleotide positions which comprise at least two characteristic sequence variants in a target sequence.

In certain embodiments, a target sequence comprises a polymorphic locus which comprises a length between one nucleotide and 100 nucleotides, wherein such a locus comprises at least two characteristic sequence variants of a target sequence.

In certain embodiments in step (1), at least two template strands are provided, wherein each template strand comprises a variant of a target sequence having a specific and characteristic sequence variant of a polymorphic locus.

In certain embodiments in step (1), at least two template strands are provided, wherein each template strand comprises an own target sequence and said target sequences of the first and a further starting nucleic acid chain are different.
2. Method according to No. 1, characterized in that the second region (overhang) of the oligonucleotide primer (P1.1) remains essentially uncopied by a polymerase used for primer extension under the selected reaction conditions.
3. Method according to any one of the preceding numbers, wherein the primer extension of the primer (P1.1) hybridized to the nucleic acid chain (M 1) takes place by a template-dependent polymerase in the presence of the controller oligonucleotide (C1.1) in the same reaction batch.
4. Method according to any one of the preceding numbers, wherein the primer extension product formed is separated from the nucleic acid chain (M 1) with participation of the controller oligonucleotide (C1.1).
5. Method according to one of the preceding numbers, wherein steps (b) to (d) are repeated at least once.
6. Method according to any one of the preceding numbers, characterized in that the method further comprises the following step:
   Hybridizing a block oligonucleotide (B1.1) to a sequence segment of the nucleic acid chain (M1) located in the 5′ direction from the segment (primer binding site), bound by the first region of the oligonucleotide primer (P1. 1), wherein the block oligonucleotide primer (B1.1) is designed to prevent synthesis of the primer extension product (P1.1-Ext) at the bound segment, thereby limiting the length of the synthesized region of the primer extension product.
7. Method according to No. 6, wherein the step of hybridizing a block oligonucleotide (B1.1) takes place before the step of primer extension.
8. Method according to any one of the preceding numbers, characterized in that the third region of the controller oligonucleotide (C1.1) is essentially complementary to the part of the synthesized region of the primer extension product and is immediately adjacent to the primer oligonucleotide portion of the primer extension product (P1.1-Ext).
9. Method according to any of the preceding numbers, characterized in that the method is carried out essentially isothermally.
10. Method according to any one of the preceding numbers, characterized in that the target sequence of the nucleic acid chain (M1) comprises a length of 20 nucleotides to 200 nucleotides.
11. Method according to any one of the preceding numbers, characterized in that the oligonucleotide primer (P1.1) comprises a length in the range of 15 nucleotides to 100 nucleotides.
12. Method according to any one of the preceding numbers, characterized in that the controller oligonucleotide (C1.1) comprises a length in the range of 20 nucleotides to 100 nucleotides.
13. Method according to any one of the preceding numbers, characterized in that the reaction conditions chosen comprise a reaction temperature in the range of 25° C. to 80° C.
14. Method according to any one of the preceding numbers, characterized in that the oligonucleotide primer (P1.1) comprises one or more modifications in the second region, particularly immediately following the first region of the oligonucleotide primer (P1.1), which prevents the polymerase used from copying the second region.
15. Kit for carrying out a method according to one of the preceding numbers, comprising:
   an oligonucleotide primer (P1.1) comprising the following regions:
      a first region that can bind, essentially sequence specifically, to a complementary segment (primer binding site) within a provided nucleic acid chain (M1) comprising a target sequence,
      a second region (overhang) adjacent to the 5′ end of the first region or connected by a linker, wherein the second region can be bound by a controller oligonucleotide (C1.1);
         wherein the first oligonucleotide primer (P1.1), when hybridized to a complementary segment of the target sequence, can be extended by a template-dependent polymerase to a primer extension product (P1.1-Ext) which comprises besides the oligonucleotide primer (P1.1) a synthesized region;
   a controller oligonucleotide (C1.1) comprising the following regions:
   a first region that can bind to the second region (overhang) of the first primer extension product (P1.1-Ext)
   a second region which is complementary to the first region of the oligonucleotide primer (P1.1), and
   a third region which is essentially complementary to at least part of the synthesized region of the primer extension product (P1.1-Ext); and
   wherein the controller oligonucleotide (C1.1) does not serve as a template for primer extension of the oligonucleotide primer (P1.1), and the controller oligonucleotide (C1.1) can bind with its first, second and third region to the primer extension product (P1.1-Ext), displacing complementary segments of the nucleic acid chain (M1); and optionally
   a block oligonucleotide (B1.1) which can bind to a segment of the nucleic acid chain (M1) located in the 5′ direction from the segment (primer binding site) bound by the first region of the primer oligonucleotide (P1. 1), wherein the block oligonucleotide primer (B1.1) is designed to block the synthesis of the primer extension product (P1.1-Ext) at the bound region and thereby limiting the length of the synthesized region of the primer extension product.
16. Kit according to No. 15, characterized in that the second region of the oligonucleotide primer (P1.1) remains essentially uncopied by a polymerase used for primer extension under the selected reaction conditions.
17. Using a kit according to No. 15 or 16 to carry out a method according to any of No. 1 to 14.
18. Method for specifically extending an oligonucleotide primer comprising the steps of:
   a) Providing at least two nucleic acid chains comprising variants of a target sequence (template strand) (M 1.1 and M 1.2), wherein provided nucleic acid chains
      comprise at least one uniform segment specific to M 1.1 and M 1.2 and identical for M 1.1 and M 1.2, and
      comprise at least one characteristic segment specific to each nucleic acid chain
   b) providing a first oligonucleotide primer (P1.1) which can bind complementarily to the uniform segment of at least two nucleic acid chains provided, wherein the primer oligonucleotide comprises
      a first region which can bind, essentially sequence-specifically, to a complementary segment (primer binding site) comprising a portion of the target sequence within a uniform segment of provided nucleic acid chains (M 1.1 and M 1.2),
      a second region (overhang) adjacent to the 5′ end of the first segment or connected by a linker, wherein the second region can be bound by a controller oligonucleotide (C 1.1 or C 1.2);
   wherein the binding to the uniform segment of the provided nucleic acid chains takes place in such way that the respectively characteristic and specific segment is located in 5′ direction of the provided nucleic acid chain from the hybridized primer (P 1.1);
   c) providing at least two controller oligonucleotides (C1.1 and C1.2), wherein each comprises the following regions:
      a first region which can bind to the second region of the oligonucleotide primer (P1.1) (overhang), and
      a second region which is complementary to the first region of the oligonucleotide primer (P1.1), and
      a third region which is complementary to at least part of the synthesized region of at least one expected first primer extension product (P1.1-Ext or P1.2-Ext); and
      wherein the controller oligonucleotides (C1.1 and C1.2) do not serve as templates for primer extension of the oligonucleotide primer (P1.1), and
      the controller oligonucleotide (C1.1) can bind with its first, second and third region the complementary segments of the primer extension product (P1.1-Ext), displacing the nucleic acid chain (M 1.1), and
      the controller oligonucleotide (C1.2) can bind with its first, second and third region to the complementary segments of the primer extension product (P1.2-Ext), displacing the nucleic acid chain (M 1.2).
   d) hybridizing a primer oligonucleotide primer (P1.1) to a segment comprising a portion of the target sequence (primer binding site) of at least two nucleic acid chains (M 1.1 and M 1.2) provided,
   e) Extension of the oligonucleotide primer (P.1.1) by a template-dependent polymerase using provided nucleic acid chains as templates for the primer extension to obtain specific primer extension products (primer extension products, P1.1-Ext and P1.2-Ext), wherein each region (P1.1) comprises, besides the oligonucleotide primer, a synthesized region essentially complementary to the respective nucleic acid chain comprising a target sequence (M1), wherein
      the primer extension product (P1.1-Ext) comprises a synthesized region complementary to the characteristic and specific segment of the first nucleic acid (M 1.1) and
      the primer extension product (P1.2-Ext) comprises a synthesized region complementary to the characteristic and specific segment of the second nucleic acid (M 1.2) and
   the synthesized primer extension products (P1.1-Ext and P1.2-Ext) and their corresponding nucleic acid chain serving as templates (M 1.1 and M 1.2) each form a double strand under reaction conditions;
   f) binding of at least one controller oligonucleotide (C1.1 and C 1.2) to the primer extension products (P1.1-Ext and P1.2-Ext) formed in step (e), wherein
      the controller oligonucleotide (C1.1) can bind with its first, second and third region to the complementary segments of the primer extension product (P1.1-Ext), displacing the nucleic acid chain (M 1.1), and
   the controller oligonucleotide (C1.2) can bind with its first, second and third regions to the complementary segments of the primer extension product (P1.2-Ext), displacing the nucleic acid chain (M 1.2).
19. Method according to No. 18, wherein nucleic acid chains comprising variants of a target sequence (template strand) (M 1.1 and M 1.2) comprise at least two uniform segments, which are identical at M1.1 and M1.2.
20. Method according to No. 19, wherein the uniform segment comprises a length of 10 to 180 nucleotides.
21. Method according to No. 18 to 20, wherein the characteristic and specific segment comprises a length of one nucleotide up to 50 nucleotides.
22. Method for specifically extending an oligonucleotide primer, comprising the steps of:
   a) Providing at least two nucleic acid chains comprising variants of a target sequence (template strand) (M 1.1 and M 1.2), wherein provided nucleic acid chains
      comprise at least one uniform segment specific to M 1.1 and M 1.2 and identical at M 1.1 and M 1.2, and
      comprise at least one characteristic segment specific to each nucleic acid chain
   b) providing at least two first oligonucleotide primers (P1.1 and P 1.2), each of which can bind completely or partially to the corresponding characteristic and specific segment of each of the nucleic acid chains provided, wherein each oligonucleotide primer
      comprises a first region which can bind sequence-specifically to a complementary segment (primer binding site) comprising a portion of the target sequence within a characteristic and specific segment of provided nucleic acid chains (M 1.1 and M 1.2),
      a second region (overhang) adjacent to the 5′ end of the first segment or connected by a linker, wherein the second region can be bound by a controller oligonucleotide (C 1.1 or C 1.2);
   c) providing at least two controller oligonucleotides (C1.1 and C1.2), wherein each comprises the following regions:
      a first region which can bind to the second region of the oligonucleotide primers (P1.1 and/or P1.2) (overhang), and a second region complementary to the first region of the respective oligonucleotide primer (P1.1 or P1.2), wherein
         C 1.1 comprises a second region complementary to the corresponding first region of P1.1, and
         C 1.2 comprises a second region complementary to the corresponding first region of P1.2
      and
      a third region which is complementary to at least part of the synthesized region of at least one expected first segment of primer extension product (P 1.1-Ext or P 1.2-Ext);
      wherein the controller oligonucleotides (C 1.1 and C 1.2) do not serve as templates for a primer extension of a used oligonucleotide primer (P 1.1 or P 1.2),
   d) hybridizing of at least one oligonucleotide primer (P1.1 and/or P1.2) to the respectively complementary segment comprising a portion of the target sequence (primer binding site) of at least one of the nucleic acid chains (M 1.1 and M 1.2) provided,
   e) extension of at least one oligonucleotide primer (P1.1 and/or P1.2) by a template-dependent polymerase using nucleic acid chains provided as templates for the primer extension to obtain at least one specific primer extension product (primer extension products, P1. 1-ext and P1.2-ext), wherein each comprises, besides the oligonucleotide primer (P1.1 or P1.2), a synthesized region essentially complementary to the respective nucleic acid chain comprising a target sequence (M1), wherein
      the primer extension product (P1.1-Ext) comprises a complementary region to the characteristic and specific segment of the first nucleic acid (M 1.1), and
      the primer extension product (P1.2-Ext) comprises a region complementary to the characteristic and specific segment of the second nucleic acid (M 1.2), and
   the synthesized primer extension products (P1.1-Ext and P1.2-Ext) and their corresponding nucleic acid chain serving as templates (M 1.1 and M 1.2) each form a double strand under reaction conditions;
   f) binding of at least one controller oligonucleotide (C1.1 and C 1.2) to the primer extension products (P1.1-Ext and P1.2-Ext) formed in step (e), wherein
      the controller oligonucleotide (C1.1) can bind with its first, second and third regions to the complementary segments of the primer extension product (P1.1-Ext), displacing the nucleic acid chain (M 1.1), and
      the controller oligonucleotide (C1.2) can bind with its first, second and third regions to the complementary segments of the primer extension product (P1.2-Ext), displacing the nucleic acid chain (M 1.2).
23. Method according to No. 22, wherein the binding of a respective first primer to the characteristic and specific segment of the nucleic acid chains provided takes place in such way that the respective characteristic and specific segment of the nucleic acid chain is bound complementarily by the 3′-terminal nucleotide of the respective first primer.
24. Method according to No. 22, wherein the binding of a respective first primer to the characteristic and specific segment of the nucleic acid chains provided takes place in such way that the respective characteristic and specific segment of the nucleic acid chain is bound complementarily by the 3′ segment of the respective first primer.
25. Method for specifically extending an oligonucleotide primer using a competitor primer (FIGS. 21-23), comprising the steps of:
   a) Providing at least two nucleic acid chains comprising variants of a target sequence (template strand) (M 1.1 and M 1.2), wherein provided nucleic acid chains
      comprise at least one uniform segment specific to M 1.1 and M 1.2 and identical at M 1.1 and M 1.2, and
      comprise at least one characteristic segment specific to each nucleic acid chain
   b) providing a first oligonucleotide primer (P1.1) which can bind completely or partially complementarily to the corresponding characteristic and specific segment of one of the nucleic acid chains provided, wherein the first primer oligonucleotide
      comprises a first region which can bind sequence-specifically to a complementary segment (primer binding site) comprising a portion of the target sequence within a characteristic and specific segment of provided nucleic acid chains (M 1.1),
      a second region (overhang) adjacent to the 5′ end of the first region or connected by a linker, wherein the second region can be bound by a controller oligonucleotide (C 1.1);
   c) providing a competitor-primer-oligonucleotide (P 5.2) which can bind completely or partially complementarily to the corresponding characteristic and specific segment of a second nucleic acid chain provided (M 1.2), wherein the competitor-primer-oligonucleotide
      comprises a first region which can bind sequence-specifically to a complementary segment (primer binding site) comprising a portion of the target sequence within a characteristic and specific segment of the provided nucleic acid chain (M 1.2), wherein
      said region is not identical to the first region of the first segment of the oligonucleotide primer
   d) providing a controller oligonucleotide (C1.1), wherein the controller oligonucleotide comprises the following regions:
      a first region which can bind to the second region of the first oligonucleotide primer (P1.1) (overhang), and
      a second region which is complementary to the first region of the first oligonucleotide primer (P1.1), and
      a third region which is complementary to at least part of the synthesized region of an expected first primer extension product (P 1.1-Ext);
      wherein the controller oligonucleotide (C 1.1) does not serve as a template for a primer extension of a used first oligonucleotide primer (P 1.1) or a competitor-primer-oligonucleotide,
   e) simultaneous hybridisation of the first oligonucleotide primer (P1.1) and the competitor-oligonucleotide-primer to the respective complementary segment comprising a portion of the target sequence (primer binding site) of at least one of the nucleic acid chains (M 1.1 and M 1.2) provided,
   f) simultaneous extension of the first oligonucleotide primer (P1.1) and the competitor oligonucleotide by a template-dependent polymerase using nucleic acid chains provided as templates for the primer extension to obtain at least one specific primer extension product (primer extension products, P1. 1-ext and P5.2-ext), wherein each comprises, besides the oligonucleotide primer (P1.1 or P5.2), a synthesized region essentially complementary to the respective nucleic acid chain comprising a target sequence (M1), wherein
      the primer extension product (P1.1-Ext) comprises a complementary region to the characteristic and specific segment of the first nucleic acid (M 1.1), and
      the primer extension product (P5.2-Ext) comprises a region complementary to the characteristic and specific segment of the second nucleic acid (M 1.2), and
   the synthesized primer extension products (P1.1-Ext and P5.2-Ext) and their corresponding nucleic acid chain serving as template (M 1.1 and M 1.2) each form a double strand under reaction conditions;
   g) selective binding of a controller oligonucleotide (C1.1) to the primer extension products (P1.1-Ext) formed in step (e), wherein
      the controller oligonucleotide (C1.1) with its first, second and third region can bind to the complementary segments of the primer extension product (P1.1-Ext) while displacing of the nucleic acid chain (M 1.1), wherein
      the controller oligonucleotide (C1.1) is not capable of binding to P5.2-Ext under the reaction conditions used and separating P5.2-Ext from its template strand.
26. Method according to No. 25, wherein the 3′-terminal nucleotide of the competitor primer can bind within the second blocking unit of the controller oligonucleotide.
   27. Method according to No. 25, wherein the 3′-terminal nucleotide of the competitor primer can bind within the fourth blocking unit of the controller oligonucleotide, wherein the fourth blocking unit is located in the third region of the controller oligonucleotide.

In certain embodiments, a first oligonucleotide primer and a corresponding controller oligonucleotide thereof are combined to form a primer extension system. Such a primer extension system comprises:
a specific controller oligonucleotide for a sequence variant of the target sequence,
a first oligonucleotide primer which can bind essentially complementary to the uniform sequence fragment for the target sequence (template strand) provided, and wherein the first oligonucleotide primer does not comprise sequence segments which are specific for a sequence variant of the target sequence. Rather, the first oligonucleotide primers can bind to the target sequence fully complementarily or essentially complementarily without distinguishing between the sequence variants.
Such a primer extension system is thus constructed in such way that the polymorphic locus of the target sequence is located in the sequence segment that lies in the 3′-primer extension direction of the first primer. The segment of the controller oligonucleotide corresponding to the polymorphic locus is thus located in its third region.

In certain embodiments, a first oligonucleotide primer and a corresponding controller oligonucleotide are combined to form a primer extension system. Such a primer extension system comprises:
a specific controller oligonucleotide for a sequence variant of the target sequence
a first sequence variant specific oligonucleotide primer which can bind at least partially (e.g. with its 3′-terminal nucleotide) fully complementarily to the specific sequence segment of a polymorphic locus of a provided target sequence.

In certain embodiments, such a primer extension system is thus constructed in such way that the sequence of the first oligonucleotide primer overlaps with at least one nucleotide position having a characteristic sequence of the polymorphic locus.

The segment of the controller oligonucleotide corresponding to the polymorphic locus is, in certain embodiments, completely located in the second region of the controller oligonucleotide.

In certain embodiments, the segment of the controller oligonucleotide corresponding to the polymorphic locus is located partially in the second and partially in the third region of the controller oligonucleotide.

In certain embodiments, a first oligonucleotide primer and a corresponding controller oligonucleotide as well as a competitor-oligonucleotide-primer are combined to form a primer extension system. Such a primer extension system comprises:
a specific controller oligonucleotide for a sequence variant of the target sequence,
a first oligonucleotide primer which can bind essentially complementary to the uniform sequence fragment for the target sequence (template strand) provided, and
a specific competitor-oligonucleotide-primer.

Thereby, the first oligonucleotide primer does not comprise sequence segments that are specific for a sequence variant of the target sequence. Rather, the first oligonucleotide primer can bind to the target sequence completely complementarily or essentially complementarily without distinguishing between the sequence variants.

Such a primer extension system is thus constructed in such way that the polymorphic locus of the target sequence is located in the sequence segment that is located in 3′-primer extension direction of the first primer. The segment of the controller oligonucleotide corresponding to the polymorphic locus is thus located in the third region of the controller oligonucleotide.

In certain embodiments, the competitor-oligonucleotide-primer used can bind completely or partially to the segment of the target sequence, which can undergo complementary binding with the first region of the first oligonucleotide primer.

In certain embodiments, said competitor-oligonucleotide-primer is fully complementary to a sequence variant of a target sequence which is not identical to the sequence variant of the template strand recognised by the first primer.

In certain embodiments of the method, a competitor-oligonucleotide-primer does not comprise a sequence segment which can bind essentially complementarily to the first region of the controller oligonucleotide.

In certain embodiments, a first oligonucleotide primer and a corresponding controller oligonucleotide and a competitor oligonucleotide are combined to form a primer extension system. Such a primer extension system comprises:
a specific controller oligonucleotide for a sequence variant of the target sequence
a first sequence variant specific oligonucleotide primer which can bind at least partially (e.g. with its 3′-terminal nucleotide) to the specific sequence segment of a polymorphic locus of a provided target sequence fully complementarily
a specific competitor-oligonucleotide- primer

In certain embodiments, such a primer extension system is thus constructed in such way that the sequence of the first oligonucleotide primer overlaps in at least one nucleotide position with a characteristic sequence of the polymorphic locus.

The segment of the controller oligonucleotide corresponding to the polymorphic locus is located in an embodiment completely in the second region of the controller oligonucleotide.

In certain embodiments, the segment of the controller oligonucleotide corresponding to the polymorphic locus is located partially in the second and partially in the third region of the controller oligonucleotide.

In certain embodiments, the competitor-oligonucleotide-primer used can bind completely or partially to the segment of the target sequence, which can undergo complementary binding with the first region of the first oligonucleotide primer.

In certain embodiments, said competitor-oligonucleotide-primer oligonucleotide is fully complementary to a sequence variant of a target sequence which is not identical to the sequence variant of the template strand recognized by the first primer.

In certain embodiments of the method, a competitor-oligonucleotide-primer does not comprise a sequence segment which can bind essentially complementary to the first region of the controller oligonucleotide.

The specification describes the production of primer extension products which can be used, for example, in exponential amplification as start nucleic acid.

The first oligonucleotide primers and controller oligonucleotides used here may be identical to or different from those used in an exponential amplification.

### DETAILED DESCRIPTION OF THE INVENTION

The technical objectives mentioned at the outset are particularly solved by providing methods and components (feedback system) that can verify the quality of synthesized primer extension products in different phases of template-dependent primer extension. Depending on the embodiment, said verification can take place either during the synthesis or just after completion of the synthesis. The verification can take place in the same reaction batch so that the actual primer extension reaction does not have to be interrupted (online control) or only after completion of the primer extension reaction (final control).

By verification of the quality, influence can be exerted on the primer extension reaction itself or on the separation of the product from the template. Said influence is exerted by the components provided. Thus, these components form a feedback system for the primer extension reaction or for the separation of the product of the primer extension reaction from the template.

The components of the feedback system can influence the primer extension reaction in such way that a decision can be made between continuation of the enzymatic synthesis or interruption or restart of the synthesis respectively. Furthermore, the components of the feedback system can influence the primer extension reaction in such way that the separation of the product of the synthesis from the template is continued depending on the matching with the controller oligonucleotide or is stopped, delayed or completely interrupted in case of deviations in the sequence.

The synthesis system in a primer extension reaction generally comprises at least one template and at least one primer which is capable of binding more or less specifically to the template and initiating a primer extension reaction. Furthermore, the synthesis system comprises at least one polymerase, as well as substrates (dNTPs) and buffer conditions under which a primer extension reaction can take place. Further oligonucleotides can be added depending on the embodiment of the method, e.g. block oligonucleotides for sequence-specific stopping of the polymerase or probe oligonucleotides which can emit a fluorescent signal.

The feedback system comprises a primer, which acts as an initiator of a primer extension reaction, as well as a further oligonucleotide, a so-called controller oligonucleotide. The primer of the feedback system is in a preferred embodiment identical to the primer of the synthesis system. The controller oligonucleotide is capable of interacting with the respective primer as well as with the sequence-specific primer extension product derived from this primer. It is capable of forming a complementary double strand under reaction conditions if the sequence matches the primer extension product. The controller oligonucleotide does not itself serve as a template for the synthesis of said primer/polymerase system. Further oligonucleotides can be added depending on the embodiment of the method, e.g. block oligonucleotides for sequence-specific stopping of the polymerase or probe oligonucleotides which can emit a fluorescent signal.

In certain embodiments, the method comprises a primer extension reaction (also called primer extension) by means of a primer in the presence of a suitable controller oligonucleotide in the same reaction batch. The controller oligonucleotide is able to interact with the primer extension product to form a complementary strand. The progress of the formation of the double strand between the controller and the primer extension product (P1-Ext) depends essentially on the matching of the sequences between the controller and the corresponding primer extension product. Thus, sequence control or sequence verification can be performed already during a primer extension or immediately after a primer extension at least over partial segments of the synthesized primer extension fragment by means of controller oligonucleotides primers.

Furthermore, the method comprises in certain embodiments a method for a template-dependent primer extension in combination with a subsequent control of the synthesized primer extension product by at least one controller oligonucleotide after completion of the primer extension reaction. Primer extension and controller interaction with the synthesized primer extension product are two time-separated reactions. Although the primer extension reaction proceeds in the same reaction batch in the presence of a respective controller oligonucleotide, the reactions are separated in time into individual reaction steps by the choice of reaction conditions.

The presence of the feedback system in the same reaction batch as the primer extension reaction makes it possible to design a homogeneous assay.

By introducing or combining the components of the feedback system in the same reaction batch as the primer extension reaction itself, i.e. simultaneous presence of both the components of the synthesis system and the feedback system, the verification or influencing of the primer extension reaction and the separation of the primer extension products from the template can be performed at very short intervals, if necessary. This allows the feedback system to react very quickly to any deviations that may occur.

The simultaneous presence of the feedback system in the primer extension approach can lead to repeated verification, such that the result of the verification can be summarized as the sum of multiple verification events. The frequency of verification can be influenced both by the appropriate choice of reaction conditions (e.g. temperature, concentration of feedback system components) and by the structures of feedback components (e.g. length and complementarity or extent to which modified nucleotides are used in the design of individual structures).

In certain cases, the feedback system should distinguish between correctly performed reactions (in many cases more than 99% of all primer extension strands of a primer extension reaction due to a high natural specificity of an enzymatic template-dependent reaction) and incorrectly synthesized primer extension products (less than 1% of all primer extension products). Thus, a feedback system can be helpful for example in trace analysis.

Due to a high sequence dependence of the interaction of the primer extension product (P1-Ext) with the controller, equilibria between individual intermediate complexes that are generated during the reaction can be individually influenced. Since these complexes comprise different accesses to certain sequence segments (single-stranded segments), many reactions can be influenced during a running primer extension reaction. Possible intermediate complexes comprise e.g. primer extension product/ template, primer extension product/controller oligonucleotide and primer extension product/ template/controller oligonucleotide and/or primer extension product/probe. This allows for exerting stronger influence on the specificity in the overall primer extension process as well as in reactions dependent on it.

Several technical effects can be achieved as a result of the sequence-dependent interaction between the controller oligonucleotide and the synthesized strand of the primer extension product (P1-Ext):
Complete or partial sequence dependent separation, dissociation or detachment of the P1-Ext from the template strand;
Sequence-specific detection of certain variants of the sequence of the P1.ext.
Sequence-specific maintenance of a double strand or, conversely, sequence-specific conversion of strand segments into a single-strand form, and related accessibility of certain sequence segments for interaction with other system components, e.g. probes, primers, etc.

In certain embodiments of the method according to the invention, the primer extension can be carried out as a single synthesis in one step or as a repeated reaction (linear amplification).

The primer extension reaction can be performed on its own or in combination with an amplification method.

Especially in the presence of several very similar sequences, which can potentially occur for a primer extension reaction starting from one primer, it is therefore advantageous to accommodate a feedback system within the reaction batch. Thus, differences can generally be separated much stronger or sharper. This can be advantageous, for example, for allele discrimination procedures or for the targeted amplification of a few copies with mutations that are present in much smaller numbers than the wild type.

The sequence verification by the method according to the invention is performed beyond the primer length (approx. 5-50 nucleotides of the synthesized segment) and thus differs from usual primer extension reactions in which the primer sequence is essentially responsible for sequence-specific binding.

The improved specificity of the detachment of the synthesized strand can be advantageous in detection methods under several aspects.
It can, for example, be used as an alternative detection method and/or component for genetic modifications, such as single nucleotide polymorphisms.
The sequence-specific synthesized strand of the primer extension product (P1 -Ext) is the desired product of the primer extension reaction. It can be used in various methods of genetic information analysis, for example in:
   the generation of one or more copies starting from a longer nucleic acid chain, e.g. a genomic DNA or its segments, or an RNA or its equivalents such as cDNA or its segments. Said copies may be used as starting nucleic acid chains in amplification methods, such as PCR or other amplification methods; or
   the detection of a specific change of a sequence in a nucleic acid fragment, e.g. an amplification fragment
The components and methods should support especially in the simultaneous presence of several, very similar variants of a template sequence, e.g. wild-type and one or more mutated sequences, an improvement of the specificity of individual method steps.
The advantages of the method are particularly evident in simultaneous presence of several very similar variants of a template sequence, e.g. wild-type and one or more mutated sequences.
Especially in case of minor genetic differences, such as single nucleotide changes (SNV) and short InDeIs (2 -10 nucleotides), the specificity of primer extension dependent methods/method steps can be improved.
In some embodiments of the method, components for a homogeneous assay should be provided, which allow the simultaneous presence of components in a reaction (one-pot reaction or homogeneous assay).

The primer extension reaction described herein and the primer extension products generated thereof can, for example, serve as start nucleic acids in an amplification method.

Such an amplification method has already been described in the PCT application (PCT/EP2017/071011) and the European application (EP-A 16185624.0). For details of execution of the amplification, the skilled person is referred to this application.

Preferably such an amplification takes place as exponential amplification, in which newly synthesized products of both primers (primer extension products) act as templates for further synthesis steps. Thereby primer sequences are at least partially copied so that complementary primer binding sites are formed, which are present as sequence segments of a double strand immediately after their synthesis. In the amplification method, synthesis steps of both strands and double-strand opening steps of the newly synthesized sequence segments are carried out alternately. Sufficient double-strand separation after synthesis is an important prerequisite for further synthesis. Overall, such an alternation of synthesis and double-strand separation steps can lead to exponential amplification.

In the amplification method, the double-strand opening of main products of the amplification (amplification of nucleic acid chains comprising target sequence) takes place, among other things, by means of an oligonucleotide called controller oligonucleotide. The controller oligonucleotide comprises sequence segments which correspond to the respective target sequence.

In detail, strand separation is achieved by using the controller oligonucleotide with predefined sequences, which preferably separate a newly synthesized double strand consisting of both specific primer extension products by means of sequence-dependent nucleic acid-mediated strand displacement. The resulting single-stranded segments of primer extension products comprise the target sequence as well as respective primer binding sites which can serve as binding sites for further oligonucleotide primers, such that an exponential amplification of nucleic acid chains to be amplified is achieved. The primer extension reactions and strand displacement reactions preferably take place simultaneously in the batch. The amplification is preferably carried out under reaction conditions that do not allow a spontaneous separation of both specific synthesized primer extension products.

A specific exponential amplification of a target sequence-encompassing nucleic acid chain comprises a repetition of synthesis steps and double strand opening steps (activation steps for primer binding sites) as a mandatory requirement for the amplification of the nucleic acid chain.

The mechanisms of strand separation using a controller described in this application are similar to those described for the “controller oligonucleotide” in the PCT application (PCT/EP2017/071011). This mechanism is briefly summarized below.

The opening of synthesized double strands is realized as a reaction step, which must be influenced sequence-specifically by the controller oligonucleotide. This opening can be carried out completely, up to the dissociation of both complementary primer extension products, or it can be partial.

According to the invention, the controller oligonucleotide comprises sequence segments which can interact with the target sequence and further sequence segments which cause or enable or promote this interaction. In the course of the interaction with the controller oligonucleotide, double-stranded segments of the synthesized primer extension products are converted into single-stranded form via sequence-specific strand displacement. This procedure is sequence-dependent: only when the sequence of the synthesized double strand comprises a certain degree of complementarity with the respective sequence of the controller oligonucleotide, a sufficient opening of the double strand occurs so that the sequence segments essentially necessary for the continuation of the synthesis, such as primer binding sites, are converted into single-stranded form, which corresponds to an “active state”. The controller oligonucleotide thus specifically “activates” the newly synthesized primer extension products, which comprise the target sequence, for further synthesis steps.

In contrast, sequence segments that do not comprise a target sequence are not converted to the single-stranded state and remain as double-strand, which corresponds to an “inactive” state, respectively. The potential primer binding sites in such a double strand are disadvantaged or completely prevented from interacting with new primers, such that further synthesis steps generally do not take place on such “inactive” strands. This missing or reduced activation (i.e. conversion into a single-stranded state) of synthesized nucleic acid strands after a synthesis step leads to that in the subsequent synthesis step only a reduced amount of primers can successfully participate in a primer extension reaction.

The reaction conditions (e.g. temperature) are designed in such way that spontaneous separation of complementary primer extension products is unlikely or significantly slowed down in the absence of a controller oligonucleotide.

The desired increase in the specificity of a primer extension reaction thus results from the sequence dependence of the separation of complementary primer extension products comprising a predefined target sequence: the controller oligonucleotide enables or promotes this double-stranded separation as a result of the matching of its sequence segments with predefined sequence segments of the primer extension products.

Predominantly sequence-specific separation of the double strand with involvement of a controller oligonucleotide can be combined with sequence-specific or fewer sequence-specific primers.

Use of predominantly specific controller oligonucleotides, which are able to distinguish between individual variants, allows for example the combination of allele-specific or mutation-specific assays.

The invention is particularly suitable for primer extension reactions of sequence variants of a known locus of a target sequence. Such a locus can comprise several occurring sequence variants of a target sequence and is therefore a polymorphic locus (FIGS. 12-15). Such a polymorphic locus comprises for example single nucleotide polymorphisms.

In order to achieve sequence specificity of a sequence variant of a target sequence locus, components that play an essential role in the specificity of primer extension should be placed in a specific arrangement. Especially advantageous are arrangements comprising a known sequence variant of a locus, wherein a predominantly sequence-specific controller oligonucleotide as well as respective primers can be designed.

The position of an expected sequence variant in the known locus is thus taken into account in the design of components to the extent that at least the controller oligonucleotide, better controller oligonucleotide in combination with at least one primer, comprises a specific sequence composition in a primer extension system suitable for primer extension of a desired variant of the target sequence.

The objective of the invention is further solved in that a primer extension system comprises at least one sequence-specific primer (e.g. allele-discriminating primer) in combination with a specific controller oligonucleotide (e.g. allele-specific controller oligonucleotide) in each case. Thus, for each specific composition of a target sequence and its specific variants (e.g. alleles), a specific combination of at least one specific primer and at least one specific controller oligonucleotide can be provided, respectively. Thereby a specific amplification of one of the possible sequence variants results in a target sequence.

Furthermore, the objective of the invention is solved in that a primer extension system comprises at least two, particularly four sequence-specific primers (e.g. allele-discriminating primers) in combination with one specific controller oligonucleotide each (e.g. allele-specific controller oligonucleotide). Thus, for each specific composition of a target sequence and its specific variants (e.g. alleles), a specific combination of at least one specific primer and at least one specific controller oligonucleotide can be provided respectively, wherein a specific primer-controller oligonucleotide is provided for all four nucleotide variants at a certain position. In presence of at least one sequence variant of the target sequence in the reaction batch, a specific primer extension of one of the possible sequence variants of a target sequence results.

Furthermore, the objective of the invention is solved by the fact that a primer extension system comprises at least one sequence-specific primer (e.g. allele-discriminating primer) in combination with a specific controller-oligonucleotide (e.g. allele-specific controller-oligonucleotide) respectively and at least one further primer (competitor-oligonucleotide-primer) which comprises a sequence composition complementary to other expected sequence variants. Thus for each specific composition of a target sequence and its specific variants (e.g. alleles) a specific combination of at least one specific primer and at least one specific oligonucleotide primer can be provided, respectively. This results in a specific primer extension of one of the possible sequence variants of a target sequence.

The objective of the invention is further solved by analyzing both strands of a double-stranded nucleic acid chain, wherein in one reaction the components of a primer extension system are provided for one strand of a target nucleic acid which is used as start nucleic acid, and in a separate batch components are provided for the complementary strand of the target nucleic acid chain which is used as start nucleic acid. Thus, at least two controller oligonucleotides are used, each of which can bind to one strand of the target nucleic acid chain (as first primer extension product). Both controller oligonucleotides are used in separate reaction batches.

Furthermore, the objective of the invention is solved in that a primer extension system comprises at least one primer which can support primer extension of several potential sequence variants (e.g. a target-sequence-specific primer but not an allele-discriminating primer, a target-sequence-specific but an allele-unspecific primer) in combination with a specific controller oligonucleotide respectively (e.g. allele-specific controller oligonucleotide). Thus, for each specific composition of a target sequence and its specific variants (e.g. alleles), a specific combination of at least one allele-unspecific primer and at least one allele-specific oligonucleotide primer can be provided, respectively. This results in a specific primer extension of one of the possible sequence variants of a target sequence.

The oligonucleotides required for a primer extension (at least one target sequence-specific first primer and at least one target sequence-specific controller oligonucleotide) are combined as a target sequence-specific primer extension system.

Since several target sequences may also be present in the batch, the use of several target sequence-specific primer extension systems in a reaction batch should enable parallel primer extension of more than one target sequence, wherein target sequence-specific components are preferably combined respectively.

The use of pre-defined controller oligonucleotide thus enables sequence-dependent verification of the contents of the primer extension products after individual synthesis steps or during primer extension and causing a selection or a selection of sequences for subsequent synthesis steps. A distinction can be made between “active” single-stranded states of newly synthesized specific primer extension products as a result of a successful interaction with a controller oligonucleotide, and “inactive” double-stranded states of newly synthesized non-specific primer extension products as a result of a defective and/or insufficient and/or reduced and/or slowed interaction with a controller oligonucleotide.

### Definitions and Certain Embodiments

Unless otherwise defined, all technical and scientific terms used herein have the same meaning as they are commonly understood by an average person skilled in the art. Standard techniques are used for molecular biological or biochemical methods (see e.g. Sambrook et al., Molecular Cloning: A Laboratory Manual, 2d ed. (1989) Cold Spring Harbor Laboratory Press, Cold Spring Harbor, N.Y. and Ausubel et al., Short Protocols in Molecular Biology (1999) 4th Ed, John Wiley & Sons, Inc.)

In the context of the present invention, the terms used have the following meaning:

If the sequences shown in the description and in the sequence listing differ from each other, the sequence shown in the description applies in case of doubt.

The term “oligonucleotide”, as used here with reference to primers, controller oligonucleotide, probes and the nucleic acid chain to be amplified, is defined as a molecule comprising two or more, particularly more than three deoxyribonucleotides and/or ribonucleotides and/or nucleotide modifications and/or non-nucleotide modifications. Its length particularly comprises ranges between 3 to 300 nucleotide units (wherein natural bases and analogues can be used together or respectively alone), particularly between 5 to 200 nucleotide units. The skilled person knows how to determine the exact magnitude depending on the final function or use of the oligonucleotides.

The term “primer”, as used here, refers to an oligonucleotide, whether it occurs naturally, e.g. in a purified restriction cleavage, or whether it is synthetically produced. A primer is able to act as an initiation point of synthesis when used under conditions that induce the synthesis of a primer extension product complementary to a nucleic acid strand, i.e. in the presence of nucleotides and an inducing agent such as DNA polymerase at a suitable temperature and pH value. The primer is preferably single-stranded for maximum primer extension efficiency. The primer must be sufficiently long to initiate the synthesis of the extension product in the presence of the inducing agent. The exact length of the primer depends on many factors, including the reaction temperature and primer source and the application of the method. For example, in diagnostic applications, the length of oligonucleotide primers is between 5 to 100 nucleotides, particularly 6 to 40 and particularly 7 to 30 nucleotides, depending on the complexity of the target sequence. Short primer molecules generally require lower reaction temperatures to perform their priming function in order to form sufficiently stable complexes with the template, or higher concentrations of other reaction components, for example DNA polymerases, so that a sufficient extension of primer-template complexes formed can carry out.

The primers used here are selected such that they are “essentially” complementary to the different strands of each specific sequence to be amplified. This means that the primers must be sufficiently complementary to hybridize with their respective strands and initiate a primer extension reaction.

Thus, for example, the primer sequence does not need to reflect the exact sequence of the target sequence. For example, a non-complementary nucleotide fragment may be attached to the 5′-end of the primer, wherein the rest of the primer sequence is complementary to the strand. In another embodiment, single non-complementary bases or longer non-complementary sequences may be inserted into a primer, provided that the primer sequence comprises sufficiently high complementarity with the sequence of the strand to be amplified to hybridize therewith to produce a primer-template complex capable of synthesizing the extension product.

In the context of the enzymatic synthesis of a strand complementary to the template, a primer extension product is generated which is fully complementary to the template strand.

The term complementary with respect to sequences refers to hybridizing sequences with reverse complementary base sequences.

Allele-specific primers are primers which, due to their sequence composition, are capable of hybridizing preferentially under stringent reaction conditions to individual sequence variants of a target sequence respectively and of being extended by a polymerase using the target sequence. Individual allele-specific primers can be combined into a group which covers all variants of a common target sequence. Such a group of allele-specific primers comprises at least two different allele-specific primers, given that a polymorphic locus at a given position in the target sequence comprises at least two sequence variants. The allele-specific primers are selected in such way that under stringent reaction conditions they preferentially bind to their respective specific perfect match template and thus use this specific perfect match template to form the respective primer extension products under catalytic action of the polymerase. Preferably, 3′-terminal segments of allele-specific primers can be used to discriminate variants of target sequences and are adapted to the respective variants in their sequence composition in such way that such primers form a perfect match double strand under stringent conditions with the respective variant. Such perfect-match double strands can generally be well detected by a polymerase and under suitable reaction conditions primers are extended. Thus, when an allele-specific primer interacts with another variant of a target sequence, a mismatch double strand is formed. Such mismatches typically lead to a delay in extension by a polymerase or to a slowdown of the overall reaction. In certain embodiments, allele-specific primers in the 3′ segment may comprise at least one phosphorothioate bond, which protects allele-specific primers from 3′-5′ nuclease degradation by a polymerase.

Several allele-specific primers thus comprise sequence segments which are essentially identical or uniform for a group of allele-specific primers as well as sequence segments which are different among the primers of a group and are characteristic for the respective sequence variant of a target sequence. By including uniform sequence segments, such primers can hybridize to the respective target sequence under reaction conditions. Including characteristic sequence segments, a respective primer can bind specifically to a sequence variant of the target sequence forming a perfect-match double strand. Preferably, the primers are designed in such way that under the reaction conditions used, binding to a target sequence is preferred to form a perfect-match double strand and binding to a target sequence is less preferred to form a mismatch double strand.

In certain embodiments, the first oligonucleotide primer is provided as an allele-specific primer in combination with a respective allele-specific controller oligonucleotide. In certain embodiments, the second primer oligonucleotide primer is provided as an allele-specific primer in combination with a respective allele-specific controller oligonucleotide.

Tm-Melting Temperature

Generally, the melting temperature of a complementary or partially complementary double strand is understood to be a measured value of a reaction temperature at which approx. half of the strands are present as double strands and the other half as single strands. The system (association and dissociation of strands) is in balance.

Due to a variety of factors that can influence the Tm of a double strand (e.g. sequence length, CG content of the sequence, buffer conditions, concentration of divalent metal cations, etc.) the determination of the Tm of a nucleic acid to be amplified should be carried out under the same conditions as the intended primer extension reaction.

Due to the dependence of the measurable melting temperature on multiple reaction parameters, e.g. on the respective buffer conditions and the respective concentrations of the reaction participants, melting temperature is understood to be a value which was measured in the same reaction buffer as the exponential amplification, at concentrations of both complementary components of a double strand of about 0.1 μmol/l to about 10 μmol/l, particularly in concentrations of about 0.3 μmol/ to about 3 μmol/l, particularly at about 1 μmol/l. The respective value of the melting temperature is a guide value which correlates with the stability of a respective double strand.

A melting temperature for a double strand can be roughly estimated. Several commercial suppliers allow a theoretical calculation of an expected melting temperature. For example, the software package OligoAnalyzer 3.1 (available online at IDT (Integrated DNA-Technologies) can be used to estimate the strength of the binding of individual oligonucleotides.

The deoxyribonucleoside triphosphates (dNTPs) dATP, dCTP, dGTP and TTP (or dUTP, or dUTP/TTP mixture) are added to the synthesis mixture in adequate quantities. In certain embodiments, at least one further type of dNTP analogue may be added to the synthesis mixture in addition to dNTPs. In certain embodiments, these dNTP analogues comprise, for example, a characteristic label (e.g. biotin or fluorescent dye), such that if incorporated into the nucleic acid strand, this label is also integrated into the nucleic acid strand. In another embodiment, these dNTP analogues comprise at least one modification of sugar-phosphate portion of the nucleotide, e.g. alpha-phosphorothioate-2′-deoxyribonucleoside triphosphates (or other modifications which confer nuclease resistance to a nucleic acid strand), 2′,3′-dideoxyribonucleoside triphosphates, acyclo-nucleoside triphosphates (or other modifications leading to termination of synthesis). In certain embodiments, these dNTP analogues comprise at least one modification of nucleobase, e.g. iso-cytosines, iso-guanosines (or other modifications of nucleobases of the extended genetic alphabet), 2-amino-adenosines, 2-thiouridines, inosines, 7-deazy-adenosines, 7-deaza-guanosines, 5-Me-cytosines, 5-propyl-urridines, 5-propyl-cytosines (or other modifications of nucleobases which can be incorporated by a polymerase in relation to natural nucleobases and lead to a change in strand stability). In certain embodiments, a dNTP analogue comprises both a modification of the nucleobase and a modification of the sugar-phosphate portion. In certain embodiments, at least one further type of dNTP analogue is added to the synthesis mixture instead of at least one natural dNTP substrate.

The nucleic acid synthesis-inducing agent may be an enzyme or a system which acts in such way that the synthesis of the primer extension products is thereby effected. Suitable enzymes for this purpose comprise e.g. template-dependent DNA polymerases such as Bst polymerase and its modifications, Vent polymerase and other-particularly heat-stable DNA polymerases which enable the nucleotides to be incorporated in the correct manner, thereby forming the primer extension products which are complementary to each synthesized nucleic acid strand. Generally, synthesis is initiated at the 3′ end of each primer and continues along the template strand until synthesis is complete or interrupted.

Preferably, polymerases capable of strand displacement are used. These include for example the large fragment of Bst polymerase or its modifications (e.g. Bst 2.0 DNA polymerase), Klenow fragment, vent exo minus polymerase, deepvent exo minus DNA polymerase, large fragment of Bsu DNA polymerase, large fragment of Bsm DNA polymerase.

In certain embodiments, polymerases which do not comprise 5′-3′ exonuclease activity or do not have 5′-3′-FEN activity are particularly used.

In certain embodiments at least two different polymerases are used, for example polymerases which are able to displace strands and those which comprise a 3′-5′ proofreading activity.

In preferred embodiments, polymerases with a hot-start function are used, which can develop their function only upon reaching a certain temperature.

First Oligonucleotide Primer:

The oligonucleotide primer comprises a first region and a second region. The first region is capable of binding to an essentially complementary sequence within the nucleic acid to be amplified or its equivalents and initiating a primer extension reaction. The second region comprises a polynucleotide tail which is capable of binding a controller oligonucleotide and thereby causing a proximity between the controller oligonucleotide and the other parts of the first primer extension product sufficient to initiate strand displacement by the controller oligonucleotide. The second region of the first oligonucleotide primer further comprises at least one modification (a nucleotide modification or a non-nucleotide modification) that prevents the polymerase from copying the polynucleotide tail by inhibiting the continuation of polymerase-dependent synthesis. This modification is located, for example, at the transition between the first and second regions of the oligonucleotide primers. Consequently, the first region of the oligonucleotide primer is copyable by a polymerase, such that a complementary sequence to this region can be generated by the polymerase during the synthesis of the second primer extension product. The polynucleotide tail of the second region of the oligonucleotide primer is preferably not copied by the polymerase.

Certain embodiments of the first oligonucleotide primer

In certain embodiments this is achieved by modification in the second region, which stops the polymerase before the polynucleotide tail. In certain embodiments this is achieved by nucleotide modifications in the second region, wherein the entire polynucleotide tail essentially consists of such nucleotide modifications and is thus uncopyable for the polymerase.

In certain embodiments, each oligonucleotide primer is specific for a nucleic acid to be amplified.

In certain embodiments, each oligonucleotide primer is specific for at least two of the nucleic acids to be amplified, which respectively comprise essentially different sequences.

In certain embodiments, the oligonucleotide primer is labelled with a characteristic marker, e.g. a fluorescent dye (e.g. TAMRA, Fluorescein, Cy3, Cy5) or an affinity marker (e.g. biotin, digoxigenin) or an additional sequence fragment, e.g. for binding a specific oligonucleotide probe for detection or immobilisation or for barcode labelling.

In certain embodiments, at least one first oligonucleotide primer is provided as an allele-specific primer in combination with at least one respective allele-specific controller oligonucleotide.

Primer Extension Product:

A primer extension product (also called primer extension product) is formed by enzymatic (polymerase-dependent) extension of an oligonucleotide primer as the result of a template-dependent synthesis which is catalysed by a polymerase.

A primer extension product comprises the sequence of the oligonucleotide primer in its 5′-segment and the sequence of the extension product (also called extension product) which has been synthesized by a polymerase in template-dependent form. The extension product synthesized by the polymerase is complementary to the template strand on which it was synthesized.

A specific primer extension product (main product) comprises sequences of the nucleic acid chain to be amplified. It is the result of a specific synthesis or correct performance of an intended primer extension reaction in which the specific nucleic acid chain to be amplified serves as a template.

The length of the extension product of a specific primer extension can be between 10 and 300 nucleotides, particularly between 10 and 180 nucleotides, particularly between 20 and 120 nucleotides, particularly between 30 and 80 nucleotides.

A non-specific primer extension product (by-product) comprises, for example, sequences which have been produced as a result of a non-specific or incorrect or unintended primer extension reaction. These include, for example, primer extension products resulting from an incorrect initiation event (false priming) or from other side reactions, including polymerase-dependent sequence changes such as base substitution, deletion, etc. The extent of sequence deviation from unspecific primer extension products generally exceeds the ability of controller oligonucleotide to successfully displace such double-stranded by-products from their templates, resulting in slower or complete absence of amplification of such by-products. The degree of acceptance or tolerance to deviations depends, for example, on reaction temperature and the type and manner of sequence deviation. Examples of non-specific primer extension products are primer dimers or sequence variants that do not correspond to the nucleic acid to be amplified, e.g. sequences that do not comprise a target sequence.

The assessment of sufficient specificity of amplification is often related to the task at hand. In many amplification methods a certain degree of unspecificity of the amplification reaction can be tolerated as long as the desired result can be achieved.

Certain Embodiments of the First Primer Extension Product:

In certain embodiments, the portion of nucleic acid chains to be amplified amounts to more than 1% of the total result of the reaction, particularly more than 10%, particularly more than 30%, measured on the total amount of newly synthesized strands.

In certain embodiments, the sequence of the synthesized primer extension products completely matches the expected sequence of a nucleic acid to be amplified. In another embodiment, deviations in the obtained sequence from the theoretically expected sequence can be tolerated. In certain embodiments, the degree of matching of the obtained sequence as a result of amplification with the sequence of the nucleic acid to be theoretically expected to be amplified is, for example, between 90% and 100%, particularly the degree of matching is above 95%, particularly the degree of matching is above 98% (measured by the portion of bases synthesized).

The first primer extension product is formed starting from a first oligonucleotide primer. A competitor-primer extension product is formed from a competitor-oligonucleotide-primer (FIGS. 21-23).

A primer extension product can serve in certain embodiments as a start nucleic acid chain comprising a target nucleic acid comprising sequence.

Target Sequence:

In certain embodiments, a target sequence is a segment of a nucleic acid chain to be amplified which can serve as a characteristic sequence of the nucleic acid to be amplified. Said target sequence can serve as a marker for the presence or absence of another nucleic acid. Said other nucleic acid thus serves as a source of the target sequence and may be, for example, genomic DNA or RNA or parts of genomic DNA or RNA (e.g. mRNA), or equivalents of genomic DNA or RNA of an organism (e.g. cDNA, modified RNA such as rRNA, tRNA, microRNA, etc.).), or comprise defined changes in the genomic DNA or RNA of an organism, for example mutations (e.g. deletions, insertions, substitutions, additions, sequence propagation, e.g. repeat propagation in the context of microsatellite instability), splice variants, rearrangement variants (e.g. T-cell receptor variants), etc. The individual target sequences may represent a phenotypic trait, e.g. antibiotic resistance or have prognostic information and thus be of interest for diagnostic assays/tests. As the source/origin of a target sequence, such a nucleic acid may comprise the target sequence as a sequence element of its strand, for example. A target sequence can thus serve as a characteristic marker for a specific sequence content of another nucleic acid.

The target sequence can be single-stranded or double-stranded. It may be essentially identical to the nucleic acid to be amplified or it may represent only a part of the nucleic acid to be amplified.

Equivalents of the target sequence comprise nucleic acids with essentially identical information content. For example, complementary strands of a target sequence have identical information content and can be called equivalent, RNA and DNA variants of a sequence are also examples of equivalent information content.

During material preparation prior to the amplification reaction, such a target sequence can be isolated from its original sequence environment and prepared for the amplification reaction.

Certain Embodiments of the Target Sequence:

In certain embodiments, a nucleic acid to be amplified comprises a target sequence. In certain embodiments, the target sequence corresponds to the nucleic acid to be amplified. In a further embodiment, a start nucleic acid chain comprises a target sequence. In certain embodiments, the target sequence corresponds to a start nucleic acid chain.

The nucleic acid to be amplified represents a nucleic acid chain which is to be amplified sequence-specifically or at least predominantly sequence-specifically by the polymerase by means of exponential amplification using primers and controller oligonucleotide.

The length of the nucleic acid to be amplified can be between 20 and 300 nucleotides, in particular between 30 and 200 nucleotides, particularly between 40 and 150 nucleotides, particularly between 50 and 100 nucleotides.

The nucleic acid chain to be amplified may comprise one or more target sequences or their equivalents. Furthermore, a nucleic acid to be amplified may comprise the sequences essentially complementary to a target sequence, which are amplified with similar efficiency as a target sequence in an amplification reaction and comprise the target sequence or its segments. In addition to a target sequence, the nucleic acid to be amplified may include further sequence segments, for example primer sequences, sequences with primer binding sites and/or sequence segments for binding of detection probes, and/or sequence segments for sequence coding of strands by barcode sequences and/or sequence segments for binding to a solid phase. The primer sequences or their sequence portions, as well as primer binding sites or their sequence portions can for example belong to sequence segments of a target sequence.

In certain embodiments, the nucleic acid to be amplified corresponds to the target sequence.

In an embodiment, the target sequence forms part of the sequence of the nucleic acid chain to be amplified. Such a target sequence can be flanked at 3′ side and/or 5′ side by further sequences. Said further sequences may, for example, comprise binding sites for primers or their portions, and/or comprise primer sequences or their portions, and/or comprise binding sites for detection probes, and/or comprise adaptor sequences for complementary binding to a solid phase (e.g. in microarrays, or bead-based analyses), and/or comprise barcoding sequences for a digital signature of sequences.

In order to start the amplification, a nucleic acid chain must be added to the reaction mixture at the beginning of the reaction, which acts as an initial template for the synthesis of the nucleic acid chain to be amplified. Said nucleic acid chain is called the start nucleic acid chain. Said start nucleic acid chain defines the arrangement of individual sequence elements which are important for the formation/synthesis/exponential amplification of a nucleic acid chain to be amplified.

In certain embodiments the initial template (start nucleic acid chain), which is fed to an amplification reaction at the beginning, or which is added to the reaction mixture, corresponds to the sequence composition of the nucleic acid chain to be amplified.

In initial stages of the amplification reaction and in its further course, the respective primers bind to the respective binding sites in the start nucleic acid chain and initiate the synthesis of specific primer extension products. Such specific primer extension products accumulate exponentially during amplification and increasingly take over the role of templates for the synthesis of complementary primer extension products in exponential amplification.

The repeated template-dependent synthesis procedures in exponential amplification thus form the nucleic acid chain to be amplified.

Towards the end of an amplification reaction, the main product of the reaction (the nucleic acid to be amplified) may be predominantly single-stranded or predominantly form a complementary double-strand. This can be determined, for example, by the relative concentration of both primers and respective reaction conditions.

Equivalents of the nucleic acid to be amplified comprise nucleic acids with essentially identical information content. For example, complementary strands of a nucleic acid to be amplified have identical information content and can be called equivalent.

Allelic variants of a target sequence/Polymorphic locus of a target sequence (FIGS. 12-24)

A target sequence (e.g. a characteristic segment of a DNA or RNA) may comprise several sequence variants occurring in nature or artificially introduced sequence changes. Such variants are often called polymorphisms of a locus or allele sequences. Furthermore, these target sequence variants can represent specific changes in the natural state of the DNA, e.g. due to mutations / mutagenesis, which are characteristic features of such a clonal multiplication process (e.g. in cancer diseases or in biotechnologically exploitable strains). Individual allele sequences of a target sequence may be sequence differences which comprise individual nucleotides (e.g. A->G or T->C substitutions or single nucleotide insertions or deletions) or a sequence difference may also comprise several nucleotides (e.g. 2 to 200 nucleotides). These can be deletions/insertions/transversions/duplications etc. A sequence segment comprising such sequence variants of a target sequence is often called a polymorphic locus. A target sequence can comprise one or more polymorphic loci.

The sequence positions of single allele variants of a target sequence are generally known and thus allow the design of sequence-specific reaction components, e.g. controller oligonucleotides, or of combinations comprising a controller oligonucleotide and a first primer or a controller oligonucleotide and a second primer.

The Embodiments of Target Sequences:

In certain embodiments, a target sequence, which may comprise several sequence variants in a polymorphic locus, further comprises at least a first target sequence segment, which is characteristic and uniform for all target sequence variants of a target sequence (target sequence group). In certain embodiments, a target sequence comprises at least two target sequence segments (a first target sequence segment and a second target sequence segment), each of said target sequence segments being characteristic and uniform for all target sequence variants of a target sequence (target sequence group). Such uniform target sequence segments are preferably located on both sides of a polymorphic locus and thus flank a polymorphic locus (with sequence variants) of a target sequence from both sides. Preferably, the first uniform target sequence segment differs in its sequence composition from the sequence composition of the second uniform target sequence segment in at least one nucleotide position.

The length of a polymorphic locus of a target sequence may comprise regions from one nucleotide up to 200 nucleotides, especially from one nucleotide up to 100 nucleotides, particularly from one nucleotide up to 50 nucleotides, particularly from one nucleotide up to 20 nucleotides. The lengths of a uniform sequence segment (first and second uniform sequence segment) can comprise, at least for one of the two segments, the following regions: from 6 to 300 nucleotides, particularly from 10 to 200 nucleotides, particularly from 15 to 100 nucleotides, particularly from 20 to 100 nucleotides.

In certain embodiments, individual sequence variants of a target sequence group can be combined, wherein such variants are defined at least for a sequence segment which is uniform and specific for said target sequence (target sequence group). Individual sequence variants of such a target sequence (target sequence group) are thus distinguished by sequence segments of the polymorphic locus.

In certain embodiments, individual sequence variants of a target sequence group can be combined, wherein such variants are defined for at least two sequence segments which are uniform and specific for said target sequence (target sequence group). Individual sequence variants of such a target sequence (target sequence group) are thus distinguished by sequence segments of the polymorphic focus. Preferably, the polymorphic locus is located between both uniform sequence segments.

In certain embodiments, two different target sequences can be specifically amplified respectively in an amplification reaction.

Different target sequences are characterized in that they can preferably be differentiated from one another by sequences that are specific and characteristic for each target sequence. Preferably, different target sequences can be differentiated in their entire length by sequences which are respectively specific and characteristic for each target sequence and thus do not comprise sequence segments which are identical for both target sequences. In certain embodiments, different target sequences can comprise at least one sequence segment which is essentially similar or even identical for at least two different target sequences. The length of such a sequence segment is equal to/or less than 19 nucleotides (counted as nucleotide positions coupled to each other), particularly equal to/or less than 14 nucleotides (counted as nucleotide positions coupled to each other), particularly equal to/or less than 9 nucleotides (counted as nucleotide positions coupled to each other), particularly equal to/or less than 5 nucleotides (counted as nucleotide positions coupled to each other).

The start nucleic acid chain constitutes a preferred embodiment of a target nucleic acid comprising a sequence segment which is to result as the product of a primer extension reaction.

In order to start the amplification, a nucleic acid chain must be added to the reaction mixture at the beginning of the reaction, which acts as an initial template for the synthesis of the nucleic acid chain to be amplified. This nucleic acid chain is called the start nucleic acid chain. Said start nucleic acid chain defines the arrangement of individual sequence elements which are important for the formation/synthesis/exponential amplification of a nucleic acid chain to be amplified.

Such a start nucleic acid chain can be single stranded or double stranded at the beginning of the reaction. If the complementary strands of the start nucleic acid chain are separated from each other, the strands can serve as templates for the synthesis of specific complementary primer extension products, regardless of whether the nucleic acid was originally double-stranded or single-stranded.

The start nucleic acid chain comprises, in an embodiment, a target sequence or its segment parts comprising at least one expected sequence variant of a polymorphic locus of the target sequence.

A start nucleic acid further comprises at least one predominantly single-stranded sequence segment to which at least one of the primers of the amplification system with its 3′ segment can bind predominantly complementarily, so that the polymerase used can extend such a primer, when hybridized to the starting nucleic acid chain, template specifically with incorporation of dNTPs.

A start nucleic acid chain comprises in an embodiment a target sequence or its parts which comprise at least one expected sequence variant of a polymorphic locus of the target sequence and at least one uniform sequence segment of a target sequence.

A start nucleic acid chain comprises in an embodiment a target sequence or its parts which comprise at least one expected sequence variant of a polymorphic locus of the target sequence and at least two uniform sequence segments of a target sequence, wherein uniform sequence segments of a target sequence flank the polymorphic locus with possible sequence variants on both sides.

Controller Oligonucleotide/Controller:

The controller oligonucleotide is a single-stranded nucleic acid chain that includes a predefined essentially complementary sequence in a part of the primer extension product that is specifically generated during the amplification of the nucleic acid to be amplified. Thereby the controller oligonucleotide can bind to the oligonucleotide primer and at least to the 5′ segment of the specific extension product of the oligonucleotide primer in an essentially complementary manner. The controller oligonucleotide comprises in its inner sequence segment in certain embodiments nucleotide modifications which prevent the polymerase from synthesizing a complementary strand using the controller oligonucleotide as template when the primer oligonucleotide is complementarily bound to the controller oligonucleotide.

In certain embodiments, identical controllers are used in the primer extension reaction as in the subsequent acid reaction.

In certain embodiments, the primer extension reaction uses a controller that differs from that used in the subsequent acid reaction.

The target sequence-specific controller oligonucleotide is preferably constructed in such way that it is capable of participating in the amplification of preferably a defined specific sequence variant of a predefined target sequence.

The allele-specific controller oligonucleotide is preferably constructed in such way that it is capable of preferably causing the amplification of a specific sequence variant of a target sequence (e.g. allele 1), wherein in the case of another sequence variant of the same target sequence (e.g. allele 2) the amplification proceeds with reduced efficiency or does not take place at all in the predetermined time or results in an insufficient yield of amplification products for detection. Thus, measurable differences in the course of an amplification reaction result, which depend on whether the sequence of a controller oligonucleotide specific for an allele variant specifically matches or does not specifically match the sequence of a nucleic acid chain provided at the beginning of the reaction, which serves as the starting nucleic acid and comprises the target sequence with a polymorphic locus. If the sequence composition of the polymorphic locus of a start nucleic acid chain and a corresponding sequence segment in the controller oligonucleotide match or are perfectly complementary, a specific characteristic amplification takes place. In the absence of matching or with mismatch complementarity between the sequence composition of the polymorphic locus of a start nucleic acid chain and a corresponding sequence segment in the controller oligonucleotide, the amplification proceeds differently than a specific characteristic amplification.

An allele-specific controller oligonucleotide is thus provided not only target sequence-specific but also allele-specific. Depending on the complexity of a polymorphic locus of a target sequence, several controller oligonucleotides specific for respective allele variants of a target sequence can thus be constructed.

Preferably, a controller oligonucleotide comprises at least one sequence segment which comprises a sequence composition complementary to a specific allele variant of a target sequence. Furthermore, a controller oligonucleotide preferably comprises at least one sequence segment which comprises a complementary sequence composition which is uniform for all sequence variants of a target sequence. In certain embodiments, a controller oligonucleotide comprises at least one sequence segment which can bind complementarily to a variant of a polymorphic locus of a target sequence.

In certain embodiments, several controller oligonucleotides are provided, which respectively comprise allele-specific sequence segments.

The length of a sequence segment of a controller oligonucleotide complementary to the polymorphic locus of a start nucleic acid chain can comprise ranges from one nucleotide up to 100 nucleotides, particularly from one nucleotide up to 50 nucleotides, particularly from one nucleotide up to 20 nucleotides. The length of at least one sequence segment of a controller oligonucleotide which is complementary to uniform sequence segments of a target sequence (first and second uniform sequence segment of a start nucleic acid) can comprise the following ranges: from 4 to 100 nucleotides, particularly from 6 to 100 nucleotides, particularly from 8 to 50 nucleotides.

In certain embodiments, a sequence segment of a controller oligonucleotide which is complementary to at least one variant of a polymorphic locus of a target sequence is located in the third region of the controller oligonucleotide.

In certain embodiments, a sequence segment of a controller oligonucleotide which is complementary to at least one variant of a polymorphic locus of a target sequence is located in the second region of the controller oligonucleotide.

In certain embodiments, a sequence segment of a controller oligonucleotide which is complementary to at least one variant of a polymorphic locus of a target sequence is partially and/or completely located in the second and third regions of the controller oligonucleotide.

The formation of base pairs amongst two nucleic acid strands can take place essentially complementarily, which means that between 70% and 100%, particularly between 90% and 100%, particularly between 95% and 100%, of nucleotides of one strand can bind to each other in a complementary manner. For example, a nucleotide position in a strand of 20 nucleotides cannot comprise complementary binding, which is equivalent to 95% complementarity.

In case of perfect match binding of two sequences, preferably 100% complementary base pairing takes place between both strands of a duplex. A perfect match duplex therefore does not comprise any mismatch between the strands.

General structural features of a controller oligonucleotide/primer system (feedback system) and a primer template system (synthesis system).

The controller oligonucleotides preferably do not serve as templates for primer extension. The extension of corresponding primers on controller oligonucleotides is preferably prevented by modifying controller strands. The controller oligonucleotide is capable of binding to an overhang on the primer (the first region of the controller oligonucleotide preferably binds specifically to the second region of the respective primer) and can undergo complementary binding with the primer (the second region of the controller oligonucleotide binds to the first region of the primer). Further, the controller can bind with its third region to the synthesized portion of the primer extension product in a predominantly sequence-specific manner.

In a specific embodiment, the primers used have a tail or overhang which cannot form a stable double strand with the template (the primer overhang remains completely or partially free when the primer binds to the primer binding site of the template) and is thus available for interaction with the first region of the controller oligonucleotide. This primer overhang serves as a binding site for a specific controller oligonucleotide strand respectively (first segment of the controller oligonucleotide). The controller oligonucleotide binds to said overhang and is thus brought into spatial proximity with the double strand. This creates the conditions under which the controller oligonucleotide can undergo a double-strand invasion to form a complementary double-strand (this procedure is also called branch migration or sequence-dependent strand displacement). The strand invasion takes place by forming a new double strand between a controller oligonucleotide and the corresponding strand of the synthesized amplification fragments. Due to the strong sequence dependence of such double strand formation, the opening of the synthesized double strands takes place with high dependence on the sequence agreement with the respective controller sequence.

In a specific embodiment the primers have a tail or overhang which is not copied by a polymerase. The polymerase is prevented from copying the overhang by respective primer modifications.

Primer Extension System:

A primer extension system comprises at least one target sequence-specific controller oligonucleotide and at least one first primer oligonucleotide as well as at least one template-dependent polymerase which is capable of supporting predominantly specific amplification of the nucleic acid chain to be amplified using a template comprising at least one characteristic target sequence or target sequence variant under suitable reaction conditions, wherein predominantly target sequence-specific first primer extension products are synthesized.

An allele-specific primer extension system comprises at least one allele-specific controller oligonucleotide and at least one first oligonucleotide primer (wherein primers can be target sequence-specific and/or allele-specific depending on the design), as well as at least one template-dependent polymerase capable of using a template comprising at least one target sequence variant to support under suitable reaction conditions a predominantly specific amplification of the target sequence variant (an allele) of the nucleic acid chain to be amplified, wherein predominantly specific first primer extension products are synthesized.

A primer extension system may comprise multiple allele-specific primer extension products, wherein each allele-specific primer extension system preferentially copies at least one of the target sequence variants.

Strand Displacement (Strand Displacement):

Hereby a process is denoted, which by the action of a suitable means leads to a complete or partial separation of a first double strand (consisting for example of A1 and B1 strand) and to the simultaneous/parallel formation of a new second double strand, wherein at least one of the strands (A1 or B1) is involved in the formation of this new second strand. A distinction can be made here between two forms of strand displacement.

In a first form of strand displacement, the formation of a new second double strand can be carried out by using an already existing complementary strand, which is generally in single-strand form at the beginning of the reaction. In this case, the means of strand displacement (for example a preformed single-stranded strand C1 comprising a sequence complementary to strand A1) acts on the first already designed double strand (A1 and B1) and forms a complementary bond with strand A1, thereby displacing strand B1 from the bond with strand A1. If the displacement of B1 is complete, the result of the Cl effect is a new double strand (A1:01) and a single strand B1. If the displacement of B1 is incomplete, the result depends on several factors. For example, a complex of partially double-stranded A1:B1 and A1:01 may be present as an intermediate product.

In a second form of strand displacement, the formation of a new second double strand can take place under a simultaneous enzymatic synthesis of the complementary strand, wherein one strand of the first pre-formed double strand acts as a template for synthesis by the polymerase. Thereby the strand displacing agent (for example polymerase with a strand displacing ability) acts on the first already preformed double strand (A1 and B1) and synthesizes a complementary strand D1 new to strand A1, wherein strand B1 is simultaneously displaced from the bond with strand A1.

The term “nucleic acid-mediated strand displacement” is used to describe a sum/series of intermediate steps which may be in equilibrium with one another and which result in the temporary or permanent opening of a first preformed duplex (consisting of complementary strands A1 and B1) and formation of a new second duplex (consisting of complementary strands A1 and C1), wherein A1 and C1 are complementary to one another.

It is known that an essential structural condition for initiating strand displacement is the establishment of spatial proximity between a duplex end (preformed first duplex of A1 and B1) and a single-stranded strand (C1) which initiates strand displacement (wherein A1 and C1 can form a complementary strand). Such proximity can preferably be achieved by means of a single-stranded overhang (in the literature examples with short overhangs are known, called “toehold”), which temporarily or permanently binds the single-stranded strand (C1) in a complementary manner, and thus brings complementary segments of the strand C1 and A1 into sufficient proximity such that successful strand displacement of strand B1 can be initiated. The efficiency of the initiation of nucleic acid-mediated strand displacement is generally higher the closer the complementary segments of strand A1 and C1 are positioned to each other.

Another essential structural prerequisite for the efficient continuation of nucleic acid-mediated strand displacement in inner segments is in high complementarity between strands (e.g. between Al and C1), which must form a new double strand. For example, single nucleotide mutations (in C1) can lead to the interruption of a strand displacement (described e.g. for branch migration).

The present invention makes use of the ability of complementary nucleic acids to form a sequence-dependent nucleic acid-mediated strand displacement.

Embodiments of the Interaction of Individual Components in Primer Extension Reactions

Synthesis of the Primer Extension Product and Interaction with the Controller in Predominantly Sequential Order:

The technical objective of this embodiment is the synthesis of a primer extension product and a subsequent separation of the primer extension product from its template. The separation of the primer extension product takes place with the participation of a controller oligonucleotide and is therefore preferably sequence-specific.

The synthesis of the primer extension product and verification by means of a controller oligonucleotide is preferably carried out in the same batch (homogeneous assay), wherein the individual reactions are, however, timely separated. First the synthesis of the P1-Ext (synthesis phase) takes place (FIG. 3) and then the opening of the double strand takes place by means of a controller (controlling phase) (FIG. 4). The two phases can be used, for example, by changing the reaction temperature of the reaction batch to a desired sequence (for homogeneous assay) or by sequential addition as a heterogeneous format as well.

Simultaneous presence of a first primer and a controller in a batch in the presence of a primer extension-catalyzing agent (a polymrease) and suitable substrates requires the use of a controller modified such that the first primer, when hybridized to the controller oligonucleotide, is not extended by a polymerase.

In the following, a preferred form is described with a homogeneous format in which at least one template strand, at least one controller oligonucleotide, at least one oligonucleotide primer, at least one polymerase and dNTPs are present as substrates in a suitable buffer system at the beginning of the reaction.

Synthesis Phase:

The aim of the synthesis phase is to synthesize a specific primer extension product by polymerase-mediated synthesis using a suitable template strand.

A primer extension reaction comprises a preferentially specific primer binding (first region of the primer oligonucleotide) to the template strand at a suitable primer binding site (sequence segment which is capable of binding preferentially complementary to the first region of the primer oligonucleotide), wherein the second region of the primer (primer overhang) preferentially does not bind to the template strand, and thus is available for later interaction with the controller. The primer binding site can be completely single-stranded or partially single-stranded, such that at least part of the first region of the primer can bind predominantly sequence-specifically to the primer binding site via formation of complementary base pairs (Watson-Crick rule).

The polymerase can now extend the oligonucleotide primers bound to the primer binding site in a template-dependent synthesis by attaching complementary nucleotides to the growing strand of the primer (3′-OH end), thereby producing a primer extension product (P1 -Ext) complementary to the template strand. The synthesis of the complementary strand is generally carried out under suitable reaction conditions until the polymerase finds sufficient substrate in the batch and until sufficient sequence segments are available in the template strand in a suitable form for the polymerase as a template. Depending on the polymerase, one of the conditions for the sequence segments can be that the template strand is single-stranded (using polymerases without strand displacement properties) or partially or even completely double-stranded (using polymerases with strand displacement properties). The synthesis often takes place over the sufficient time, such that a primer extension product can be synthesized from more than 5%, particularly more than 50%, particularly more than 90% of the template strands.

Immediately after synthesis, the primer extension product (P1-Ext) is located in complex with the template strand in double-stranded form (P1-Ext/template strand complex). This double-stranded complex comprising P1-Ext and the template strand is preferably stable under selected reaction conditions of the primer extension phase and cannot spontaneously dissociate into its constituents, or this spontaneous dissociation takes place very slowly and can thus be neglected.

In certain embodiments, primer binding takes place under reaction conditions that allow thermodynamically stable binding of the first region of the oligonucleotide primer to the respective complementary position in the template. Such conditions are present, for example, at reaction temperatures that are significantly below the melting temperature (Tm) of such a complex (P1/template strand). The Tm should have been measured under identical buffer conditions. For example, the reaction temperatures can be located in the regions from Tm minus 50° C. to Tm minus 5° C. If the 3′-segment of the primer and the template are sufficiently complementary, the extension of the primer by a polymerase is generally carried out quickly and easily if the reaction conditions are also well suited for the polymerase and sufficient amounts of dNTPs are present in the batch.

In a preferred embodiment, the complex comprising primer/controller oligonucleotide is also present in double-stranded form under such reaction conditions. Due to the fact that oligonucleotide primer and controller oligonucleotide interact in both the first region of the primer and in the second region of the primer (overhang/primer tail), the binding of a controller oligonucleotide to the first segment is generally more stable than that of the first region and the template. Under such reaction conditions, the primer should be in excess such that sufficient primer in single-stranded form remains in the batch and can react with the primer binding site of the template strand so that a primer extension reaction can take place. For example, the concentration of a controller oligonucleotide is about 1 μmol/, then the concentration of the first primer can be from about 1.1 to 5 μmol/. Under reaction conditions that require a stable duplex formation between a controller and a first primer, the controller is saturated by the primer. However, there will still be enough free single-stranded first oligonucleotide primers in the solution to interact with a suitable template.

During the synthesis phase, the controller oligonucleotide is thus preferably present in a complex comprising primer/controller oligonucleotide and is thus in an inert form. In this embodiment, the controller does not interfere with the synthesis procedure. In this state, the first and second regions of the controller oligonucleotide are occupied by the oligonucleotide primer (corresponding to its first and second region) by forming a complementary strand and are thus not available for interactions with formed primer extension products during the synthesis phase.

In a further preferred embodiment, primer binding takes place under reaction conditions that do not allow thermodynamically stable binding of the first region of the oligonucleotide primers to the respective complementary position in the template. Thus, there is a balance of binding events and dissociation events. During binding events, complexes are formed comprising the first region of the primer and the respective primer-binding positions of the template. During dissociation events, said complexes separate into individual components such that no primer extension reaction using polymerase can start. Such conditions are present, for example, at reaction temperatures that are located around the melting temperature (Tm) of such a complex (P1/template strand). The melting temperature (Tm) should be measured under identical buffer conditions and concentrations. For example, the reaction temperatures can be in the range from Tm−5° C. to Tm+5° C., in specific embodiments in the range from Tm−5° C. to about Tm+15° C., in a further specific embodiment in the range from Tm−5° C. to Tm+30° C. Even under such conditions, extension of the primer by a polymerase can take place with sufficient complementarity of the 3′ segment of the primer with the template. However, the reaction may be somewhat slower or significantly slower compared to reaction conditions where stable primer binding takes place. At reaction temperatures above Tm+30° C. (P1/template) the primer extension reaction is generally too inefficient that such conditions are rarely used in primer extension reactions. Even at reaction temperatures from Tm−5° C. to about Tm+15° C., it can be advantageous to have an excess of primers so that enough primer remains in single-stranded form and can react with the template strand so that a primer extension reaction can take place.

The structure of the first primers and the controller oligonucleotides as well as the reaction conditions of the synthesis phase are such that the double-stranded complex comprising primer/controller oligonucleotide is predominantly in double-stranded form. As a result of primer excess, the controller oligonucleotide can be saturated and thus preferably be excluded from interactions with formed primer extension products. Since primer oligonucleotide and controller oligonucleotide interact in both the first region of the primer and in the second region of the primer (overhang/primer tail), the binding of a primer to the controller is generally more stable than that between the first region of the primer and the template. Thus, the melting temperature (Tm) of the complex comprising primer/controller oligonucleotide is generally higher than that of the complex comprising primer/template.

Polymerases:

Both thermostable polymerases such as Taq polymerase/Pfu polymerase and thermolabile (such as Klenow polymerase fragment or MMLV reverse transcriptase) or mesophilic polymerases (e.g. Bst polymerase) can be used. The polymerases may or may not comprise a 3′ exonuclease activity (proofreading polymerases). Depending on the type of template used, DNA-dependent polymerases or RNA-dependent polymerases can be used.

Polymerases can use enzymes that are identical to natural enzymes (e.g. Pfu Polymerase or Bst DNA Polymerase) as well as their modifications or fragments or fusions with other proteins (e.g. AmpliTaq, Bst Large Fragment Polymerase, Klenow Large Fragment or Phusion Polymerase).

Polymerase with (e.g. Bst polymerase and its modification, such as Bst large fragment) or without strand displacement properties (such as Taq polymerase or Pfu polymerase or MMLV reverse transcriptase) can be used.

Both predominantly processive polymerases or their modifications (e.g. Bst-Large Fragment or Phusion Polymerase) and less processive polymerases (Taq Polymerase or Pfu Polymerase) can be used.

Since the synthesis phase and controlling phase are separated in time, the synthesis reaction takes place (synthesis phase) (FIG. 3) until the desired amount of primer extension product is available. Only then is the controlling phase carried out (FIG. 4). This gives the reaction sufficient time to form a sufficient amount of product, wherein the polymerases can generally be present in catalytic quantities.

Template Strand:

The template strand comprises a primer binding site, such that under selected reaction conditions binding of the first region of the first primer can take place and synthesis of the strand mediated by the polymerase can take place. Furthermore, a template strand comprises a target sequence. Said target sequence generally comprises the sequence segment of the template strand, which takes over the template function for the synthesis of the complementary strand. Preferably, the template strand and the primer oligonucleotide are designed to be complementary to each other in such way that the second region of the oligonucleotide primer does not bind complementarily to the template strand or at least does not undergo stable complementary binding and is thus potentially capable of interacting with the first segment of the controller oligonucleotide. Generally, both DNA and RNA can occur as template strands.

The primer binding site of the template is preferably located in a single-stranded form and allows sequence-specific binding of the first region of the oligonucleotide primer to the template. In certain embodiments, the target sequence of the template strand comprises at least one segment of the primer binding site such that the 3′ segment of the first primer can bind complementarily to the target sequence.

The segment of the template strand, which serves as a template for the synthesis of the complementary strand by the polymerase, can be completely or predominantly (more than 50%) single-stranded under reaction conditions, in certain embodiments. In an embodiment of another type, said segment is predominantly (more than 50%) or completely double-stranded. The polymerases used in the synthesis must be chosen accordingly: in single-stranded templates, polymerases without strand displacement can be used, e.g. Taq polymerase or Pfu polymerase. In the case of predominantly double-stranded segments of the template, polymerases with strand displacing properties are preferred, e.g. Bst-Polymerase Large Fragment or Vent-Polymerase or Klenow-Fragment. Combinations of several polymerases can also be used, e.g. Taq polymerase and vent polymerase or Taq polymerase and Bst polymerase, or Pfu polymerase and Bst polymerase. For polymerases used, their selection depends on the manufacturer's specifications. Naturally occurring polymerases as well as their partial components (e.g. Klenow Fragment or Bst-Polymerase Large Fragment) or modifications, e.g. Bst-2.0 Polymerase or AmpliTaq-Polymerase can be used.

In certain embodiments, the primer binding site for the primer may be located in the 3′ segment of the template. In an embodiment of this type, it is advantageous if the oligonucleotide primer comprises a modification (called first blocking unit) that prevents the polymerase from copying the second region of the primer.

In an embodiment of another type, the template can extend significantly beyond the primer binding site of the primer in its 3′ segment. The length of such an overhang can range from a few nucleotides to several kilobases. The structure of such a template strand means that the primer overhang, starting from the 3′-end of the template, is not copied by the polymerase. Therefore, in such an embodiment the primer does not necessarily have to comprise a block group (called first blocking unit) in the second region of the primer. Thus, the second region can immediately merge into the first region and be linked to the 5′-end of the first region via 5′-3′-phosphate linkage.

The synthesis of the primer extension product takes place via polymerase as template-dependent synthesis. In some embodiments it is advantageous to limit the progress of the synthesis of the primer extension product and thus to limit the length of a desired primer extension product.

The length of the primer extension product is preferentially limited by the availability of a sequence segment of the template which can be used by the polymerase for synthesis. This can be achieved, for example, by limiting the template strand in the 5′ sequence segment which is available as a template for complementary synthesis. This limitation prevents the polymerase used from continuing the synthesis beyond this obstacle. This limitation can be achieved, for example, by a strand end (strand break) or by using one or more modifications in the strand (e.g. spacers or linkers or abasic sugar-phosphate residues) which prevent the polymerase from continuing the synthesis, or by nucleotide modifications (e.g. nucleobase modification, such as Iso-C or Iso-G, or the sugar-phosphate portions, such as 2′-O-Me, 2-MOE, morpholino, PNA etc.).

By using long template strands, e.g. genomic DNA or mRNA or long PCR fragments, the synthesis of the polymerase can be stopped or halted by using additional oligonucleotides at predetermined sequence segments. In such an embodiment, the progress of the synthesis of the primer extension product is limited by the use of at least one so-called block oligonucleotide (FIGS. 3,4, 6-8). Under the reaction conditions of the synthesis phase, such a block oligonucleotide can preferably remain predominantly sequence specifically hybridized to the template strand via complementary binding. Such a block oligonucleotide, if complementarily bound to the template strand, prevents the polymerase from reading off the template strand in the sequence segment bound by this oligonucleotide and, respectively, beyond. Depending on the type of polymerase used and its configuration, different embodiments of block oligonucleotides can be considered. The selection of the oligonucleotide structure, the polymerase and the reaction conditions are based on the fact that the progressive synthesis by the polymerase is prevented or blocked. The length of the block oligonucleotide and the nature of the sequence composition are based on the fact that the block oligonucleotide must remain stably bound to its complementary position on a template strand under the reaction conditions of the synthesis phase and preferably cannot dissociate spontaneously from the template. Such an oligonucleotide is generally brought into contact with the template strand in effective concentrations in the 3′ direction from the first oligonucleotide primer before the start of a synthesis phase of a primer extension reaction and incubated for a sufficient time under suitable reaction conditions such that it can bind to its complementary position in the strand.

Examples of such oligonucleotides (FIGS. 3,4, 6-8) leading to the stopping of a polymerase-dependent synthesis are known to a skilled person.
For example, oligonucleotides with exonuclease-resistant modifications (e.g. phosphorothioates (PTO) or 2′-O-Me modifications, which are predominantly positioned in the 5′ segment of the oligonucleotide) can lead to, for example, that the Taq polymerase cannot break down the block oligonucleotide (with its 5′-3′ exonuclease activity) and thus cannot continue the synthesis via the binding site of the oligonucleotide. For example, oligonucleotides complementary to the template with a length of about 15-70 nucleotides with about 2-50 PTO modifications located predominantly in the 5′-segment of such a block oligonucleotide can prevent the Taq polymerase from cleaving or detaching this oligonucleotide from the template via 5′-'3 exonuclease dependent degradation. Such a nucleotide is generally capable of preventing a Taq polymerase from reading off the template strand segment bound by the block oligonucleotide.
Another example is the inhibition of polymerases with strand displacement properties. Oligonucleotides with several double-strand stabilising modifications, e.g. LNA or PNA, generally lead to the polymerase being unable to continue the synthesis via the binding site of the oligonucleotide. For example, oligonucleotides complementary to the template with a length of about 15-50 nucleotides with about 7-30 LNA modifications, for example, can prevent the Bst polymerase (large fragment) from detaching said oligonucleotide from the template via polymerase-dependent strand displacement. Further examples of such block oligonucleotides are known. For example, PNA modifications or LNA modifications have been used in clamping PCR methods to block polymerase synthesis.

The length of the P1-Ext generated during template-dependent synthesis and the length of the matching controller oligonucleotide can be selected differently.

In certain embodiments, the length of the controller oligonucleotide and the length of the expected primer extension product are matched in such way that the controller oligonucleotide covers the entire region synthesized by the polymerase. In this embodiment, a controller oligonucleotide can completely detach the P1-Ext from the template. No free nucleotides remain in the 3′ segment of the P1-Ext for interaction with the template during the controlling phase (see below).

In certain embodiments, the length of the controller oligonucleotide and the length of the expected primer extension product are matched in such way that the length of the synthesized region of the primer extension product exceeds the length of the controller oligonucleotide. The 3′ segment of the primer extension product thus comprises a sequence segment which is not verified by the controller oligonucleotide. Thus, when the P1-Ext interacts with the controller oligonucleotide, a double-stranded segment of P1-Ext and template remains, which is not verified by the controller oligonucleotide. This double-stranded segment can dissociate spontaneously under the reaction conditions of the controlling phase, e.g. as a result of a temperature-related strand opening (e.g. at temperatures of approx. 50-80° C.). The length of this 3′-segment of the primer extension product (without interaction with controller oligonucleotide) can comprise for example sequence lengths which are in the following ranges: from 1 to 10 nucleotides, from 10 to 20 nucleotides, from 30 to 40 nucleotides, from 40 to 50 nucleotides, from 50 to 70 nucleotides, from 70 to 100 nucleotides.

The synthesized primer extension product is now to be detached from the template by means of controller oligonucleotide and thus made available for further steps.

Controlling Phase (FIGS. 4, 7-11):

The goal of the controlling phase is to verify the synthesized primer extension product for matching with the given sequence. The sequence dependence of the double-strand formation of primer extension product and controller oligonucleotide is used for verification of the synthesis quality during the primer extension reaction.

As a result of the controlling phase, the primer extension product is completely or partially detached from the template depending on the sequence. Thereby formation of a double strand comprising primer-extension product/controller oligonucleotide takes place simultaneously.

Thus, the successful formation of the complex of primer extension product and controller strand (P1-Ext/Controller) is a prerequisite for the opening of the double strand (from template/primer extension fragment) after primer extension. In this embodiment, the control of the sequences takes place not until after the synthesis of the primer extension product in a separate reaction step (controlling phase). The controller oligonucleotide should form a double strand with the P1-Ext synthesized in the synthesis phase.

Preferably, controller oligonucleotide is already in the reaction batch at the beginning of the synthesis reaction. Due to an excess of the oligonucleotide primer, the controller oligonucleotide is present as a complex comprising primer/controller.

The formation of the complex of controller oligonucleotide/primer extension product requires a release of the controller oligonucleotide from the primer/controller complex. For this purpose, for example, the temperature of the reaction batch can be increased such that the primer/controller complexes become thermodynamically unstable. However, the reaction temperature should be chosen in such way that the completely double-stranded complexes of primer extension product and template strand do not spontaneously disintegrate.

By increasing the reaction temperature, the controller oligonucleotides are released from complexes (primer/controller) and are in equilibrium with the new formation of these complexes (e.g. at temperatures around the Tm of the complexes of primer/controller±3 to 8° C.).

The controller oligonucleotides now freely available in the batch can interact with complexes of primer extension products and template strands in the second region (primer overhang). The interaction starts from the primer overhang. This creates a spatial proximity between the controller strand and the end of the double-stranded region between primer extension product/template strand. This allows the double strand formation to transfer between the controller and the first region of the primer (sequence dependent strand displacement). For a given sequence matching with the synthesized region of the primer extension product, the double strand formation between the controller oligonucleotide and the primer extension product continues to propagate.

The progression of said interaction between the synthesized primer extension product and the controller oligonucleotide leads to an increasing release of the template strand from the complex with the primer extension product. Preferably, when there is sufficient matching in complementarity, dissociation of the complex of the template and primer extension product occurs. A new complex is formed which comprises the primer extension product and the controller oligonucleotide.

In certain embodiments, a 3′ segment of the primer extension product remains in binding to the template strand, even if the controller oligonucleotide has fully bound to the primer extension product. The dissociation of this complex (comprising the 3′-segment of the primer extension product and the respective complementary regions of the template) depends on the chosen reaction conditions during the controlling phase (see below). The successful interaction of components in the interaction of the controller oligonucleotide with the synthesized primer extension product and the spontaneous dissociation of the complex of the 3′-segment of the P1-Ext and the template can lead to the separation of the primer extension product (in the complex of P1-Ext and Controller strand) from the template.

Discriminatory effect of the controller oligonucleotide:

However, if there are any deviations in the complementarity between the P1-Ext and the controller oligonucleotide, the process of sequence-dependent strand displacement can be massively hindered. The higher the sequence differences between the synthesized primer extension product and the respective controller oligonucleotide are, the more the formation of a complex of controller oligonucleotide and P1-Ext is affected. The extent of the influence can range from a slight slowdown to a complete stop in the progress of the complex formation.

Due to a reversibility in the double strand formation, the controller oligonucleotide can be completely or partially detached by the template strand from the binding with the primer extension product (displacement by template). In certain embodiments, such displacement can take place completely. This can take place, for example, if under the reaction conditions used in the controlling phase, the stability of the complex from the second region of the primer and the first region of the controller oligonucleotide is not sufficiently high and this complex can dissociate spontaneously into its partial components. Thus, the template strand acts against the controller oligonucleotide. Since the reaction conditions are such that the complex of template and P1-Ext is not supposed to dissociate spontaneously, only those primer extension products from this complex can be detached by the controller oligonucleotide which comprise a sufficiently complementary binding of a controller oligonucleotide. Such a discriminating effect can be exploited from several points of view.

Examples of potential sources of variation in primer extension products.

Deviations in the sequence of a primer extension product can be due to several causes, e.g. the presence of multiple template strands with identical or similar primer binding sites and sequence variants at one or more positions in the segment of the template strand, e.g. in the presence of a polymorphic locus in the target sequence segment. Another cause for deviations can be, for example, synthesis errors/assembly errors introduced by polymerases.

If there are sequence deviations in the primer extension products from the sequence composition of the controller oligonucleotide, such primer extension products preferably remain in double-stranded complexes with the template during the controlling phase. Thus, such double-stranded complexes comprising a template strand and a primer extension product (which could not be detached from the controller) cannot be an active form for further analysis steps. Thus, a differentiation between primer extension products can take place: primer extension products that have been detached from the template represent an active form of primer extension products. Primer extension products in complex with their template preferably represent an inactive form.

The use of a primer extension product in further reactions may require, for example, that certain regions of the P1-Ext are present in single-stranded form. This may be necessary for binding a primer or probe, for example. This embodiment can therefore be used, for example, to convert the sequences in the 3′ segment of the P1-Ext into single-stranded form.

The primer extension product is only detached from the template if the synthesis is performed correctly or if there is a certain matching in complementarity with the controller oligonucleotide. If there are any deviations in the sequence, the primer extension product remains in complex with the template and the sequences in the 3′ segment of the P1-Ext cannot be converted to the single-stranded state (under conditions used).

This discriminatory effect of the controller oligonucleotide after a primer extension reaction (synthesis phase) can be used for several technical objectives. The availability of controller oligonucleotide in a batch (homogeneous assay format) allows both a low-contamination integration into other methods of molecular analysis and a combination with other methods, e.g. with amplification methods or detection methods. Some embodiments will therefore be presented in more detail.

For example, a fully complementary primer extension product bound to a controller can be partially or fully protected from nuclease action if the nature of the controller strand is chosen accordingly. For example, when using nuclease-resistant modifications on certain sequence segments of the third region of the controller or throughout the entire third region of the controller, e.g. PTO or 2′-O-alkyl modifications, such a controller can protect the complementary primer extension product from nuclease cleavage.

In contrast, the remaining primer extension products bound to the template strand can be selectively cleaved enzymatically by the action of a nuclease (e.g. restriction endonuclease or an exonuclease). In this way specific primer extension products with a given sequence can be isolated with higher specificity.

A controller can further comprise an affinity marker, e.g. a biotin residue (e.g. covalently coupled to the 3′ end or 5′ end). Thus, a primer extension product bound to a controller can be isolated by using a solid phase coated for example with streptavidin.

Several different template strands with essentially uniform primer binding site and a uniform primer

The embodiment is based on the sequence-specific detachment/separation of the primer extension product from its template strand in the presence of several different primer extension products in the same reaction batch.

Template Strands:

In this embodiment several template strands (at least two) with essentially the same primer binding site but differing in the sequence of the sequence segment serving as template are used. The differences between template strands are preferably located in the 3′-direction from the 3′-end of the primer. The differences may comprise, for example, single nucleotide variations (e.g. SNVs: substitutions, deletions, insertions), or also insertions, deletions, duplications, inversions of sequence segments with several nucleotides (e.g. 2 to 200 nucleotides).

Thus a template strand comprises a target sequence which comprises at least one polymorphic locus and further comprises at least one sequence segment which is uniform for all sequence variants of a target sequence. For example, the primer binding site may be located in the uniform sequence segment. The polymorphic locus is thus located in the 3′ direction from the 3′ primer end.

Primer:

Preferably, a uniform oligonucleotide primer oligonucleotide is used, which is able to bind predominantly complementarily to uniform primer binding sites of all template strands present in the batch and initiate the start of a synthesis reaction by a polymerase.

Controller:

At least one controller oligonucleotide is used which is constructed in its third region in such way that it is capable of binding only with a specific variant of the sequence of a possible primer extension product complementarily to the synthesized region of the primer extension product. If several controller oligonucleotides are used, each controller oligonucleotide is constructed sequence-specifically to a particular variant of the sequence.

Synthesis Phase:

During the synthesis phase, primer binding (by the first region of the oligonucleotide primer) can occur essentially without discrimination between individual template strands. The polymerase is capable of initiating synthesis from such bound primers and synthesizes complementary strands based on the respective template strand. This results in primer extension products which are essentially complementary to the respective template strand and thus comprise complementary segments to individual sequence variants of the template strands. During synthesis, the polymerase cannot distinguish between these sequence differences of the template strands.

The controller oligonucleotide is located in the primer/controller complex in double-stranded form during the synthesis phase and does not interfere with the synthesis procedure.

Controlling Phase (FIGS. 4, 7-11):

After completion of the synthesis phase, the release of the controller oligonucleotide from the double-stranded complex primer/controller takes place, e.g. by increasing the temperature (at a temperature at which the primer/controller complexes are at least partially dissociated, but complexes comprising primer extension products/template strands are essentially stable). The controller oligonucleotide, which is now at least partially in single-stranded form, can interact with the second region of the primer through its first region, thereby initiating strand displacement. The progress of the formation of the double strand between the first primer extension product and the controller oligonucleotide depends on the matching of the sequences of the third region of the controller oligonucleotide and the synthesized portion of the respective primer extension products. The second region of the controller is fully complementary to the first region of the first primer.

The synthesis products (primer extension products) differ depending on the template strand used. These differences can range from differences in only one nucleobase to completely different sequence composition of the primer extension product. Due to these differences in the primer extension products, the result of the controlling phase is different. Primer extension products with a sufficient matching in complementarity with the third region of the controller are predominantly detached from their template strand. Thus, a differentiation between synthesized primer extension products is made in the controlling phase: The controller oligonucleotides preferably form double-stranded complexes with complementary primer extension products. In case of sequence deviation, this double-strand formation does not take place sufficiently. Due to selected reaction conditions (a temperature at which primer/controller complexes are at least partially dissociated, but stable complexes comprising primer extension products/template strands are present), primer extension products with sequence differences to the controller strands remain predominantly bound to their template strands.

The now completely or partially detached primer extension product can be used in further analyses. For example, it can be isolated from the reaction mixture (in bound form to the controller oligonucleotide). It can also be used as a binding partner for other oligonucleotides, e.g. probes. Furthermore, it can itself act as a template strand, e.g. after binding a new primer in the 3′ segment of the primer extension product.

Several Different Template Strands with Allele Discriminating Primers

The embodiment is based on the sequence-specific detachment/separation of the primer extension product from its template strand in the presence of several different primer extension products in the same reaction batch.

Template Strands:

Also in this embodiment several template strands (at least two) with one primer binding site are used, wherein the primer binding site differs for individual templates. The differences between template strands are preferentially located at a position that preferentially interacts with the 3′ segment of the primer (for example positions 0, −1, −2, −3, −4, −5). The differences may comprise, for example, single nucleotide variations (e.g. SNVs: substitutions, deletions, insertions), or insertions, deletions, duplications, or inversions of sequence segments of several nucleotides (e.g. 2 to 10 nucleotides).

First Oligonucleotide Primer:

Several first oligonucleotide primers (more than two) are used in the primer extension reaction. The respective first oligonucleotide primers are constructed in their 3′ segment of the first region of the primer strand in such way that each primer oligonucleotide used comprises a sequence that is specific and complementary to a particular sequence variant of the template strands in question (e.g. allele-specific primers). Such first primers are capable of predominantly complementary binding to their specific primer binding sites of the respective template strands and of initiating a start of a synthesis reaction by a polymerase.

The first oligonucleotide primers can comprise uniform or different second regions (primer overhang, oligonucleotide tail). The composition of the respectively specific controller oligonucleotides in the respective first region is adjusted in such a way that a specific pairing of an allele-specific first primer and a corresponding controller is possible and each controller in such a pair comprises a specific base sequence in the first region of the controller and each first primer oligonucleotide in such a pair comprises a corresponding complementary sequence in the second region. This allows the oligonucleotide primers to interact with controller oligonucleotides predominantly specifically during the controlling phase.

Competitor-Oligonucleotide-Primer (FIGS. 21-24):

In the primer extension reaction, at least one additional primer can be used which is not a first oligonucleotide primer. Such a primer is called competitor-oligonucleotide-primer. It is capable of binding to at least one sequence variant of a polymorphic locus of a target sequence complementarily and thereby forming a perfect match duplex, wherein said target sequence is preferably a sequence which is capable of forming a mismatch with the first oligonucleotide primer at least at one site.

The respective competitor-oligonucleotide-primers are preferably constructed in their 3′ segment in such way that each competitor-oligonucleotide-primer used comprises a sequence which is specific and complementary to a particular sequence variant of the template strands in question (e.g. allele-specific primers). Such competitor primers are able to bind predominantly complementarily to their specific primer binding sites of the respective template strands and to initiate the start of a synthesis reaction by a polymerase.

Essentially, competitor-oligonucleotide-primers are also allele-specific primers which do not interact with the first region of the respectively specific controller oligonucleotide and thus cannot initiate strand separation.

Competitor primers are essentially intended to compete with the first primer-oligonucleotide for binding to the primer-binding site of the template strand and, if they bind to its complementary variant of the template strand in an essentially complementary manner, to be extended by a polymerase so that a competitor-primer extension product is formed.

The choice of the sequence of a competitor-oligonucleotide-primer is mainly based on the need to specifically exclude a specific sequence variant of a polymorphic locus from the interaction with the first oligonucleotide primer and thus to prevent or minimize the unspecific interaction between a specific first oligonucleotide primer and a primer binding site of another sequence variant which deviates in sequence.

A competitor-oligonucleotide-primer preferably comprises no sequence segment portion which can interact with the controller oligonucleotide specific for the first oligonucleotide primer.

Controller:

At least one controller oligonucleotide is used which is constructed in its second region in such way that it is capable of binding only with a specific variant of the sequence of a sequence-variant-specific first oligonucleotide primer and thus only with a specific variant of the sequence of a possible first primer extension product complementary in the first segment of the primer extension product. If several controllers are used, each controller oligonucleotide is constructed sequence-specifically for a particular variant of the sequence.

Several controller oligonucleotides may be used, which further differ in that they comprise in their first region a specific sequence which is capable of predominantly complementarily binding to the specific first oligonucleotide primer. Thus, multiplex coding with different primer/controlling oligonucleotide pairs is possible. Both partners comprise respectively sequence segments specific for said pair in the first primer region and in the second primer region, as well as respectively in the first and in the second region of the controller oligonucleotide.

Synthesis Phase:

During the synthesis phase, primer binding (by the first region of the first oligonucleotide primer) can take place essentially/preferably by discriminating between the individual sequence variants of template strands. For this purpose, suitable reaction conditions are preferably used (so-called stringent reaction conditions). The polymerase is capable of starting a synthesis from such correctly complementarily bound primers and synthesizes respectively complementary strands based on the respective template strand. This results in primer extension products which are essentially complementary to the respective template strand. During synthesis, the polymerase cannot distinguish between these differences of the template strands. The controller oligonucleotide is located in the primer/controller complex in double-stranded form during the synthesis phase and does not interfere with the synthesis procedure.

In the embodiment with competitor oligonucleotides, a competitor-primer binding and extension also takes place in the synthesis phase:

During the synthesis phase, the competitor-primer binding can essentially/preferably take place by discriminating between the individual sequence variants of template strands. For this purpose, suitable reaction conditions are preferably used (so-called stringent reaction conditions). The polymerase is able to start a synthesis of such correctly complementary bound competitor primers and synthesizes respectively complementary strands based on the respective template strand. This results in competitor-primer extension products which are essentially complementary to the respective template strand. During synthesis, the polymerase cannot distinguish between these differences of the template strands.

Controlling-Phase (FIGS. 4, 7-11):

After completion of the synthesis phase, the release of the controller oligonucleotide from the double-stranded complex primer/controller takes place, e.g. by increasing the temperature (at a temperature at which the primer/controller complexes are at least partially dissociated, but stable complexes comprising primer extension products/template strands are present). The controller oligonucleotide, which is now at least partially in single-stranded form, can interact with the second region of the primer through its first region and thus initiate strand displacement. The progress of the formation of the double strand between the primer extension product and the controller depends on the matching of the sequences of the second region of the controller and the synthesized portion of the respective primer extension products.

The synthesis products (primer extension products) differ depending on the primers and template strands used. Due to these differences in the primer extension products, the result of the controlling phase is different. Primer extension products with a sufficient matching in complementarity with the second region of the controller oligonucleotide are mainly detached from their template strand. This results in discrimination between the synthesized primer extension products in the controlling phase: The controller oligonucleotides preferably form double-stranded complexes with complementary primer extension products. In case of sequence deviation, this double-strand formation takes place insufficiently. Due to the chosen reaction conditions (at a temperature at which primer/controller complexes are at least partially dissociated and stable complexes comprising primer extension products/template strands are present), the primer extension products with sequence differences to controller strands remain predominantly bound to their template strands.

The specifically formed competitor-primer extension products cannot be released from their template strands by the action of a controller because the competitor primers lack respective second regions (primer overhang) which could interact complementarily with the first region of the controller.

The now completely or partially detached sequence-specific primer extension product can be used in further analyses. For example, it can be isolated from the reaction mixture (in bound form to the controller oligonucleotide). It can also be used as a binding partner for other oligonucleotides, e.g. probes. Furthermore, it can itself act as a template strand, e.g. after binding a new primer in the 3′ segment of the primer extension product.

The specific primer extension products synthesized in a primer extension reaction can, for example, be used as a start nucleic acid in subsequent amplification methods.

Synthesis of the Primer Extension Product and Interaction with the Controller Take Place in Parallel (On-Line Controlling)

A faulty synthesis of primer extension products by polymerases poses a significant problem for genetic analyses. For example, individual nucleotides are exchanged by the polymerase during synthesis (e.g. instead of a correct C, a T is inserted, or instead of an A, a G is inserted, etc.). Said polymerase errors can falsify the results of the analysis, as they alter the content of the sequences. The present embodiment is intended to reduce the synthesis errors in fully synthesized primer extension products in a primer extension reaction.

The technical objective of this embodiment is solved by the fact that the synthesis of a primer extension product and the elimination of the incorrectly synthesized strands takes place in parallel to synthesis. The elimination of the incorrectly synthesized strands takes place by separating the primer extension product from its template before the primer extension product has reached its full length. This separation of the primer extension products takes place with the participation of a controller oligonucleotide.

The synthesis of the primer extension product and the verification by means of a controller oligonucleotide take place in the same batch (homogeneous assay), wherein the individual reactions are essentially parallel to each other. Thus, the controller oligonucleotide takes influence on the synthesis during the procedure. The synthesis of the primer extension product (P1-Ext) during the synthesis phase and the opening of the double strand by means of a controller oligonucleotide (controlling phase) are adapted to each other in such way that predominantly a correct primer extension that takes place during the process in a complete synthesis results in a primer extension product that is complementary to the template strand. In contrast, in a non-processed, mainly defective primer extension or in a synthesis with a defect introduced by the polymerase into the synthesized strand, the partially extended primer is predominantly prematurely detached from the template strand by the controller oligonucleotide, wherein the synthesis of the respective primer extension product is thereby prematurely interrupted. The product of the prematurely interrupted synthesis is preferably bound to a controller oligonucleotide and forms with it a double-stranded complex which is stable under reaction conditions. Thus this incomplete product is removed from the further primer extension reaction. Preferably, complete primer extension products should be provided which comprise a higher complementarity to the template strand.

As a result, complete primer extension products should be synthesized with higher precision. The defective primer extension products comprising at least one insertion error introduced by a polymerase during the synthesis should be selected.

In the following, a preferred embodiment with a homogeneous format is described, in which at least one template strand, at least one controller oligonucleotide, at least one oligonucleotide primer, at least one polymerase and dNTPs are present as substrates in a suitable buffer system at the beginning of the reaction.

Synthesis Phase:

The aim of the synthesis phase is to synthesize a specific primer extension product by polymerase-mediated synthesis using a template strand.

A primer extension reaction comprises a preferably specific primer binding (first region of the oligonucleotide primer) to the template strand at a suitable primer binding site (sequence segment capable of complementarily binding the first region of the oligonucleotide primer), wherein the second region of the primer (primer overhang) preferentially does not bind to the template strand and is thus available for later interaction with the controller. The primer binding site can be completely single-stranded or partially single-stranded, so that at least part of the first region of the primer can bind sequence specifically to the primer binding site by forming complementary base pairs (Watson-Crick rule).

The polymerase can extend such oligonucleotide primers bound to the primer binding site in a template-dependent synthesis by attaching complementary nucleotides to the growing strand of the primer (3′-OH end). This results in a primer extension product (P1-Ext) that is complementary to the template strand. The synthesis of the complementary strand generally takes place as long as the polymerase finds sufficient substrate in the batch and until sufficient sequence segments in the template strand are available in a suitable form for the polymerase as a template. Depending on the polymerase, one of the conditions for the sequence segment of the template can be that the template strand is single-stranded (using polymerases without strand displacement properties) or partially double-stranded (using polymerases with strand displacement properties). The synthesis reaction is often carried out over a sufficient time (total reaction time) so that a complete primer extension product can be synthesized on more than 1%, particularly on more than 10%, particularly on more than 30%, particularly on more than 50% of the template strands.

In a preferred embodiment, primer binding takes place under reaction conditions that do not allow thermodynamically stable binding of the first region of the oligonucleotide primer to the respective complementary position in the template. There is thus an equilibrium of binding events and dissociation events between the first region of the oligonucleotide primer and the respective primer binding site in the template strand. During binding events, complexes are formed comprising the first regions of the primer and the respective primer-binding sites on the template strand. During dissociation events, these complexes separate into individual components so that no primer extension reaction can be initiated by polymerase. Such conditions are present, for example, at reaction temperatures that lie around the melting temperature (Tm) of such a complex (P1/template strand). The melting temperature (Tm) should be measured under identical buffer conditions and concentrations. For example, the reaction can be carried out at reaction temperatures in the range from Tm−5° C. to Tm+30° C., in special embodiments in the range from Tm−5° C. to about Tm+20° C., in a further specific embodiment in the range from Tm−5° C. to Tm +10° C. Even under such conditions, extension of the primer by a polymerase can take place with sufficient complementarity of the 3′ segment of the primer with the template.

The structure of the primers and controller oligonucleotides as well as the reaction conditions of the synthesis phase are such that the complex comprising primer/controller oligonucleotide is located predominantly in equilibrium with its individual components (complex formation and complex dissociation take place parallel to each other). Such conditions are present, for example, at reaction temperatures which are located around the melting temperature (Tm) of such a complex (primer/controller oligonucleotide). The melting temperature (Tm) should be measured under identical buffer conditions and concentrations. For example, the reaction can be carried out at reaction temperatures in the range from Tm−5° C. to Tm+30° C., in specific embodiments between about Tm−5° C. to about Tm+20° C., in a further specific embodiment between about Tm−5° C. to about Tm+10° C., in specific embodiments between about Tm−5° C. to about Tm+5° C., in a further specific embodiment between about Tm−5° C. to about Tm−2° C. The choice of such conditions can be determined experimentally in individual cases by measuring the melting temperatures (Tm) for such complexes.

Even if the primer is used in excess to the controller oligonucleotide, preferably no complete saturation of the controller oligonucleotide by the primer oligonucleotide takes place.

Under such reaction conditions, both formation and dissociation of primer/primer binding site complexes and formation and dissociation of primer/controller oligonucleotide complexes take place. The structures and reaction conditions are adapted to each other in such way that under such conditions extension of the primer by a polymerase can take place with sufficient complementarity of the 3′-segment of the primer with the template.

Advantageous here are combinations of structures of the individual components and the reaction conditions which, even with a primer extension of only a few nucleotides, lead to that even an incompletely extended primer extension product can be more firmly bound to the controller oligonucleotide than an as yet unextended primer. This generally takes place in that the third region of the controller oligonucleotide can undergo complementary binding of a controller oligonucleotide to a primer extension product and thus comprise additional binding positions compared to a primer/controller complex. Due to such a more stable binding, even incompletely extended primers can form complexes with controller oligonucleotides and are withdrawn from the reaction due to sufficient stability of these complexes. Since the controller oligonucleotide does not support template function (e.g. by modification), incompletely extended primers bound to the controller oligonucleotide are not completed. For this reason, preferably the entire third region of the controller oligonucleotide comprises such modifications. The continuation of the synthesis at such incompletely extended primer/controller complexes can only take place when they are released again from the complex with the controller oligonucleotide. However, due to advantageous reaction conditions, this takes place only to a very limited extent. Thus, incompletely extended primer extension products can be bound during the synthesis reaction in complexes with controller oligonucleotides.

The controller oligonucleotide can interact with primer oligonucleotides, partially extended primers and fully extended primers during the primer extension reaction (parallel to said reaction). This interaction starts at the second region of the primer.

Polymerases:

In said preferred embodiment, sufficiently processive polymerases are predominantly used, e.g. Bst-Large-Fragment, Phi29 polymerase or polymerases with improved processivity (e.g. phusion polymerase is a result of coupling a thermostable polymerase with a protein which prolongs the binding of the polymerase to the DNA) or their modifications. As a result, strand synthesis takes place rapidly, so that any errors in incorporation that occur during synthesis are also quickly overcome. Preferably, controller oligonucleotides are used in concentrations of about 0.1 μM to about 20 μM. This allows sufficiently frequent interaction with primer extension products, so that an influence on the quality of the primer extension products can be expected.

In another embodiment, polymerases with lower processivity (e.g. Taq Polymerase, Vent-Polymerase, Pfu-Polymerase) are used. Thus the synthesis generally proceeds more slowly and requires repeated binding events of the polymerase. Therefore, controller concentrations of about 5 nM to about 1 μM are preferred.

Polymerases are predominantly capable of binding several nucleotides (e.g. more than 15 nucleotides, better more than 30 nucleotides, preferably more than 50 nucleotides) to the growing end of the primer or primer extension product in only one binding event to a primer/template strand complex. In contrast, low-processive polymerases, such as Taq polymerase and Klenow polymerase, or entirely distributive polymerases, attach only a few nucleotides at a binding event (generally less than 8 to 15 nucleotides). A predominantly processive synthesis is thus associated with the synthesis within a binding event of the polymerase to the primer template complex.

Furthermore, the high processivity of the polymerase is favoured by sufficient complementarity of the 3′-end of the growing primer extension product. Generally, the synthesis takes place at high speed and is particularly advantageous when the 3′-end of the strand to be extended fulfils a structural requirement for complementarity specified by the template (this generally refers to complementary, perfect match situations in nucleobases between the 3′-end or 3′-segment of the strand to be extended and the respective template). Deviations from this, i.e. mismatches between the 3′-end or 3′-segment of the strand to be extended (e.g. positions −1 to −5) and the template strand, generally lead to a more or less pronounced deceleration or even pause of the synthesis by the polymerase. This slowing down in the catalysis speed of the polymerase is known to a skilled person. The extent of such a slowdown or pause in the catalysis depends on the polymerase as well as on the reaction conditions (e.g. concentration of dNTPs) as well as on the type of mismatch. Continuing the synthesis beyond such a mismatch generally requires a longer reaction time.

Since all polymerases perform template-dependent catalysis with only limited precision, a more or less pronounced mis-synthesis of a primer extension product takes place with each primer extension. During a template-dependent synthesis, misincorporation events happen (e.g. a dCTP is incorporated instead of a dTTP versus an adenosine in the template strand). This results in more or less pronounced pauses in the synthesis.

Using a sufficiently long reaction time, such a mismatch position (generally comprising one or two nucleotides) can be overcome by the polymerase and template-dependent synthesis can generally be continued. The extent of overcoming depends on both the type of mismatch and the polymerase.

Polymerases can comprise a 3′-exonuclease activity (proofreading polymerases, e.g. phusion polymerase) or not (Bst-large fragment). Depending on the type of template used, DNA-dependent polymerases or RNA-dependent polymerases can be used.

Template Strand:

The template strand comprises a primer binding site so that under selected reaction conditions binding of the first region of the primer can take place and polymerase mediated synthesis of the strand can take place. Furthermore, a template strand comprises a target sequence. Said target sequence generally represents the sequence segment of the template strand, which takes over the template function for the synthesis of the complementary strand. Preferably, the template strand and the oligonucleotide primer are designed to be matched to each other in such way that the second region of the oligonucleotide primer does not bind to the template strand or at least does not form a stable bond and is thus potentially capable of interacting with the first region of the controller oligonucleotide. In general, both DNA and RNA can occur as template strands.

The primer binding site of the template is preferably located in a single-stranded form and allows sequence-specific binding of the first region of the oligonucleotide primer to the template.

The segment of the template strand, which serves as the template for the synthesis of the complementary strand by the polymerase, can be completely or predominantly (more than 50%) single-stranded in an embodiment under reaction conditions. In an embodiment of another type, said segment is predominantly (more than 50%) double-stranded. The polymerases used in the synthesis must be selected respectively. In single-stranded templates, polymerases without pronounced strand displacement can be used, e.g. phusion polymerase. For predominantly double-stranded segments of the template, polymerases with strand displacing properties are preferred, e.g. Bst-Polymerase Large Fragment.

Primer:

In certain embodiments, the primer binding site for the primer can be located in the 3′ segment of the template. In such an embodiment, it is advantageous if the oligonucleotide primer comprises a modification that prevents the polymerase from copying the second region of the primer.

In another embodiment, the template can extend significantly beyond the primer binding site of the primer in its 3′ segment. The length of such an overhang can range from a few nucleotides to several kilobases. The structure of such a template strand means that the primer overhang is not copied by the polymerase, starting from the 3′-end of the template. Therefore, in such an embodiment the primer does not necessarily have to comprise a block group in the second region of the primer.

The synthesis of the primer extension product takes place via the polymerase as template-dependent synthesis. It is advantageous if the progress of the synthesis of the primer extension product is limited and thus a desired primer extension product is limited in length.

The length of the complete primer extension product is preferably limited by the availability of a sequence segment of the template which can be used by the polymerase for the synthesis. This can be achieved, for example, by limiting the template strand in the 5′ segment. This limitation prevents the polymerase used from continuing the synthesis. Such a limitation can be achieved, for example, by a strand end (strand termination achieved e.g. by physical action or by an enzyme, e.g. an endonuclease or a restriction endonuclease) or by using one or more modifications in the strand (e.g. e.g. spacers or linkers) which prevent the polymerase from synthesizing, or by nucleotide modifications (e.g. nucleobase modification, such as Iso-C or Iso-G, or the sugar-phosphate portions, such as 2′-O-Me, 2′-MOE, morpholino, PNA etc.).

By using long template strands, e.g. genomic DNA or mRNA or long PCR fragments, it is advantageous to stop or halt the synthesis of the polymerase by using additional oligonucleotides at predetermined segments. In an embodiment of this kind, the progress of the synthesis of the primer extension product is limited by the use of a so-called block oligonucleotide. Such a block oligonucleotide can hybridize under reaction conditions of the synthesis phase preferably predominantly sequence-specifically to the template strand via complementary binding. Such a block oligonucleotide, when bound to the template strand, prevents the polymerase from reading off the template strand beyond said oligonucleotide. Depending on the type of polymerase used and its configuration, different oligonucleotides can be used. The selection of the oligonucleotide structure, the polymerase and the reaction conditions are based on the fact that the progressive synthesis is prevented or blocked by the polymerase.

Examples of such oligonucleotides leading to a stop are known to a skilled person.
An example constitutes the prevention of polymerases with strand displacement properties. Oligonucleotides with several double strand stabilizing modifications, e.g. LNA or PNA, generally result in the polymerase being unable to continue the synthesis via the binding site of the oligonucleotide. For example, oligonucleotides complementary to the template with a length of approx. 20-50 nucleotides with approx. 7-30 LNA modifications can prevent, for example, a Bst polymerase (large fragment) from detaching this oligonucleotide from the template via polymerase-dependent strand displacement.

The length of the complete P1-Ext generated during template-dependent synthesis and the length of the matching controller oligonucleotide can be selected differently.

In certain embodiments, the length of the controller oligonucleotide and the length of the expected fully synthesized primer extension product are matched in such way that the controller oligonucleotide completely covers the regions synthesized by the polymerase. In said embodiment, a controller oligonucleotide can completely detach the P1-Ext from the template. In the 3′-segment of the P1-Ext no free nucleotides remain for interaction with the template during the controlling phase (see below).

In a further preferred embodiment, the length of the controller oligonucleotide and the length of the expected primer extension product are matched in such way that the length of the synthesized region of the primer extension product exceeds the length of the controller oligonucleotide. The 3′ segment of the primer extension product thus comprises a sequence segment that is not verified by the controller oligonucleotide. Thus, when the P1-Ext interacts with the controller, a double-stranded segment of P1-Ext and template remains, which is not verified by the controller.

In certain embodiments, this double-stranded segment can dissociate spontaneously under the reaction conditions of the controlling phase, e.g. as a result of a temperature-related strand opening (e.g. at temperatures of approx. 50-70° C.). The length of this 3′-segment of the primer extension product (without interaction with controller oligonucleotide) can comprise, for example, sequence lengths that are in the following ranges: from 1 to 10 nucleotides, from 10 to 20 nucleotides, from 30 to 40 nucleotides, from 40 to 50 nucleotides, from 50 to 70 nucleotides, from 70 to 100 nucleotides.

In certain embodiments this double-stranded segment cannot dissociate spontaneously under the reaction conditions of the controlling phase, e.g. due to strand opening caused by temperature (e.g. at temperatures of approx. 50-70° C.). The length of this 3′-segment of the primer extension product (without interaction with controller oligonucleotide) can comprise, for example, sequence lengths that are in the following ranges: more than 20 nucleotides, more than 30 nucleotides, more than 40 nucleotides, more than 50 nucleotides, more than 100 nucleotides, more than 200 nucleotides, more than 500 nucleotides. The maximum length can reach up to about 10,000 nucleotides.

Controlling Phase (FIGS. 4, 7-11).

The aim of the controlling phase is to verify that the synthesized primer extension product matches the given sequence during the synthesis process (real-time control). The correct, fully complementary primer extension products should be synthesized in full length. Thereby, the polymerase should exclude the misincorporation events occurring during the synthesis from the further extension reaction. In particular, mismatches between primer extension products during the synthesis should be prevented by a polymerase from extending the template strand. Therefore, a complex of such a mismatch comprising primer extension product and a controller oligonucleotide is formed during the primer extension reaction. The terminal mismatch comprising primer extension products bound to the controller oligonucleotide should preferably remain in said complexes under reaction conditions. Thus, primer extension products in which the progress of the synthesis reaction is slowed down (e.g. due to mismatch incorporation by polymerase, i.e. polymerase defects) are prematurely detached from their template strand before their full length synthesis can be completed. This prevents the complete synthesis of a faulty primer extension product.

Repeated interactions between the controller oligonucleotide and the oligonucleotide primers as well as between the controller oligonucleotide and the primer extension products (the interaction begins with the binding of a controller to the primer overhang) during their formation lead to competition between the synthesis procedure and a strand displacement procedure by the controller oligonucleotide. Said reactions can be adapted to each other by changing the concentrations of the individual components. For example, the concentration of controller oligonucleotides can be varied between 0.001 μmol/l and 50 μmol/l, particularly between 0.01 μmol/l and 10 μmol/l. Thus, for example, the extent of controller-dependent strand displacement can be influenced while keeping the primer extension reaction constant.

The controller oligonucleotides freely available in the batch can interact with the second region (primer overhang). Among other things, this leads to interactions with complexes comprising primer extension products and template strands. The interaction starts from the primer overhang. This creates a spatial proximity between the controller strand and the end of the double-stranded region between primer extension product/template strand. This allows the double strand formation between the controller oligonucleotide and the first region of the primer to transfer (sequence-dependent strand displacement). For a given sequence matching with the synthesized region of the primer extension product, the double strand formation between the controller oligonucleotides and the primer extension product continues to propagate.

The progression of this interaction between the synthesized primer extension product and the controller oligonucleotide leads to an increasing release of the template strand from the complex with the primer extension product. An incomplete primer extension product leads to dissociation of the complex of template and incomplete primer extension product. A new complex of primer extension product and the controller oligonucleotide is formed. The progression of the template-dependent synthesis of such a primer extension product is thus prevented.

The use of process polymerases generally results in the primer extension reaction by the polymerase with complete matching of the synthesized growing strand to its template being faster than the formation of a double-stranded complex of primer extension product and the controller oligonucleotide.

Furthermore, the time delay of the enzymatic synthesis is exploited, which generally happens in a strand extension process if this strand comprises a mismatch as a result of a polymerase error. Strands comprising a perfect match are generally synthesized faster than those comprising a mismatch in the primer extension product.

This results in a temporary slowdown or even a pause in the synthesis after a mismatch in the growing strand, so that the procedure of double strand formation between the controller oligonucleotide and the primer extension product can detach this “paused strand” from the template before further synthesis can take place. The binding of such an incomplete primer extension product to the controller oligonucleotide is preferably sufficiently stable under the reaction conditions used, so that this incomplete product can be withdrawn from further template-dependent synthesis by the polymerase. Since the controller oligonucleotide does not serve as a template, such an incomplete primer extension product remains in complex with the controller oligonucleotide.

In a preferred embodiment, the length of the controller oligonucleotide and the length of the expected complete primer extension product are aligned in such way that the length of the synthesized region of the primer extension product extends beyond the length of the controller oligonucleotide. The 3′ segment of the primer extension product thus comprises a sequence segment that is not verified by the controller oligonucleotide. Thus, when the P1-Ext interacts with the controller, a double-stranded segment of P1-Ext and template remains. In said embodiment, said double-stranded segment can dissociate spontaneously under the reaction conditions of the controlling phase, e.g. due to strand opening caused by temperature (e.g. at temperatures of approx. 50-70° C.). The length of this 3′-segment of the primer extension product (without interaction with the controller oligonucleotide) may comprise sequence lengths that are in the following ranges: from 1 to 10 nucleotides, from 10 to 20 nucleotides, from 30 to 40 nucleotides, from 40 to 50 nucleotides. The combined effect of the interaction of the controller oligonucleotide with the synthesized primer extension product and the spontaneous dissociation of the 3′-segment complex of the P1-Ext and the template can lead to the separation of the primer extension product (in the complex of P1-Ext and controller strand) from the template.

The 3′-segment can play a functional role in subsequent analyses/reactions. In a preferred embodiment, the formed complexes of controller oligonucleotide and incomplete primer extension product are preferably sufficiently stable under the selected reaction conditions, so that said incomplete primer extension product can be excluded from continuing its synthesis on the template strand, or it is strongly prevented from continuing the template-dependent synthesis reaction by the complex form with the controller. Thus, the synthesis of defective primer extension products up to the full synthesis length can be prevented. Due to insufficient length, such incomplete P1-Ext preferably do not comprise a required 3′-sequence segment to participate in other or subsequent reactions. Said missing 3′ segment may be missing for interaction with a probe or another primer, for example. As a result, defective primer extension products cannot provide the full required functionality in subsequent reactions, e.g. in a detection or amplification procedure.

Discriminating Effect of the Controller Oligonucleotide:

Also during simultaneous synthesis reaction and controlling phase, the interaction between the controller oligonucleotide and the primer extension product depends on the complementarity between the primer extension product and the controller oligonucleotide. In case of deviations, the procedure of sequence-dependent strand displacement can be massively hindered. The higher the sequence differences between the synthesized primer extension product and the respective controller oligonucleotide, the more the formation of a complex of controller oligonucleotide and P1-Ext is affected. The extent of the influence can range from a slight slowdown to a complete stop in the progress of the complex formation. For that reason, it is advantageous if the controller oligonucleotide is adapted to the desired primer extension product and is capable of essentially complementary binding of a controller oligonucleotide to the synthesized sequence segment of the primer extension product with its third region.

After completion of the primer extension reaction, the correctly synthesized primer extension product can be detached from the template, for example as shown in an embodiment above.

The use of a primer extension product in further reactions may require, for example, that certain regions of the primer extension product are present in single-stranded form. This may be necessary for the binding of a primer or probe. Said embodiment can therefore be used to convert the sequences in the 3′ segment of the primer extension product into the single-stranded state.

This discriminating effect of the controller after a primer extension reaction can be used for several technical objectives. The availability of controller oligonucleotides in a batch (homogeneous assay format) allows both a low-contamination integration into other methods of molecular analysis and a combination with other methods, e.g. with amplification methods or detection methods.

The specific primer extension products synthesized in a primer extension reaction can, for example, be used as a starting nucleic acid in subsequent amplification methods.

Preferred Embodiments of Structures Of Individual Components:

The primer extension reaction described here comprises at least one first primer and at least one controller oligonucleotide.

The product of the primer extension reaction, the primer extension product, can, for example, be used in a subsequent amplification reaction, which also takes place with the participation of a controller oligonucleotide.

For this reason, it is advantageous that components of the primer extension reaction (first primer and controller oligonucleotide) can also be used in an amplification reaction.

Therefore, the following section describes embodiments that can be suitable for both reactions, primer extension reaction and specific amplification. However, if necessary, different components can be used in individual reactions.

Reaction Conditions

Reaction conditions include buffer conditions, temperature conditions, reaction time and concentrations of the respective reaction components.

The reaction comprising the synthesis of the extension products can be carried out as long as necessary to produce the desired amount of specific nucleic acid sequence. The method according to the invention is preferably carried out continuously. In a preferred embodiment, the amplification reaction is carried out at the same reaction temperature, wherein the temperature is particularly located between 50° C. and 75° C. In another embodiment, the reaction temperature can also be variably controlled so that individual steps of the primer extension reaction take place at different temperatures respectively.

Particularly no helicases or recombinases are used in the reaction mixture to separate the newly synthesized double strands of the nucleic acid to be amplified.

In a preferred embodiment, the reaction mixture does not contain biochemical energy donating compounds such as ATP.

The amount of nucleic acid to be amplified at the beginning of the reaction can vary from a few copies to several billion copies in one batch. In diagnostic applications, the quantity of a template comprising target sequence may be unknown.

Other nucleic acids not to be amplified can be present in the reaction as well. Said nucleic acids can be derived from natural DNA or RNA or their equivalents. In certain embodiments, control sequences are in the same batch, which should be amplified in parallel to the nucleic acid to be amplified as well.

A molar excess of approximately 10³:1 to approximately 10¹⁵:1 (ratio primer:template) of the primers and the controller oligonucleotide used can be added to the reaction mixture, which comprises template strands.

The quantity of the target nucleic acids may not be known when the method according to the invention is used in diagnostic applications, such that the relative quantity of the primer and the controller oligonucleotide with respect to the complementary strand cannot be determined with certainty. The quantity of primer added will generally be in molar excess with respect to the quantity of the complementary strand (template) if the sequence to be amplified is contained in a mixture of complicated long chain nucleic acid strands. A large molar excess is preferred to improve the efficiency of the method.

For example, the concentrations of primer-1 and controller oligonucleotides use are present in ranges between 0.01 μmol/l and 100 μmol/l, especially between 0.1 μmol/l and 100 μmol/l, especially between 0.1 μmol/land 50 μmol/l, especially between 0.1 μmol/land 20 μmol/l. The high concentration of components can increase the speed of amplification. The respective concentrations of individual components can be varied independently to achieve the desired reaction result.

The concentration of polymerase is in ranges between 0.001 μmol/l and 50 μmol/l, particularly between 0.01 μmol/l and 20 μmol/l, particularly between 0.1 μmol/l and 10 μmol/l.

The concentration of individual dNTP substrates is in ranges between 10 μmol/l and 10 mmol/l, particularly between 50 μmol/l and 2 mmol/l, particularly between 100 μmol/l and 1 mmol/l. The concentration of dNTP can influence the concentration of divalent metal cations. If necessary, it is adjusted accordingly.

For example, Mg2+ is used as divalent metal cations. The respective anion can be CI, acetate, sulfate, glutamate, etc.

The concentration of divalent metal cations, for example, is adjusted to the optimal range for the respective polymerase and comprises ranges between 0.1 mmol/l and 50 mmol/l, particularly between 0.5 mmol/l and 20 mmol/l, particularly between 1 mmol/l and 15 mmol/l.

Enzymatic synthesis generally takes place in a buffered aqueous solution. As buffer solutions dissolved conventional buffer substances, such as Tris-HCI, Tris-acetate, potassium glutamate, HEPES buffer, sodium glutamate in common concentrations can be used. The pH value of these solutions usually is between 7 and 9.5, particularly around 8 to 8.5. The buffer conditions can be adjusted, for example, according to the recommendation of the manufacturer of the polymerase used.

Other substances, such as so-called Tm-depressors (e.g. DMSO, betaine, TPAC) etc. can be added to the buffer. Such substances reduce the melting temperature (“Tm depressors”) of double strands and can thus have a positive influence on the opening of double strands. Polymerase stabilizing components such as Tween 20 or Triton 100 can also be added to the buffer in usual amounts. EDTA or EGTA can be added in conventional quantities to complex heavy metals. Polymerase stabilizing substances, such as trehalose or PEG 6000 can be added to the reaction mixture as well.

Preferably, the reaction mixture does not contain any inhibitors of the strand displacement reaction and no inhibitors of polymerase-dependent primer extension.

In certain embodiments, the reaction mixture contains DNA-binding dyes, particularly intercalating dyes such as EvaGreen or SybrGreen. Such dyes can, if required, enable the detection of the formation of new nucleic acid chains.

The reaction mixture can further contain proteins or other substances, which for example originate from an original material and which preferably do not influence the amplification.

The reaction temperatures of the individual steps of the amplification reaction can be in the ranges from approx. 15° C. to approx. 85° C., particularly in the ranges from approx. 15° C. to approx. 75° C., particularly in the ranges from approx. 25° C. to approx. 70° C.

Generally, the reaction temperature can be optimally adjusted for each individual reaction step, such that such a temperature is achieved for each reaction step. The amplification reaction thus comprises a repetitive change of temperatures which are repeated cyclically. In certain embodiments of the method, reaction conditions for several reaction steps are standardized such that the number of temperature steps is less than the number of reaction steps. In an embodiment of the invention, at least one of the amplification steps takes place at a reaction temperature which is different from the reaction temperature of other amplification steps. Thus, the reaction does not proceed isothermally, but the reaction temperature is cyclically changed.

For example, during the amplification at least two temperature ranges are used, which are alternately induced (cyclic change of temperatures between individual temperature ranges). In certain embodiments, the lower temperature range comprises, for example, temperatures between 25° C. and 60° C., especially between 35° C. and 60° C., particularly between 50° C. and 60° C., and the upper temperature range comprises, for example, temperatures between 60° C. and 75° C., particularly between 60° C. and 70° C.

In certain embodiments, the lower temperature region comprises for example temperatures between 15° C. and 50° C., particularly between 25° C. and 50° C., particularly between 30° C. and 50° C., and the upper temperature region comprises for example temperatures between 50° C. and 75° C., particularly between 50° C. and 65° C.

In certain embodiments, the lower temperature range comprises for example temperatures between 15° C. and 40° C., particularly between 25° C. and 40° C., particularly between 30° C. and 40° C., and the upper temperature range comprises for example temperatures between 40° C. and 75° C., particularly between 40° C. and 65° C.

The temperature can be kept constant in the respective ranges or changed as a temperature gradient (decreasing or increasing).

Further explanations of temperature settings are given in detail for embodiments in the following sections.

Each induced temperature can be maintained for a certain time, resulting in an incubation step. The reaction mixture can thus be incubated for a certain time during amplification at a selected temperature. Said time can be different for each incubation step and can depend on the progress of the respective reaction at a given temperature (e.g. primer extension or strand displacement etc.). The time of an incubation step can comprise the following ranges: between 0.1 sec and 10,000 sec, particularly between 0.1 sec and 1000 sec, particularly between 1 sec and 300 sec, particularly between 1 sec and 100 sec.

By changing the temperature in such way, individual reaction steps can preferably be carried out at a selected temperature. Thus, yields of a respective reaction step can be improved. Within a synthesis cycle a temperature change or a temperature change between regions can be performed several times. A synthesis cycle can therefore comprise at least one temperature change. Such a temperature change can, for example, be executed routinely in a PCR device/thermal cycler as a time program.

In certain embodiments, an amplification method is preferred in which at least one of the steps comprising strand displacement and at least one of the steps comprising primer extension reactions take place simultaneously or in parallel and under identical reaction conditions. In an embodiment of this kind, for example, a primer extension reaction of at least one oligonucleotide primer (e.g. of the first oligonucleotide primer) can take place preferably at temperature conditions in the lower temperature ranges. In contrast, strand displacement with the participation of controller oligonucleotides and a further primer extension reaction (e.g. of the second primer oligonucleotide primer) preferably takes place in the reaction step in the upper range.

In certain embodiments, an amplification method is preferred in which at least one of the steps comprising strand displacement by the controller oligonucleotide and at least one of the steps comprising the primer extension reaction are carried out at different temperatures. In an embodiment of this type, for example, primer extension reactions of at least one oligonucleotide primer (e.g. of the first oligonucleotide and/or of the second primer oligonucleotide) can take place preferably at temperature conditions in the lower temperature range. In contrast, strand displacement with the participation of controller oligonucleotide preferably takes place in the reaction step in the upper temperature range.

In a further preferred embodiment, all steps of an amplification reaction take place under the same reaction conditions.

In such an embodiment, the amplification method can be carried out under isothermal conditions, i.e. no temperature changes are required to perform the method. In such a specific embodiment of the invention, the entire amplification reaction takes place under constant temperature, i.e. the reaction is isothermal. The time of such a reaction comprises for example the following range: between 100 sec and 30,000 sec, particularly between 100 sec and 10,000 sec, particularly between 100 sec and 1000 sec.

Individual method steps can be carried out one after the other respectively by adding individual components. In an advantageous embodiment, all reaction components required for the amplification are present in a reaction mixture at the beginning of the amplification.

The start of an amplification reaction can take place by adding a component, e.g. by adding a nucleic acid chain comprising a target sequence (e.g. a start nucleic acid chain), or a polymerase or divalent metal ions, or by creating reaction conditions necessary for the amplification, e.g. setting a required reaction temperature for one or more process steps.

The amplification can be carried out until the desired amount of nucleic acid to be amplified is reached. In an embodiment, the amplification reaction is carried out for a time that would have been sufficient to obtain a sufficient quantity of the nucleic acid to be amplified. In an embodiment, the amplification reaction is carried out over a sufficient number of synthesis cycles (doubling times), which, in the presence of a nucleic acid to be amplified, would have been sufficient to obtain a sufficient quantity.

The reaction can be stopped by various interventions. For example, by changing the temperature (e.g. cooling or heating, wherein the polymerase is disturbed in its function) or by adding a substance that stops a polymerase reaction, e.g. EDTA or formamide.

After amplification, the amplified nucleic acid chain can be used for further analyses. Synthesized nucleic acid chains can be analyzed by different detection methods. For example, fluorescence-labeled oligonucleotide probes can be used or sequencing methods (Sanger sequencing or next-generation sequencing), solid-phase analyses such as microarray or bead array analyses, etc. The synthesized nucleic acid chain can be used as substrate/template in further primer extension reactions.

In certain embodiments the progress of the synthesis reaction is monitored during the reaction. This can take place, for example, by using intercalating dyes, e.g. Sybrgreen or Evagreen, or by using labeled primers (e.g. Lux-primer or Scorpion-primer) or by using fluorescence labeled oligonucleotide probes.

The detection of the change in fluorescence during amplification is implemented in a detection step of the method. The temperature and duration of this step can be adjusted to the respective requirements of an oligonucleotide probe. For example, the temperatures of the detection step comprise ranges between 20° C. and 75° C., particularly between 40 and 70° C., and particularly between 55 and 70° C.

During the detection step, illumination of the reaction takes place with light of a wavelength that is capable of exciting a fluorophore used in the detection system (a donor or a fluorescence reporter). Signal acquisition generally takes place in parallel with excitation, wherein the specific fluorescence signal is detected and its intensity is quantified.

The amplification method can be used to verify the presence of a target nucleic acid chain in a biological material or a diagnostic material as part of a diagnostic method.

In certain embodiments, reaction conditions of at least one reaction step, in which at least one allele-specific primer is to hybridize to an allele-specific sequence variant and a primer extension reaction by a polymerase is to take place, are chosen such that a predominantly specific hybridization of such a primer to its primer binding site can take place. Such conditions can also be called stringent conditions. For example, the temperature in such a step is at Tm of the respective primer/primer binding site or the temperature is above such Tm. For example, the temperature can be at about Tm+5 to about Tm+15° C.

Preferred Embodiments of the First Oligonucleotide Primer (Primer-1)

The first oligonucleotide primer (primer-1) is a nucleic acid chain which includes at least the following regions:
A first region in the 3′ segment of the first oligonucleotide primer, which can bind to a strand of a nucleic acid chain to be amplified essentially sequence specifically
A second region coupled directly or via a linker to the 5′ end of the first primer region of the first oligonucleotide primer which comprises a polynucleotide tail suitable for binding of a controller oligonucleotide and supporting strand displacement (step c) by the controller oligonucleotide, wherein the polynucleotide tail remains essentially single-stranded under reaction conditions, i.e. does not form a stable hairpin structure or ds structures, and is preferably not copied by the polymerase.

The total length of the first oligonucleotide primer is between 10 and 80, especially between 15 and 50, especially between 20 and 30 nucleotides or their equivalents (e.g. nucleotide modifications). The structure of the first oligonucleotide primer is adapted in such a way that it can undergo reversible binding of a controller oligonucleotide under selected reaction conditions. Furthermore, the structure of the first primer oligonucleotide is adapted to its primer function. Furthermore, the structure is adapted in such a way that strand displacement can be carried out by means of the controller oligonucleotide. In total, the structures of the first and second regions are adapted to each other so that exponential amplification can be performed.

In certain embodiments of the invention, the first and second region of the primer are coupled in a conventional 5′-3′ arrangement. In certain embodiments of the invention, the coupling of both sections takes place via a 5′-5′ bond, so that the second region has a reverse direction than the first region.

The coupling of regions between/under each other takes place preferably covalently. In certain embodiments, the coupling between the first and second regions is a 5′-3′-phosphodiester coupling, which is conventional for DNA. In certain embodiments it is a 5′-5′-phosphodiester coupling. In certain embodiments it is a 5′-3′-phosphodiester coupling, wherein at least one linker (e.g. a C3, C6, C12 or a HEG linker or an abasic modification) is positioned between adjacent terminal nucleotides or nucleotide modifications of the two regions.

Individual regions can include different nucleotide modifications. Individual elements of nucleotides can be modified: Nucleobase and backbone (sugar portion and/or phosphate portion). Furthermore, modifications can be used which are missing or modified at least one component of the standard nucleotide building blocks, e.g. PNA.

In certain embodiments, a second region of the first region of the oligonucleotide primers comprises further sequences that do not bind to the controller oligonucleotide. Said sequences can be used for other purposes, e.g. binding to the solid phase. These sequences are preferably located at the 5′ end of the polynucleotide tail.

In certain embodiments, a first oligonucleotide primer may comprise a characteristic label. Examples of such labels are dyes (e.g. FAM, TAMRA, Cy3, Alexa 488 etc.) or biotin or other groups that can be specifically bound, e.g. digoxigenin.

The First Primer Region of the First Oligonucleotide Primer

The sequence length lies between approx. 3-30 nucleotides, particularly between 5 and 20 nucleotides, wherein the sequence is predominantly complementary to the 3′ segment of one strand of the nucleic acid chain to be amplified. Specifically, said regions must be capable of specifically binding to the complementary 3′ segment of a second primer extension product. It should be possible to copy this first region during reverse synthesis and it serves as a template for the second strand as well. The nucleotide building blocks are preferably linked to each other via the usual 5′-3′ phosphodieser bond or phosphothioester bond.

The first region of the primer is preferably comprised of nucleotide monomers that have little or no influence on the function of the polymerase, such as
natural nucleotides (dA, dT, dC, dG etc.) or their modifications without altered base pairing
Modified nucleotides, 2-amino-dA, 2-thio-dT or other nucleotide modifications with different base pairing.

In a preferred embodiment, the 3′-OH end of this region is preferably free of modifications and has a functional 3′-OH group that can be recognized by the polymerase. The first region of the primer is the initiator of the synthesis of the first primer extension product in the amplification process. In an additional preferred embodiment, the first region comprises at least one phosphorothioate compound, so that no degradation of the 3′-end of the primer by the 3′-exonuclease activity of polymerases can take place.

The sequence of the first region of the first oligonucleotide primer and the sequence of the second region of the controller oligonucleotide are preferably complementary to each other.

In certain embodiments, the first region of the primer or its 3′ segment can bind to the sequence segments of a target sequence.

In Certain Embodiments, an Allele-specific First Primer is Used In Combination with an Allele-Specific Controller Oligonucleotide.

The first region of the first primer can bind to the complementary position of a starting nucleic acid or a nucleic acid chain to be amplified. Particularly a first region comprises at least one sequence segment, which can bind preferably and specifically to an allele-specific sequence variant of the target nucleic acid under the reaction conditions used, wherein the polymerase is capable of extending a perfect-match complex thus formed, resulting in a first primer extension product.

The position of an allele-specific sequence in the primer comprises in certain embodiments the 3′-terminal nucleotide. In a further embodiment, the position of an allele-specific sequence in the primer comprises at least one of the positions −1 to −6 nucleotides in the 3′-terminal segment of the first region of the first primer. In another execution form, the position of an allele-specific sequence in the primer comprises at least one of the positions from −6 to at least −15 in the 3′-terminal segment of the first region of the first primer.

Such a primer further comprises sequence segments which can bind complementarily to all allele variants of a target sequence uniformly. Thus, the first region of a first allele-specific primer comprises sequence segments that are both target sequence-specific and allele-specific.

Preferably, a combination of an allele-specific primer and an allele-specific controller oligonucleotide is used, wherein the first region of the first oligonucleotide primer is fully complementary to the second region of the controller oligonucleotide.

Individual allele-specific primers can be combined into a group that covers all variants of a target sequence. Such a group of allele-specific primers comprises at least two different allele-specific primers, since a polymorphic locus at a given position in the target sequence comprises at least two sequence variants. The allele-specific primers are designed to form a perfect-match bond under stringent reaction conditions, preferably with their respective specific template, and thus use this specific perfect-match template to form the respective primer extension products under the catalytic action of the polymerase. Preferably, 3′-terminal nucleotides and/or 3′-terminal segments of allele-specific primers can be used to discriminate variants of target sequences and are adapted to the respective variants in their sequence composition in such way that such primers form a perfect match double strand under stringent conditions with the respective variant. Such perfect-match double strands can generally be well detected by a polymerase and under suitable reaction conditions primer extension occurs. The interaction of an allele-specific primer with another variant of a target sequence results in a mismatch double strand. Such mismatches generally lead to a delay in extension by a polymerase or to a slowdown of the overall reaction. In certain embodiments, allele-specific primers in the 3′ segment may comprise at least one phosphorothioate bond that protects the allele-specific primers from 3′-5′ nuclease degradation by a polymerase.

Thus, multiple allele-specific primers comprise sequence segments that are essentially identical or uniform for a group of allele-specific primers as well as sequence segments that differ among the primers of a group and are characteristic for the respective sequence variant of a target sequence. By including uniform sequence segments, such primers can hybridize to the respective target sequence under reaction conditions. Including characteristic sequence segments, a respective primer can specifically bind to a sequence variant of the target sequence forming a perfect match double strand. Preferably, the primers are designed in such a way that under the reaction conditions used, binding to a target sequence is preferred to form a perfect match double strand and binding to a target sequence is less preferred to form a mismatch double strand.

In certain embodiments, a target sequence-specific first primer (but not an allele-specific first primer) is used in combination with an allele-specific controller oligonucleotide.

The first region of the first primer can bind to the respective complementary position of a starting nucleic acid or a nucleic acid chain to be amplified. In brief, a first region comprises at least one sequence segment that can bind preferably sequence-specifically under used reaction conditions to sequence segments of a target nucleic acid chain (e.g. comprising a start nucleic acid and/or the nucleic acid chain to be amplified), wherein said binding takes place essentially independently of potentially existing sequence differences in the polymorphic locus, wherein the polymerase is capable of extending a perfect-match complex formed thereby, resulting in a first primer extension product.

The binding of such a primer takes place essentially in the sequence segment of the target nucleic acid, which is uniform for at least two allele variants of these target nucleic acid chains. Preferably, primer binding takes place in the sequence segment of the target nucleic acid, which is uniform for all allele variants of this target nucleic acid.

The binding takes place in such a way that the polymorphic locus of the target sequence is located in 3′ direction from the first region of the first primer, so that in a primer extension reaction this polymorphic locus is copied by the polymerase. A resulting first primer extension product thus comprises a complementary sequence to the polymorphic locus. This sequence is located in 3′ direction from the first primer.

Such a primer thus comprises sequence segments which can bind preferentially complementary to all allele variants of a target sequence uniformly. In order for a differentiation of allele variants to take place, such a primer must be combined with at least one allele-specific oligonucleotide primer. Allele discrimination thus takes place by action of the controller oligonucleotide. The positioning of the polymorphic locus in the 3′ direction of the primer determines its localization in the third region of the oligonucleotide primer.

The Second Region of the First Oligonucleotide Primer

The second region of the first oligonucleotide primer is preferably a nucleic acid sequence which comprises at least one polynucleotide tail which preferably remains uncopied by the polymerase during the synthesis reaction and which is capable of binding with the first region of the controller oligonucleotide. The segment of the second region, which predominantly undergoes this binding of a controller oligonucleotide, can be called polynucleotide tail.

Furthermore, the second region of the first oligonucleotide primer must not only specifically bind the controller oligonucleotide under reaction conditions, but also participate in the procedure of strand displacement by means of the controller oligonucleotide. Consequently, the structure of the second region must be suitable for inducing a spatial proximity between the controller oligonucleotide and the respective double strand end (in detail, the 3′ end of the second primer extension product).

The design of the structure of the second region of the first oligonucleotide primer is described in more detail in several embodiments. The arrangement of the oligonucleotide segments and the modifications used are taken into account, which lead to a stop in the polymerase-catalyzed synthesis.

The length of the second region is between 3 and 60, particularly between 5 and 40, particularly between 6 and 15 nucleotides or their equivalents.

The sequence of the second region can be arbitrary. Preferably, it is not complementary to the nucleic acid to be amplified and/or to the second oligonucleotide primer and/or to the first region of of the first primer oligonucleotide. Furthermore, it preferably does not contain self-complementary segments such as hairpins or stem loops.

The sequence of the second region is preferably matched to the sequence of the first region of a controller oligonucleotide, so that both sequences can undergo binding under reaction conditions. In an embodiment of choice, said binding is reversible under reaction conditions: there is a balance between bound and unbound components.

The sequence of the second region of the first oligonucleotide primer is preferably such that the number of complementary bases that can bind to the first region of the controller oligonucleotide is between 1 and 40, particularly between 3 and 20, particularly between 6 and 15.

The function of the second region is, among other things, the binding of a controller oligonucleotide. In certain embodiments, this binding is preferably specific, so that a second region of a first segment of a first oligonucleotide primer can bind a specific controller oligonucleotide. In an embodiment other than this, a second region can bind more than one controller oligonucleotide under reaction conditions.

There is generally no need for a perfect match in sequence between the second region of the first oligonucleotide primer and the first segment of the controller oligonucleotide. The degree of complementarity between the second region of the first oligonucleotide primer and the first region of the controller oligonucleotide can be between 20% and 100%, particularly between 50% and 100%, particularly between 80% and 100%. The respectively complementary regions can be positioned immediately adjacent to each other or comprise non-complementary sequence segments in between.

In certain embodiments, specific sequence segments are preferably used in the second region of the first primer. These sequence-specific and thus characteristic sequence segments are preferably not complementary to the target nucleic acid chain and are adapted to the sequence segments of the first region of an allele-specific controller oligonucleotide in such way that predominantly specific complementary duplexes can be formed between the first region of the controller oligonucleotide and the second region of the first oligonucleotide primer. Thus, for example, in the presence of several controller oligonucleotides (e.g., which are allele-specific), pair formation can take place from a specific first primer and a specific controller oligonucleotide. The second region of the first oligonucleotide primer can thus be used for characteristic coding, particularly in multiplex analyses.

By using predominantly sequence-specific sequence segments in the second primer region and a simultaneous use of respective complementary sequence segments in the first region of the controller oligonucleotide, an improved assignment of potentially resulting primer extension products and characteristic controller oligonucleotides can take place.

In certain embodiments, the second region of the first oligonucleotide primers can include at least one Tm-modifying modification. By introducing such modifications, the stability of the binding between the second region of the first oligonucleotide primer and the first region of the controller oligonucleotide can be modified. For example, Tm-increasing modifications (nucleotide modifications or non-nucleotide modifications) can be used, such as LNA nucleotides, 2-amino adenosines or MGB modifications. On the other hand, Tm-lowering modifications can also be used, such as inosine nucleotides. In the structure of the second region, linkers (e.g. C3, C6, HEG-linkers) can also be integrated.

For strand displacement, the controller oligonucleotide must be placed in close proximity to the double strand end of the nucleic acid to be amplified. This double-strand end consists of segments of the first region of the first primer extension product and a respective complementary 3′ segment of the second primer extension product.

The polynucleotide tail predominantly binds complementarily to the controller oligonucleotide under reaction conditions and thus causes a temporary approaching of the second region of the controller oligonucleotide and the first region of a controller oligonucleotide extension product, so that a complementary binding between these elements can be initiated in a strand displacement process.

In certain embodiments, the binding of a controller oligonucleotide to the polynucleotide tail of the first oligonucleotide primer results in immediate contact. This means that the polynucleotide tail and the first region of the first oligonucleotide primer must be coupled to each other immediately. Thanks to such an arrangement, after binding of a controller oligonucleotide in its first region, immediate contact can occur between complementary bases of the second region of the controller oligonucleotide and respective bases of the first segment of the primer region, so that strand displacement can be initiated.

In certain embodiments, between structures of the polynucleotide tail and the first primer region, other structures of the second region of the first oligonucleotide primer are located. Thus, after binding of a controller oligonucleotide to the polynucleotide tail, the tail is not positioned immediate to the first region of the primer, but at a certain distance from it. The structures between the uncopyable polynucleotide tail and the copyable first regions of the oligonucleotide primer can generate such a distance. Said distance has a value which is between 0.1 and 20 nm, particularly between 0.1 and 5 nm, particularly between 0.1 and 1 nm.

Such structures represent linkers (e.g. C3 or C6 or HEG linkers) or segments that are not complementary to the controller oligonucleotide (e.g. in the form of non-complementary, non-copyable nucleotide modifications). The length of said structures can generally be measured in chain atoms. Said length is between 1 and 200 atoms, particularly between 1 and 50 chain atoms, particularly between 1 and 10 chain atoms.

In order that the polynucleotide tail remains uncopyable by the polymerase under amplification conditions, the second region of the first oligonucleotide primer generally comprises sequence arrangements or structures that cause the polymerase to stop the synthesis of the second primer extension product after the polymerase has successfully copied the first primer region. Said structures are designed to prevent the copying of the polynucleotide tail of the second region. The polynucleotide tail is therefore preferably left uncopied by the polymerase.

In certain embodiments such structures are located between the first region and the polynucleotide tail.

In certain embodiments, the sequence of the polynucleotide tail can include nucleotide modifications that lead to the stopping of the polymerase. Thus, a sequence segment of the second region of the first region of the oligonucleotide primer can comprise both functions: it is a polynucleotide tail as well as a sequence of nucleotide modifications leading to the stop of the polymerase.

Modifications in the second region of the first oligonucleotide primer, which lead to a synthesis stop and thus leave the polynucleotide tail uncopyable, are summarized in this application under the term “first blocking unit or first segment”.

Following further embodiments of structures are stated, which can lead to a stop in the synthesis of the second strand.

Several building blocks in oligonucleotide synthesis are known to prevent the polymerase from reading off the template and lead to termination of polymerase synthesis. For example, non-copyable nucleotide modifications or non-nucleotide modifications are known. There exist also synthesis types/arrangements of nucleotide monomers within an oligonucleotide which lead to the termination of the polymerase (e.g. 5′-5 arrangement or 3′-3′ arrangement). Oligonucleotide primers with a non-copyable polynucleotide tail are also known in the state of the art (e.g. Scorpion primer structures or primers for binding to the solid phase). Both primer variants describe oligonucleotide primer structures that are capable of initiating the synthesis of a strand such that a primer extension reaction can take place. The result is a first strand, which also integrates the primer structure with tail in the primer extension product. In the synthesis of a complementary strand to the first primer extension product, e.g. in a PCR reaction, the second strand is extended to the “blocking unit/stop structure” of the primer structure. Both described primer structures are designed in such way that the 5′ portion of the oligonucleotide primer remains single-stranded and is not copied by the polymerase.

In certain embodiments, the second region of the oligonucleotide primer comprises a polynucleotide tail, which comprises a conventional 5′- to 3′-arrangement along its entire length and includes non-copyable nucleotide modifications. Such non-copyable nucleotide modifications include, for example, 2′-O-alkyl RNA modifications, PNA, morpholino. Said modifications can be distributed differently in the second region of the primer.

The portion of non-copyable nucleotide modifications in the polynucleotide tail can be between 20% and 100%, especially more than 50% of the nucleotide building blocks. Particularly said nucleotide modifications are located in the 3′-segment of the second segment and thus adjoin the first region of the first oligonucleotide primer.

In certain embodiments, the sequence of non-copyable nucleotide modifications is at least partially complementary to the sequence in the template strand, so that primer binding to the template takes place involving at least a part of these nucleotide modifications. In certain embodiments, the sequence of non-copyable nucleotide modifications is not complementary to the sequence in the template strand.

The non-copyable nucleotide modifications are preferably covalently coupled to each other and thus represent a sequence segment in the second region. The length of said segment comprises between 1 and 40, particularly between 1 and 20 nucleotide modifications, particularly between 3 and 10 nucleotide modifications.

In certain embodiments, the second region of the first oligonucleotide primer comprises a polynucleotide tail, which comprises a conventional 5′-3′ arrangement over its entire length and includes non-copyable nucleotide modifications (e.g. 2′-O-alkyl modifications) and at least one non-nucleotide linker (e.g. C3, C6, HEG linker). A non-nucleotide linker has the function of covalently connecting adjacent nucleotides or nucleotide modifications and at the same time to interrupt the synthesis function of the polymerase in a site-specific manner.

Such a non-nucleotide linker should not separate the structures of the polynucleotide tail and the first region of the primer too far from each other. Rather, the polynucleotide tail should be in close proximity to the first primer region. A non-nucleotide linker is defined as a modification that is not longer than 200 chain atoms, particularly not longer than 50 chain atoms, particularly not longer than 10 chain atoms. The minimum length of such a linker can be one atom. An example of such non-nucleotide linkers are straight or branched alkyl linkers with an alkyl chain including at least one carbon atom, particularly at least 2 to 30, particularly 4 to 18. Such linkers are well known in oligonucleotide chemistry (e.g. C3, C6 or C12 linkers) and can be introduced during solid phase synthesis of oligonucleotides between the sequence of the polynucleotide tail and the sequence of the first region of the first oligonucleotide primer. Linear or branched polyethylene glycol derivatives are another example of such non-nucleotide linkers. A well-known example in oligonucleotide chemistry is hexaethylene glycol (HEG). Other examples of such non-nucleotide linkers are abasic modifications (e.g. THF modification, as an analog of dRibose).

If one or more of such modifications are integrated into a second region, they can effectively interfere with a polymerase in its copy function during its synthesis of the second primer extension product, so that segments located in the 3′ direction (FIG. 40) remain uncopied after such a modification. The number of such modifications in the second region can be between 1 and 100, particularly between 1 and 10, particularly between 1 and 3.

The position of such a non-nucleotide linker can be located at the 3′ end of the second region and thus represent the transition from the first region to the second region of the oligonucleotide primer.

The position of the non-nucleotide linker in the middle segment of the second region can also be used. This will divide a polynucleotide tail into at least two segments. In an embodiment like this, the 3′ segment of the polynucleotide tail includes at least one, particularly several, e.g. between 2 and 20, particularly between 2 and 10 non-copyable nucleotide modifications. Said non-copyable nucleotide modifications are preferably located at the transition between the first and second regions of the oligonucleotide primer.

In certain embodiments, the second region of the oligonucleotide primer comprises a polynucleotide tail, which comprises a 5′-to-3′-order along its entire length and includes at least one nucleotide monomer in an “inverted” 3′-to-5′-order, which is positioned at the transition between the first and second region of the first primer oligonucleotide.

In certain embodiments, the second region of the oligonucleotide primer comprises a polynucleotide tail, wherein such a polynucleotide tail consists entirely of nucleotides that are directly adjacent to the first region of the first oligonucleotide primer in an inverted arrangement such that coupling of the first and second regions takes place by 5′-5′ position. An advantage of such an arrangement is that after copying the first region, the polymerase encounters an “inverted” arrangement of nucleotides, which typically leads to termination of the synthesis at this position.

In case of an “inverted” arrangement of nucleotides in the total length of the polynucleotide tail, preferably the 3′-terminal nucleotide of the polynucleotide tail should be blocked at its 3′-OH end to prevent side reactions. Alternatively, a terminal nucleotide that does not comprise a 3′-OH group at all, e.g. a dideoxy nucleotide, can be used.

In an embodiment of this kind, the respective nucleotide arrangement in the controller oligonucleotide should of course also be adapted. In such a case, the first and second regions of the controller oligonucleotide must be linked in 3′-3′ arrangement.

In certain embodiments, the second region of the oligonucleotide primer comprises a polynucleotide tail which comprises a conventional 5′-to-3′-arrangement along its entire length and includes at least one nucleotide modification which is not a complementary nucleobase for the polymerase when the synthesis is performed with exclusively natural dNTP (dATP, dCTP, dGTP, dTTP or dUTP).

For example, iso-dG or iso-dC nucleotide modifications can be integrated as single, but particularly several (at least 2 to 20) nucleotide modifications in the second region of the first oligonucleotide primer. Further examples of nucleobase modifications are various modifications of the extended genetic alphabet. Such nucleotide modifications preferably do not support complementary base pairing with natural nucleotides, so that a polymerase does not (at least theoretically) incorporate a nucleotide from the series (dATP, dCTP, dGTP, dTTP or dUTP). In reality, however, rudimentary incorporation may still occur, particularly at higher concentrations of dNTP substrates and prolonged incubation times (e.g. 60 min or longer). Therefore, particularly several such nucleotide modifications positioned at adjacent sites should be used. The stop of polymerase synthesis is caused by the lack of suitable complementary substrates for these modifications. Oligonucleotides with Iso-dC or Iso-dG can be synthesized by standard methods and are available from several commercial suppliers (e.g. Trilink-Technologies, Eurogentec, Biomers GmbH). Optionally, the sequence of the first region of the controller oligonucleotide can be adapted to the sequence of such a second region. In this case, complementary nucleobases of the extended genetic alphabet can be integrated into the first region of the controller oligonucleotide during chemical synthesis, respectively. For example, Iso-dG can be integrated in the second region of the first primer, its complementary nucleotide (Iso-dC-5-Me) can be placed at the appropriate position in the first region of the controller oligonucleotide.

In summary, the termination of the synthesis of the polymerase in the second region can be achieved in different ways. However, said blocking preferably takes place only after the polymerase has copied the first region of the first oligonucleotide primer. This ensures that a second primer extension product has a matching primer binding site in its 3′ segment. This primer binding site is exposed during strand displacement and is thus available for rebinding another first oligonucleotide primer.

During the synthesis of the complementary strand to the first primer extension product, the primer extension reaction stops in front of the polynucleotide tail. Since this polynucleotide tail thus remains single-stranded for interaction with the controller oligonucleotide and is thus available for binding, it supports the initiation of the strand displacement reaction by the controller oligonucleotide by bringing the respective complementary segments of the controller oligonucleotide into immediate proximity with the matching duplex end. The distance between the complementary part of the controller oligonucleotide (second region) and the complementary part of the extended oligonucleotide primer (first region) is thereby minimized. Such spatial proximity facilitates the initiation of strand displacement.

In a schematic representation of a nucleic acid-mediated strand displacement reaction, a complementary sequence of controller oligonucleotide is now located immediate near the matching duplex end. Thereby, competition for binding to the first region of a controller oligonucleotide oligonucleotide occurs between the strand of the controller oligonucleotide and the template strand complementary to the primer. The nucleic acid-mediated strand displacement procedure is initiated by repetitive closure and formation of the base pairing between the primer regions and the complementary segment of the controller oligonucleotide (second region of the controller nucleotide) or the complementary segment of the template strand.

Generally, the yield of initiation of strand displacement is higher the closer the respective complementary sequence segment of the controller oligonucleotide is located to the complementary segment of the primer region. In contrast, increasing this distance decreases the yield of strand displacement initiation.

In the context of the present invention, it is not absolutely necessary that the initiation of strand displacement works with maximum yield. Therefore, distances between the 5′-segment of the first segment of the first oligonucleotide primer, which undergoes binding with a complementary strand of the template and forms a complementary duplex, and a respective complementary sequence segment in the controller oligonucleotide when bound to the polynucleotide tail of the second region of the first primonucleotide oligonucleotide can be in the following ranges: between 0.1 and 20 nm, particularly between 0.1 and 5 nm, particularly between 0.1 and 1 nm. In the preferred case, said distance is less than 1 nm. Expressed in other units, said distance corresponds to a distance of less than 200 atoms, particularly less than 50 atoms, particularly less than 10 atoms. In the preferred case, said distance is one atom. The information about the distance shall only serve for orientation and illustrate that shorter distances between said structures are preferred. In many cases, measuring this distance is only possible by analyzing the exact structures of oligonucleotides and measuring sequence distances or linker lengths.

The first primer can also comprise further sequence segments that are not needed for interaction with the controller oligonucleotide or template strand. Such sequence segments can bind further oligonucleotides, which are used as detection probes or immobilization partners when binding to the solid phase.

Primer Function of the First Oligonucleotide Primer

The first oligonucleotide primer can be used in several steps. Primarily, it takes over a primer function in the amplification. The primer extension reaction is carried out using the second primer extension product as template. In certain embodiments, the first oligonucleotide primer can use the starting nucleic acid chain as a template at the beginning of the amplification reaction. In certain embodiments, the first oligonucleotide primer can be used to create/provide a start nucleic acid chain.

During the amplification process, the first primer serves as an initiator for the synthesis of the first primer extension product using the second primer extension product as a template. The 3′ segment of the first primer comprises a sequence that can bind predominantly complementarily to the second primer extension product. The enzymatic extension of the first oligonucleotide primer using the second primer extension product as template leads to the formation of the first primer extension product. Such first primer extension product comprises the target sequence or its sequence portions. In the course of the synthesis of the second primer extension product, the sequence of the copyable portion of the first oligonucleotide primer is recognized by the polymerase as template, wherein a respective complementary sequence is synthesized, resulting in a respective primer binding site for the first oligonucleotide primer. The synthesis of the first primer extension product takes place up to and including the 5′ segment of the second oligonucleotide primer. Immediately after the synthesis of the first primer extension product, this product is bound to the second primer extension product and forms a double-stranded complex. The second primer extension product is displaced from this complex by the controller oligonucleotide sequence-specifically. Thereby the controller oligonucleotide binds to the first primer extension product. After successful strand displacement by the controller oligonucleotide, the second primer extension product can itself serve as a template for the synthesis of the first primer extension product. The now freed 3′-segment of the first primer extension product can bind another second oligonucleotide primer, so that a new synthesis of the second primer extension product can be initiated.

Furthermore, the first oligonucleotide primer can serve as an initiator for the synthesis of the first primer extension product starting from the starting nucleic acid chain at the beginning of the amplification. In certain embodiments, the sequence of the first primer is completely complementary to the respective sequence segment of a start nucleic acid chain. In certain embodiments, the sequence of the first oligonucleotide primer is only partially complementary to the respective sequence segment of a start nucleic acid chain. However, this deviating complementarity should not prevent the first oligonucleotide primer from starting a predominantly sequence-specific primer extension reaction. The respective differences in the complementarity of the first oligonucleotide primer to the respective position in the start nucleic acid chain are preferably located in the 5′ segment of the first region of the first oligonucleotide primer, so that in the 3′ segment a predominantly complementary base pairing and initiation of the synthesis is possible. For the initiation of the synthesis, for example, particularly the first 4-10 positions in the 3′ segment shall be fully complementary to the template (start nucleic acid chain). The remaining nucleotide positions may differ from perfect complementarity. The degree of perfect complementarity in the remaining 5′-segment of the first region of the first oligonucleotide primers can thus comprise ranges between 50% and 100%, particularly between 80% and 100% of the base composition. Depending on the length of the first region of the first oligonucleotide primers, the sequence variations comprise 1 to a maximum of 15 positions, particularly 1 to a maximum of 5 positions. Thus, the first oligonucleotide primer can initiate a synthesis from a starting nucleic acid chain. In a subsequent synthesis of the second primer extension product, copyable sequence segments of the first oligonucleotide primer are copied by the polymerase, so that in subsequent synthesis cycles a fully complementary primer binding site within the second primer extension product is designed for binding of the first oligonucleotide primer and is available in subsequent synthesis cycles.

In certain embodiments, the first oligonucleotide primer can be used in the preparation of a start nucleic acid chain. Such a first oligonucleotide primer can bind to a nucleic acid (e.g. a single-stranded genomic DNA or RNA or its equivalents comprising a target sequence) predominantly/preferably sequence-specifically and initiate a template-dependent primer extension reaction in the presence of a polymerase. The binding position is chosen such that the primer extension product comprises a desired target sequence. The extension of the first oligonucleotide primer results in a nucleic acid strand which comprises a sequence complementary to the template. Such a strand can be detached from the template (e.g. by heat or alkali) and thus be converted into a single-stranded form. Such a single-stranded nucleic acid chain can serve as a starting nucleic acid chain at the beginning of the amplification. Such a start nucleic acid chain comprises in its 5′-segment the sequence parts of the first oligonucleotide primer, further it comprises a target sequence or its equivalents and a primer binding site for the second oligonucleotide primer. Further steps are explained in the section “Start nucleic acid chain”.

The synthesis of the first primer extension product is a primer extension reaction and forms a substep in the amplification process. The reaction conditions during this step are adjusted accordingly. The reaction temperature and the reaction time are chosen in such way that the reaction can take place successfully. The respectively preferred temperature in said step depends on the polymerase used and the binding strength of the respective first oligonucleotide primer to its primer binding site and comprises for example ranges from 15° C. to 75° C., particularly from 20 to 65° C., particularly from 25° C. to 65° C. The concentration of the first oligonucleotide primer comprises ranges from 0.01 μmol/lto 50 μmol/l, particularly from 0.1 μmol/lto 20 μmol/l, particularly from 0.1 μmol/l to 10 μmol/l.

In certain embodiments, all steps of the amplification process are performed under stringent conditions, which prevent or slow down the formation of unspecific products/by-products. Such conditions include higher temperatures, for example above 50° C.

If more than one specific nucleic acid chain has to be amplified in an embodiment, sequence-specific oligonucleotide primers are preferred embodiments for the amplification of respective target sequences respectively.

Preferably, sequences of the first oligonucleotide primer, the second oligonucleotide primer and the controller oligonucleotide are matched to each other in such way that side reactions, e.g. primer dimer formation, are minimized. For this purpose, for example, the sequence of the first and the second oligonucleotide primer are matched to each other in such way that both oligonucleotide primers are not capable of initiating or supporting an amplification reaction in the absence of a suitable template and/or target sequence and/or starting nucleic acid chain. This can be achieved for example in that the second oligonucleotide primer does not comprise a primer binding site for the first oligonucleotide primer and the first oligonucleotide primer does not comprise a primer binding site for the second oligonucleotide primer. Furthermore, it should be avoided that the primer sequences comprise extended self-complementary structures.

In certain embodiments, the synthesis of the first and second primer extension product proceeds at the same temperature. In certain embodiments, the synthesis of the first and second primer extension products is performed at different temperatures. In certain embodiments, the synthesis of the first primer extension product and the strand displacement by the controller oligonucleotide proceeds at the same temperature. In certain embodiments, the synthesis of the first primer extension product and the displacement of the strand by the oligonucleotide controller takes place at different temperatures.

Use of a first oligonucleotide primer in combination with a first competitor-oligonucleotide-primer:

Preferred Embodiments of the First Competitor-Oligonucleotide-Primer (Primer-1)

A first competitor-oligonucleotide-primer competes with the first oligonucleotide primer for binding to a sequence segment of a nucleic acid chain comprising a target sequence which comprises at least two variants of a target nucleic acid chain.

The first oligonucleotide primer binds to the desired or expected sequence variant and is extended preferably under stringent reaction conditions by a polymerase. The first competitor oligonucleotide binds to the second potential or expected sequence variant and is extended preferably by a polymerase under stringent reaction conditions. Thus, two primer extension products are formed: one from the first oligonucleotide-primer, which is to be further multiplied in the amplification and the other from the first competitor-oligonucleotide-primer, which is not to be multiplied in an amplification.

By competition of two primers for a primer binding sequence in the target nucleic acid chain the specificity of the analysis can be improved.

The two primers (the first primer and the first competitor primer) thus represent allele-specific primers with respect to potential or expected sequence variants. The essential difference is that the competitor primer is not able to initiate strand separation with the participation of an allele-specific controller oligonucleotide.

In certain embodiments, a first competitor-oligonucleotide-primer (competitor-primer-1) comprises a nucleic acid chain which incorporates at least the following properties:
A first competitor-oligonucleotide-primer region in the 3′ segment of the first competitor-oligonucleotide-primer which can bind to a strand of a nucleic acid chain to be amplified essentially sequence-specifically and thus can compete with the first primer oligonucleotide for its primer binding site.
a 3′ end of the competitor primer, which after binding to the primer binding site in the target sequence can be extended sequence-specifically by the polymerase to form a competitor primer extension product
3′ end of the competitor, which is prevented from extending by a polymerase by the “second blocking moiety” and/or by the “fourth blocking unit” of the controller oligonucleotide when the first competitor-oligonucleotide-primer is complementarily bound to the controller oligonucleotide

The total length of the first competitor-oligonucleotide-primer is between 10 and 80, particularly between 15 and 50, particularly between 20 and 30 nucleotides or their equivalents (e.g. nucleotide modifications). The length of the 3′ segment, which can undergo complementary binding to an expected sequence variant of a target sequence, comprises essentially the same regions as the first segment of the first primer. Hence, it is ensured that both primers can bind competitively to the target sequence and that said binding takes place in approximately the same temperature ranges. The competitive binding and particularly the competitive primer extension using allele-specific sequence segments of the target nucleic acid generally leads to a further increase in specificity.

The structure of the first competitor-oligonucleotide-primer is adapted in such way that it can undergo reversible binding of a controller oligonucleotide under selected reaction conditions. In certain embodiments, the first competitor-oligonucleotide-primer comprises sequence segments that are not intended to bind to the first region of a controller oligonucleotide complementarily. Thus, this first competitor oligonucleotide cannot act as an initiator of strand displacement by a controller oligonucleotide.

In an advantageous embodiment of the invention, the first competitor-oligonucleotide-primer is a DNA oligonucleotide with a conventional 5′-3′ arrangement of nucleotides.

In certain embodiments, a competitor-oligonucleotide-primer comprises further sequences that do not bind to the controller oligonucleotide. These sequences can be used for other purposes, such as binding to the solid phase. These sequences are preferably located at the 5′ end of the 3′ segment.

The First Primer Region of the First Competitor-Oligonucleotide-Primer

The sequence length is between approx. 15-30 nucleotides, particularly between 5 and 20 nucleotides, wherein the sequence is predominantly complementary to the 3′ segment of a strand of the allelic variant of a target nucleic acid to be suppressed in the amplification. The first primer region can include nucleotide monomers that have no or negligible influence on the function of the polymerase, such as for example:
natural nucleotides (dA, dT, dC, dG etc.) or their modifications without altered base pairing
M modified nucleotides, 2-amino-dA, 2-thio-dT or other nucleotide modifications with deviating base pairing

In certain embodiments, an allele-specific first primer is used in combination with an allele-specific controller oligonucleotide and an allele-specific competitor-oligonucleotide-primer.

Preferred Embodiments of the Controller Oligonucleotide

A controller oligonucleotide (FIGS. 2, 7-24, 29) comprises
A first single-stranded region which can bind to the polynucleotide tail of the second region of the first oligonucleotide primer,
a second single-stranded region which can bind to the first region of the first oligonucleotide primer essentially complementarily,
a third single-stranded region which is essentially complementary to at least one segment of the extension product of the first primer extension product,
and the controller oligonucleotide does not serve as a template for primer extension of the first or second oligonucleotide primer.

Generally, the sequence of the third region of the controller oligonucleotide is adapted to the sequence of the nucleic acid to be amplified, as this is crucial as template for the sequence of nucleotides in the extension product of a first primer. The sequence of the second region of the controller oligonucleotide is adapted to the sequence of the first segment of the primer. The structure of the first region of the controller oligonucleotide is adapted to the sequence of the second region of the first oligonucleotide primer, particularly to the structure of the polynucleotide tail.

A controller oligonucleotide can also include further sequence segments which do not belong to the first, second or third region. Said sequences can, for example, be attached as flanking sequences at the 3′ and 5′ end. Preferably, said sequence segments do not interfere with the function of the controller oligonucleotide.

The structure of the controller oligonucleotide preferably comprises the following properties:

The individual regions are covalently bound to each other. The binding can take place via conventional 5′-3′ binding, for example. For example, a phospho-diester bond or a nuclease-resistant phospho-thioester bond can be used.

A controller oligonucleotide can bind to the polynucleotide tail of the first oligonucleotide primer by means of its first region, wherein binding is mediated predominantly by hybridization of complementary bases. The length of said first region is 3-80 nucleotides, particularly 4-40 nucleotides, particularly 6-20 nucleotides. The degree of sequence matching between the sequence of the first region of the controller oligonucleotide and the sequence of the second region of the first oligonucleotide primer can be between 20% and 100%, particularly between 50% and 100%, particularly between 80% and 100%. The binding of the first region of a controller oligonucleotide should preferably take place specifically to the second region of the first oligonucleotide primer under reaction conditions.

The sequence of the first region of the controller oligonucleotide is preferably selected such that the number of complementary bases which can bind complementarily to the second region of the first oligonucleotide primer is between 1 and 40, particularly between 3 and 20, particularly between 6 and 15.

Since the controller oligonucleotide is not a template for the polymerase, it can include nucleotide modifications that do not support the polymerase function, which can be base modifications and/or sugar-phosphate backbone modifications. For example, the controller oligonucleotide can include nucleotides and/or nucleotide modifications selected from the following list in its first region: DNA, RNA, LNA (“locked nucleic acids”: analogues with 2′-4′ bridge binding in the sugar residue), UNA (“unlocked nucleid acids”: without binding between 2′-3′ atoms of the sugar residue), PNA (“peptide nucleic acids” analogues), PTO (phosphorothioates), morpholino analogues, 2′-O-alkyl-RNA modifications (such as 2′-OMe, 2′-O-propargyl, 2′-O-(2-methoxyethyl), 2′-O-propyl-amine), 2′-halogen-RNA, 2′-amino-RNA etc. Said nucleotides or nucleotide modifications are linked to each other, for example by conventional 5′-3′ binding or 5′-2′ binding. For example, a phospho-diester bond or a nuclease resistant phospho-thioester bond can be used.

The controller oligonucleotide can include nucleotides and/or nucleotide modifications in its first region, wherein the nucleobases are selected from the following list: Adenines and its analogues, Guanines and its analogues, Cytosines and its analogues, Uracil and its analogues, Thymines and its analogues, Inosines or other universal bases (e.g. nitroindole), 2-amino adenine and its analogues, Iso-cytosines and its analogues, Iso-guanines and its analogues.

The controller oligonucleotide can include in its first region non-nucleotide compounds selected from the following list: Intercalating substances that can influence the binding strength between the controller oligonucleotide and the first oligonucleotide primers, e.g. MGB, naphthalenes, etc. The same elements can be used in the second region of the first primer as well.

The controller oligonucleotide can include non-nucleotide compounds in its first region, e.g. linkers such as C3, C6, HEG linkers, which link individual segments of the first region.

By means of its second region, the controller oligonucleotide can bind to the first region of the first oligonucleotide primer, wherein the binding is mediated essentially by hybridization of complementary bases.

The length of the second region of the controller oligonucleotide is adapted to and preferably matches the length of the first region of the first oligonucleotide primer. It is between about 3-30 nucleotides, particularly between 5 and 20 nucleotides. The sequence of the second region of the controller oligonucleotide is preferably complementary to the first region of the first oligonucleotide primer. The degree of complementarity is between 80% and 100%, particularly between 95% and 100%, particularly at 100%. The second region of the controller oligonucleotide preferably includes nucleotide modifications that prevent the polymerase from extending the first primer oligonucleotide but do not block or do not essentially prevent the formation of complementary double strands, for example 2′-O-alkyl RNA analogs (e.g. 2′-O-Me, 2′-O-(2-methoxyethyl), 2′-O-propyl, 2′-O-propargyl nucleotide modifications), LNA, PNA or morpholino nucleotide modifications. Single nucleotide monomers are preferably linked via 5′-3′ linkage, but an alternative 5′-2′ linkage between nucleotide monomers can be used as well.

The sequence length and its nature of the first and second region of the controller oligonucleotide are preferably chosen such that the binding of said regions to the first oligonucleotide primer is reversible under reaction conditions at least in one reaction step of the method. This means that although the controller oligonucleotide and the first oligonucleotide primer can bind specifically to each other, this binding shall not lead to the formation of a complex of both elements that is permanently stable under reaction conditions.

Rather, an equilibrium between a bound complex form of controller oligonucleotide and the first oligonucleotide primer and a free form of individual components under reaction conditions should be sought or made possible at least in one reaction step. This ensures that at least a portion of the first oligonucleotide primer is present in free form under reaction conditions and can interact with the template to initiate a primer extension reaction. On the other hand, it ensures that respective sequence regions of the controller oligonucleotide are available for binding of an extended oligonucleotide primer.

The portion of free, single-stranded and thus reactive components can be influenced by temperature selection during the reaction: by lowering the temperature, first oligonucleotide primers bind to the controller oligonucleotides, so that both participants bind a complementary double-stranded complex. This allows the concentration of single-stranded forms of individual components to be decreased. An increase in temperature can lead to dissociation of both components into single-stranded forms. In the region of the melting temperature of the complex (controller oligonucleotide/first segment oligonucleotide primer), about 50% of the components are present in single stranded form and about 50% as double stranded complex. By using respective temperatures, the concentration of single-stranded forms in the reaction mixture can be influenced.

In an embodiment of the amplification method, which comprises a temperature change between individual reaction steps, the desired reaction conditions can be achieved during the respective reaction steps. For example, by using temperature regions approximately equal to the melting temperature of complexes of controller oligonucleotide/first oligonucleotide primer, portions of respectively free forms of individual components can be influenced. The temperature used leads to a destabilization of complexes comprising controller oligonucleotide/first primer oligonucleotide, so that during this reaction step individual complex components become at least temporarily single-stranded and are thus enabled to interact with other reaction partners. For example, a first sequence region of the controller oligonucleotide can be released from the double-stranded complex with a non-extended first primer and can thus interact with the second sequence region of an extended first primer oligonucleotide and thus initiate strand displacement. On the other hand, the release of a first, non-extended oligonucleotide primer from a complex comprising controller oligonucleotide/first oligonucleotide causes the first region of the primer to become single-stranded and can interact with the template to initiate primer extension by a polymerase.

The temperature used does not have to be exactly the same as the melting temperature of the complex of controller oligonucleotide/first oligonucleotide primer. It is sufficient if the temperature in one reaction step is approximately in the range of the melting temperature. For example, the temperature in one of the reaction steps comprises regions of Tm+/−10° C., particularly Tm+/−5° C., particularly Tm+/−3° C. of the complex of controller oligonucleotide/first segment primer oligonucleotide.

Such a temperature can be set, for example, within the reaction step, which comprises a sequence-specific strand displacement by the controller oligonucleotide.

In an embodiment of the amplification method that does not comprise any temperature changes between individual reaction steps and the amplification proceeds under isothermal conditions, reaction conditions are maintained throughout the entire duration of the amplification reaction under which an equilibrium between a complex form of controller oligonucleotide/first oligonucleotide primer and a free form of individual components is possible.

The ratio between a complex form of controller oligonucleotide and the first oligonucleotide primer and free forms of individual components can be influenced both by reaction conditions (e.g. temperature and Mg2+ concentration) and by structures and concentrations of individual components.

The sequence length and its composition of the first and second region of the controller oligonucleotide are selected in an embodiment such that under given reaction conditions (e.g. in the reaction step of a strand displacement by the controller oligonucleotide) the ratio between a portion of a free controller oligonucleotide and a portion of a controller oligonucleotide in complex with a first oligonucleotide primer comprises the following ranges: from 1:100 to 100:1, particularly from 1:30 to 30:1, particularly from 1:10 to 10:1. The ratio between a portion of a free first oligonucleotide primer oligonucleotide and a portion of a first oligonucleotide primer complexed with a controller oligonucleotide comprises ranges from 1:100 to 100:1, particularly from 1:30 to 30:1, particularly from 1:10 to 10:1.

In certain embodiments, the concentration of the first oligonucleotide primer is higher than the concentration of the controller oligonucleotide. Therefore, an excess of the first primer is present in the reaction and the controller oligonucleotide must be released for its effect from binding of first primer by selecting the respective reaction temperature. This generally takes place by increasing the temperature until sufficient concentrations of free forms of the controller oligonucleotide are present.

In certain embodiments, the concentration of the first oligonucleotide primer is lower than the concentration of the controller oligonucleotide. Thus, an excess of the controller oligonucleotide is present and the first oligonucleotide primer must be released from binding of a controller oligonucleotide by selecting the respective reaction temperature. This generally takes place by raising the temperature until sufficient concentrations of free forms of the first oligonucleotide primer are present.

Under isothermal conditions, an equilibrium is present: certain portions of the first oligonucleotide primer and controller oligonucleotide are bound to each other, while others are present in the reaction as single-stranded forms.

The controller oligonucleotide can bind to at least one segment of the specifically synthesized extension product of the first oligonucleotide primer by means of its third region. The binding takes place preferably by hybridization of complementary bases between the controller oligonucleotide and the extension product synthesized by the polymerase.

To support the strand displacement reaction, the sequence of the third region should preferably comprise a high complementarity to the extension product. In certain embodiments, the sequence of the third region is 100% complementary to the extension product.

The binding of the third region preferably takes place to the segment of the extension product that immediately adjoins the first region of the first oligonucleotide primer. Thus, the segment of the extension product is preferably located in the 5′ segment of the entire extension product of the first oligonucleotide primer.

The binding of the third region of the controller oligonucleotide preferably does not take place over the entire length of the extension product of the first oligonucleotide primer. Preferably, one segment at the 3′ end of the extension product remains unbound. This 3′ end segment is necessary for binding the second oligonucleotide primer.

The length of the third region is adjusted respectively so that the third region binds to the 5′ standing segment of the extension product and does not bind to the 3′ standing segment of the extension product.

The total length of the third region of the controller oligonucleotide is from 2 to 100, particularly from 6 to 60, particularly from 10 to 40 nucleotides or their equivalents. The controller oligonucleotide can undergo a complementary binding with the segment of the extension product over this length and thus displace this 5′ standing segment of the extension product from the binding with its complementary template strand.

For example, the length of the 3′-standing segment of the extension product that is not bound by the controller oligonucleotide comprises ranges between 5 and 200, particularly between 5 and 100, particularly between 5 and 60 nucleotides, particularly between 10 and 40, particularly between 15 and 30 nucleotides.

This 3′ standing segment of the extension product is not displaced by the controller oligonucleotide from binding with the template strand. Even if a fully bound third region of the controller oligonucleotide is attached to its complementary segment of the extension product, the first region of the primer extension product can remain bound to the template strand via its 3′-standing segment. The binding strength of this complex is preferably chosen such that it can dissociate spontaneously under reaction conditions (step e), for example. This can be achieved, for example, by ensuring that the melting temperature of this complex from the 3′-standing segment of the extension product of the first oligonucleotide primer and its template strand is approximately in the range of the reaction temperature or below the reaction temperature in a respective reaction step (reaction step e). If the stability of said complex in the 3′ segment of the extension product is low, a complete binding of a third region of a controller oligonucleotide to the 5′ segment of the extension product leads to a rapid dissociation of the first primer extension from its template strand.

Overall, the controller oligonucleotide has a suitable structure to perform its function: under respective reaction conditions, it is able to displace the extended first oligonucleotide primer from binding of a controller oligonucleotide to the template strand sequence-specifically, thereby converting the template strand into single-stranded form and making it available for further binding with a new first oligonucleotide primer and its target sequence-specific extension by the polymerase.

In order to fulfill the function of strand displacement, regions one, two and three of the controller oligonucleotide should be predominantly in single-stranded form under reaction conditions. Therefore, double-stranded self-complementary structures (e.g. hairpins) in these regions should be avoided if possible, as they can reduce the functionality of the controller oligonucleotide.

The controller oligonucleotide should not act as a template in the method according to the invention, therefore the first oligonucleotide primer should not be extended by the polymerase when annealed to the controller oligonucleotide under reaction conditions.

This is preferably achieved by using nucleotide modifications which prevent the polymerase from copying the strand. Preferably, the 3′-end of the first oligonucleotide primer will not be extended when the first oligonucleotide primer binds to the controller oligonucleotide under reaction conditions.

The degree of blockage/impediment/slowing/ramping down of the reaction can be located between complete expression of said property (e.g. 100% blockage under given reaction conditions) and partial expression of said property (e.g. 30-90% blockage under given reaction conditions). Nucleotide modifications are preferred, which individually or in a series coupled together (e.g. as a sequence fragment consisting of modified nucleotides) can prevent the extension of a first primer by more than 70%, particularly by more than 90%, particularly by more than 95%, and particularly by 100%.

The nucleotide modifications can comprise base modifications and/or sugar-phosphate residue modifications. Sugar-phosphate modifications are preferred, since any complementary sequence of a controller oligonucleotide can be assembled by combining it with conventional nucleobases. For example, nucleotides with modifications in the sugar-phosphate residue that can hinder or block the synthesis of the polymerase include: 2′-O-alkyl modifications (e.g. 2′-O-methyl, 2′-O-(2-methoxyethyl), 2′-O-propyl, 2′-O-propargyl nucleotide modifications), 2′-O-amino-2′-deoxy nucleotide modifications, 2′-O-aminoalkyl-2′-deoxy nucleotide modifications, PNA, morpholino modifications, etc.

The blockade can take place either by a single nucleotide modification or by coupling several nucleotide modifications in series (e.g. as a sequence fragment consisting of modified nucleotides). For example, at least 2, particularly at least 5, particularly at least 10 such nucleotide modifications can be coupled side by side in the controller oligonucleotide.

A controller oligonucleotide can comprise a uniform type of nucleotide modifications or also comprise at least two different types of nucleotide modification.

The position of such nucleotide modifications in the controller oligonucleotide should preferably prevent the polymerase from extending the 3′ end of a first oligonucleotide primer bound to the controller oligonucleotide.

In certain embodiments, such nucleotide modifications are located in the second region of the controller oligonucleotide. In certain embodiments, such nucleotide modifications are located in the third region of the controller oligonucleotide. In certain embodiments, such nucleotide modifications are located in the second and third region of the controller oligonucleotide.

For example, the second region of the controller oligonucleotide consists of at least 20% of its positions of such nucleotide modifications, particularly at least 50%.

For example, the third region of the controller oligonucleotide consists of at least 20% of its positions of such nucleotide modifications, particularly at least 50%, particularly at least 90%. In certain embodiments, the entire third region comprises nucleotide modifications that prevent a polymerase from extending a primer bound to such a region using the controller oligonucleotide as template. In certain embodiments, the entire third and second region comprise such nucleotide modifications. In certain embodiments, the entire first, second and third regions comprise such nucleotide modifications. Thus, the controller oligonucleotide can consist entirely of such nucleotide modifications. Such modified controller oligonucleotides can be used, for example, in multiplex analyses where additional primers are used. This is to prevent unintentional primer extension reactions at one or more controller oligonucleotides.

The sequence of the nucleobases of these nucleotide modifications is adapted to the sequence requirements in the respective region.

The remaining portion can be natural nucleotides or nucleotide modifications that do not or only insignificantly hinder the polymerase function, e.g. DNA nucleotides, PTO nucleotides, LNA nucleotides, RNA nucleotides. Further modifications, e.g. base modifications such as 2-amino adenosine, 2-aminopurine, 5-methyl cytosine, inosine, 5-nitroindole, 7-deaza-adenosine, 7-deaza-guanosine, 5-propyl cytosine, 5-propyl uridine or non-nucleotide modifications such as dyes or MGB modifications etc. can be used, e.g. to adjust the binding strengths of individual regions of the controller oligonucleotides. The individual nucleotide monomers can be coupled to each other via conventional 5′-3′ binding, or alternatively via 5′-2′ binding.

A segment of the controller oligonucleotide with nucleotide modifications that prevent the polymerase from extending from the 3′ end of a first oligonucleotide primer bound to the controller oligonucleotide is called the “second blocking unit”. The length of said segment can include from 1 to 50 nucleotide modifications, particularly from 4 to 30. For example, this segment can be located in the controller oligonucleotide such that the 3′ end of the bound first oligonucleotide primer is located in said segment. Thus, this segment can span the regions two and three. When using further primers (e.g. competitor-oligonucleotide-primers), which can also bind complementarily to the controller oligonucleotide, further blocking units can be introduced, for example a fourth blocking unit, which can be located at the same position in the controller oligonucleotide as the second blocking unit or comprise a different position. For example, the fourth blocking unit can be located in 5′ direction from the second blocking unit. The primer blockade by such a fourth blocking unit takes place according to the same principle as with the second blocking unit: a primer that is able to bind complementarily to the controller oligonucleotide is not extended by polymerase. Thus it is prevented that the controller oligonucleotide serves as a template for further primers, e.g. a competitor-primer.

A segment of the controller oligonucleotide with nucleotide modifications that prevent the polymerase from extending from the 3′ end of another oligonucleotide primer (e.g., competitor-primer) bound to the controller oligonucleotide is called the “fourth blocking unit”. The composition of such a “fourth blocking unit” can be analogous to the “second blocking unit”.

In certain embodiments, preferably no linker structures or spacer structures, such as C3, C6, HEG-linker, are used to prevent the extension of the 3′-end of a first oligonucleotide primer bound to the controller oligonucleotide.

In addition to regions one, two and three, the controller oligonucleotide can comprise further sequence segments, which for example in the 5′ segment or 3′ segment of the controller oligonucleotide flank the above-mentioned regions. Such sequence elements can be used for other functions, such as interaction with probes, binding to a solid phase, etc. Such regions preferably do not interfere with the function of regions one to three. The length of these flanking sequences can, for example, be between 1 and 50 nucleotides. Furthermore, a controller oligonucleotide can comprise at least one element for immobilization on a solid phase, e.g. a biotin residue. Furthermore, a controller oligonucleotide can comprise at least one element for detection, e.g. a fluorescent dye.

In the presence of a controller oligonucleotide, newly synthesized sequences are verified for their sequence contents by interaction with the controller oligonucleotide.
In case of correct matching with given sequence contents of the controller oligonucleotide, strand displacement takes place, wherein the template strands are replaced by newly synthesized strands. Thereby, primer binding sites are converted to the single-stranded state and are available for a new interaction with the primers and thus for further synthesis. Thus, the system is converted from both primer extension products into an active state. The controller oligonucleotide thus has an activating effect on the system.
In the absence of matching with given sequence contents of the controller oligonucleotide, the strand displacement of the template strands is influenced by newly synthesized strands. The strand displacement and/or separation is either quantitatively slowed down or completely eliminated. Thus, primer binding sites are not or less frequently transferred into the single-stranded state. Thus, no or quantitatively less primer binding sites are available for a new interaction with primers. Thus, the system is less often put into an active state from both primer extension products or an active state is not reached at all.

The efficiency of the double-strand opening of the newly synthesized primer extension products after each individual synthesis step affects the potentially achievable yields in subsequent cycles: the fewer free/single-stranded primer binding sites of a nucleic acid chain to be amplified are made available at the beginning of a synthesis step, the smaller the number of newly synthesized strands of the nucleic acid chain to be amplified in this step. In other words, the yield of a synthesis cycle is proportional to the amount of primer binding sites available for interaction with respective complementary primers. In general, a regulatory cycle can thus be realized.

This control loop generally corresponds to a respective real-time/on-line control of synthesized fragments: sequence control takes place in the reaction batch while amplification takes place. This sequence control takes place according to a predefined pattern and the oligonucleotide system (by strand-opening action of controller oligonucleotide) can decide between “correct” and “incorrect” states without external interference. In the correct state, the synthesis of sequences is continued, in the non-correct state the synthesis is either slowed down or completely prevented. The resulting differences in the yields of “correct” and “incorrect” sequences after each step affect the overall amplification, which comprises a large number of such steps.

In exponential amplification, this dependence is exponential, so that even small differences in efficiency within a single synthesis cycle due to sequence differences can significantly delay the overall amplification or cause the complete absence of detectable amplification in a given time frame.

This effect of real-time control of newly synthesized nucleic acid chains is associated with the use of the controller oligonucleotide.

Sequence-Variant-specific Controller Oligonucleotides:

A target sequence-specific controller oligonucleotide is preferably constructed in its regions in such way that it is capable of participation in the amplification of at least one predefined sequence variant of a target sequence.

In a preferred form, an amplification system comprising at least one controller oligonucleotide is designed in such way that the expected position of an allele variant of a target sequence corresponds to a corresponding segment or a corresponding position in the controller oligonucleotide.

An allele-specific controller oligonucleotide is preferably designed in such way that it is capable of preferably causing the amplification of a sequence variant of a target sequence (e.g. allele 1), wherein in the case of another sequence variant of the same target sequence (e.g. allele 2) the amplification does not occur at all or only with reduced efficiency or does not occur at all in the specified time or results in an insufficient yield of amplification products for detection.

An allele-specific controller oligonucleotide is thus provided not only target sequence-specific but also allele-specific. Such a controller oligonucleotide thus comprises target sequence-specific sequence segments as well as allele-specific sequence segments. Depending on the complexity of a polymorphic locus of a target sequence, several controller oligonucleotides specific for the respective allele variant of a target sequence can be constructed.

Preferably, a controller oligonucleotide comprises at least one sequence segment which comprises a sequence composition complementary to a specific allelic variant of a target sequence. Furthermore, a controller oligonucleotide preferably comprises at least one sequence segment which comprises a sequence composition which is complementary to a specific allele variant of a target sequence. In certain embodiments, a controller oligonucleotide comprises at least one sequence segment which can bind complementarily to a variant of a polymorphic locus of a target sequence.

In certain embodiments, several controller oligonucleotides are provided, which respectively comprise allele-specific sequence segments.

The length of a sequence segment of a controller oligonucleotide which is completely complementary to the polymorphic locus of a start nucleic acid chain can comprise ranges from one nucleotide up to 100 nucleotides, particularly from one nucleotide up to 50 nucleotides, particularly from one nucleotide up to 20 nucleotides. The length of at least one sequence segment of a controller oligonucleotide which is complementary to uniform sequence segments of a target sequence (first and second uniform sequence segment of a start nucleic acid) can comprise the following ranges: from 4 to 100 nucleotides, particularly from 6 to 100 nucleotides, particularly from 8 to 50 nucleotides.

In certain embodiments, a sequence segment of a controller oligonucleotide, which is complementary to at least one variant of a polymorphic locus of a target sequence, is located in the third region of the controller oligonucleotide.

In certain embodiments, a sequence segment of a controller oligonucleotide, which is complementary to at least one variant of a polymophic locus of a target sequence, is located in the second region of the controller oligonucleotide.

In certain embodiments, a sequence segment of a controller oligonucleotide, which is complementary to at least one variant of a polymophic locus of a target sequence, is partially and/or completely located in the second and third regions of the controller oligonucleotide.

The position of allele-specific sequence segments of a controller oligonucleotide can thus be divided into three groups. The first group comprises allele-specific sequence segments that are detected in both the first oligonucleotide primer and the controller oligonucleotide. This position thus at least partially comprises the second region of the controller oligonucleotide and the first segment of the first oligonucleotide primer.

The second group comprises allele-specific corresponding sequence segments which are only covered by the controller oligonucleotide, both primers (the first primer and the second primer) are only target sequence-specific, but not allele-specific. Thus, the position at least partially comprises the third region of the controller oligonucleotides, which is intended to bind complementarily with the 5′-segment of the polymerase synthesized portion of the first primer extension product.

The third group comprises allele-specific sequence segments that are detected in both the second oligonucleotide primer and the controller oligonucleotide. Thus, this position comprises at least partially the third region of the controller oligonucleotide predominantly in the 5′ segment of the controller oligonucleotide.

Discrimination between allele variants of a said target sequence comprises different mechanisms in these groups, which have to be considered, for example, when choosing reaction conditions.

In the first and third group, primers play an essential role in discrimination. Complementary primer binding to a respective primer binding site of an allele variant of the target sequence and initiation of a synthesis of a primer extension product by the polymerase determine the extent of the different amplifications. The controller oligonucleotide interacts only with the first primer (first group) and with the complementary sequence segment to the 3′ segment of the second primer (third group).

Successful strand displacement by the controller oligonucleotide thus depends primarily on the respective complementary design of these portions of primers and of the controller oligonucleotide. The synthesized segment portion of the respective primer extension product, which is located between both primers, represents a uniform sequence composition for an allele variant of a common target sequence. Therefore, it is advantageous to perform the amplification under stringent hybridization conditions that support allele-specific initiation of a synthesis. The use of an additional allele-specific competitor primer can further enhance a specific initiation of the synthesis.

In the second group, the initiation of the synthesis of primer extension products plays a more secondary role, since the primers used bind to primer binding sites that represent a uniform composition for all potential allele variants of a target sequence. Discrimination takes place only with the participation of the controller oligonucleotide, which participates by sequence-specific or predominantly specific strand displacement in the separation of the first and second primer extension products.

In certain embodiments, a fully complementary sequence of the third region of the controller oligonucleotide is preferred, which can form a perfect match with at least one allele variant of the target sequence. The amplification of different allele variants thus requires in said embodiment a combination of allele-specific controller oligonucleotides.

Generally, a deviation from fully complementary sequence compositions leads to deviating amplification efficiencies. At the corresponding sequence segments of a controller oligonucleotide a competition for binding of the first primer extension product (consisting predominantly of DNA) with the second primer extension product (also comprising DNA) takes place. Allele-specific sequence design of said sequence segment of the controller oligonucleotide corresponding to an allele variant allows for both competing strands to bind to the first primer extension product in a perfect match/perfect match manner. Thus, a controller oligonucleotide with its corresponding sequence segment is able to successfully displace the second primer extension product from binding with the first primer extension product. The separation of both primer extension products allows exponential amplification.

In case of a deviation in the composition of the corresponding segment of the controller oligonucleotide (e.g., binding to a non-complementary allele variant of a primer extension product), the situation generally changes. The interaction between the perfect match of the second primer extension product (consisting of DNA) and the first primer extension product (consisting of DNA) can only be overcome with difficulty or not at all by a segment of a controller oligonucleotide comprising a mismatch. Thus, preferential binding of a perfect match double strand between both primer extension products does not allow sufficient strand separation to take place with the participation of a controller oligonucleotide, which generally results in insufficient or slower amplification.

In a preferred embodiment, the sequence segment corresponding to an allele variant is located in the third region of the controller oligonucleotide, which mainly or exclusively comprises DNA monomers.

In certain embodiments, the sequence segment corresponding to an allele variant is located in the third region of the controller oligonucleotide, which comprises both DNA and DNA modifications.

In certain embodiments, the sequence segment corresponding to an allele variant is located in the third region of the controller oligonucleotide, which predominantly or exclusively comprises DNA modifications. For example, if a sequence segment corresponding to an allele variant is located within a second blocking unit that comprises several 2′-O-alkyl modifications of nucleotides, such modifications can influence the binding of the controller oligonucleotide to allele-specific variants of primer extension products.

In a preferred embodiment, the segment of a controller oligonucleotide corresponding to an allele variant is located in the third region of the controller oligonucleotide, wherein said segment of the strand of the controller oligonucleotide is predominantly composed of DNA nucleotides. Preferably, said segment is located in 5′ direction from the second blocking unit. The length of said segment corresponding to the polymorphic locus comprises particularly 1 to 20 nucleotides. The composition of the nucleotides in this fully complementary corresponding segment of the controller oligonucleotide comprises at least 70% DNA nucleotides, particularly 80%, particularly more than 95% DNA nucleotides.

Preferably, an allele variant that comprises only one nucleobase (e.g. SNP or a point mutation), the corresponding position in the middle region of the controller oligonucleotide strand, which is surrounded on both sides of this corresponding position by DNA monomers complementary to the target sequence, wherein the strand of the controller oligonucleotide comprises at least 4 DNA monomers, particularly at least 6 DNA nucleotides, particularly at least 10 DNA nucleotides in both directions of the expected SNP or point mutation. Such arrangements of DNA monomers around an expected SNP site or point mutation or single nucleotide allele variant enables the maintenance of a single strand conformation, which is characteristic for the B form (so-called single strand persistence length). Thus, strand displacement by the controller oligonucleotide takes place using a corresponding segment comprising DNA monomers.

In certain embodiments, a controller oligonucleotide is used, which can undergo a complementary binding of a 5′-end with the first primer extension product. In allele variants which comprise only one nucleobase, e.g. SNP or point mutations, the corresponding sequence segment is preferably positioned at a distance from the 5′ end of the controller oligonucleotide, which comprises lengths of 10 to 60 nucleotides, particularly 20 to 50 nucleotides, particularly 30 to 40 nucleotides.

However, the use of DNA nucleotides as monomers of controller oligonucleotides must not result in the controller oligonucleotide being used as a template for a primer extension by a polymerase used with one of the primers used.

The use of DNA nucleotides (e.g. A, C, T or G) within the segment in the controller oligonucleotide strand corresponding to an allele variant has several advantages, so that the discriminatory behavior of an amplification system is easier to estimate.

For example, the behavior of perfect-match variants of a controller oligonucleotide to certain allele variants in the case of point mutations can be estimated more easily. Discrimination between allele variants follows the rules of Watson-Crick base pairing: Controller oligonucleotides that can form complementary base pairs (A:T, G:C) with the first primer extension products generally lead to amplification reactions in good yields.

However, potential mismatches between an allele-specific controller oligonucleotide and a first primer extension product (e.g. A:C or G:G) lead to insufficient or delayed amplification. In the presence of allele variants that can make up more than one nucleotide difference to the controller oligonucleotide in a potential first primer extension product (e.g. short InDels), sufficient amplification is generally not achieved.

The effect of a mismatch on an amplification can be estimated by determining the stability of potential duplexes comprising an allele-specific controller oligonucleotide with a defined sequence and a potential primer extension product or its portions, e.g. only the synthesized portion of a primer extension product. Duplexes are formed in the absence of second primer extension products. When hybridizing an allele-specific controller oligonucleotide to a potential primer extension product with full complementarity (perfect match), the binding of such duplexes is more stable than the binding of duplexes comprising at least one mismatch position. This stability can be determined by measuring the melting temperature. Differences between amplification reactions of allele variants are generally greater, the greater the difference in stability between a perfect match duplex and a mismatch duplex comprising an allele-specific oligonucleotide primer and a potential first primer extension product. Such differences between reactions can be measured, for example, by the time required for a reaction to reach a certain amount of product. Such differences in the time between amplification reactions of perfect-match and mismatch allele variants can also be interpreted as a capacity for discrimination: the greater the time difference, the higher the discrimination between individual allele variants.

Due to the use of modifications in the controller oligonucleotide, e.g. in the region of the second blocking unit to prevent the extension of the first primer or a competitor-oligonucleotide primer, a different discrimination or even tolerance to certain mismatches can take place. For example, 2′-O-alkyl modifications can be used within the second blocking unit. Such nucleotide modifications can change the conformation of a double strand (from B-form of a DNA:DNA duplex to A-like form of a DNA:RNA or DNA:mod. DNA duplex). Particularly in the base pairing between G:C and G:U or G:T a change of discrimination can take place.

For example, the use of modifications in the controller oligonucleotide leads to a change in the discriminatory behavior of individual base pairs. A similar behavior is known from the study of RNA: the behavior of G:U changes depending on the strand shape: in RNA, a G:U base pair generally forms a sufficiently stable base pairing, in DNA, a G:T mismatch generally leads to a weakening of the binding of both strands.

In this application for example 2′-alkyl nucleotide comprising modifications in the controller oligonucleotide are used (e.g. within the second blocking unit). A 2′-O-Me cytosine (C*) or 2′-O-Me adenosine (A*) is used. Such modified nucleotides allow good discrimination between sequence variants (wherein perfect match comprises C*:dG and A*:U or A*:T and mismatch comprises e.g. C*:dA or A*:dC).

In contrast, the use of 2′-O-Me-G (G*) or 2′-O-Me-U (U*) leads to different results. For example, amplification using controller oligonucleotides with a G* at the corresponding site in the strand leads to simultaneous amplification of allelic variants with dC and dU at corresponding sites in the first primer extension product.

Thus, not only an allele-discriminating but also an allele-tolerant function of a controller oligonucleotide can be shown.

When using nucleotide modifications that allow insufficient discrimination of allele variants, several allele variants can thus be amplified simultaneously. In addition to a 2′-O-Me guanosine or 2′-O-Me uridine, universal bases such as inosines or 5-nitro-indole monomers also belong to candidates with tolerance to the allele composition. Such modifications of a controller oligonucleotide strand can be arranged at expected corresponding positions to certain allele variants.

Thus, this lack of discrimination can be taken into account for some modifications as follows:

In certain embodiments, the sequence segment corresponding to an allele variant is located in the third region of the controller oligonucleotide, which predominantly or exclusively comprises 2′-O-alkyl modifications.

In certain embodiments, the sequence segment of a controller oligonucleotide corresponding to an allele variant is located in the third region of the controller oligonucleotide, wherein said segment of the strand of the controller oligonucleotide is predominantly composed of 2′-O-alkyl modifications. Preferably, said segment is located within the second blocking unit or in 5′ direction from the second blocking unit. The length of said segment corresponding to the polymorphic locus comprises particularly 1 to 20 nucleotides. The composition of the nucleotides in said fully complementary corresponding segment of the controller oligonucleotide comprises at least 50% 2′-O-alkyl modifications, particularly 80%, particularly more than 95% 2′-O-alkyl modifications.

Particularly, even in the case of an allele variant that comprises only one nucleobase (e.g. SNP or a point mutation), the corresponding position is located in the middle region of the controller oligonucleotide strand. Said controller oligonucleotide strand is surrounded on both sides of this corresponding position by 2′-O-alkyl modifications complementary to the target sequence, wherein the strand of the controller oligonucleotide comprises at least four 2′-O-alkyl modifications, particularly at least six 2′-O-alkyl modifications, particularly at least 10 2′-O-alkyl modifications in both directions from the expected SNP or point mutation. Such an arrangement of DNA monomers around an expected SNP site or point mutation or single nucleotide allele variant enables the maintenance of a single-stranded conformation, which can be described as A-like form. Thus, strand displacement by the controller oligonucleotide takes place using a corresponding segment comprising nucleotides with 2′-O-alkyl modifications.

In certain embodiments, a controller oligonucleotide is used, which can undergo a complementary binding of a 5′-end with the first primer extension product. For allele variants which comprise only one nucleobase, e.g. SNP or point mutations, the corresponding sequence segment is preferably positioned at a distance from the 5′ end of the controller oligonucleotide, which comprises lengths of 10 to 60 nucleotides, particularly 20 to 50 nucleotides, particularly 30 to 40 nucleotides.

If a sequence segment expected to be an allele variant is localized within such a segment comprising several 2′-O-alkyl modifications, a possibly deviating base pairing is taken into account (mainly concerns alternative base pairing between DNA structures (B-Like structure) and A-Like structures of the RNA (or 20me variants). In an embodiment, the following general rule can be applied:

Controller oligonucleotides comprising 2′-O-alkyl modifications of cytosine can generally form sufficient base pairing with dG nucleotide at corresponding sites in the first primer extension product and thus support the amplification of such allele variants.

Controller oligonucleotides comprising 2′-O-alkyl modifications of adenosine can generally form sufficient base pairing with dT or dU nucleotide at corresponding sites in the first primer extension product to support the amplification of such allelic variants.

Controller oligonucleotides comprising 2′-O-alkyl modifications of guanosine can generally form sufficient base pairing with dC and dU nucleotide at corresponding sites in the first primer extension product to support the amplification of both allelic variants.

Controller oligonucleotides comprising 2′-O-alkyl modifications of uridine can generally form sufficient base pairing with dA and dG nucleotide at corresponding sites in the first primer extension product to support the amplification of both allelic variants.

Due to strand complementarity of a double-stranded target nucleic acid, strand-specific amplification systems can be created, wherein at corresponding sites respectively either 2′-O-alkyl modifications of cytosine or 2′-O-alkyl modifications of adenosine are used. Thus, the missing discrimination of G and U nucleotide analogs can be bypassed.

Reaction Conditions in the Strand Displacement Reaction

The displacement of the second primer extension product from binding with the first primer extension product by means of sequence-dependent strand displacement by the controller oligonucleotide forms a partial step in the amplification. The reaction conditions during this step are adjusted accordingly. The reaction temperature and the reaction time are chosen in such way that the reaction can take place successfully.

In a preferred embodiment, strand displacement by the controller oligonucleotide proceeds until the second primer extension product is separated/dissociated from the binding of the first primer extension product. Such dissociation of the 3′-segment of the first primer extension product from complementary portions of the second primer extension product can take place spontaneously in a temperature-dependent/temperature-related separation of both primer extension products. Such a dissociation has a favorable effect on the kinetics of the amplification reaction and can be influenced by the choice of reaction conditions, e.g. by temperature conditions. The temperature conditions are therefore chosen in such way that successful strand displacement by complementary binding of a controller oligonucleotide favors dissociation of the second primer extension product from the 3′ segment of the first primer extension product.

In a further certain embodiment, strand displacement by the controller oligonucleotide proceeds until the detachment/dissociation of a 3′ segment of the second primer extension product (P2.1-Ext) or template strand (M1) from complementary binding with the first primer extension product (P1.1-Ext). Thereby, said segment of the second primer extension product (P2.1-Ext) or the template strand (M1) comprises at least one region complementary to the first primer and one segment complementary to the first primer extension product (P1.1 -Ext), which is formed during the enzymatic synthesis. Thereby, a complex (C1.1/ P1.1-Ext/P2.1.-Ext) or (C1.1/ P1.1-Ext/M1) is formed, which comprises the first primer extension product (P1.1-Ext), the second primer extension product (P2.1-Ext)/or template strand (M1) as well as the controller oligonucleotide (C1.1). In such a complex, the 3′-segment of the second primer extension product is present at least temporarily in a single-stranded form, as it can be displaced by the controller oligonucleotide from its binding of a controller oligonucleotide with the first primer extension product. The 3′ segment of the first primer extension product (P1.1-Ext) is present in such a complex, complementarily hybridized to the second primer extension product (P2.1-Ext) or to a template strand (M1).

Due to a partially open primer binding site for the first oligonucleotide primer (3′-segment of the second primer extension product), a new primer extension product (P1.2) can bind to one of these single-stranded sequence segments of the complex (P2.1-Ext) under reaction conditions and thus initiate a synthesis of a new first primer extension product (P1.2-Ext) by a polymerase. Generally, the initiation of this reaction proceeds with reduced efficiency, since the 3′-segment of the P2.1-Ext is not permanently single-stranded, but is in competitive behavior with the controller oligonucleotide and thus comprises alternating single-stranded and double-stranded states by binding of a controller oligonucleotide to the P1.1-Ext.

Continuing this newly started synthesis of P1.2-Ext using the second primer extension product as template (P2.1 -Ext), a polymerase-related strand displacement can also lead to dissociation of the complex (C1.1/P1.1-Ext/P2.1.-Ext). The controller oligonucleotide, the temperature-dependent double-strand destabilization and the strand displacement by the polymerase act synergistically and complementarily. The result is a dissociation of the 3′ segment of the first primer extension product (P1.1-Ext) from complementary portions of the second primer extension product (P2.1-Ext).

Such a dissociation has a favorable effect on the kinetics of the amplification reaction and can be influenced by the choice of reaction conditions, e.g. by temperature conditions. The participation of polymerase-mediated synthesis-dependent strand displacement in the dissociation of P1.1-Ext and P2.1-Ext has a favorable effect on strand separation.

For example, the temperature in this step comprises regions from 15° C. to 75° C., particularly from 30° C. to 70° C., particularly from 50° C. to 70° C.

For a given length of the first region of the controller oligonucleotide and the second region of the first oligonucleotide primer (comprising for example regions of 3 to 25 nucleotide monomers, particularly 5 to 15 nucleotide monomers), a strand displacement reaction can generally be initiated successfully. If the controller oligonucleotide is fully complementary to respective portions of the first primer extension product, the controller oligonucleotide can bind to the first primer extension product up to the 3′ segment of the first primer extension product and displace the second primer extension product. The second primer extension product thus remains in binding with the 3′ segment of the first primer extension product. The strength of said binding can be influenced by temperature. When a critical temperature is reached, said binding can dissociate and both primer extension products can dissociate. The shorter the sequence of the 3′-segment, the more unstable the binding is and the lower the temperature can be, which causes spontaneous dissociation.

A spontaneous dissociation can be achieved, for example, in the temperature range which is approximately at the melting temperature. In certain embodiments, the temperature of the strand displacement steps by the controller oligonucleotide is approximately at the melting temperature (Tm+/−3° C.) of the complex comprising the 3′ segment of the first oligonucleotide primer extension product, which is not bound by the controller oligonucleotide, and the second oligonucleotide primer or the second primer extension product.

In certain embodiments, the temperature of the strand displacement steps by the controller oligonucleotide is approximately at the melting temperature (Tm+/−5° C.) of the complex comprising the 3′ segment of the first primer extension product which is not bound by the controller oligonucleotide and the second oligonucleotide primer or second primer extension product.

In certain embodiments, the temperature of the strand displacement steps by the controller oligonucleotide is above the melting temperature of the complex comprising the 3′-segment of the first primer extension product not bound by controller oligonucleotide and the second oligonucleotide primer or second primer extension product, respectively. Such a temperature comprises temperature ranges from about Tm+5° C. to Tm+20° C., particularly from Tm+5° C. to Tm+10° C. By using a higher temperature, the equilibrium in this reaction step can be shifted towards dissociation. Thus, the kinetics of the reaction can be favorably influenced. The use of too low temperatures in the strand displacement step using a controller oligonucleotide can lead to a significant slowdown of the amplification.

In certain embodiments, a first primer extension product comprises a 3′ segment that is not bound by the controller oligonucleotide, and which comprises sequence lengths from 9 to about 18 nucleotides. In said embodiment, spontaneous dissociation can generally be achieved already at temperature ranges between 40° C. and 65° C. Higher temperatures also lead to dissociation.

In certain embodiments, a first primer extension product comprises a 3′ segment which is not bound by the controller oligonucleotide and which comprises sequence lengths from 15 to about 25 nucleotides. In said embodiment, spontaneous dissociation can generally be achieved already at temperature ranges between 50° C. and 70° C. Higher temperatures also lead to dissociation.

In certain embodiments, a first primer extension product comprises a 3′ segment which is not bound by the controller oligonucleotide and which comprises sequence lengths from 20 to about 40 nucleotides. In said embodiment, spontaneous dissociation can generally be achieved in temperature ranges between 50° C. and 75° C. Higher temperatures also lead to dissociation.

The composition of the 3′-segment of the first primer extension product and, if necessary, an introduction of melting temperature influencing oligonucleotide modifications (e.g. MGB) or reaction conditions (e.g. TPAC, betaines) can influence the choice of temperature. A respective adjustment can therefore be made.

In certain embodiments, all steps of the amplification take place under stringent conditions that prevent or slow down the formation of unspecific products/by-products. Such conditions include higher temperatures, for example above 50° C.

In certain embodiments, the individual steps of strand displacement by controller oligonucleotides take place at the same temperature as the synthesis of the first and second primer extension product. In certain embodiments, the individual strand displacement steps by controller oligonucleotides take place at a temperature that differs from the temperature of the respective synthesis of the first and second primer extension products. In certain embodiments, the synthesis of the first primer extension product and the strand displacement by the controller oligonucleotides takes place at the same temperature. In certain embodiments, the synthesis of the second primer extension product and the displacement of the strand by the controller oligonucleotide takes place at the same temperature.

The concentration of the controller oligonucleotide comprises ranges from 0.01 μmol/l to 50 μmol/l, particularly from 0.1 μmol/l to 20 μmol/l, particularly from 0.1 μmol/l to 10 μmol/l.

Oligonucleotide Primer Comprising Additional Sequence Segments:

The above-mentioned structures of the first primer and the second primer can be understood as so-called “basic structure of the primer” and “minimal structure of the primer, respectively.

Such basic structures of oligonucleotides with primer function (e.g. first oligonucleotide primer, second oligonucleotide primer, if necessary third oligonucleotide primer, if necessary fourth oligonucleotide primer, etc.) comprise sequence segments which are advantageous for the execution of the amplification method, for example the first and second regions of the first primer.

Such a basic structure of the primers can be extended by further, additional sequence segments. Such additional sequence segments comprise structures which are not necessary for the execution of the amplification method, but may be useful for other objectives.

Such additional segments can optionally be introduced into a primer and used for further functions or reactions. Thus, the primer extension products synthesized by the polymerase (starting e.g. from the first and/or second primer) can be bound to such sequence segments. This allows the integration of such additional sequence segments and primer extension products into a molecular structure. Such an integration can be advantageous in certain embodiments. A variety of applications for primer sequences with additional sequence segments are known to a skilled person.

Several functions are known to a skilled person, which are supported by additional sequence segments of the primer.

For example, the introduction of additional structures can be used as a means to mediate intermolecular or intramolecular binding. Several examples of such structures are known to a skilled person. For example, probes can be designed according to such a principle of intramolecular binding, e.g. in Scorpion primers. Furthermore, sequence segments can be used to bind additional oligonucleotides. Sequence-specific intermolecular binding can be achieved by using stringent conditions. Such interactions can be used, for example, to bind amplification products to a solid phase by complementary binding to immobilized oligonucleotides.

Another example is the introduction of so-called adaptor sequences and/or the use of further segments for the unique coding or sequence-specific labeling of primers and primer extension products (so-called primer barcoding). This is used for example for NGS-Library Preparation (St\dot{a}hlberg et al Nucleic Acids Res. 2016 Jun. 20; 44(11): e105). For the sequence analysis of primer extension products, such a label can be used for sequence assignment to take place later on.

Another example is the use of further sequence segments to introduce specific sequences with binding of certain proteins, e.g. restrictions endonucleases etc.

Another example is the use of further sequence segments to introduce spacer sequences, which are not intended to bind a specific interaction partner, but mainly serve to increase the distance between neighboring sequences.

Such additional sequences can either be positioned at the copyable portion of the primer or be added to the uncopyable portion of the primer. Several factors play a role in determining whether a sequence segment is copied or not. For example, positioning of the sequence segment in the respective oligonucleotide, used nucleotide modifications (e.g. C3, HEG, 2′-Ome etc.) can decide whether a sequence segment is used as a template during a method step or not.

In an embodiment, an additional sequence segment is introduced into copyable regions of the primer, e.g. at the 5′ segment of the copyable portion of the second primer, so that, for example, when the primer sequence is read off during a synthesis procedure of a target sequence, additional sequence segments are read off by the polymerase as well. The length of such an additional sequence segment comprises ranges from 3 to 50 nucleotides. The composition of these sequence segments allows in said embodiment the synthesis by a polymerase, said segment serves as a template for polymerase-dependent synthesis. In such a segment natural nucleotides are used, e.g. dA, dG, dC, dT.

In a further embodiment, additional sequence segments can be positioned, for example, at the 5′ terminus of the primer that shall not be copied during the synthesis of specific amplification fragments comprising a target sequence. This can be achieved, for example, by positioning one or more modifications or chemical groups that prevent the polymerase from synthesizing a complementary strand (e.g. HEG, C3, a segment comprising 4-10 nucleotides with 2′-Ome modifications, etc.). Such a modification can, for example, be positioned at the 5′ terminus of the copyable portion of the second primer and hinder the continuation of the synthesis. For example, a HEG group can be introduced at the 5′ end of the copyable segment of the second primer, and then an additional sequence segment can be introduced afterwards.

Furthermore, an additional sequence segment can be positioned at the 5′ terminus of the second region of the first primer. Such localization of additional segments prevents the synthesis of a complementary strand during a regular synthesis of specific amplification products comprising a target sequence.

The length of such an additional sequence segment comprises ranges from 3 to 50 nucleotides. The base composition may comprise natural nucleobases (A,G, C, T, U, inosines) or modifications at different positions of nucleotides (e.g. at the bases, such as 2-amino adenine, iso-guanine, iso-cytosines, 5-propargyl-uridines, 5-propargyl-cytosines or at the sugar-phosphate backbone, such as LNA, 2′-Ome, 2′-halogens etc.). In a certain embodiment, a first primer and additional sequence segments are combined to an oligonucleotide. In a certain further embodiment, a second primer and additional sequence segments are combined to an oligonucleotide.

Such additional sequence segments in an oligonucleotide are designed in such way that they do not prevent the amplification method of target sequences. This is achieved, for example, by avoiding or reducing inhibitory interactions with the structures of the primers or controllers essential for the method. In certain embodiments, additional structures can form complementary double strand segments with other regions under the selected reaction conditions. Particularly such double strand segments do not prevent a specific amplification of a target sequence. In certain embodiments, such additional sequence segments do not interact or bind to the first or second region of the first primer. In certain embodiments, such additional sequence segments do not interact with the controller oligonucleotide. In certain embodiments, such additional sequence segments do not interact with other primers in the reaction. In certain embodiments such additional sequence segments do not interact with P1.1-Ext or P2.1-Ext or other amplification fragments comprising a target sequence. In certain embodiments, such additional sequence segments do not form double strand segments with the first or second region of the first primer that are stable under reaction conditions and which completely prevent the function of the first or second region.

In certain embodiments, such additional sequence segments do not interact or bind to the second primer. In certain embodiments, such additional sequence segments particularly do not interact with the 3′ segment of the second primer.

In certain embodiments, the first primer comprises an additional segment of the first primer (additional sequence variant P1) at its 5′ end of the second region. Said segment optionally comprises a sequence of 10-50 nucleotides, which does not interfere with the amplification method of target sequences (e.g. does not form secondary structures with primers). Furthermore, said segment optionally comprises a sequence of about 5 to 15 nucleotides of the copyable first region of the first primer. The additional sequence variant P1 comprises natural nucleotides as monomers (A, C, G,T) and can potentially serve as a template for a polymerase.

In certain embodiments the second primer comprises at its 5′ terminus an additional sequence segment of the second primer (additional sequence variant P2). Optionally, said segment comprises a sequence of 10-50 nucleotides which does not interfere with the amplification method of target sequences (e.g. does not form secondary structures with primers). Furthermore, said segment optionally comprises a sequence of about 5 to 15 nucleotides of the copyable region of the second primer. The additional sequence variant P2 comprises natural nucleotides as monomers (A, C, G,T) and can potentially serve as a template for a polymerase.

It has been observed that such oligonucleotides comprising a first primer and additional sequence variant P1 or oligonucleotides comprising a second primer and additional sequence variant P2 are less susceptible to side reactions than oligonucleotides comprising only a first primer or oligonucleotides comprising only a second primer. In certain embodiments, for example, the generation and/or amplification of unspecific primer-dimer structures can be delayed. In such a side reaction, the formation of by-products comprising no target sequence can be optionally reduced or delayed. Thus, for example, the premature consumption of primers can be reduced or delayed. The use of such oligonucleotides is advantageous, for example, if primer-dimers comprising first primers (PD P1) or primer-dimers comprising second primers (PD P2) are generated by side reactions and lead to premature consumption of primers in the reaction. The use of primers with such additional structures (first primer with additional sequence variant P1 and/or second primer with additional sequence variant P2) is advantageous in certain embodiments when non-specific reactions are observed in an amplification reaction. Such side reactions can be favored by several factors, including:
longer response times (e.g. in ranges between 1 hr and 100 hr)
higher concentrations of primers are used (e.g. in ranges between 1 μmol/l and 1 mmol/l)
higher concentrations of polymerase are used (e.g. in ranges above 10 unit/10 μl)
Multiplex reactions (e.g. amplification of more than 10 different target sequences in one batch).
high concentrations of complex nucleic acid chains in the reaction batch (e.g. concentrations above 1 μg hgDNA in 50 μl)

Individual factors alone or in combination with other factors can promote side reactions.

Generally, side reactions (e.g. non-specific primer-dimer formation) can be prevented by optimizing reaction components and/or reaction conditions, for example by reducing concentrations of individual components, shorter reaction times, sequence design of primer sequences, selection of more stringent reaction conditions. The additional sequence segments listed in an advantageous embodiment (oligonucleotides comprising a first primer and additional sequence variant P1 or oligonucleotides comprising a second primer and additional sequence variant P2) represent a further possible way to delay certain side reactions.

In exemplary embodiments, oligonucleotide primers with additional sequence segments are shown. In said exemplary embodiments additional sequence segments are used, which do not participate in the specific amplification of a target sequence and contribute to delay side reactions. Thus oligonucleotides comprising a first primer and additional sequence variant P1 and oligonucleotides comprising a second primer and additional sequence variant P2 are used.

A skilled person will recognize that an oligonucleotide can comprise additional to a primer structure, which is advantageous for the specific amplification of a target sequence (said structure can also be called “basic structure” or “minimal structure”), additional sequence segments (e.g. additional sequence variant P1 or additional sequence variant P2). Such additional sequence segments can provide a variety of other advantageous or useful properties or functions.

Examples of Embodiments of the Present Invention

### DESCRIPTION OF FIGURES

FIG. 1 shows the sequence components of a preferred embodiment of the invention. Primer 1.1 can bind predominantly complementary with its first region (in 3′-segments) to the respective primer binding site on the template strand (M1.1). The polynucleotide tail (primer overhang) of the second region does not bind to the template strand (M1.1). The controller (1.1.) comprises a first, second and third region.

FIG. 2 shows schematically the topography of the complete primer extension product (P1-Ext.), wherein the extension product comprises a region formed by primer 1.1 and a segment synthesized by the polymerase.

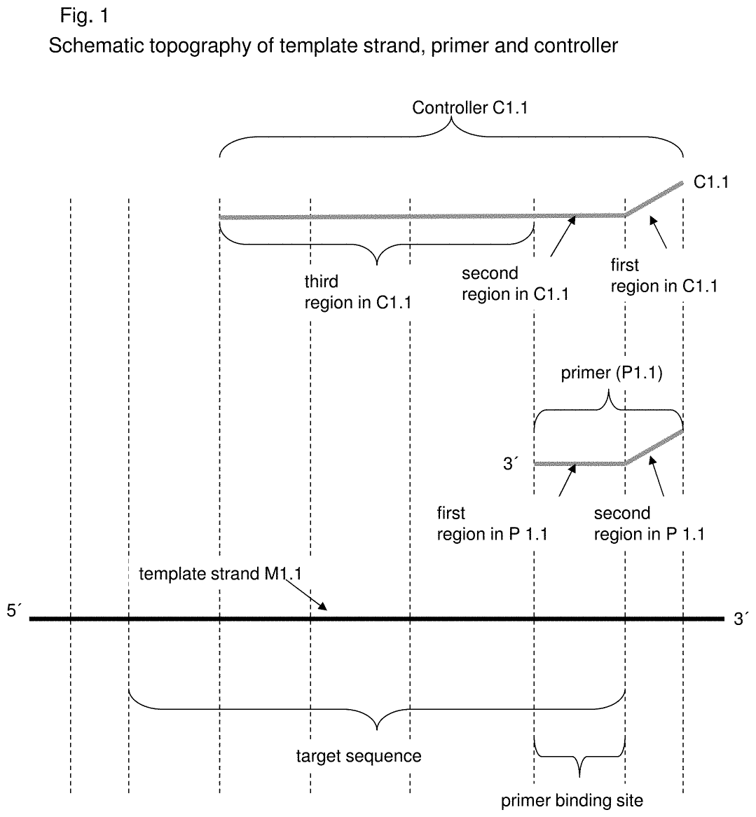

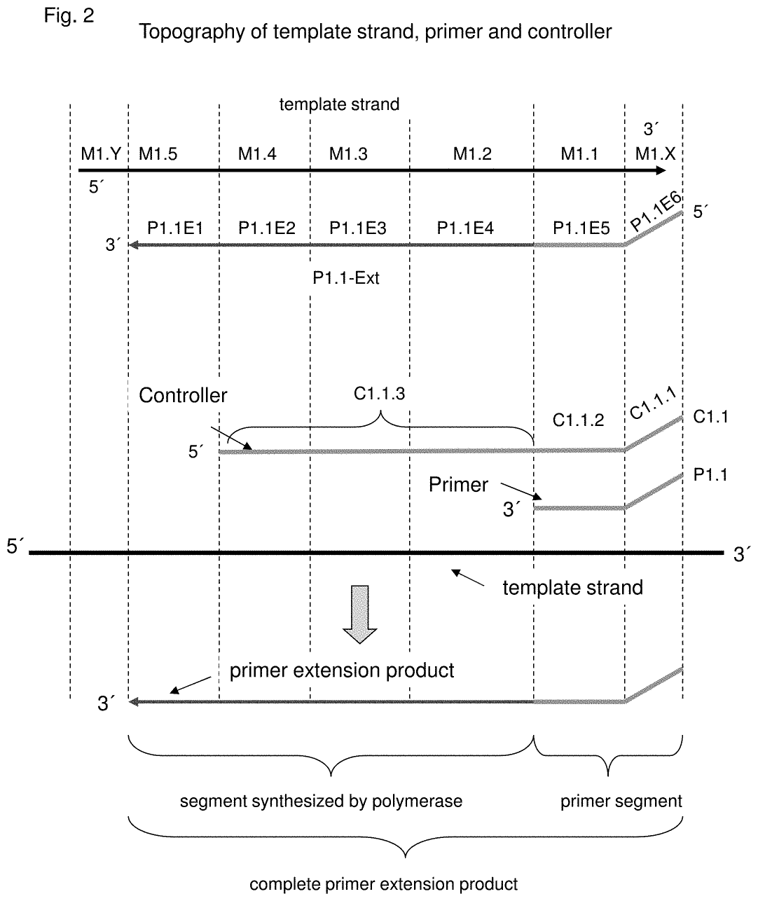

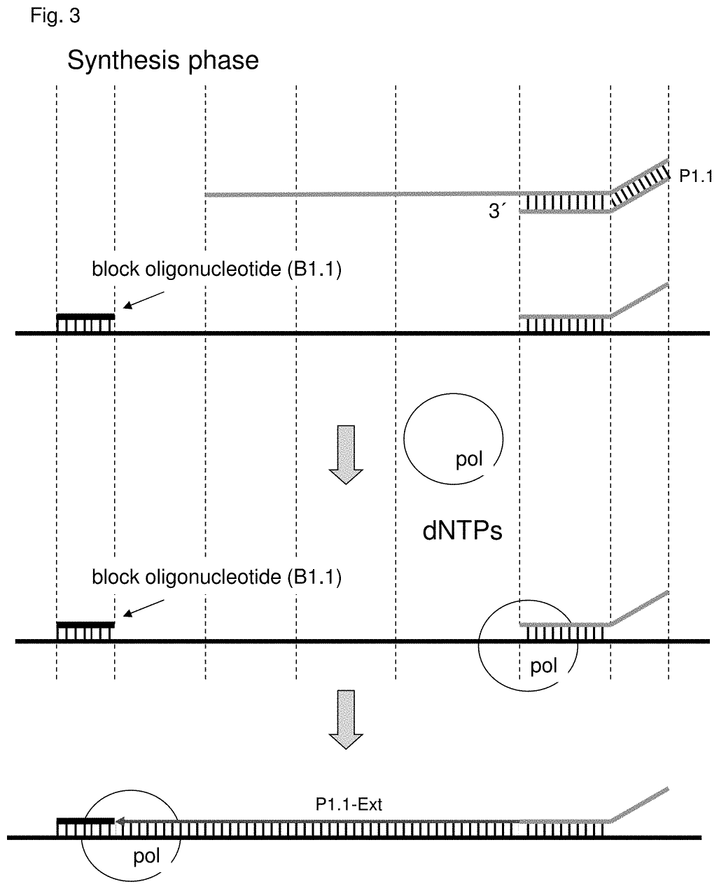

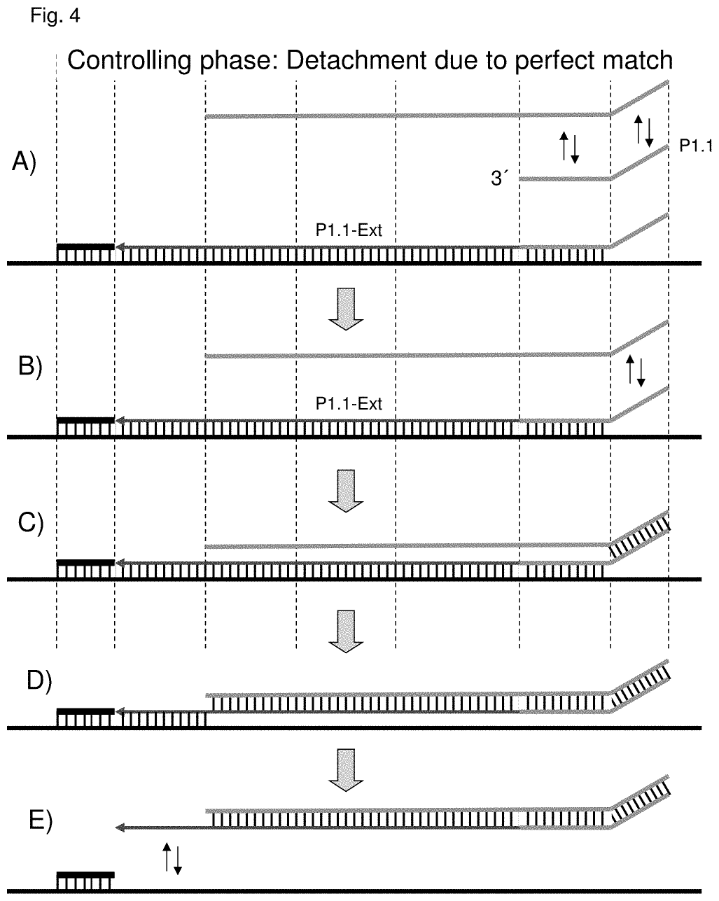

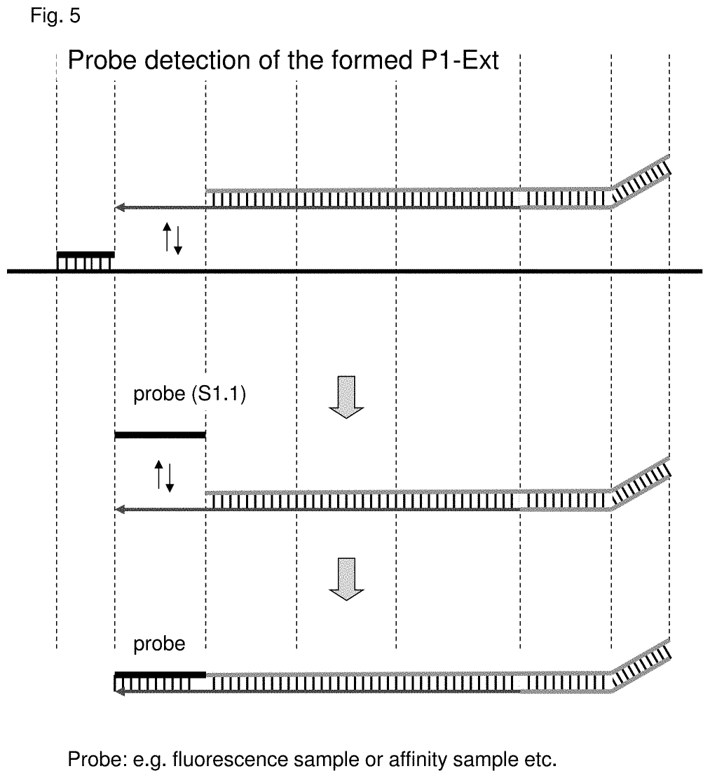

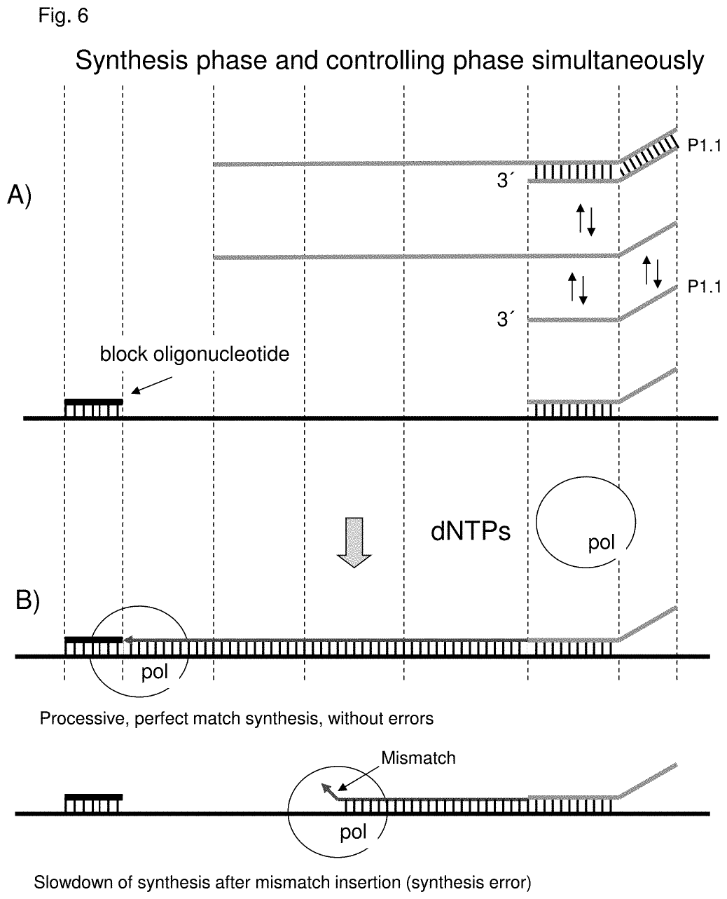

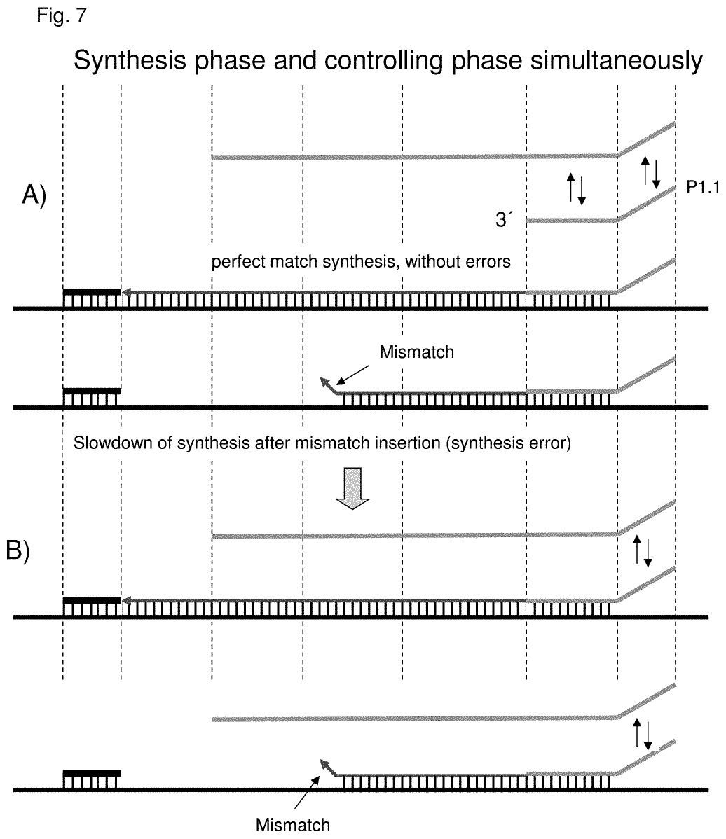

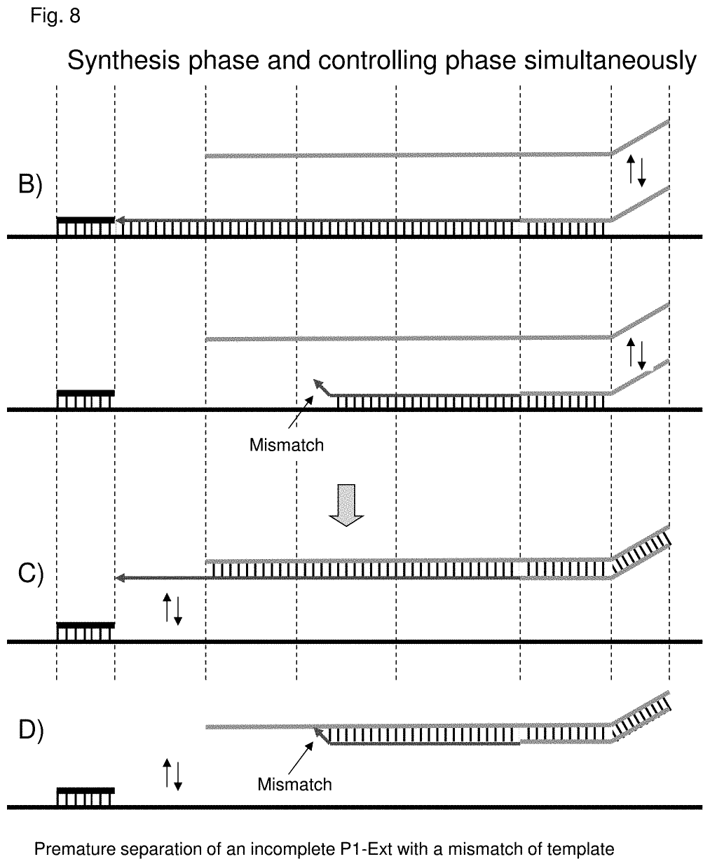

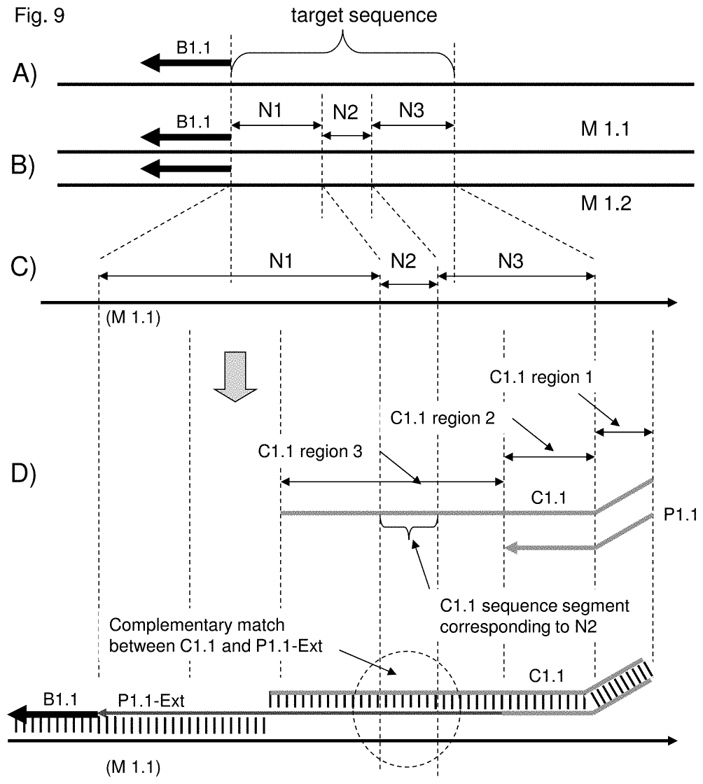

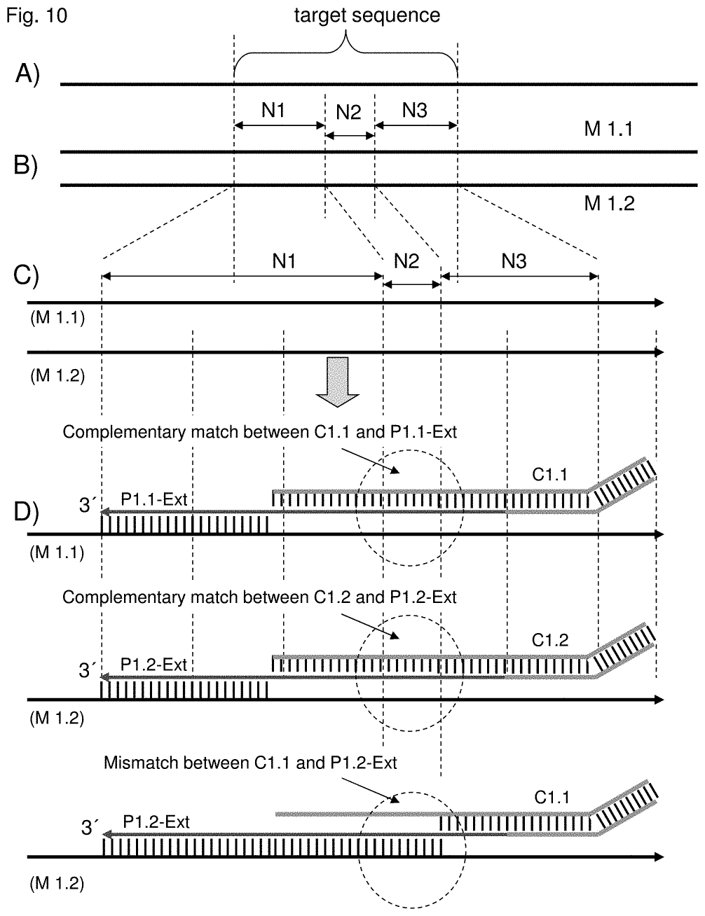

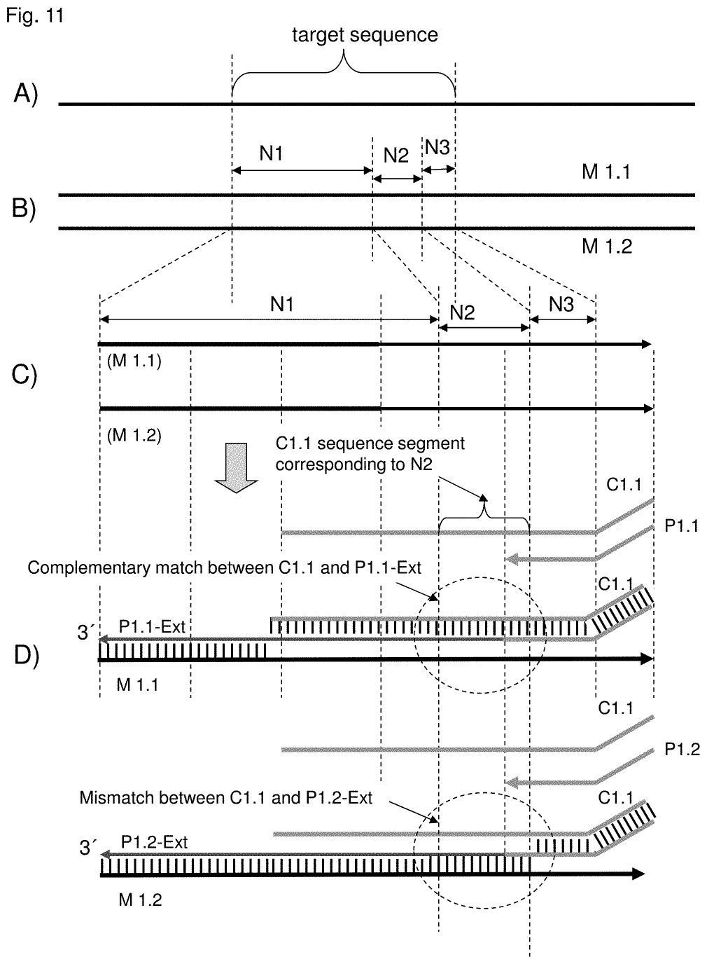

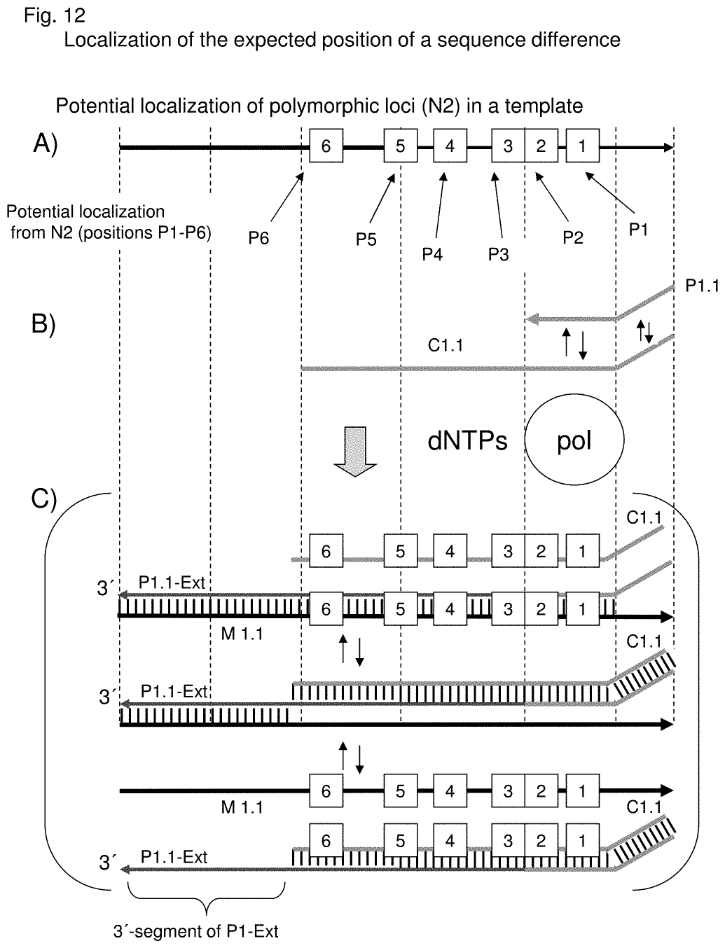

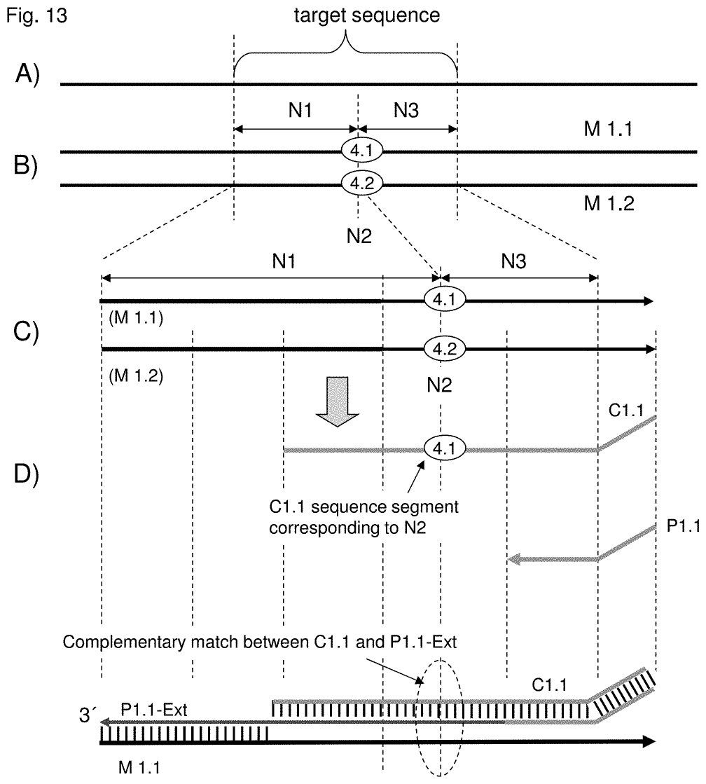

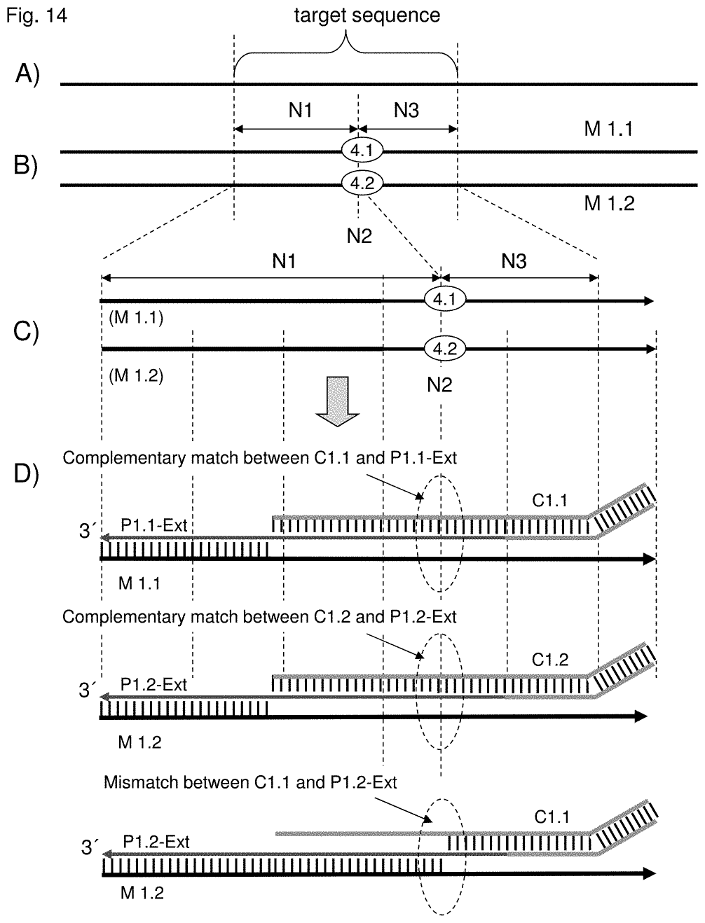

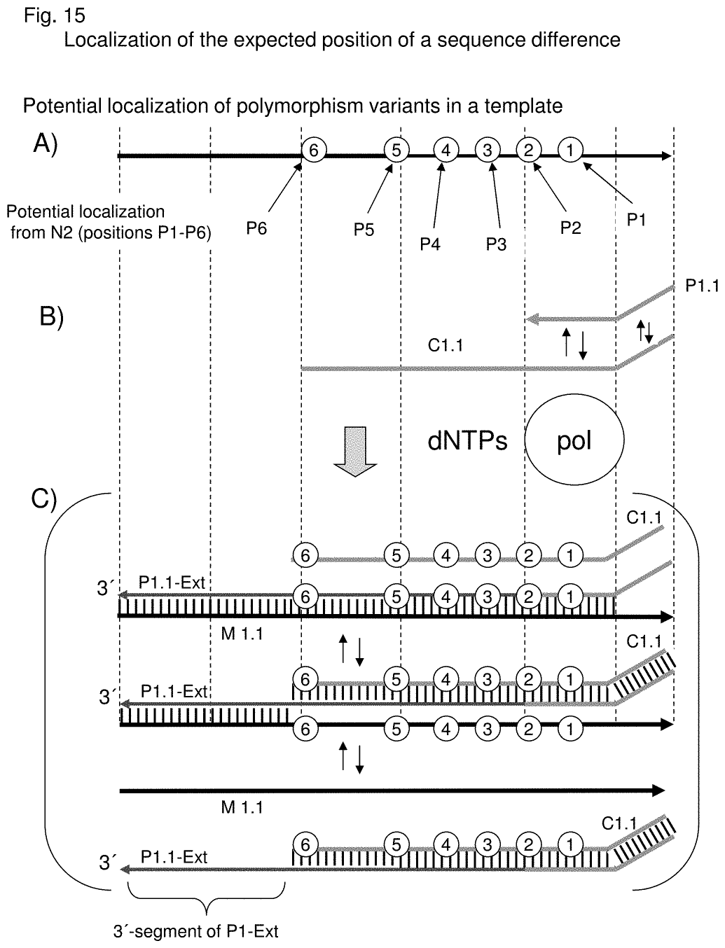

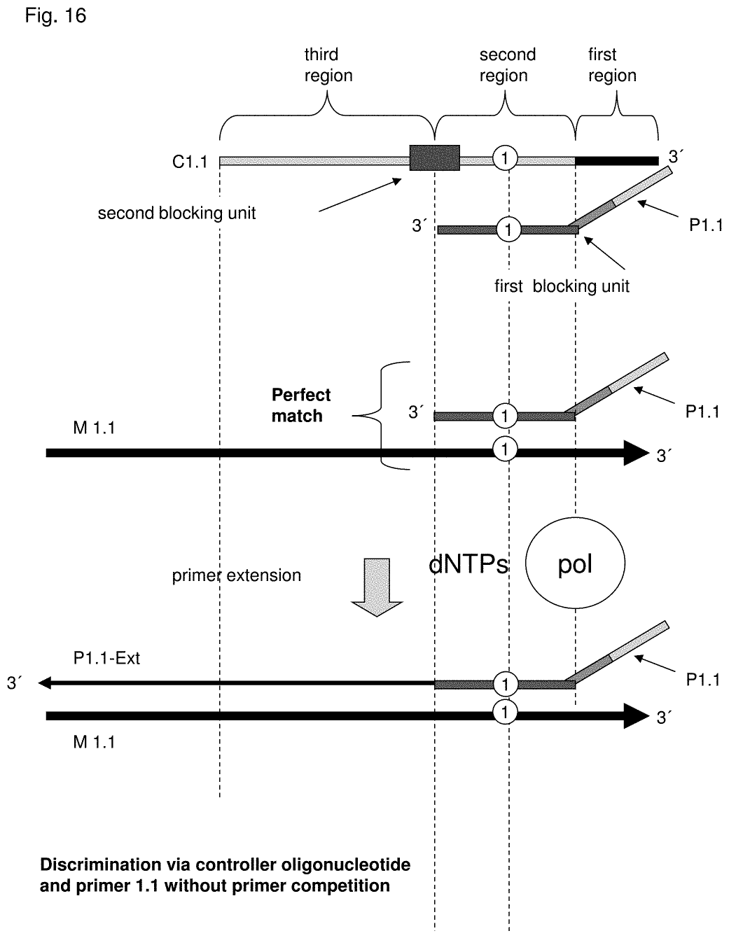

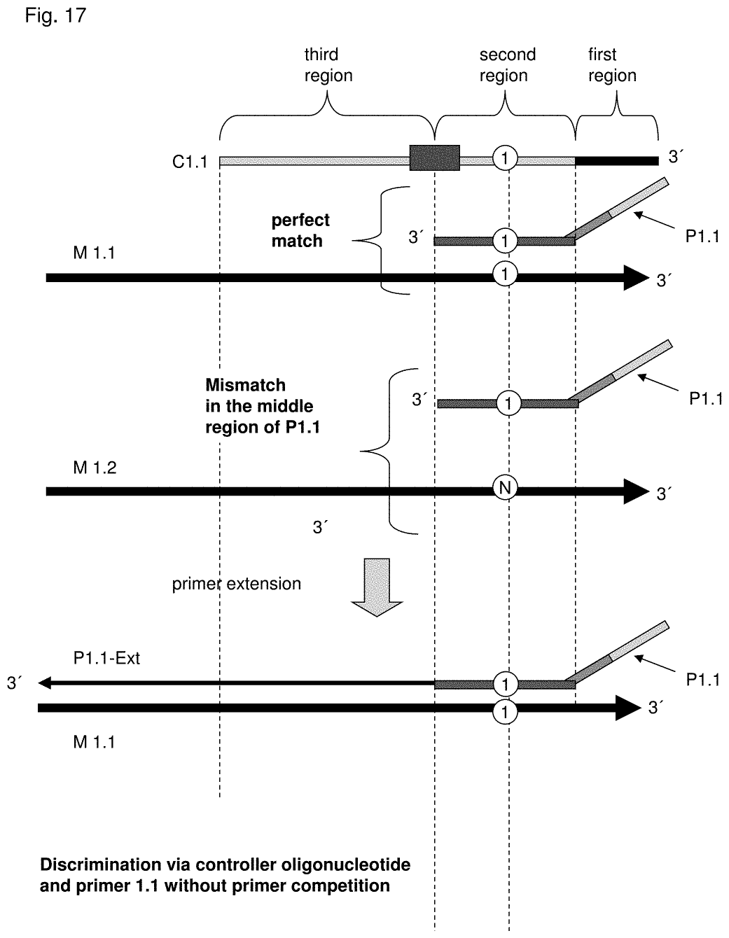

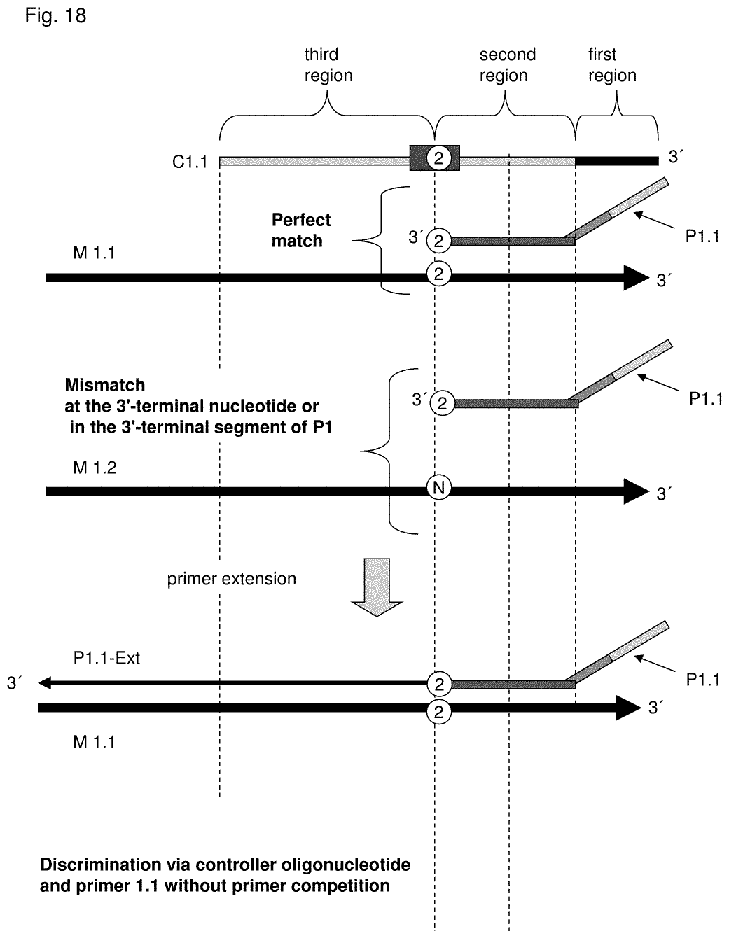

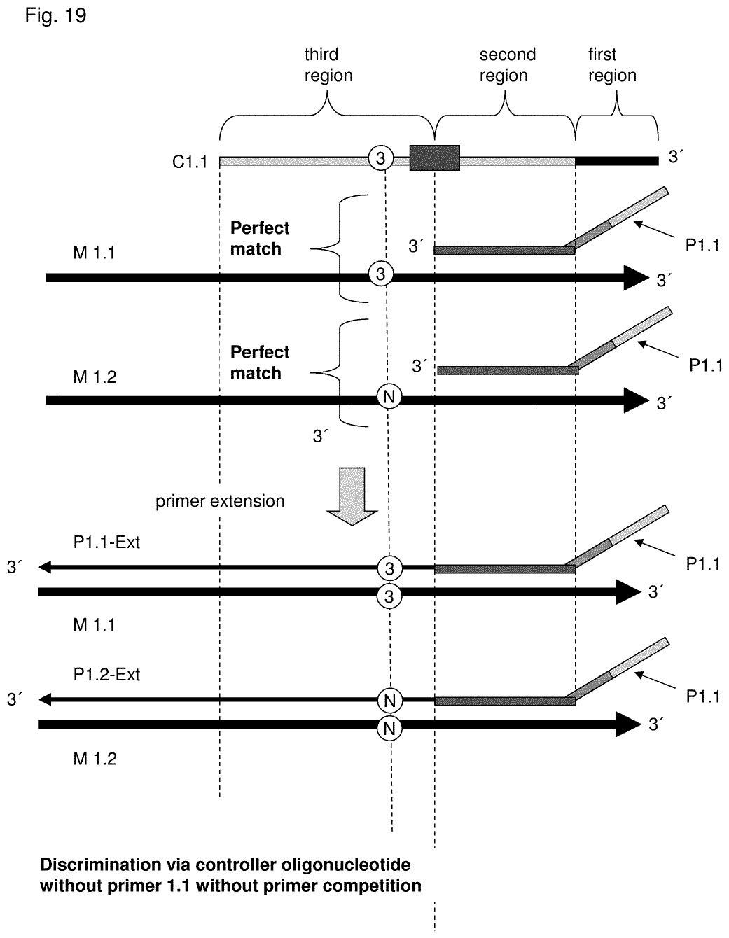

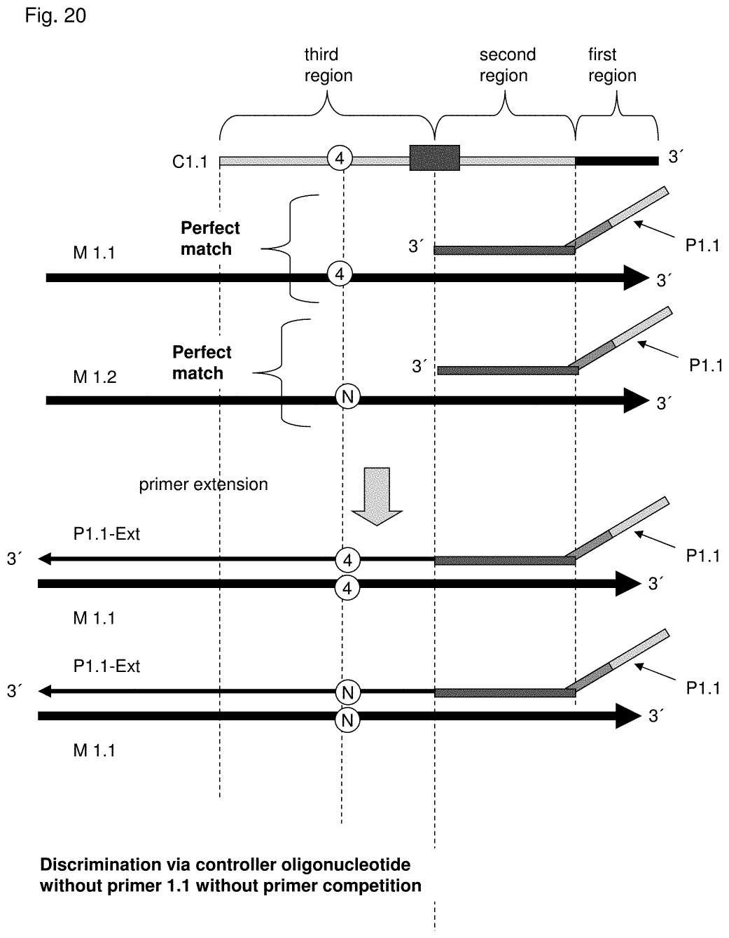

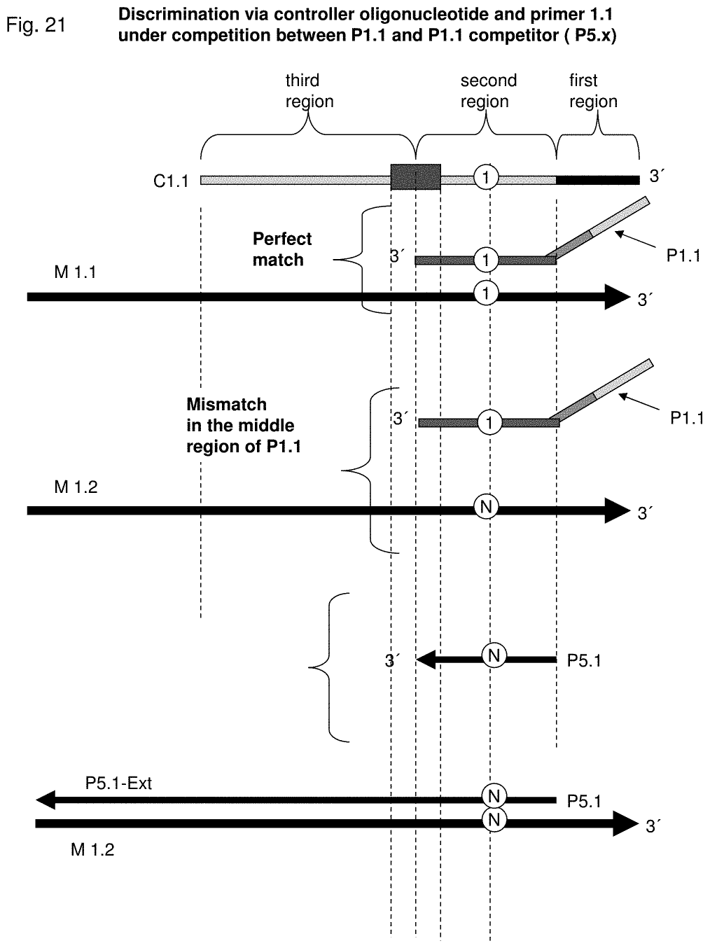

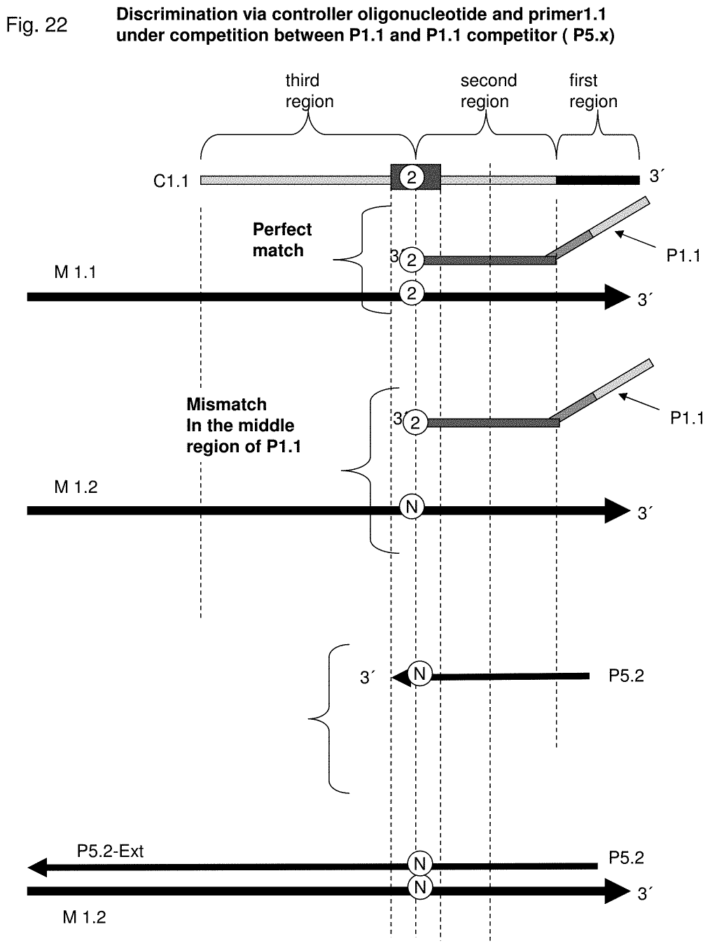

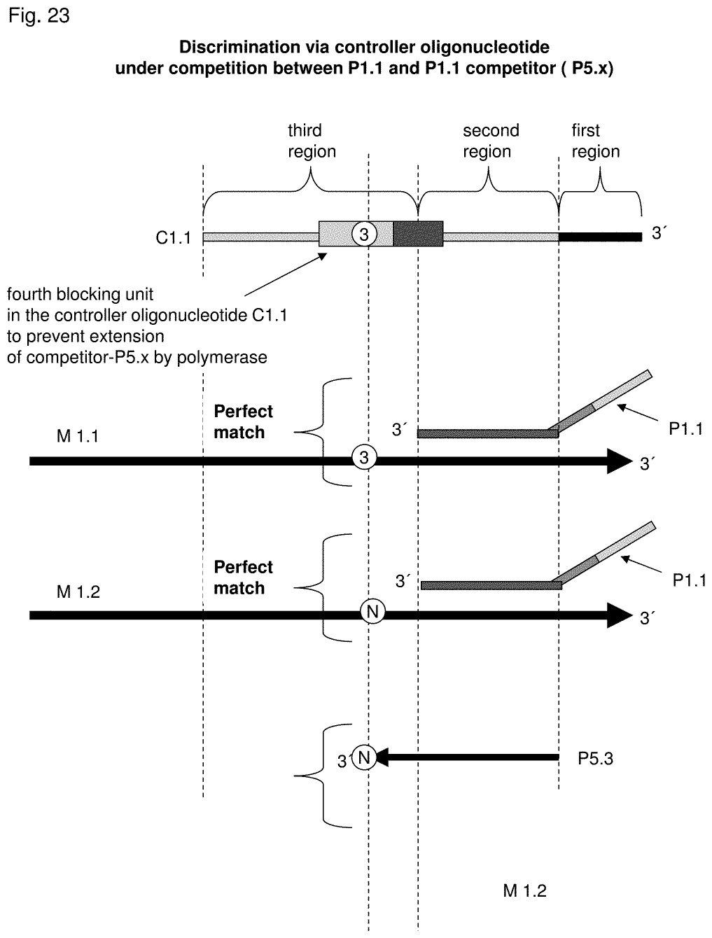

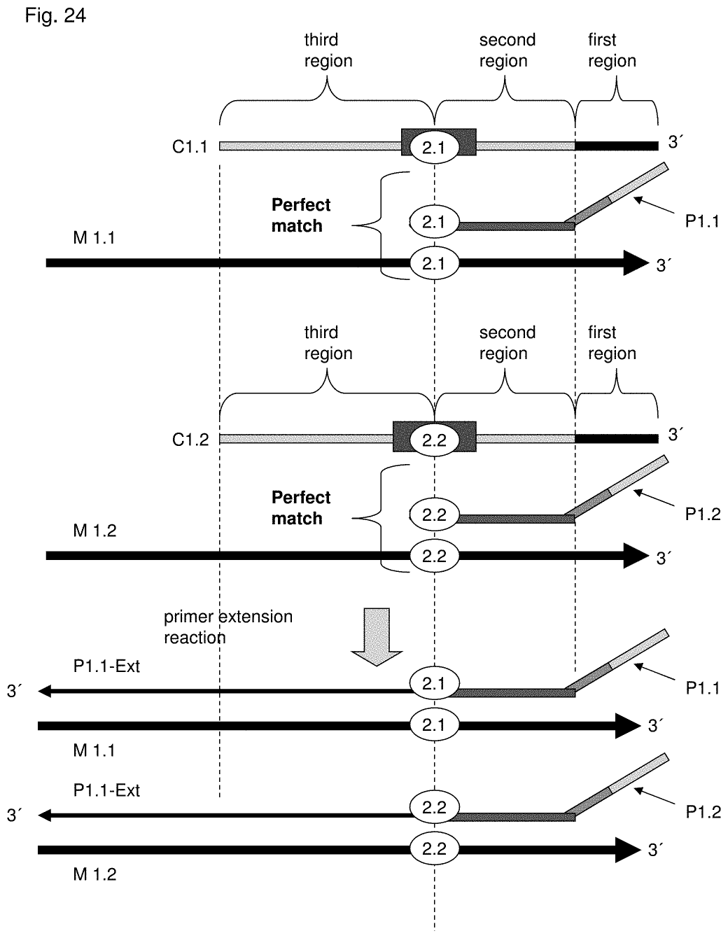

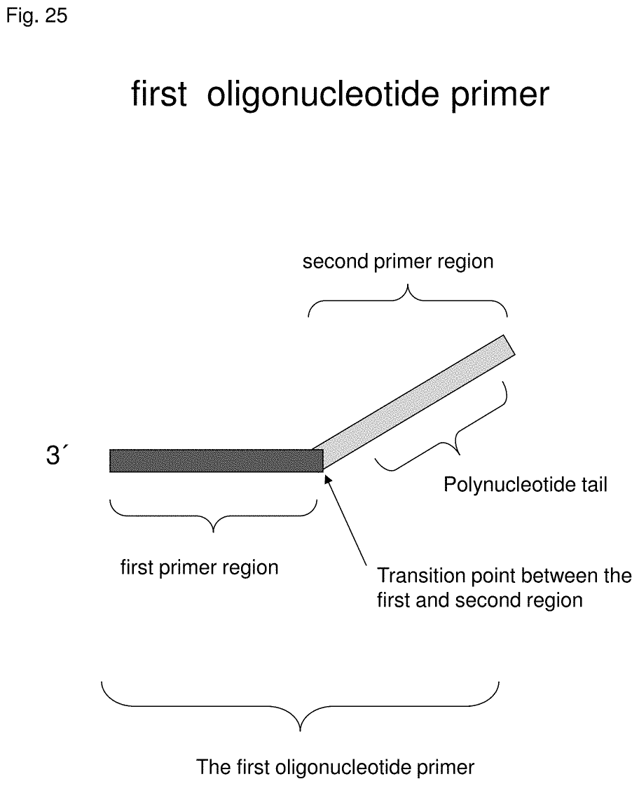

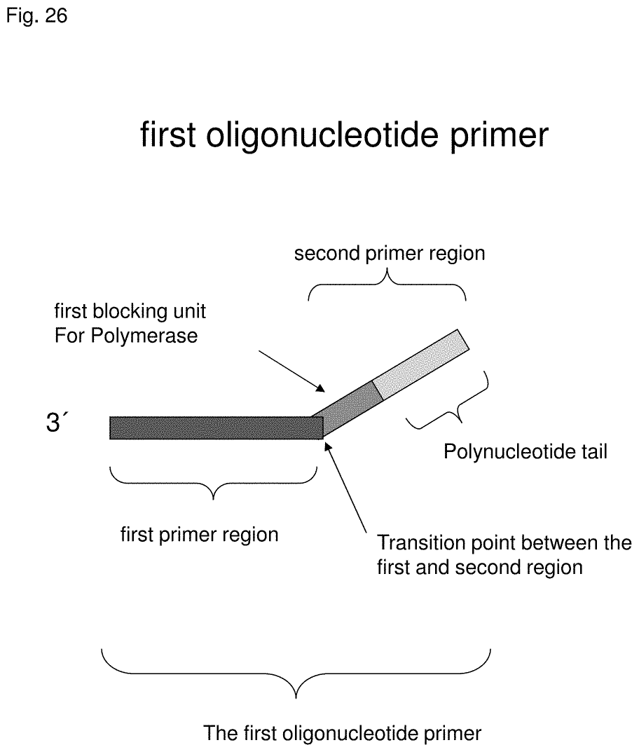

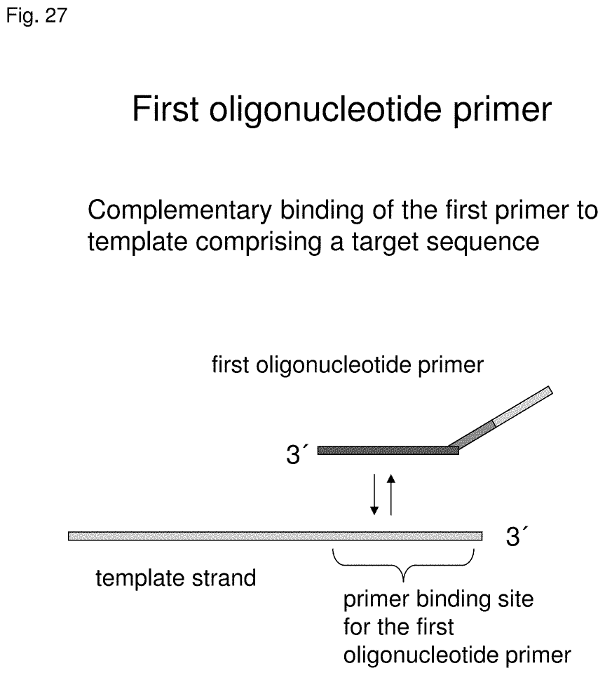

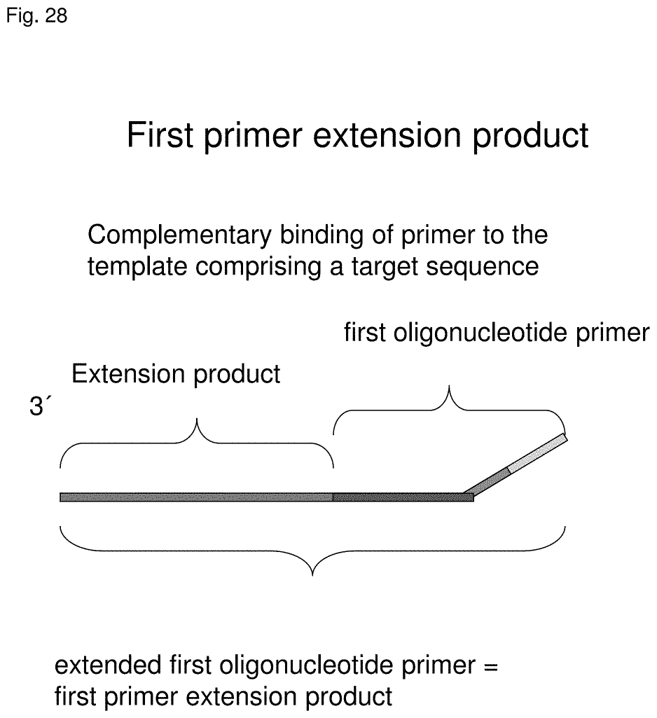

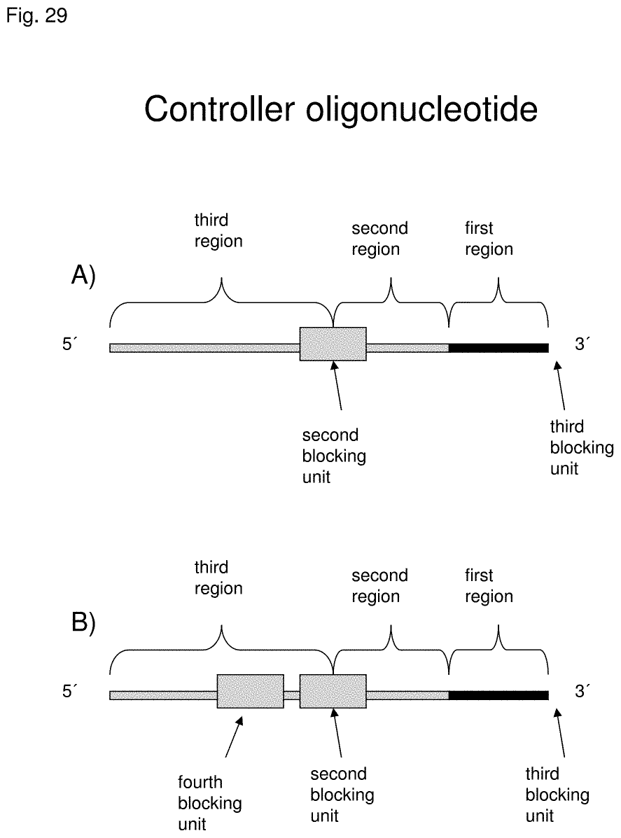

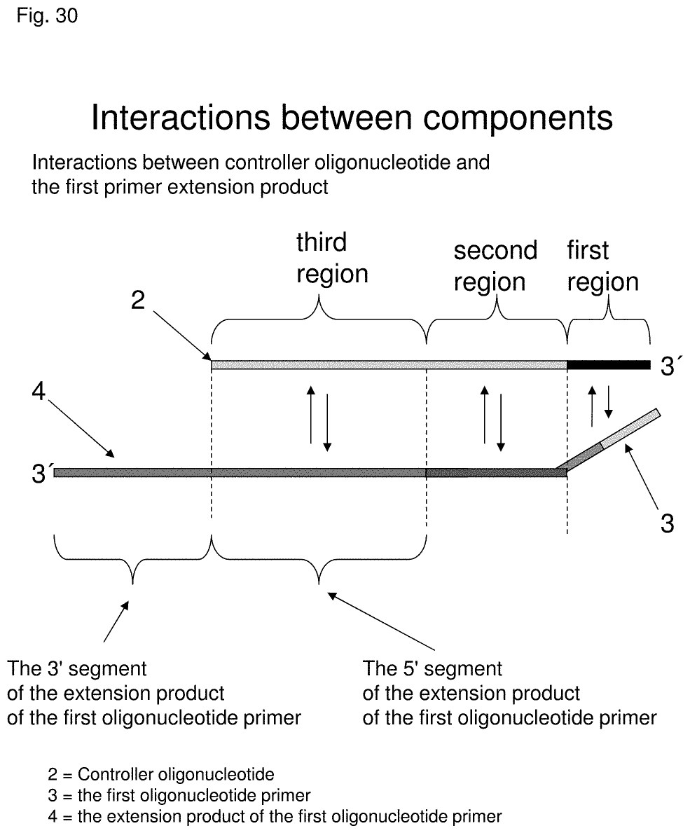

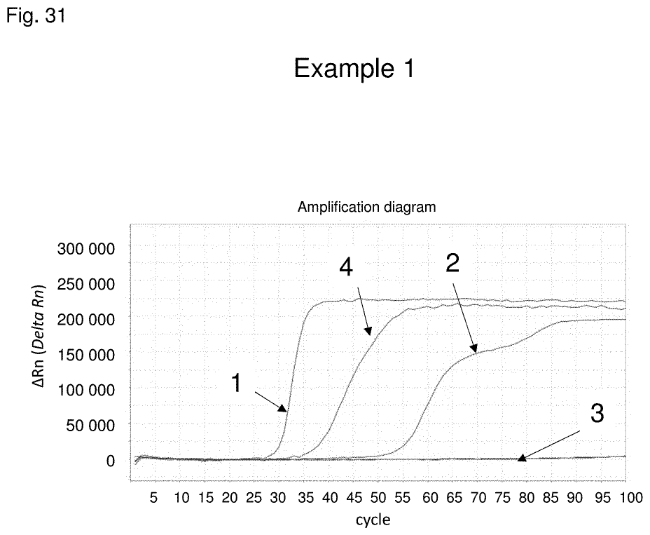

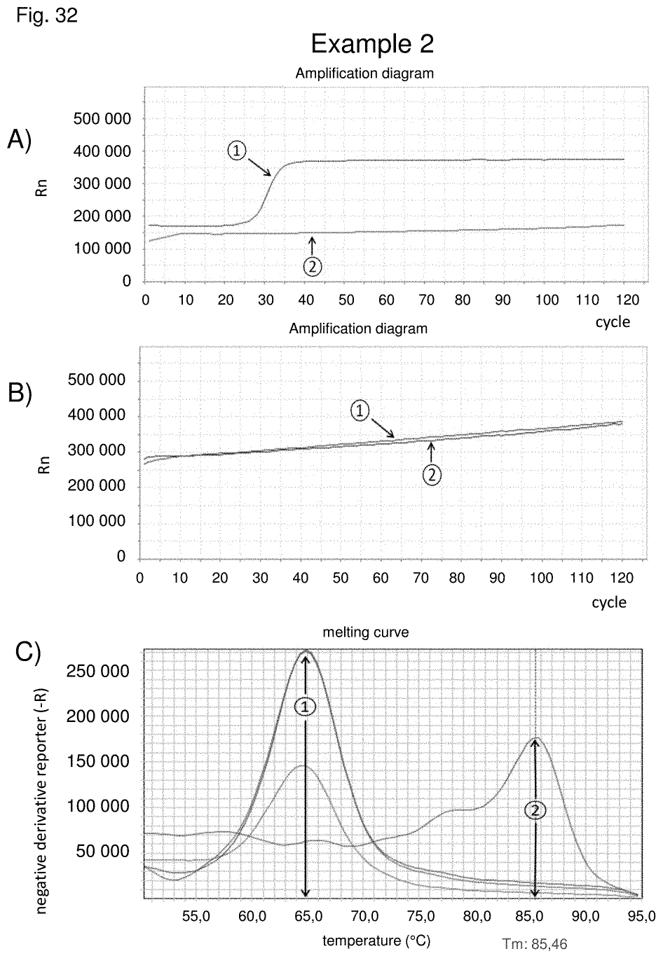

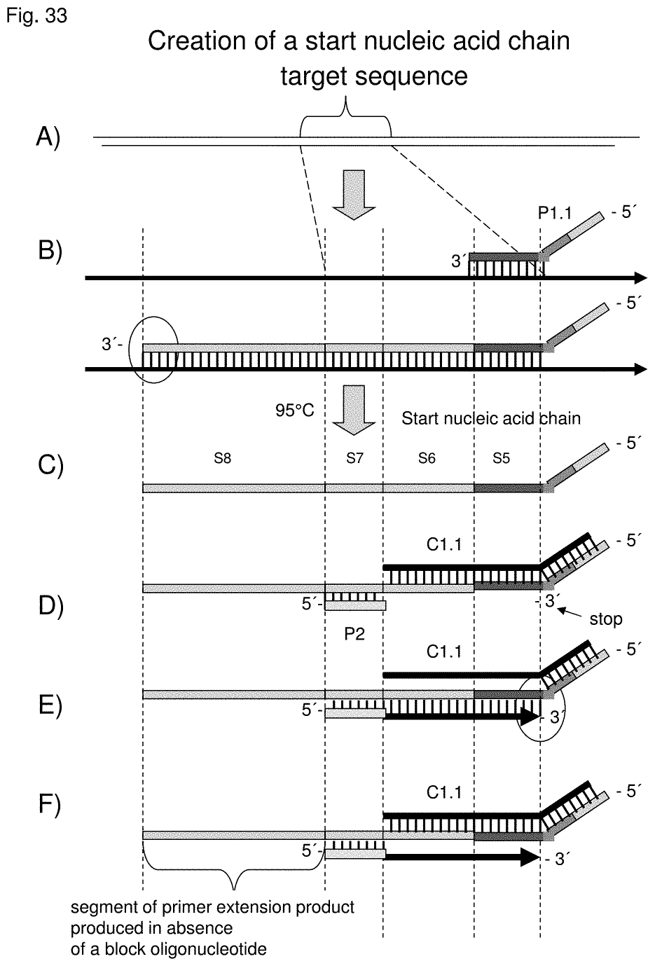

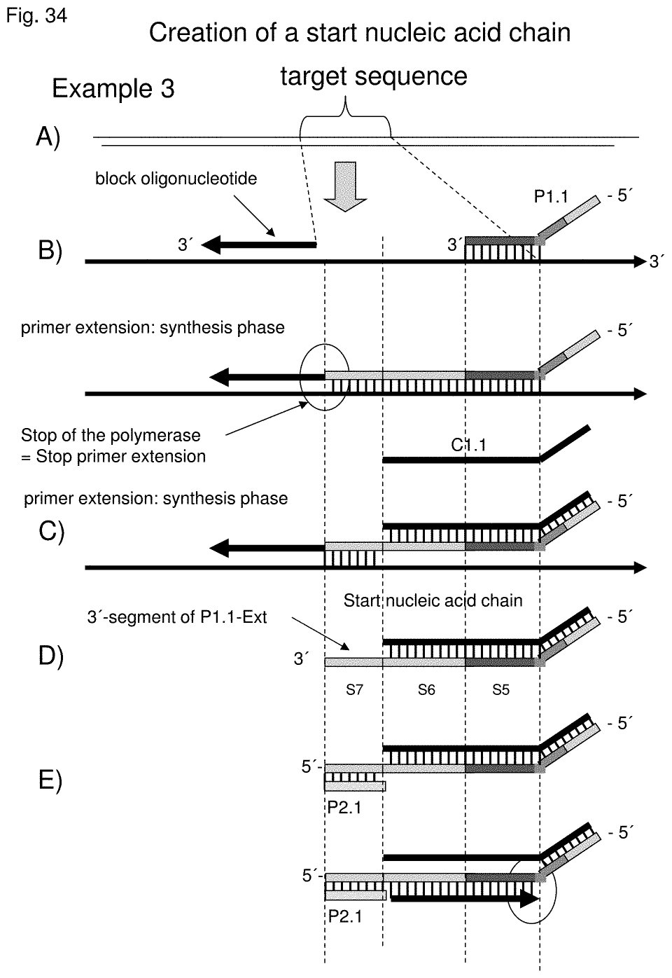

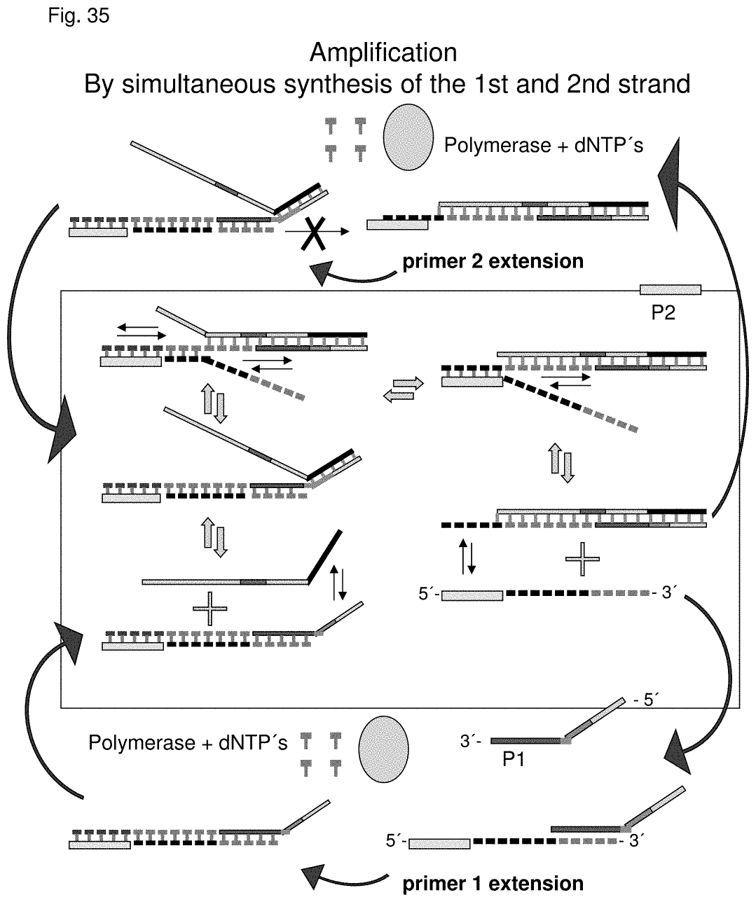

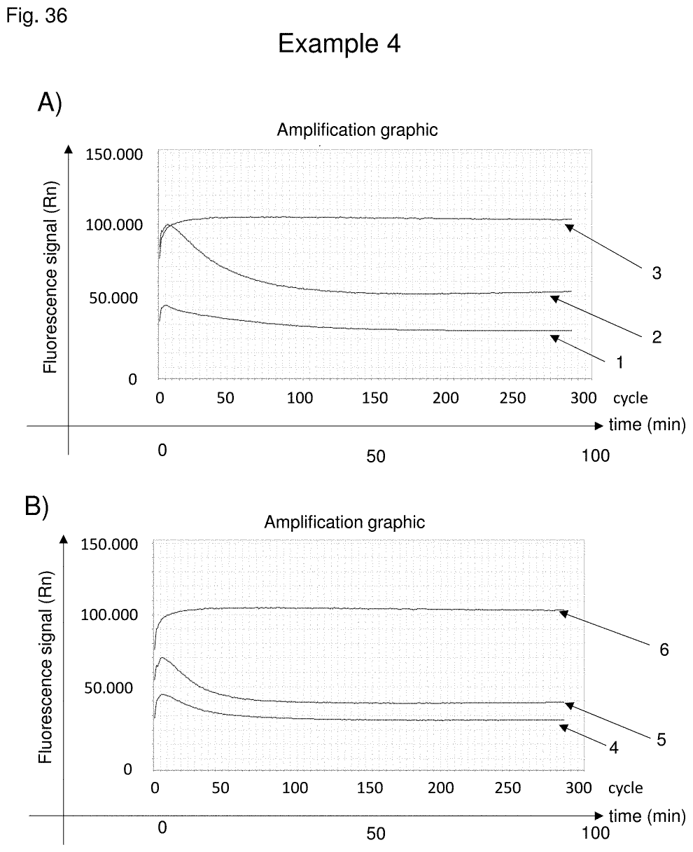

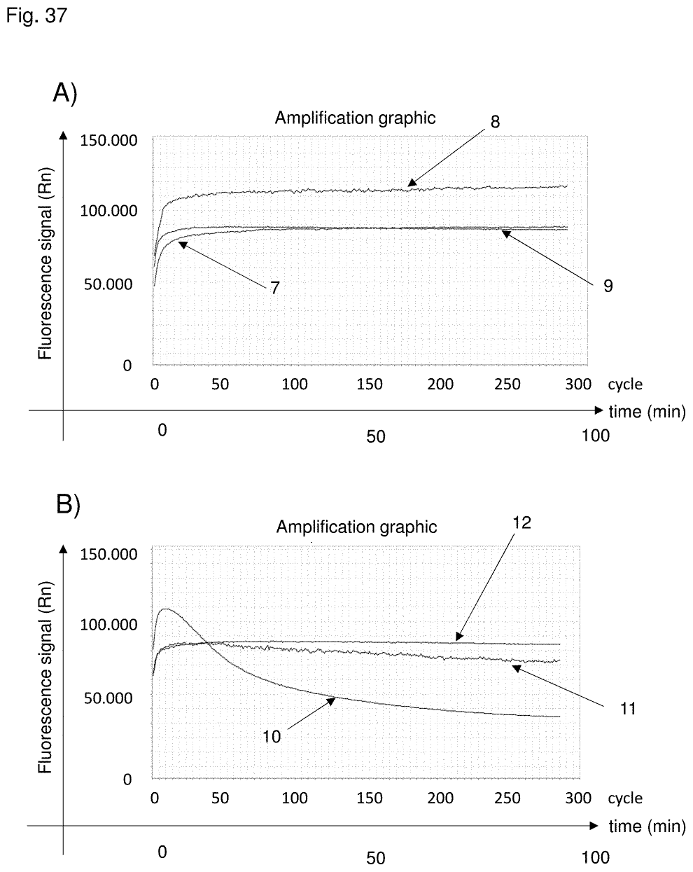

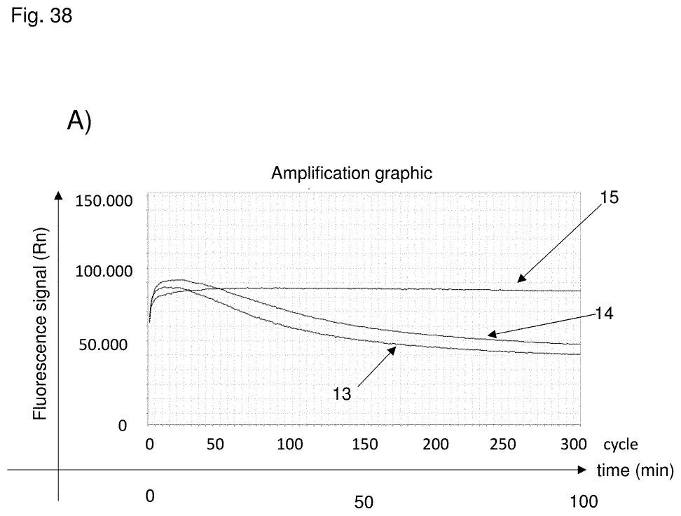

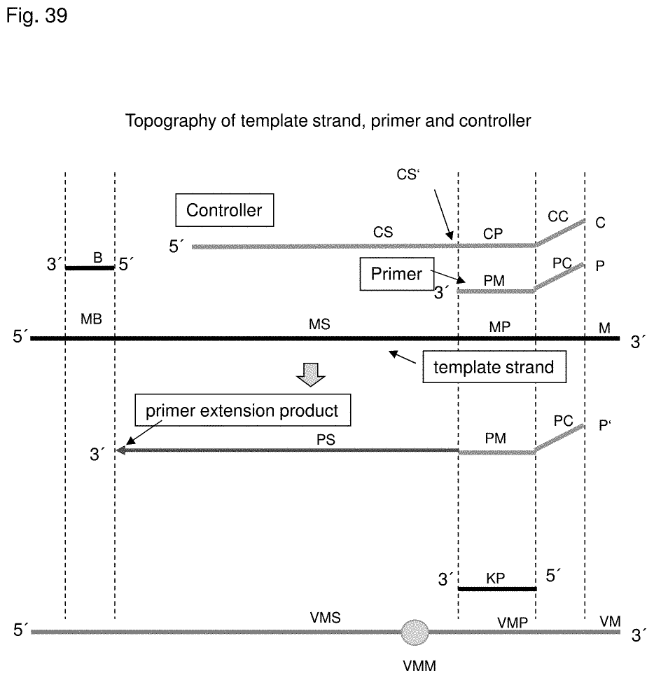

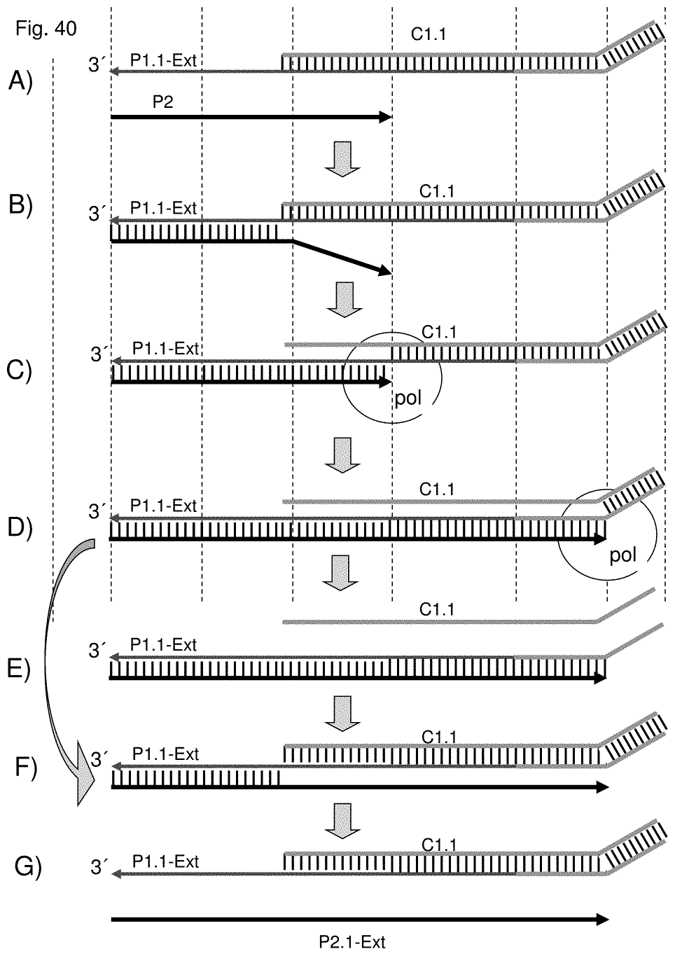

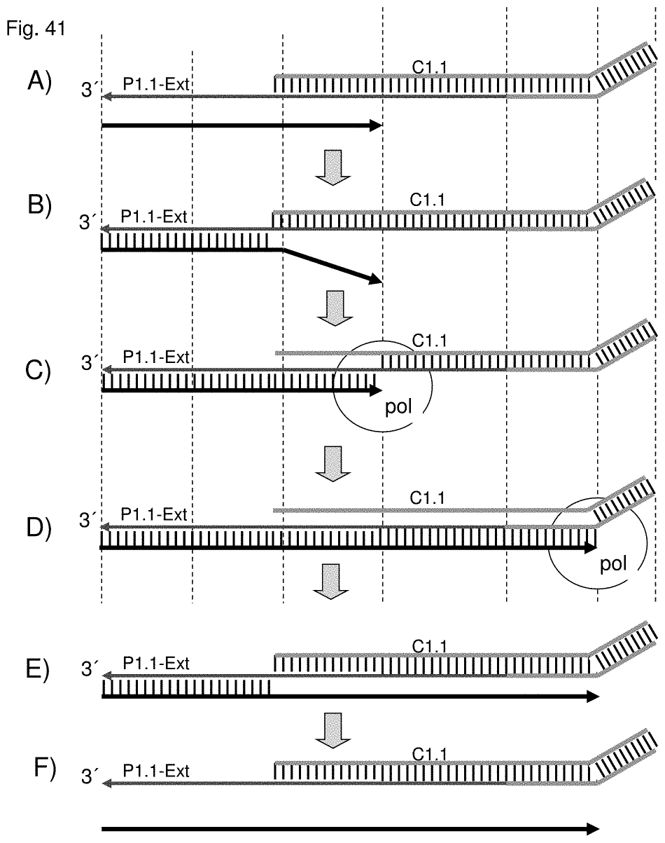

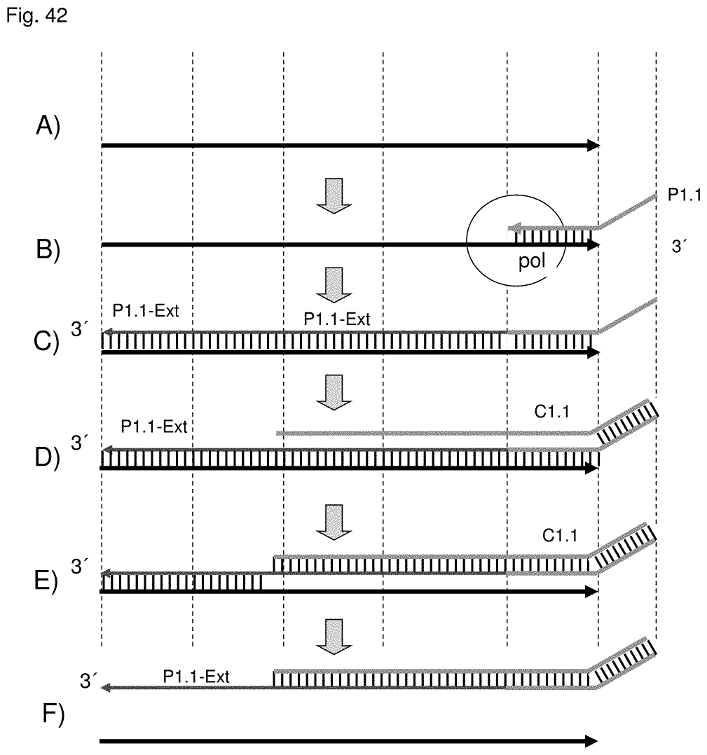

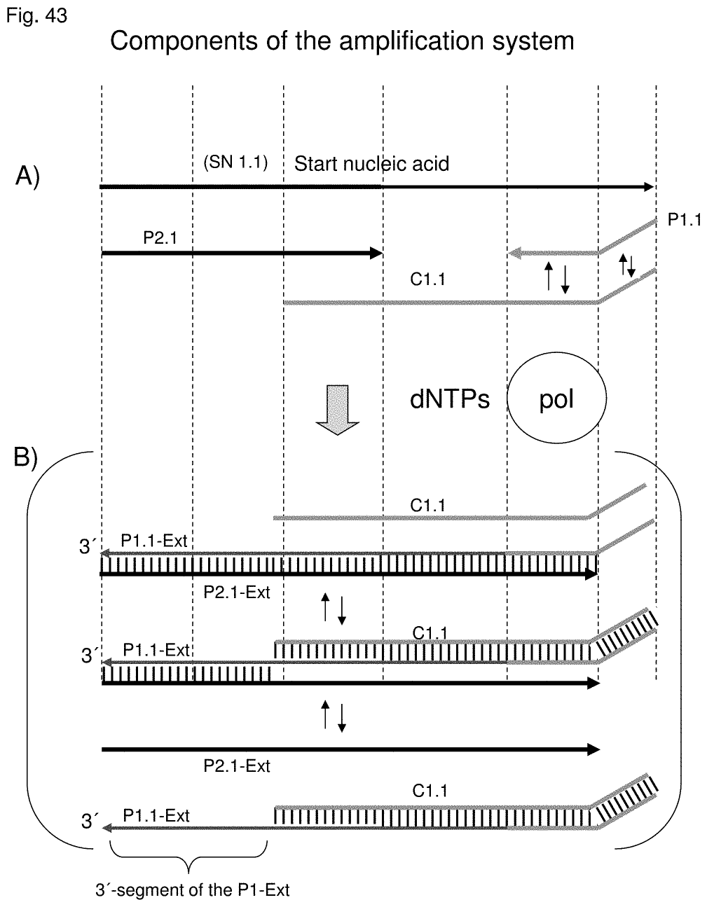

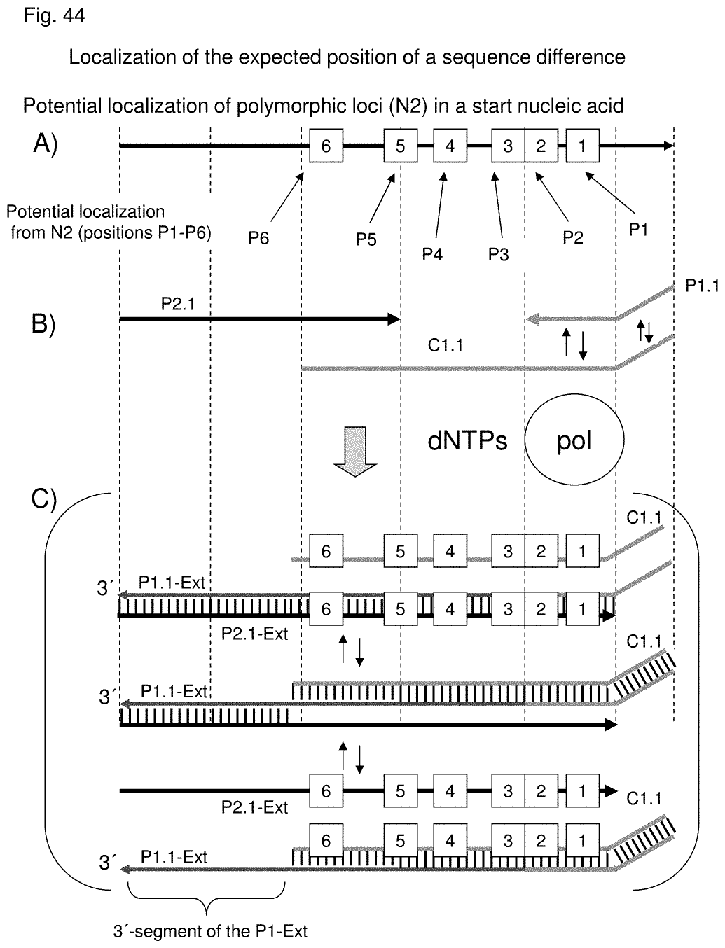

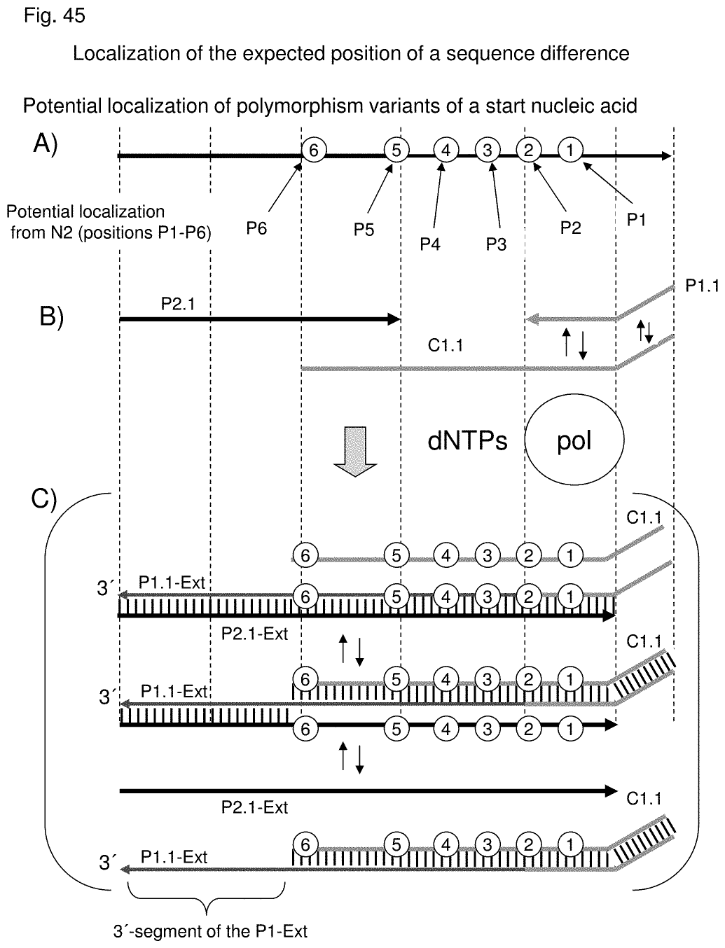

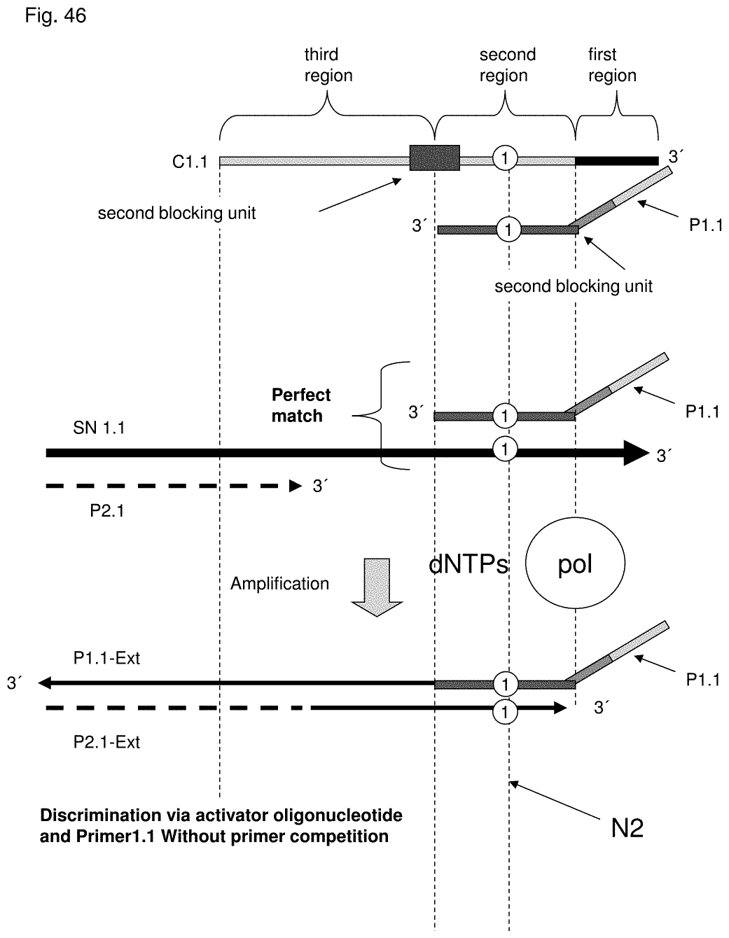

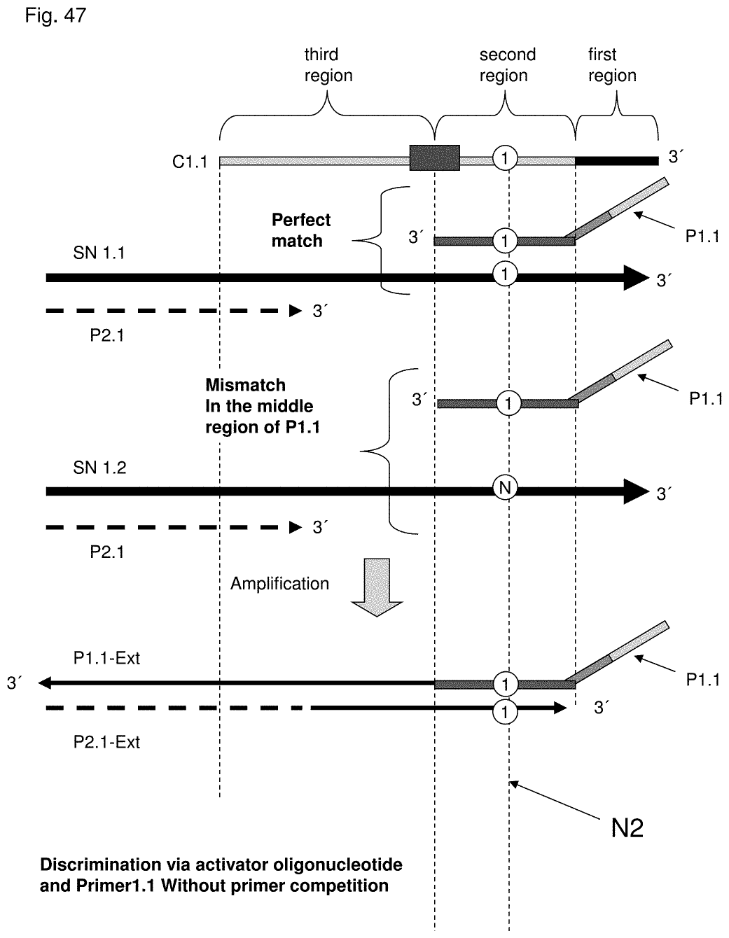

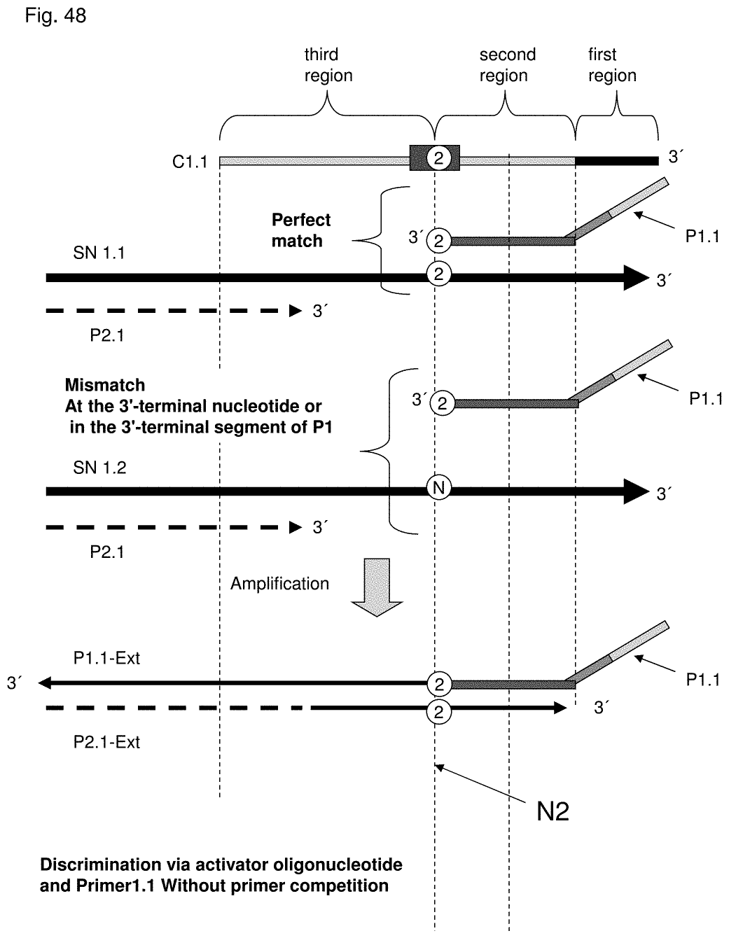

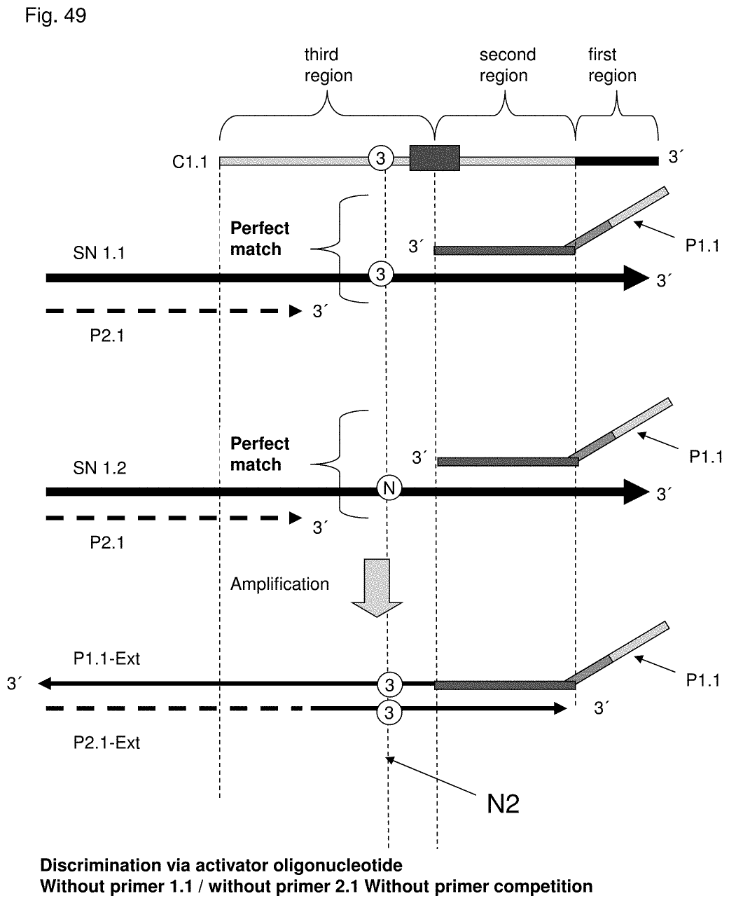

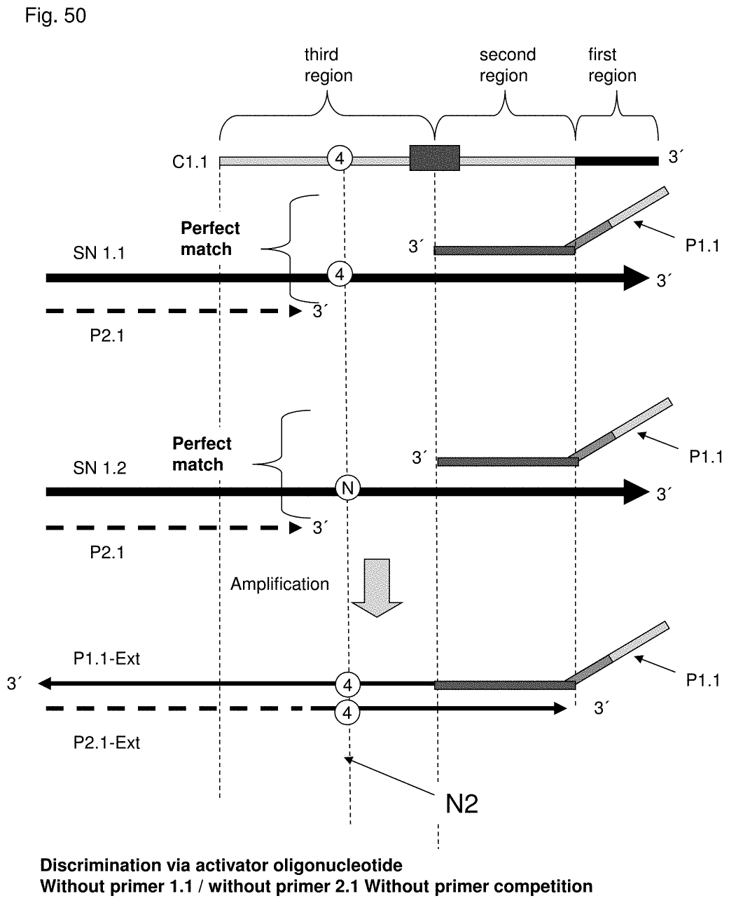

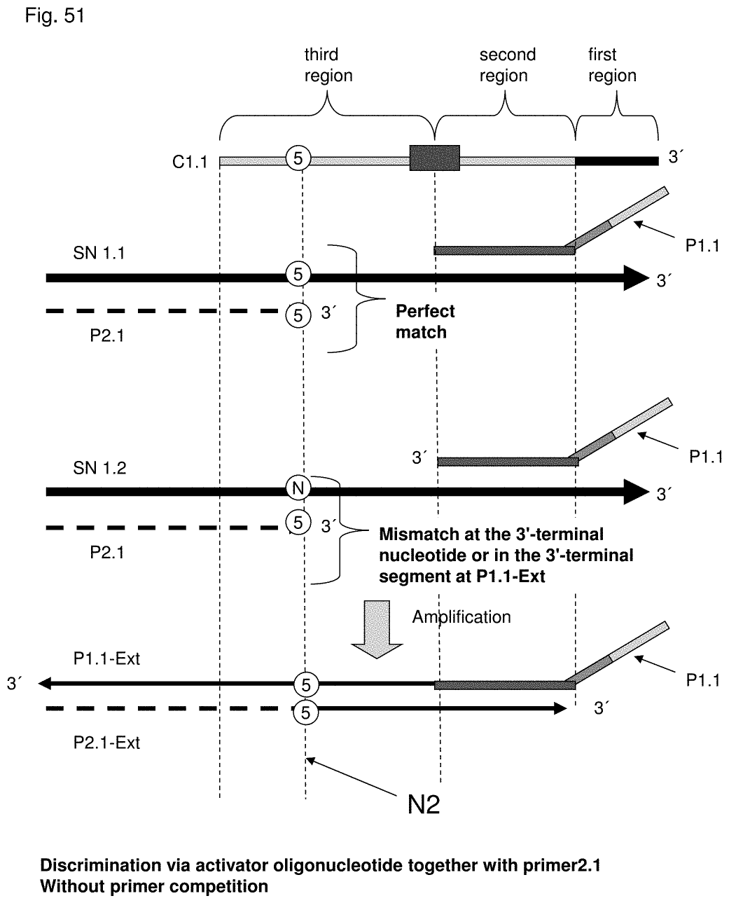

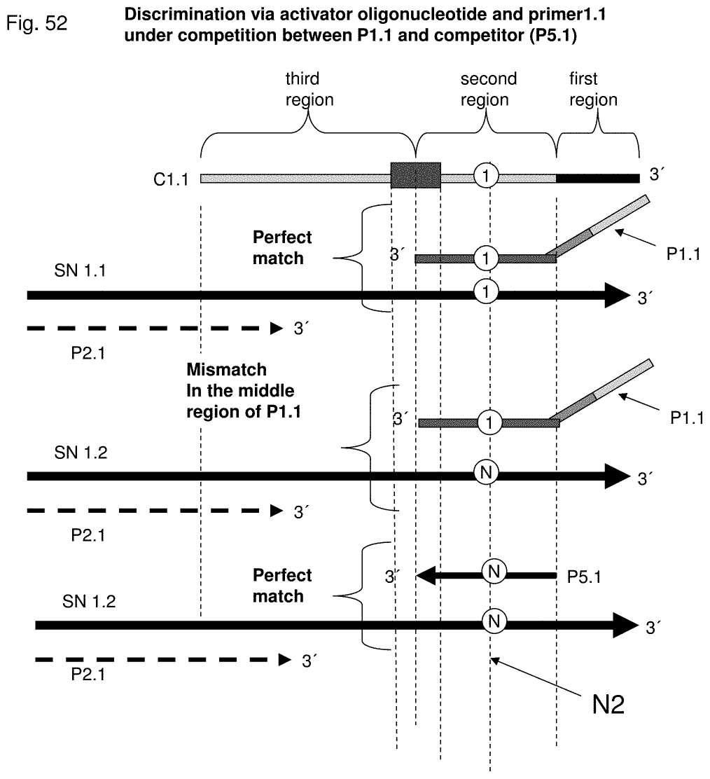

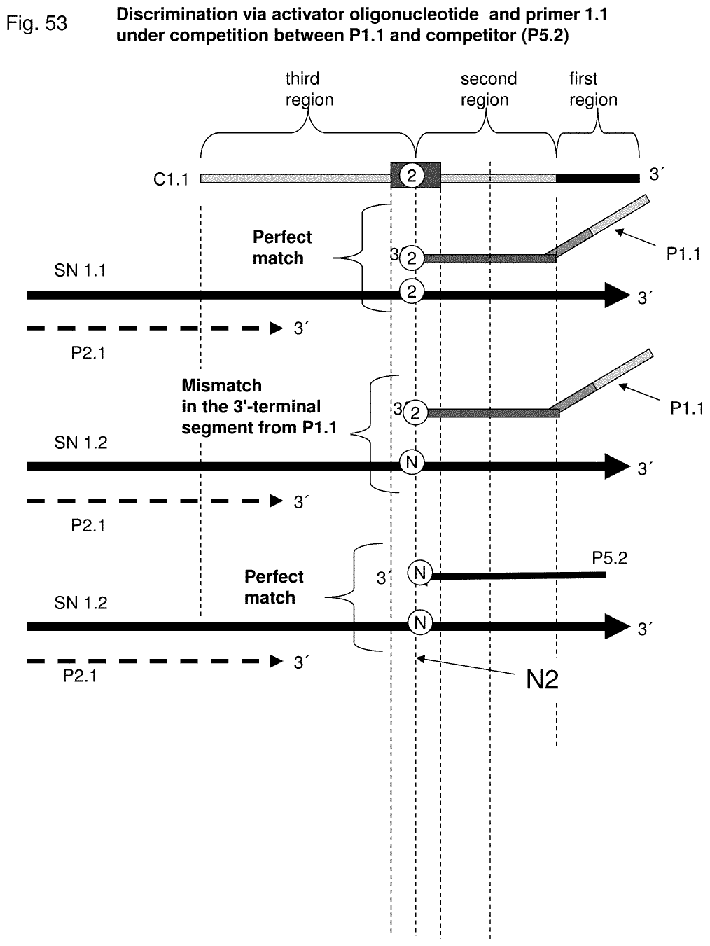

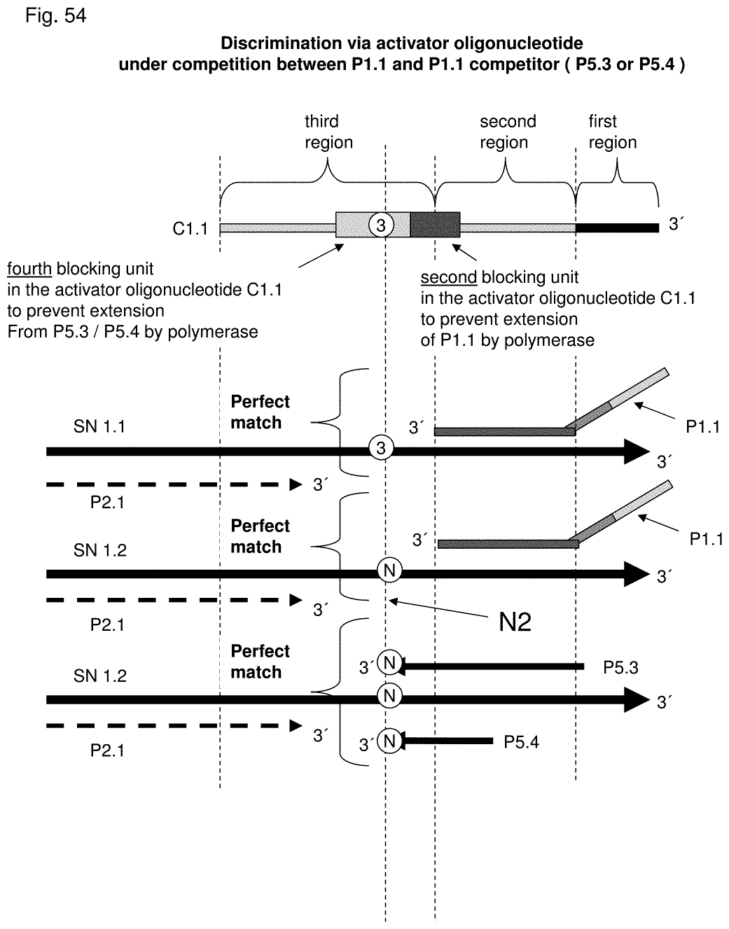

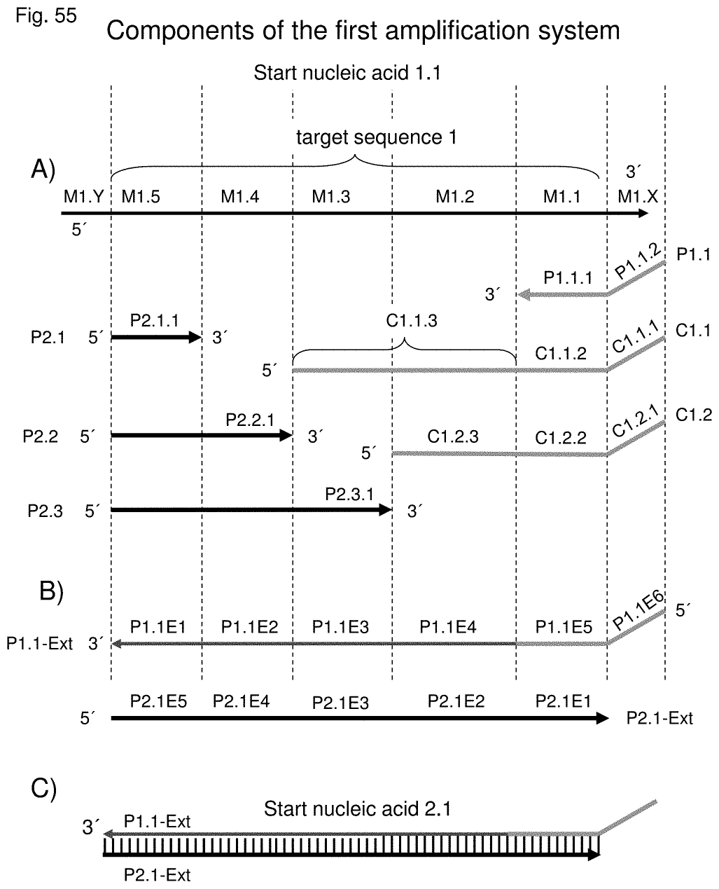

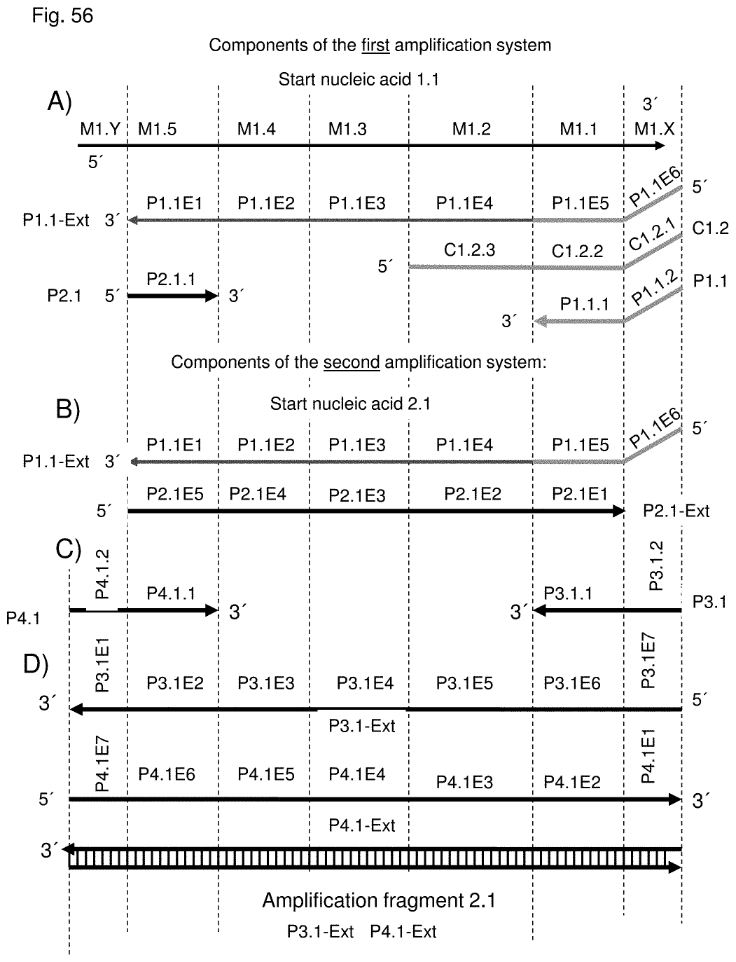

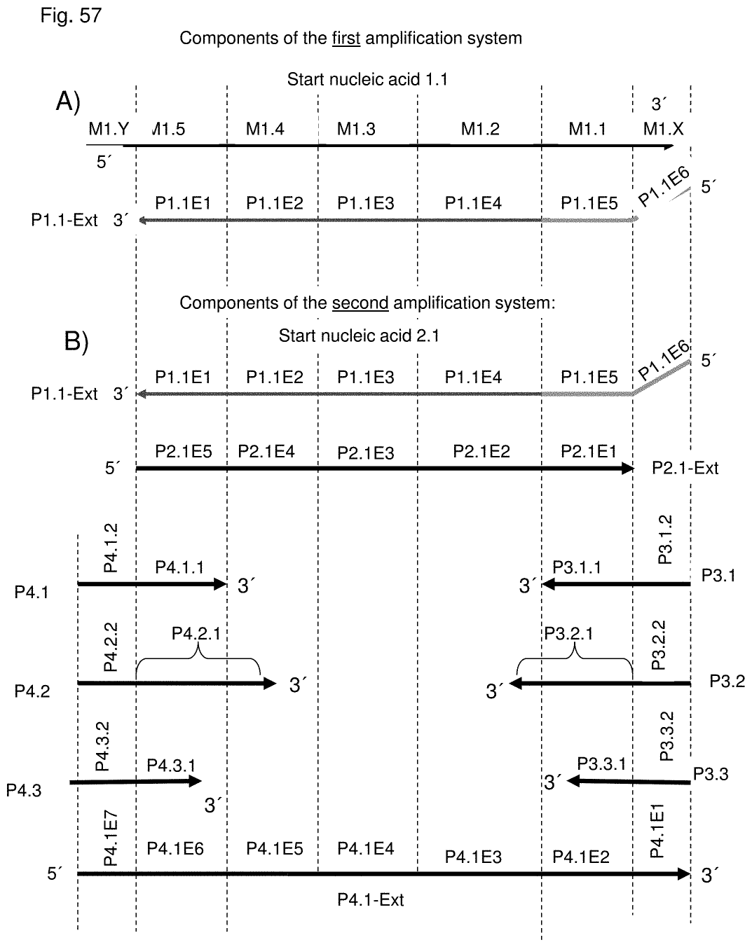

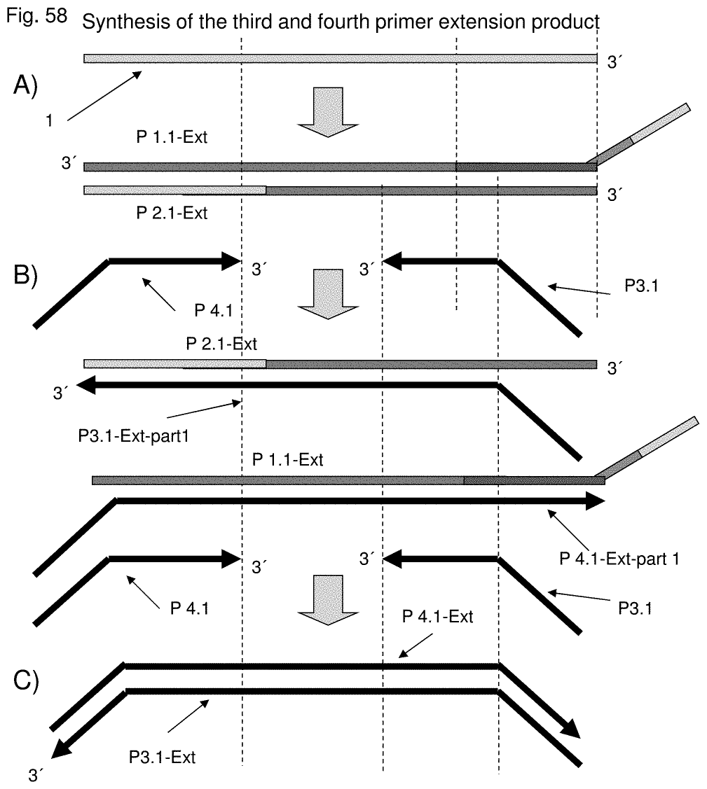

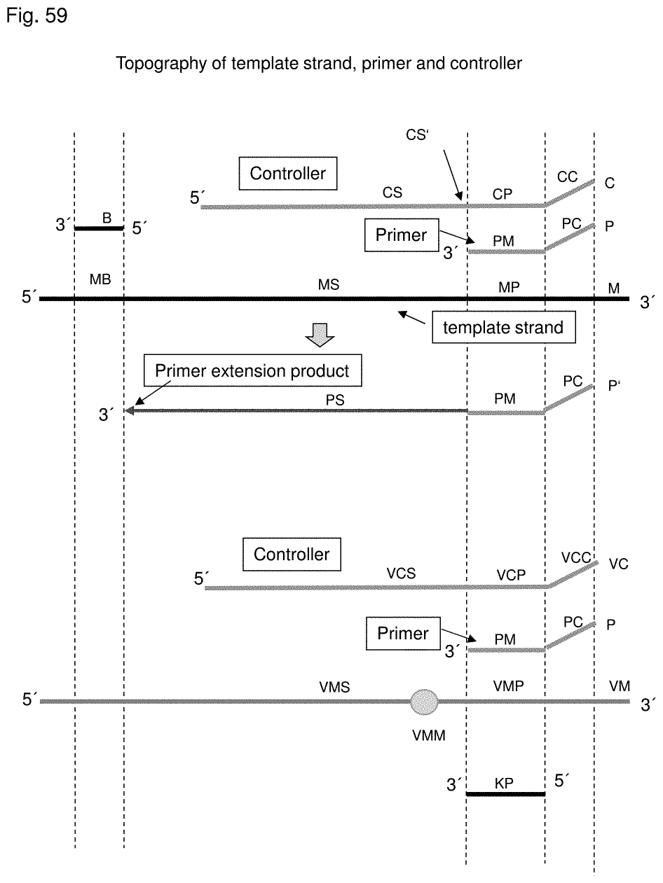

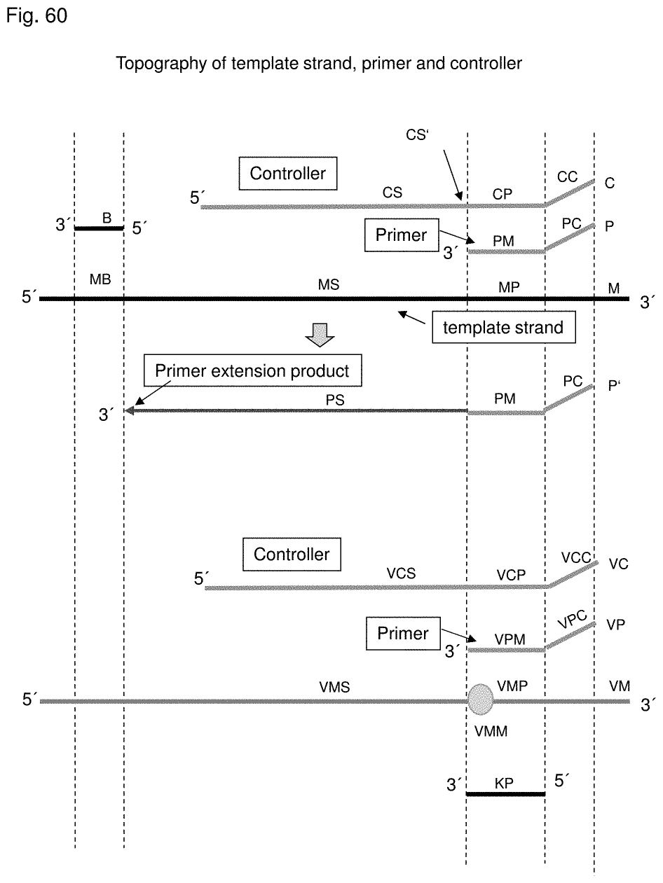

The synthesis of a P1-Ext primer extension product is shown schematically in FIG. 3. The synthesis progress is stopped sequence specifically by a block oligonucleotide. The primer/controller complex is stable during the synthesis phase under the reaction conditions used. The synthesis of the P1-Ext by the polymerase proceeds up to the block oligonucleotide (B1.1), so that the length of the synthesized segment of the P1-Ext is limited.

The sequence-specific control of the primer extension is particularly mediated by the binding of a controller oligonucleotide. The controller binding to the primer extension product during the controlling phase is schematically shown in FIG. 4, wherein A) illustrates the release of the controller out of the complex: primer/controller; B-C) the binding of the controller to the second region of the primer on the primer extension product, and D-E) the sequence-specific strand displacement by the controller with release of the template strand.

FIG. 5 schematically illustrates probe binding to the primer extension product after its release from the template strand. The release of the template strand allows the 3′ segment of the P1-Ext to interact with another oligonucleotide, e.g. a fluorescence probe (S1.1), or other primers or an affinity probe. Binding can, for example, result in the increase of a fluorescence signal.

As a probe, for example, an oligonucleotide complementary to the 3′ segment of the formed primer extension product can be used with a quencher (e.g. BHQ1) and a reporter (e.g. FAM). A molecular beacon with self-quenching properties can be used as reporter. The signal is only generated when a complementary binding occurs. This allows to distinguish for example complete P1-Ext from incomplete P1-Ext.

The synthesis phase and the controlling phase can take place simultaneously, as illustrated by the reaction setup shown in FIG. 6. Controller and primer as well as their complex are present in equilibrium during the reaction (FIG. 6 A)). The synthesis of the primer extension product is initiated by the addition of a polymerase. When primer binds to the primer binding site, the polymerase can initiate the synthesis of a complementary primer extension product (FIG. 6B)). The synthesis of fully complementary products catalyzed by the polymerase takes place faster than the synthesis of products which comprise a mismatch due to an error occurring during the synthesis. The mismatch strands are synthesized more slowly.

FIGS. 7 and 8 show further schematic representations of a reaction setup with simultaneous synthesis phase and controlling phase. Controller and primer as well as their complex are present in equilibrium during the reaction (FIG. 7A), FIG. 8A)). The free controllers in the batch can interact with the primer extension products during the synthesis phase. The interaction starts at the second primer region of a respective primer extension product (FIG. 7B), FIG. 8B)). The result of the interaction of the controllers with the primer extension products varies depending on the synthesis progress at the primer extension product: if P1-Ext is fully synthesized, said product can be detached. A fully synthesized P1-Ext can interact with other oligonucleotides through its 3′-segment, e.g. probes, primers etc. (FIG. 8C)). In the case of incompletely synthesized P1 -Ext, this product can also detach from the template strand. The complex formed is sufficiently stable under reaction conditions, so that this incomplete primer extension product can be excluded from continuing its synthesis on the template strand or is strongly prevented from continuing the template-dependent synthesis reaction by the complex form with the controller. Thus, the synthesis of defective primer extension products up to the full synthesis length can be prevented. Due to insufficient length, such incomplete P1 -Ext, for example, do not comprise the necessary sequence segments to participate in other reactions. For example, a 3′ segment is missing in the primer extension product (FIG. 8D)).

FIG. 9A to C show schematically the topography of a template strand (comprising a target sequence) with a polymorphic locus (N2) and two uniform target sequence segments (N1 and N3). The template strand with the first variant (M 1.1) and the template strand with the second variant (M 1.2) schematically show the topography of a target sequence within template strands. Both template strands comprise target sequence segments N1 and N3, which are identical and uniform in both template strands. The respective sequence segment N2 is characteristic and specific for each template strand, so that both template strands can be distinguished. N2 comprises at least two sequence variants of a polymorphic locus of a target sequence.

In said embodiment, a target sequence which can comprise several sequence variants in a polymorphic locus (N2), further comprises at least a first target sequence segment (N1) which is characteristic and uniform for all target sequence variants of a target sequence (target sequence group comprising here M 1.1 and M 1.2). Furthermore, a target sequence comprises at least one second target sequence segment (N3), which is characteristic and uniform for all target sequence variants of a target sequence (target sequence group comprising here M 1.1 and M 1.2). Such uniform target sequence segments are preferably located on both sides of a polymorphic locus and thus flank a polymorphic locus (with sequence variants) of a target sequence from both sides. Preferably, the first uniform target sequence segment (N1) differs in its sequence composition from the sequence composition of the second uniform target sequence segment (N3) in at least one nucleotide position.

FIG. 10(A-C) shows schematically the topography of a template strand with a polymorphic locus (N2) and two uniform target sequence segments (N1 and N3) as shown in FIG. 9.

FIG. 10D shows the result of a strand separation after primer extension by two controller oligonucleotides (C1.1 and C1.2) which are characteristic and specific for a sequence variant respectively, wherein each of said controller oligonucleotides comprises a sequence segment corresponding to the polymorphic locus of the target sequence (N2), wherein C1.1 and C1.2 differ in said sequence segment. This results in a specific and characteristic pairing of a specific and characteristic sequence variant (an allele variant) of a specific target sequence and a controller oligonucleotide characteristic and specific for said sequence variant.

The figure summarizes schematically what effect the presence of the fully complementary strand (template strand) has on the possible mismatch duplex: Due to the fully complementary nature of the primer extension product synthesized by a polymerase, both primer extension products formed during the synthesis comprise fully complementary sequences respectively. Binding of such fully complementary sequences to each other takes place more effectively than in sequences comprising a mismatch. Thus, strand separation only occurs over a shorter distance and is less effective than with fully complementary strands.

FIG. 11 shows schematically a different topography of an N2 of a target nucleic acid chain with respect to the controller oligonucleotide and the first primer.

In said examplary embodiment, the sequence segment of the controller oligonucleotide corresponding to the N2 partly comprises the third region and partly the second region. The 3′ segment of the first primer is also designed sequence-specific and characteristic for a specific and characteristic sequence variant of the target nucleic acid. Thus, both the controller oligonucleotide and the first oligonucleotide primer are part of the allele discrimination. Thus, for each specific sequence variant of the polymorphic locus, both a specific controller oligonucleotide and a specific first oligonucleotide primer can be constructed.

FIG. 12 shows schematically potential positions of a polymorphic locus within a target sequence (positions 1 to 6) and their corresponding sequence segment positions in the individual components of a primer extension system. The schematically shown polymorphic locus of a target sequence comprises 2 to 50 nucleotides, particularly 4 to 30 nucleotides, which are characteristic and specific for an allelic variant of a target sequence. Altogether, the arrangement of individual amplification components can be designed in such way that several variants/combinations are possible:

Array 1 (P1): A polymorphic locus (N2) of the target sequence overlays the first primer (in the first region) and the controller oligonucleotide (in the second region). Said components of the amplification system can thus be designed specifically and characteristically for the respective sequence variations of the target sequence.

Array 2 (P2): A polymorphic locus (N2) of the target sequence overlays the first primer (in the first region) and the controller oligonucleotide (in the second region). These components of the amplification system can thus be constructed specifically and characteristically for the respective sequence variations of the target sequence. Array P2 differs from P1 mainly in that N2 is located predominantly in the 3′-terminal sequence segment of the first primer. This can lead to a better specificity of the amplification.

Array 3 (P3): A polymorphic locus (N2) of the target sequence overlays only the controller oligonucleotide (in the third region), wherein the corresponding sequence segment of the controller oligonucleotide is located in the 5′-terminal sequence segment of the synthesized portion of the first primer extension product. The controller oligonucleotide of the amplification system can thus be constructed specifically and characteristically for the respective sequence variation of the target sequence. In said array, a target sequence specific first primer is used, which is not sequence variant specific. Due to the possible proximity of the second blocking moiety, the binding of a controller oligonucleotide to the first primer extension product can be influenced by nucleotide modifications of the second blocking moiety.

Array 4 (P4-P6): A polymorphic locus (N2) of the target sequence overlays only the controller oligonucleotide (in the third region). The controller oligonucleotide of the amplification system can thus be constructed specifically and characteristically for the respective sequence variations of the target sequence. In said array, a target sequence-specific first primer is used, but said primers are not sequence variant-specific. Said sequence segment of the controller oligonucleotide is located in the 5′ direction from the second blocking unit and may comprise several DNA nucleotide monomers, e.g. from 5 to 30.

FIGS. 13-14 show schematically the topography of a target nucleic acid chain with a polymorphic locus (N2) and two uniform target sequence segments (N1 and N3), wherein the polymorphic locus comprises only one nucleotide position (e.g. an SNV or an SNP or a point mutation etc.). Thus, the differences between individual sequence variants account only one nucleotide (referred to here as 4.1 and 4.2). Such a locus (N2) can lead to similar arrays of individual components as a locus with at least 2 nucleotides. Thus, when binding of a controller oligonucleotide to a complementary first primer extension product (perfect match), strand separation can take place after primer extension. When a mismatch is formed, strand separation is inhibited or slowed down or even interrupted.

FIG. 15 shows schematically potential positions of a polymorphic locus with sequence variance of only one nucleotide within a target sequence (positions 1 to 6) of a template strand and their corresponding sequence segment positions in individual components of a primer extension system.

Overall, the array of individual amplification components can be designed in such way that several variants/combinations are possible:

Array 1 (P1): A polymorphic locus (N2) of the target sequence overlays the first primer (in the first region) and the controller oligonucleotide (in the second region). Said components of the amplification system can thus be designed specifically and characteristically for the respective sequence variant of the target sequence.

Array 2 (P2): A polymorphic locus (N2) of the target sequence overlays the first primer (in the first region) and the controller oligonucleotide (in the second region). Said components of the amplification system can thus be constructed specifically and characteristically for the respective sequence variants of the target sequence. Array P2 differs from P1 mainly in that N2 can be located predominantly in the 3′-terminal sequence segment of the first primer or even comprises the 3′-terminal nucleotide. This can result in a further increase in the specificity of the amplification.

Array 3 (P3): A polymorphic locus (N2) of the target sequence overlays only the controller oligonucleotide (in the third region), wherein the corresponding sequence segment of the controller oligonucleotide is located in the 5′-segment of the synthesized portion of the first primer extension product. The controller oligonucleotide of the amplification system can thus be constructed specifically and characteristically for the respective sequence variant of the target sequence. In said array a target sequence specific first primer is used, which is not sequence variant specific. Due to the possible proximity of the second blocking unit, the binding of a controller oligonucleotide to the first primer extension product can be influenced by nucleotide modifications of the second blocking unit.

Array 4 (P4-P6): A polymorphic locus (N2) of the target sequence overlays only the controller oligonucleotide (in the third region). The controller oligonucleotide of the amplification system can thus be constructed specifically and characteristically for the respective sequence variant of the target sequence. In said array a target sequence specific first primer is used, which is not sequence variant specific. Said sequence segment of the controller oligonucleotide is located in 5′ direction from the second blocking unit. Said segment of the controller oligonucleotide may comprise several DNA nucleotide monomers, e.g. from 5 to 30, wherein the N2 corresponding sequence segment can be flanked by at least 3 to 15 DNA nucleotide building blocks on both sides.

FIG. 16 shows schematically an embodiment with topography of a specific primer extension system and a template strand with a polymorphic locus (N2) in certain embodiments in which the segment of the oligonucleotide primer corresponding to N2 is located in the second region. Under reaction conditions, a specific P1.1 can bind preferably specifically to the complementary position of a specific sequence variant of the template strand (S1.1) and initiate the synthesis of the P1.1-Ext. No competitor primer is used. P1.1-Ext can bind complementarily to C1.1 and with its participation can be detached from the template strand.

FIG. 17 shows schematically an embodiment with topography of a specific primer extension system and a template strand with a polymorphic locus (N2) in certain embodiments in which the segment of the controller oligonucleotide corresponding to N2 is located in the second region. Under reaction conditions, a specific P1.1 can bind preferably specifically to the complementary position of a specific sequence variant of the template strand (M 1.1) and initiate the synthesis of the P1.1 Ext.

In the course of primer extension, P1.1-Ext is formed. No competitor primer is used. Binding to another sequence variant (M 1.2) takes place less efficiently due to mismatch with position (N) within the primer binding site of M 1.2. Thus, primer extension is started less efficiently under stringent reaction conditions.

FIG. 18 shows schematically an embodiment with topography of a specific primer extension system and a template strand with a polymorphic locus (N2) in certain embodiments in which the segment of the controller oligonucleotide corresponding to N2 is located in the second region (2). The N2 locus also comprises the 3′-terminal nucleotide of the first primer. A specific P1.1 with a complementary 3′-terminal nucleotide can preferably bind specifically to the complementary position of a specific sequence variant of the template strand (M 1.1) under reaction conditions and initiate the synthesis of the P1.1 Ext. During primer extension, P1.1-Ext is formed and no competitor primer is used. Binding to another sequence variant (SN 1.2) takes place less efficiently due to the mismatch with position (N) within the primer binding site of M 1.2. Thus, primer extension is started less efficiently under stringent reaction conditions.

FIG. 19 shows schematically an embodiment with topography of a specific primer extension system and a template strand with a polymorphic locus (N2) in certain embodiments in which the segment of the controller oligonucleotide corresponding to N2 is located in the third region (3). Target-sequence-specific but not sequence variant-specific first primers are used. A uniform P1.1 for all sequence variants of a target nucleic acid can bind to the template strand and initiate the synthesis of P1.1-Ext. In the course of primer extension, P1.1-Ext is formed and no competitor primer is used. The separation of primer extension products P1.1-Ext from the template strand preferably takes place specifically by complementary binding of a P1.1-Ext to the controller oligonucleotide. The separation of strands of another sequence variant, which were synthesized using (M 1.2), takes place less efficiently due to the mismatch with position (N) to the corresponding sequence segment of the controller oligonucleotide. The position of the sequence segment (3) corresponding to N2 of the target sequence is located near the second blocking unit, so that the interaction between the controller oligonucleotide and the respective first primer extension product can be influenced by modifications used in the second blocking unit.

FIG. 20 shows schematically an embodiment with topography of a specific primer extension system and a template strand with a polymorphic locus (N2) in certain embodiments in which the segment of the controller oligonucleotide corresponding to N2 is located in the third region (4). Target-sequence-specific but not sequence-variant specific first primers are used. A uniform P1.1 for all sequence variants of a target nucleic acid can bind to the template strands and initiate the synthesis of P1.1-Ext. During primer extension, P1.1-Ext is formed and no competitor primer is used. The separation of primer extension products P1.1-Ext from the template strand preferably takes place specifically by complementary binding of a P1.1-Ext to the controller oligonucleotide. The separation of strands of another sequence variant, which were synthesized using (M 1.2), takes place less efficiently due to the mismatch with position (N) to the corresponding sequence segment of the controller oligonucleotide. The position of the segment (4) corresponding to N2 of the target sequence is located at a distance from the second blocking unit so that the interaction between the controller oligonucleotide and the respective first primer extension product is essentially unaffected by modifications used in the second blocking unit. The segment of the controller oligonucleotide corresponding to N2 preferably consists of DNA monomers, wherein between 4 to 20 DNA monomers are used and at least 4 to 10 DNA monomers are located around position 4 on both sides.

FIG. 21 shows schematically an embodiment with topography of a specific primer extension system and a template strand with a polymorphic locus (N2) in certain embodiments in which the segment of the controller oligonucleotide corresponding to N2 is located in the second region. A specific P1.1 can bind preferably specifically to the complementary position of a specific sequence variant of the template strand (M 1.1) under reaction conditions and initiate the synthesis of the P1.1-Ext. During primer extension, P1.1-Ext is formed. Binding to another sequence variant (M 1.2) takes place less efficiently due to the mismatch with position (N) within the primer binding site of M 1.2. Thus, primer extension is started less efficiently under stringent conditions.

To further increase the specificity, a competitor-oligonucleotide-primer (P 5.1) is added to the reaction, which is capable of predominantly complementary binding to the sequence variants of the target sequence and whose copy creation with subsequent separation from the template strand must be suppressed. Due to complementary binding with such primer binding sites, a competitor primer can bind preferentially and be extended by the polymerase. The resulting product (here P5.1-Ext) blocks the single-stranded primer binding sites for an interaction of the first primer.

In certain embodiments, the 3′-end of a competitor-oligonucleotide-primer binds within the second blocking site of the controller oligonucleotide, so that no extension of this primer can take place at the controller oligonucleotide. The competitor-oligonucleotide-primer does not comprise a sequence segment that can interact with the first region of the controller oligonucleotide. This prevents the extension product of the competitor oligonucleotide from being detached from the template.

FIG. 22 shows schematically an embodiment with topography of a specific primer extension system and a template strand with a polymorphic locus (N2) in certain embodiments in which the segment of the oligonucleotide primer corresponding to N2 is located in the second region (2). The N2 locus also comprises the 3′-terminal nucleotide of the first primer. A specific P1.1, with a complementary 3′-terminal nucleotide can bind preferably specifically to the complementary position of a specific sequence variant of the template strand (M 1.1) under reaction conditions and initiate the synthesis of the P1.1-Ext. During primer extension, P1.1-Ext is formed. Binding to another sequence variant (M 1.2) takes place less efficiently due to the mismatch with position (N) within the primer binding site of SN 1.2. Thus, primer extension is initiated less efficiently under stringent conditions.

To increase specificity, a competitor-oligonucleotide-primer (P5.2) is added to the reaction, which is capable of predominantly complementary binding to the sequence variants of the target sequence and whose primer extension and subsequent separation from the template strand must be suppressed. Due to complementary binding with such primer binding sites, a competitor-oligonucleotide-primer can bind preferably to certain sequence variants (here designated N) and be extended by the polymerase. The resulting product (P5.2-Ext) blocks the single-stranded primer binding sites for interaction with the first primer.

In certain embodiments, the 3′-end of a competitor-oligonucleotide-primer binds within the second blocking site of the controller oligonucleotide, so that no extension of this primer can take place at the controller oligonucleotide. The competitor-oligonucleotide-primer does not comprise a sequence segment with which it can interact with the first region of the controller oligonucleotide. This prevents the extension product of the competitor oligonucleotide from being detached from the template.

FIG. 23 shows schematically an embodiment with topography of a specific primer extension system and a template strand with a polymorphic locus (N2) in an embodiment where the segment of the controller oligonucleotide corresponding to N2 is located in the third region (3). Target-sequence-specific but not sequence-variant-specific first primers are used. A uniform P1.1 for all sequence variants of a target nucleic acid can bind to the template strands and initiate the synthesis of P1.1-Ext. In the course of primer extension, P1.1-Ext is formed. The separation of synthesized P1.1-Ext primer extension products from the template strand preferably takes place specifically by complementary binding of a P1.1-Ext to the controller oligonucleotide. The separation of strands of another sequence variant synthesized using (M 1.2) takes place less efficiently due to mismatch with position (N) to the corresponding sequence segment of the controller oligonucleotide. The position of the sequence segment (3) corresponding to N2 of the target sequence is located near the second blocking unit, so that the interaction between the controller oligonucleotide and the respective first primer extension product can be influenced by modifications used in the second blocking unit.

To increase specificity, a competitor-oligonucleotide-primer (P5.3) is added to the reaction, which is capable of predominantly complementary binding (N) to sequence variants of the target sequence, and whose copy creation and subsequent strand separation must be suppressed. Due to complementary binding with such primer binding sites, a competitor-oligonucleotide-primer can bind preferably to certain sequence variants (here designated N) and be extended by the polymerase. The resulting product blocks the single-stranded primer binding sites for interaction with the first primer.

In certain embodiments, the competitor-oligonucleotide-primer (P5.3) is longer than the first oligonucleotide primer so that its 3′-end can bind within the fourth blocking unit of the controller oligonucleotide (the fourth blocking unit is composed analogously to the second blocking unit and blocks a primer extension at the controller oligonucleotide), so that no extension of this primer can take place at the controller oligonucleotide. The competitor-oligonucleotide-primer does not comprise a sequence segment that can interact with the first region of the controller oligonucleotide. This prevents the extension product of the competitor oligonucleotide from being detached from the template.

FIG. 24 shows schematically a selective primer extension reaction of several sequence variants using several selective controller oligonucleotides (C1.1 and C1.2).

FIGS. 25-29 show schematically the structure of the first oligonucleotide primer and the controller oligonucleotide, as well as the interaction between the first oligonucleotide primer and the template, as well as the synthesis of the first primer extension product.

FIG. 30 shows schematically the interaction between structures during primer extension of the first oligonucleotide primer.

FIG. 31 shows results of example 1.

FIG. 32 shows results of example 2.

FIG. 33 shows schematically some embodiments of structures of a start nucleic acid chain and their use as templates at the beginning of the reaction. Using P1.1, without block oligonucleotide.

FIG. 34 shows schematically some embodiments of structures of a start nucleic acid chain and their use as templates at the beginning of the reaction. Using P1.1, with block oligonucleotide.

FIG. 35 shows schematically the interaction of the structures during nucleic acid amplification by the amplification of the first oligonucleotide primer and the second oligonucleotide primer on the one hand, and the action of the controller oligonucleotide and the resulting strand displacement on the other hand.

FIGS. 36-38 show results of example 4.

FIG. 39 shows schematically an embodiment with topography of a specific primer extension system and a template strand with a polymorphic locus (N2) in certain embodiments. In addition to other illustrations, the following nomenclature of reaction components and sequence segments is used here:

a) a sample comprising a nucleic acid polymer which comprises the target sequence M, wherein the target sequence in 5′-3′ orientation comprises the sequence segments MS and MP, and MP is located immediately in the 3′ of MS, is brought into contact with the following components:

b) an oligonucleotide primer P, which in 5′-3′ orientation comprises the sequence segments PC and PM, and PM is located immediately in the 3′ direction of PC, wherein PM comprises the sequence complementary [hybridizing] to the sequence segment MP [can bind essentially sequence-specifically] and PC cannot bind to M [or a sequence in 3′ immediately following MP];

c) a controller oligonucleotide C, which in 5′-3′ orientation comprises the sequence segments CS, CP and CC, wherein CP is located immediately in 3′ direction of CS and immediately in 5′ direction of CC, and wherein CS is identical to at least the 3′ segment of MS (immediately in 5′ direction of MP), and CP and CC comprise the [hybridizing] sequence complementary to the sequence segment PM and PC, and wherein CS comprises modified nucleotide building blocks so that CS cannot serve as a template for the activity of the template-dependent nucleic acid polymerase;

d) a template-dependent nucleic acid polymerase, particularly a DNA polymerase, as well as substrates of the DNA polymerase (particularly ribonucleoside triphosphates or deoxyribonucleoside triphosphates) and suitable cofactors,

wherein a primer extension product P′ is obtained, which in addition to the sequence regions PC and PM comprises a synthesized region PS, which is essentially complementary to the target sequence MS,

and wherein

either the reaction conditions are chosen, and/or

the lengths and the melting temperature of PC and, if necessary, MS are chosen

so that P′ can form a double strand with M and P′ can form a double strand with C and the formation of the double strand of P′ and C is preferred over the formation of the double strand of P′ with M.

The method according to certain embodiments, wherein the sequence segment CS contains at its 3′ end immediately in the 5′ direction of the sequence segment CP a sequence segment CS' of 5 to 15 nucleotide positions in length, which consists of nucleic acid analogues, particularly of 2′0-alkyl ribonucleotides. The method according to any of the preceding embodiments, wherein M comprises a sequence segment MB immediately in 5′ of MS, and wherein further a block oligonucleotide B, which can hybridize to MB, is brought into contact with the sample, and wherein

a. B comprises nucleoside analogues which are selected such that a hybrid of B with MB does not allow the template-dependent nucleic acid polymerase to react on MB, or

b. the template-dependent nucleic acid polymerase is selected such that a hybrid of B with MB [even if B consists of natural nucleosides or deoxynucleotides] does not allow the template-dependent nucleic acid polymerase to react on MB.

The terms used in said embodiment illustrate certain variants of the structures described in the application.

A nucleic acid polymer (an embodiment of the template) comprises the target sequence M, which in 5′-3′ orientation comprises the sequence segments MS and MP and MP and is located immediately in 3′ of MS. In an embodiment, the target sequence comprises a polymorphic locus (N2).

An oligonucleotide primer P (corresponding to a first oligonucleotide primer) comprises in 5′-3′ orientation the sequence segments PC (corresponding to a second region of the first primer oligonucleotide) and PM (corresponding to a first region of the first oligonucleotide primer).

A controller oligonucleotide C comprises in 5′-3′ orientation the sequence segments CS (corresponding to a third region of the controller oligonucleotide), CP (corresponding to a second region of the controller oligonucleotide) and CC (corresponding to a first region of the controller oligonucleotide).

A primer extension product P′ (corresponding to a first primer extension product) is obtained by template-dependent extension of the first oligonucleotide primer. In addition to the sequence regions PC and PM, the primer extension product comprises a synthesized region PS, which is essentially complementary to the target sequence MS.

A variant VM of the target sequence M is different from the sequence M in at least one position VMM. Said position particularly comprises one or more substitution(s), insertion(s) and/or deletion(s). Said position VMM in said embodiment is a sequence variant of the polymorphic locus (N2) of the target sequence. In an embodiment VMM is located at the 5′-end of VMP. In other words, the 3′-end of VPM hybridizes with VMM.

A second controller oligonucleotide VC comprises in 5′-3′ orientation the sequence segments VCS (corresponding to a third region of the controller oligonucleotide), VCP (corresponding to a second region of the controller oligonucleotide) and VCC (corresponding to a first region of the controller oligonucleotide).

A second oligonucleotide primer VP (corresponding to a variant of a first oligonucleotide primer) comprises in 5′-3′ orientation the sequence segments VPC (corresponding to a second region of the first oligonucleotide primer) and VPM (corresponding to a first region of the first oligonucleotide primer) and VPM is located immediately in 3′ of VPC, wherein VPM comprises the [the hybridizing] sequence complementary to the sequence segment VMP of the target sequence VM [can bind essentially sequence-specifically] and VPC cannot bind to VM [or any sequence in 3′ immediately following VMP]. In an embodiment VMM (polymorphic locus N2) of the target sequence is located at the 5′ end of VMP and the 3′ end of VPM (of the first oligonucleotide primers) can hybridize specifically to VMM.

A competitor oligonucleotide KP can hybridize with the target sequence. In an embodiment, KP can hybridize to VMP of the target sequence.

A block oligonucleotide B can hybridize in an embodiment to a sequence segment immediately in the 5′ direction of MS of the target sequence MB, wherein B comprises nucleoside analogues selected as such that a hybrid (complex) of B with MB does not allow a template-dependent nucleic acid polymerase to react on MB.

FIGS. 40-58 show schematically certain embodiments for using a primer extension product as a start nucleic acid chain for an amplification method.

FIGS. 59-60 show schematically certain embodiments with topography of a specific primer extension system and a template strand with a polymorphic locus (N2) in an embodiment. In addition to other illustrations, the following nomenclature of reaction components and sequence segments is used here (see nomenclature FIG. 39).

### EXAMPLES

Material and Methods:

Reagents were purchased from the following commercial suppliers:

unmodified and modified oligonucleotides (Eurofins MWG, Eurogentec, Biomers, Trilink Technologies, IBA Solutions for Life Sciences); polymerases NEB (New England Biolabs); dNTP's: Jena Bioscience; intercalating EvaGreen dye: Jena Bioscience; buffer substances and other chemicals: Sigma-Aldrich; plastic goods: Sarstedt

Solution 1 (amplification reaction Solution 1):

Potassium glutamate, 50 mmol/l, pH 8.0; magnesium acetate, 10 mmol/l; dNTP (dATP, dCTP, dTTP, dGTP), 200 μmol/l each; polymerase (Bst 2.0 WarmStart, 120. 000 U/ml NEB), 12 units/10 μl; Triton X-100, 0.1% (v/v); EDTA, 0.1 mmol/l; TPAC (Tetrapropylammonium chloride), 50 mmol/l, pH 8.0; EvaGreen dye (dye was used in dilution 1:50 according to the respective manufacturer's instructions).

Solution 2 (amplification reaction Solution 2):

1× Isothermal buffer (New England Biolabs); in single concentration the buffer contains: 20 mM Tris-HCl; 10 mM (NH4)2SO4 ; 50 mM KCl; 2 mM MgSO4; 0.1% Tween® 20;pH 8.8@25° C.); dNTP (dATP, dCTP, dUTP, dGTP), each 200 μmol/l;

EvaGreen dye (dye was used in dilution 1:50 according to respective manufacturer's instructions)

All concentrations are indications of the final concentrations in the reaction. Deviations from the standard reaction are indicated respectively.

The melting temperature (Tm) of components involved was determined at concentrations of 1 μmol/l of respective components in solution 1 or 2. Deviating parameters are indicated respectively.

General Information on Reactions

Primer extension reactions and amplification were performed at two reaction temperatures of 55° C. and/or 65° C. as standard. Deviations are indicated.

The reaction start took place by heating the reaction solutions to reaction temperature, since Bst 2.0 polymerase warm start at lower temperatures is largely inhibited in its function by a temperature-sensitive oligonucleotide (an “aptamer” according to the manufacturer's specifications). The polymerase becomes increasingly active at temperatures above 45° C. At a temperature of 65° C. no differences between Polymerase Bst 2.0 and Bst 2.0 warm start could be detected. To prevent the extensive formation of by-products (e.g. primer-dimers) during the preparation phase of a reaction, Polymerase Bst 2.0 Warmstart was used. Deviations from this are specifically indicated.

The reaction stop took place by heating the reaction solution to over 80° C., e.g. 10 min at 95° C. At this temperature the Polymerase Bst 2.0 is irreversibly denatured and the result of the synthesis reaction cannot be changed afterwards.

Reactions were performed in a thermostat with a fluorescence measuring device. For this purpose, a commercial real-time PCR device, StepOne Plus (Applied Biosystems, Thermofischer) was used. The standard reaction volume was 10 Deviations from this volume are indicated.

Both endpoint determination and kinetic observations were performed. For endpoint determinations the signal was detected e.g. from dyes bound to nucleic acids, e.g. from TMR (Tetramethyl-Rhodamine, also called TAMRA) or from FAM (Fluorescein). The wavelengths for excitation and measurement of the fluorescence signals of FAM and TMR are stored as factory settings in the StepOne Plus Real-Time PCR device. Also an intercalating dye (EvaGreen) was used for endpoint measurements (e.g. measurement of a melting curve). EvaGreen is an intercalating dye and is an analogue of the frequently used dye Sybrgreen, but with slightly lower inhibition of polymerases. The wavelengths for excitation and measurement of the fluorescence signals of SybrGreen and EvaGreen are identical and are stored as factory settings in the StepOne Plus Real-Time PCR device. The fluorescence can be detected continuously, i.e. “online” or “real-time” by means of built-in detectors. Since the polymerase synthesizes a double strand during its synthesis, said technique could be used for kinetic measurements (Real-Time Monitoring) of the reaction. Due to a certain cross-talk between color channels in the StepOne Plus device, increased basal signal intensity was sometimes observed in measurements using TMR-labeled primers in concentrations above 1 μmol/l (e.g. 10 μmol/l). It was observed that the TMR signal in the SybrGreen channel leads to elevated basal values. These elevated baseline values were taken into account in calculations.

Kinetic observations of reaction courses were routinely recorded using fluorescence signals from fluorescein (FAM-TAMRA Fret pair) or intercalating dyes (EvaGreen). Time-dependence of the signal was recorded (real-time signal acquisition with StepOne plus PCR device). An increase of the signal during a reaction compared to a control reaction was interpreted according to the structure of the batch. For example, an increase in the signal when using Evagreen dye was interpreted as an indication of an increase in the quantity of double-stranded nucleic acid chains during the reaction, and thus as the result of a synthesis taking place by the DNA polymerase.

For some reactions, a melting curve determination was generally performed after the reaction. Such measurements allow conclusions to be drawn about the presence of double strands, which can, for example, absorb intercalating dyes and thus significantly amplify the signal intensity of dyes. With increasing temperature, the portion of double strands decreases and the signal intensity decreases as well. The signal depends on the length of nucleic acid chains and on the sequence composition. This technique is sufficiently known to a skilled person.

When using melting curve analysis in connection with reactions containing significant portions of modified nucleic acid chains (e.g., controller oligonucleotides or primers), it was found that the signal from the EvaGreen dye, for example, can behave differently between the B-form of DNA and the A-form of modified nucleic acid chains. For example, a higher signal intensity was observed for the B form of double-stranded nucleic acid chains (usually assumed for classical DNA segments) than for double-stranded nucleic acid chains with the same sequence of nucleobases that can adopt a conformation similar to A form (e.g. by several 2′-O-Me modifications of nucleotides). This observation was taken into account when using intercalating dyes.

If necessary, the reaction was analyzed by capillary electrophoresis and the length of formed fragments was compared to a standard. In preparation for capillary electrophoresis, the reaction mixture was diluted in a buffer (Tris-HCl, 20 mmol/l, pH 8.0, and EDTA, 20 mmol/l, pH 8.0) so that the concentration of labeled nucleic acids was approximately 20 nmol/l. The capillary electrophoresis was carried out by GATC-Biotech (Constance, Germany) as a contract service. According to the supplier, capillary electrophoresis was performed on an ABI 3730 Cappilary Sequencer under standard conditions for Sanger sequencing using POP7 gel matrix, at approx. 50° C. and at constant voltage (approx. 10 kV). The conditions used led to the denaturation of double strands, so that in capillary electrophoresis the single-stranded form was separated from nucleic acid chains. Electrophoresis is a standard technique in genetic analysis. Today, automated capillary electrophoresis is routinely used in Sanger sequencing. The fluorescence signal is continuously recorded during capillary electrophoresis (usually using virtual filters), resulting in an electrophorogram in which the signal intensity correlates with the duration of the electrophoresis. For shorter fragments, e.g. unused primers, an earlier signal peak is observed; for longer fragments, there is a time shift of the signals proportional to the length of the extended region. The length of extended fragments can be measured by using controls with known lengths. This technique is known to a skilled person and is also used as standard for fragment length polymorphism.

Unless otherwise defined with respect to a particular sequence, both upper and lower case letters agct and AGCT denote the deoxyribonucleotide building blocks of DNA, or the respective ribonucleotides or base analogues. In some exemplary embodiments, uracil nucleobases are used in modified sequence segments, therefore 2′-OMe modifications were used as sugars (ribose with 2′-O-methyl modification). Other 2′-O-alkyl modifications are also possible. In batches with dUTP particularly the first amplification reaction) amplification fragments are generated, which comprise dUMP (as protection against contamination). In general, however, uracil bases can be used with 2′-deoxyribose as well as with ribose; the skilled person takes from the revelation as a whole the teaching related to the respective sequence segments of the sequences used here to determine at which points classical DNA backbones, RNA or base analogues can be usefully applied.

### Example 1

A primer extension reaction for preparing a primer extension product using sequence variants of a target sequence and subsequent use of the synthesized primer extension product in an amplification reaction.

In said example, the influence of sequence differences in two templates on the interaction with the controller after a synthesis phase was investigated. Two templates were provided, which comprised a sequence difference at one nucleotide position. When extending the first oligonucleotide primer, a complementary strand was formed which contained a sequence complementary to the respective template and thus also comprised this sequence difference in the sequence. Both templates comprised a uniform primer binding site, so that a uniform primer could be used. Discrimination between individual sequence variants of the target sequence thus took place using a specific controller oligonucleotide.

The primer extension products generated during the synthesis phase can be specifically separated from template strands by controllers. The primer extension products comprise a 3′-segment, which is not bound by a controller oligonucleotide. This 3′-segment of the first primer extension product can be bound complementarily by another oligonucleotide, e.g. a primer. First primer extension products generated in this way can be amplified in a subsequent amplification reaction using another, second primer. The amplification reaction thus serves to illustrate the effect of a controlling phase (strand separation by a controller) after a synthesis phase.

Following templates were used:

A template with sequence composition which results in a first primer extension product with a perfect match with the controller oligonucleotide:

M2SF5-M001-200

(SEQ ID NO: 001)
5′ GCT CATA CTACAATGTCA CT TA CTGTAA GAGCAGATCC
CTGGACAGGC AA GGAATACAGGTA AAAAA 3′

A=2′-deoxy-Adenosin; C=2′-deoxy-Cytosin; G=2′-deoxy-Guanosin; T=2′-deoxy-Thymidin (Thymidin)

The binding sequence for the first oligonucleotide primer is underlined.

Template with sequence composition resulting in a first primer extension product that forms a mismatch with the controller oligonucleotide at a single base position (in bold):

M2SF5-WT01-200

(SEQ ID NO: 002)
5′ GCT CATA CTACAATGTCA CT TA CTGTAA GAGCAGATCC
CTGGACAGGC GA GGAATACAGGTA AAAAA 3′

A=2′-deoxy-Adenosin; C=2′-deoxy-Cytosin; G=2′-deoxy-Guanosin; T=2′-deoxy-Thymidin (Thymidin)

The binding sequence for the first oligonucleotide primer is underlined.

Following primers were used:

The first oligonucleotide primer: P1F5-200-AE2053

(SEQ ID NO: 003)
5′ AACTCAGACAAGATGTGATTTTTTTACCTGTAT[CUCU
GAUGCUUC] 1TACCTGTATTCC 3′

The segment used as primer in the reaction is underlined.

A=2′-deoxy-Adenosin; C=2′-deoxy-Cytosin; G=2′-deoxy-Guanosin; T=2′-deoxy-Thymidin (Thymidin)

This oligonucleotide comprised the following modifications:

1=C3-linker

The segment of the primer in square brackets [CUCU GAUGCUUC] comprised 2′-O-Me modifications and served as a second primer region for binding of the first region of the controller oligonucleotide:

A=(2′-O-Methyl-Adenosine), G=(2′-O-Methyl-Guanosine); C=(2′-O-Methyl-Cytosine);

U=(2′-O-Methyl-Uridine)

This oligonucleotide primer comprises the first region (positions 1-12 from the 3′-end), the second region (C3 linker, as well as positions 13-24 from the 3′-end), as well as a segment with an additional sequence variant P1 (positions 25-57 from the 3′-end). The first region and the second region are necessary for performing a specific amplification and can be summarized as “basic structure of the first primer” or “minimal structure of the first primer. The additional sequence variant P1 is an example of additional segments which can be integrated at the first oligonucleotide primer. Positions 1-12 serve as templates for the synthesis of the second primer extension product. C3 modification and the second region prevent continuation of the synthesis at positions 25-57 during synthesis of the second primer extension product.

Following controller oligonucleotide was used: AD-F5-1001-503

(SEQ ID NO: 004)
5′ [UAAUCUGUAA GAGCAGAUCC CUGGACAGGC AA GGAAUAC]AGGTAGAAGCATC
AGAG X 3′

A=2′-deoxy-Adenosin; C=2′-deoxy-Cytosin; G=2′-deoxy-Guanosin; T=2′-deoxy-Thymidin (Thymidin)

The 5′ segment of the oligonucleotide in square brackets [UAAUCUGUAA GAGCAGAUCC CUGGACAGGC AA GGAAUAC] comprised 2′-O-Me-nukleotide modifications:

Modifications: A=(2′-O-Methyl-Adenosine), G=(2′-O-Methyl-Guanosine); C=(2′-O-Methyl-Cytosine); U=(2′-O-Methyl-Uridine)

X=3′-phosphate group to block a possible extension by polymerase.

The nucleotides and nucleotide modifications are linked together with phosphodiester bonds. The 3′-end of the controller oligonucleotide is blocked with a phosphate group to prevent a possible extension by the polymerase.

The primer extension reaction was directly continued as amplification reaction (homogeneous assay).

For this purpose, another primer (primer 2) was added to the reaction mixture before the primer extension reaction started. This second primer can bind complementarily to the 3′ segment of the primer extension product synthesized in the primer extension reaction. Furthermore, this second primer can support an amplification reaction starting from the first primer extension product, which was synthesized during the synthesis phase and which is subsequently detached from its template by the controller during the controlling phase.

Primer 2: P2G3-5270-7063

(SEQ ID NO: 005)
5′ CTACAGAACTCAGACAAGATGTGAACTACAATGTT 6
GCTCATACTACAATGTCACTTACTGTAAGAGCAGA 3′

Said oligonucleotide comprises the following modifications:

6=HEG-linker

The segment used as primer in the reaction is underlined. Said segment is capable of binding complementarily to the 3′ segment of the first primer extension product and initiating a second primer extension reaction using the first primer extension product as template.

Said oligonucleotide primer comprises a copyable region and a non-copyable region. The copyable region comprises (positions 1-13 from the 3′ end, which can bind complementarily to sequence of the FVL gene within hgDNA and positions 14-35, which do not bind complementarily to sequence of the FVL gene but can bind to the first segment of the primer extension product during amplification). The copyable regions can be summarized as “basic structure of the second primer” or “minimal structure of the second primer”.

The non-copyable region (positions 36-70 from the 3′ end, which do not bind to the sequence of FVL gene complementarily) is separated from the copyable region by HEG modification, which prevents the continuation of the synthesis at positions 36-70 during a synthesis of the first segment of the primer extension product. The non-copyable region is an example of an additional sequence variant P2, which can be integrated at the second oligonucleotide primer.

The first primer extension reaction and the second primer extension reaction result in a double strand comprising the first and second primer extension product. Further use of both primers and the controller oligonucleotide (and repeated temperatures of 55° C. and 65° C.) resulted in an exponential amplification comprising both primer extension steps as well as strand separation using controller oligonucleotides. During the amplification phase, the first oligonucleotide primer continued to serve as the amplification primer.

Four batches were prepared:

Batch 1 contains the template M2SF5-M001-200 (perfect match situation) in a concentration of 300 fmol/l (corresponds to approx. 2e6 copies/ batch).

Batch 2 contains the template M2SF5-M001-200 (perfect match situation) in a concentration of 300 amol/l (corresponds to approx. 2×10\hat{ }3 copies/ batch).

Batch 3 does not contain a template and is therefore a control.

Batch 4 contains the template M2SF5-WT01-200 (single mismatch situation) in a concentration of 300 pmol/l (approx. 2×10\hat{ }9 copies per batch).

Primer 1 was tested with 5 μmol/l, the controller oligonucleotide with 2 μmol/l and primer 2 with 1 μmol/l is inserted. The further reaction conditions were: Amplification solution 2.

To replicate the presence of genomic DNA in the assay, 100 ng freshly denatured fish DNA (salmon DNA) was added per reaction.

A primer extension reaction (synthesis phase and controlling phase).

The thermal reaction conditions were 2 min at 55° C. (synthesis phase) and then 5 min at 65° C. (controlling phase).

The synthesis phase of the primer extension reaction of the first primer took place primarily at 55° C. At this temperature, the controller was present in complex with the first primer and was therefore in a non-active state. The controlling phase took place at 65° C. At this temperature, the controller dissociated at least partially from the first oligonucleotide primer and was thus able to interact with the primer extension products formed. The melting temperature of the first region of the first primer with its primer binding site on the template was about 45° C. in amplification solution 2. Thus, the synthesis phase was performed at a reaction temperature of about Tm+10° C., based on Tm of template/first primer complex. Due to a higher concentration of the first primer (5 μmol), a sufficient quantity of the first primer was present in the synthesis phase to bind to the template and start the reaction. When the temperature was increased to 65° C., the yield of the synthesis phase decreased significantly. At said temperature the controller was partially released from its binding with the primer (Tm of the complex comprising the first primer and controller in amplification solution 1 was 63° C.). Therefore, the controlling phase was performed at approx. Tm+2° C. related to Tm of controller/first primer-complex.

Amplification reaction (Repetition of primer extension reactions)

The thermal reaction conditions were cyclically alternating temperature changes, wherein a 2 min time interval at 55° C. was followed respectively by a 5 min time interval at 65° C. The amplification was monitored over 100 cycles. Detection took place at 65° C. for EvaGreen fluorescence signal.

The successful synthesis of primer extension products (here referred to as amplification) was determined by an increase in EvaGreen fluorescence signal over time. Temperature changes and real-time monitoring were performed with Thermofisher's StepPne Plus Real-Time PCR device.

FIG. 31 shows a typical curve of the EvaGreen signal. The Y-axis shows the increase of the fluorescence signal (Delta Rn) and the X-axis shows the reaction time (as cycle number). Arrows mark individual reaction preparations. The marked positions belong to the following batches:

| | |
|---|---|
| Arrow 1: 300 fmol/l perfect match template | (approx. 2 × 10\hat{ }6 |
| | copies/batch) |
| Arrow 2: 300 amol/l perfect match template | (approx. 2 × 10\hat{ }3 |
| | copies/batch) |
| Arrow 3: no template | |
| Arrow 4: 300 pmol/l mismatch template | (approx. 2 × 10\hat{ }9 |
| | copies per batch). |

The increase of the fluorescence signal can be seen in both perfect match and mismatch variant of the template, wherein the signal of the mismatch variant (4) appears later despite a 1000-fold excess. In single mismatch, a delay of about 15 cycles is observed. The time delay (=number of cycles) is an immediate measure of the discrimination due to the action of the controller. When using a perfect match template, a complementary strand of a primer extension product is synthesized. Said extension product is complementary to both the perfect match template and the controller oligonucleotide used. In contrast, using a mismatch sequence, the synthesis of the first segment of the primer extension product results in the generation of a complementary strand of the extension product, which comprises complete complementarity to the mismatch template, but thereby deviates from complementarity with the third region of the oligonucleotide controller. Said deviation takes place in the 5′-strand segment of the extension product, which should react with the controller oligonucleotide in order to allow the strand displacement process to proceed. As shown in said example, the mismatch interferes with strand separation by the controller oligonucleotide.

Said result illustrates the importance of the base composition in the controller oligonucleotide: deviations from the complementarity between the controller oligonucleotide and the primer extension product can cause the amplification to slow down or even stop.

In said example it was shown that although sequence ends of the perfect match template and mismatch template were completely matching and thus the potential for binding of both oligonucleotide primers was the same, both reactions proceeded completely different: in case of complete complementarity between the controller oligonucleotide and the 5′ segment of the extension product of the first oligonucleotide primer, the amplification proceeded according to plan. An interruption of the strand separation during the controlling phase by a non-complementary sequence variant (in this case by a mismatch) led to a reduction of the strand separation, which was reflected in the suppression of the amplification kinetics.

Thus, a primer extension product produced in a primer extension reaction can be used as a starting nucleic acid in an amplification method.

### Example 2

Influence of a Mismatch Between a First Primer and a Controller on the Strand Separation (Controlling Phase).

Similar to the first example, an amplification reaction was used to illustrate an effect of a controlling phase (strand separation by a controller) after a synthesis phase.

The synthesis phase takes place under reaction conditions that allow a primer extension reaction on a template. However, different controllers were used for the respective strand separation (controller phase): Perfect match and mismatch controller oligonucleotide to the primer used during the synthesis phase. To demonstrate the effect of such a single mismatch between a first primer and a controller, repeated primer extension reactions (synthesis phases) and controlling phases were performed under cyclic temperature conditions. This resulted in an amplification of a first primer extension product.

The amplification was used as a detection method for the controlling phase: only if the strand separation is successful and the second oligonucleotide primer can bind to the 3′-segment of the first primer extension product, an amplification takes place. Due to the high sensitivity of said method (due to exponential character) and a quantification possibility (time dependence of signal detection depending on concentration), amplification was used as a detection method for successful or unsuccessful strand separation during a controlling phase. Real-time monitoring of the synthesis of resulting amplification products took place in this example with the intercalating dye Eva Green.

The following template was used:
Template with sequence composition resulting in a first primer extension product with a perfect match matching with the controller oligonucleotide:

M2SF5-M001-200

(SEQ ID NO: 001)
5′ GCT CATA CTACAATGTCA CT TA CTGTAA GAGCAGATCC
CTGGACAGGC AA GGAATACAGGTA AAAAA 3′

The binding sequence for the first oligonucleotide primer is underlined.

The second oligonucleotide primer binds to the reverse complement of the double underlined sequence.

Following primers were used:

The first oligonucleotide primer (SEQ ID NO:2), served as primer extension primer and as amplification primer.

P1 F5-200-AE2053

(SEQ ID NO: 003)
5′ AACTCAGACAAGATGTGATTTTTTTACCTGTAT[CUCU
GAUGCUUC] 1TACCTGTATTCC 3′

The segment used as primer in the reaction is underlined.

A=2′-deoxy-Adenosin; C=2′-deoxy-Cytosin; G=2′-deoxy-Guanosin; T=2′-deoxy-Thymidin (Thymidin)

Said oligonucleotide comprises following modifications:

1=C3-linker was used to terminate the synthesis of the second primer extension product.

Segment of the primer [CUCU GAUGCUUC] comprised 2′-O-Me modifications and served as a second primer primer region to bind the first region of a controller oligonucleotide:

A=(2′-O-Methyl-Adenosine), G=(2′-O-Methyl-Guanosine); C=(2′-O-Methyl-Cytosine);

U=(2′-O-Methyl-Uridine)
The second oligonucleotide primer served as a second primer for amplification: P2G3-5270-7063

(SEQ ID NO: 005)
5′ CTACAGAACTCAGACAAGATGTGAACTACAATGTT 6
GCTCATACTACAATGTCACTTACTGTAAGAGCAGA 3′

Modifications:

6=HEG-linker

Both the first primer and the second primer are capable of supporting amplification with a perfect match oligonucleotide primer.

Following perfect match controller oligonucleotide was used:

AD-F5-1001-503

(SEQ ID NO: 004)
5′[UAAUCUGUAA GAGCAGAUCC CUGGACAGGC AA
GGAAUAC]AGGTAGAAGCATC AGAG X 3′

A=2′-deoxy-Adenosin; C=2′-deoxy-Cytosin; G=2′-deoxy-Guanosin; T=2′-deoxy-Thymidin (Thymidin)

The 5′ segment of the oligonucleotide set in square brackets [UAAUCUGUAA GAGCAGAUCC CUGGACAGGC AA GGAAUAC] comprised 2′-O-Me-nucleotide-modifications:

Modifications: A=(2′-O-Methyl-Adenosine), G=(2′-O-Methyl-Guanosine); C=(2′-O-Methyl-Cytosine);

U=(2′-O-Methyl-Uridine)

X=3′-phosphate group to block a possible extension by the polymerase.

Following mismatch controller oligonucleotide was used:MD2AD-F5-011-5

(SEQ ID NO: 007)
5′-GCTC TAATCTGTAA GAGCAGATCC CTG [GAC AGGC AA [symbol]
GAAUACAGG] TA
GAAGCATC AGAG X 3′

A=2′-deoxy-Adenosin; C=2′-deoxy-Cytosin; G=2′-deoxy-Guanosin; T=2′-deoxy-Thymidin (Thymidin)

The middle segment of the oligonucleotide set in square brackets [GAC AGGC AA AGAAUACAGG] comprised 2′-O-Me- nucleotide-modifications: A=(2′-O-Methyl-Adenosine), G=(2′-O-Methyl-Guanosine); C=(2′-O-Methyl-Cytosine); U=(2′-O-Methyl-Uridine)

X=3′-phosphate group to block a possible extension by the polymerase.

The nucleotides and nucleotide modifications are linked to one another with phosphodiester bonds. The 3′-end is blocked with a phosphate group to prevent a possible extension by the polymerase.

In the mismatch controller oligonucleotide, an A nucleotide (mismatch variant) was inserted at position −24 (from the 3′ end) instead of a G nucleotide (perfect match variant). The nucleotide is highlighted in bold. This position forms a mismatch with the 3′-terminal nucleotide of the inserted first primer.

The template was used in concentrations of 1-3 pmol/l. No template was used in the control reaction (=negative control). Primer 1 was used at 5 μmol/l, the respective controller oligonucleotide at 2 μmol/l and primer 2 at 1 μmol/l.

The other reaction conditions were:
Amplification solution 2
To replicate the presence of genomic DNA in the assay, 100 ng freshly denatured fish DNA (salmon DNA) was added per reaction.

The synthesis phase of the primer-extension reaction of the first primer took place primarily at 55° C. At said temperature the controller was present in complex with the first primer and was therefore in a non-active state. The controlling phase took place at 65° C. At said temperature, the controller dissociated at least partially from the first oligonucleotide primer and was thus able to interact with formed primer extension products. The melting temperature of the first region of the first primer with its primer binding site on the template was about 45° C. in amplification solution 2. Thus, the synthesis phase was performed at a reaction temperature of about Tm+10° C., based on Tm of template/first primer complex. Due to a higher concentration of the first primer (5 μmol), a sufficient amount of the first primer was present in the synthesis phase to bind to the template and start the reaction. When the temperature was increased to 65° C., the yield of the synthesis phase decreased significantly. At this temperature the controller was partially released from its binding with the primer (Tm of the complex comprising first primer and controller in amplification solution 1 was 63° C.). Therefore, the controlling phase was performed at approx. Tm+2° C. related to Tm of controller/first primer-complex.

In the amplification, the thermal reaction conditions were cyclically alternating temperature changes, wherein a 1 min time interval at 55° C. was followed respectively by a 5 min time interval at 65° C. The amplification was typically monitored over 120 cycles, i.e. 120×(1 min 55° C.+5 min 65° C.)=120×6 min=12 h. Successful amplification was determined by an increase in EvaGreen fluorescence signal over time.

Analysis of the Primer Extension Reaction (And Amplification Reaction)

The following techniques were used to analyze the amplification reaction and to evaluate the resulting amplification products:
Fluorescence signal of an intercalating dye (EvaGreen)
Melt curve analysis of the resulting amplification products

FIG. 32A shows a typical course of the EvaGreen fluorescence signal during an allele-specific amplification reaction using a perfect match controller oligonucleotide. The Y-axis shows the fluorescence signal of the EvaGreen dye and the X-axis shows the reaction time (as cycle number). Arrow 1 marks a perfect match amplification approach in FIG. 32 A. Due to the perfect match controller oligonucleotide, the template can be amplified and the fluorescence signal increases from cycle 27 on. Arrow 2 marks a negative control batch that does not contain a template and therefore does not generate an amplification signal during the experiment.

FIG. 32B shows a typical course of the EvaGreen fluorescence signal during an allele-specific amplification reaction using a mismatch controller oligonucleotide. The Y-axis shows the fluorescence signal of the EvaGreen dye and the X-axis shows the reaction time (as cycle number). Arrow 1 marks a mismatch amplification approach. Due to the mismatch controller oligonucleotide, the template cannot be amplified. Arrow 2 marks a negative control batch that does not contain a template and therefore does not generate an amplification signal during the experiment. The fluorescence signal of the mismatch controller oligonucleotide batch and the negative control are almost identical, which shows how strongly amplification is suppressed in the mismatch situation.

FIG. 32C shows the melting curves of the allele-specific amplification products from FIG. 32A and 32B. The Y-axis shows the derivative of the fluorescence signal against temperature and the X-axis shows the temperature. Two characteristic peaks were found, marked with arrows 1 and 2. The peak at position 1 with a melting point near 65° C. is part of the mismatch controller oligonucleotide batch. The signal pattern cannot be distinguished from negative control batches (=amplification reaction without template). In contrast, the peak at position 2 has a melting point near 85° C. Said peak belongs to the perfect match controller oligonucleotide batch and is specific for the amplification product formed. The results suggest that specific amplification products with a defined melting point near about 85° C. are formed in the perfect match situation. In contrast, mismatch controller oligonucleotides suppress the amplification reaction. The fluorescence signals of the mismatch controller oligonucleotide batches could not be distinguished from the negative control in this example. This shows how strong the amplification suppression is in the mismatch situation.

In summary: a nucleotide mismatch in the controller oligonucleotide positioned in the second region of the controller oligonucleotide to the corresponding first region of the primer can prevent amplification.

Based on said example, it can be seen that sequence variant-specific amplification systems can be assembled comprising a sequence variant-specific primer and a respective complementary sequence variant-specific controller oligonucleotide.

### Example 3

Primer Extension Reaction Using a Template and Block Oligonucleotides with LNA Modifications to Limit a Primer Extension Reaction.

Said example shows the use of human genomic DNA (hgDNA) as a source of a target sequence, wherein the primer extension reaction is performed using a first oligonucleotide primer and a controller oligonucleotide. To limit the synthesis progress, block oligonucleotides were used, which were bound complementarily to the template strand in 3′ direction from the primer before starting a primer extension reaction by hybridization. The synthesis was performed for 15 min at 55° C. During this synthesis phase, the first region of the primer binds to the primer binding site in the template and is extended by the polymerase, resulting in a primer extension product. The controller was added before the primer extension reaction started and was present in a double-stranded complex with the primer during said synthesis phase in a non-active form in the batch.

After completion of the synthesis phase, the reaction temperature was increased to 65° C. for 5 minutes, so that the complex comprised the controller and the primer at said temperature and the controller could interact with the primer extension product formed during the synthesis phase and separate it from the template strand.

A sequence segment of the factor V Leiden gene (Homo sapiens coagulation factor V (F5), mRNA, here called FVL gene) was chosen as the target sequence.

Target Sequence:

(SEQ ID NO: 008)
5′TGACGTGGAC ATCATGAGAG ACATCGCCTC TGGGCTAATA
GGACTACTTC TAATCTGTAA GAGCAGATCC CTGGACAGGC
AAGGAATACA GGTATTTTGT CCTTGAAGTA ACCTTTCAGA 3′

The binding sequence for the first oligonucleotide primer is underlined (primer binding site). The binding site for the block oligonucleotide is double underlined.

The Block Oligonucleotide:

P1-EXB-1000-101
(SEQ ID NO: 009)
5′ {CCA GAGGCGATG}T ATCTCATGAT GTCCACAACA
CTGTAGTATG GTCTTGTTAA GCAAAAA
3′ X

The sequence segment in curly brackets {CCA GAGGCGATG} comprises LNA modifications.

The first primer as well as controller oligonucleotide were designed and synthesized for FVL mutation variant of the gene.

The First Oligonucleotide Primer:

P1F5-200-AE2053
(SEQ ID NO: 003)
5′ AACTCAGACAAGATGTGATTTTTTTACCTGTAT [CUCU
GAUGCUUC] 1TACCTGTATTCC 3′

The segment used as primer in the reaction is underlined.

A=2′-deoxy-Adenosin; C=2′-deoxy-Cytosin; G=2′-deoxy-Guanosin; T=2′-deoxy-Thymidin (Thymidin)

Said oligonucleotide comprises the following modifications:

1=C3-linker was used to terminate the synthesis of the second primer extension product.

The segment of the primer [CUCU GAUGCUUC] comprised 2′-O-Me modifications and served as a second primer region to bind the first region of a controller oligonucleotide: Said sequence cannot bind complementarily to the template in the region next to the primer binding site and is therefore available for interaction with the controller oligonucleotide.

Modifications: A=(2′-O-Methyl-Adenosine), G=(2′-O-Methyl-Guanosine); C=(2′-O-Methyl-Cytosine); U=(2′-O-Methyl-Uridine)

The first primer comprises in its first region a sequence which can bind specifically to the sequence of the Factor V Leiden gene within the genomic DNA, so that a synthesis can be initiated by a polymerase. The second region of the first primer comprises a sequence that does not specifically hybridize to the sequence of the FVL gene. Furthermore, the first primer comprises a further sequence segment which links to the 5′ end of the second region. Said segment does not participate in the specific primer extension of the Factor 5 Leiden segment. The function of said segment is mainly seen in the delay of side reactions.

The following controller oligonucleotide was used:

AD-F5-1001-503
(SEQ ID NO: 004)
5′ [UAAUCUGUAA GAGCAGAUCC CUGGACAGGC AA GGAAUAC]
AGGTAGAAGCATC
AGAG X 3′

A=2′-deoxy-Adenosin; C=2′-deoxy-Cytosin; G=2′-deoxy-Guanosin; T=2′-deoxy-Thymidin (Thymidin)

The 5′ segment of the oligonucleotide set in square brackets [UAAUCUGUAA GAGCAGAUCC CUGGACAGGC AA GGAAUAC] comprised 2′-O-Me-nucleotide-modifications:

Modifications: A=(2′-O-Methyl-Adenosine), G=(2′-O-Methyl-Guanosine); C=(2′-O-Methyl-Cytosine); U=(2′-O-Methyl-Uridine)

X=3′-phosphate group to block a possible extension by the polymerase.

The nucleotides and nucleotide modifications are linked together with phosphodiester bonds. The 3′-end of the controller oligonucleotide is blocked with a phosphate group to prevent a possible extension by the polymerase. This controller can bind complementarily with its segment (positions underlined above) to the first expected primer extension product:

(SEQ ID NO: 010)
[UAAUCUGUAA GAGCAGAUCC CUGGACAGGC AA GGAAUAC] AGGTA

The controller oligonucleotide has been constructed in such way that a perfect match to the sequence of the Factor V Leiden mutation of the FVL gene results. The controller oligonucleotide comprises a first, second and third region.

All reactions were performed in amplification solution 2.

The dNTPs used comprised: dATP, dGTP, dCTP, dUTP (instead of dTTP).

The polymerase used was Bst 2.0 Warm-Start Polymerase from NEB.

The primer extension reaction was prepared as follows:

About 50,000 haploid genomic equivalents (HGE), 150 ng hgDNA in 40 μl, FVL-WHO standard, double-stranded, were brought into contact with a block oligonucleotide (0.3 μmol/l) in reaction solution 2 and first denatured by heating (5 min at 95° C.) and then cooled to room temperature.

Then the first primer (2 μmol/l) together with the controller oligonucleotide (1 μmol/L) was added to the reaction and then Bst-2.0 warm start polymerase (about 1 unit) as well as dNTPs (about 250 μmol/l) were added and the reaction volume was brought to 50 μl. The resulting reaction mixture was incubated under stringent primer extension conditions (amplification solution 2, temperature of about 55° C.) for about 15 min. During said phase an extension of the first primer took place, wherein the genomic DNA served as template. The result is a primer extension product. Since during this phase the controller oligonucleotide is complexed with the first primer (and is therefore in an inactive state), the primer extension product formed was located in the double-stranded complex with the template. In order to remove said product from the template sequence specifically, the reaction mixture was heated to 65° C. and incubated for about 5 min. During said phase, the primer extension product was at least partially displaced from the binding with the template by the controller. The 3′ segment of the primer extension product dissociated spontaneously under selected reaction conditions. Such a primer extension product can be used, for example, in a subsequent amplification reaction in analogy to example 1.

Such an array of block oligonucleotides on a template and thus a limitation of the length of a primer extension product can be used, for example, to provide a nucleic acid fragment to be used as the starting nucleic acid in a specific exponential amplification reaction. It is true that such a start nucleic acid as a primer extension reaction can also take place without a block oligonucleotide (FIGS. 33A and B). However, the undefined length of a primer extension fragment that can be produced when the template strand is copied can require sequence-unspecific, e.g. temperature-related strand separation (FIG. 33C). The binding of a second amplification primer takes place in the middle region of such a primer extension product (FIGS. 33C and D).

In contrast, the use of a block oligonucleotide primer allows an almost arbitrary specific limitation of the length of a primer extension product, regardless of the length of a template strand (FIGS. 34A and B). Furthermore, the block oligonucleotide favors a sequence-dependent separation of formed primer extension products even before the start of an amplification. For example, a synthesis phase of a primer extension takes place first and then a controlling phase (sequential arrangement of steps). In an embodiment, the synthesis phase and the controlling phase with the separation of the primer extension product can be performed in one step and take place parallel to each other).

As a result, a specific primer extension product is generated and separated sequence-specifically from the template with the participation of a controller, without the need for an unspecific denaturing step (FIGS. 34B and C). Such a primer extension product can be used as start nucleic acid for further amplification (FIGS. 34D and E). This can increase the specificity of the generation of a start nucleic acid.

The block oligonucleotide preferably does not serve as a primer itself. This can take place, for example, by blocking its 3′-OH group. The block oligonucleotide primer thus comprises at least one sequence segment that is complementary to the template strand on which the primer extension reaction also takes place. Preferably, said segment (blocking segment) is sufficiently long or comprises modifications, if necessary, to form a stable double strand under reaction conditions of a primer extension reaction with the template strand. Furthermore, such a block oligonucleotide can also comprise sequence segments that are not complementary to the template strand. Such sequence segments can, for example, flank the segment that is to bind to the template and block the primer extension reaction on both sides. For example, the block oligonucleotide can be located in a sequence segment of a target nucleic acid or placed at one of its ends. The block oligonucleotide is thus positioned in such way that it is located in the 3′ direction from the expected primer extension product on the template and prevents the polymerase from further copying the template strand.

The aim of a primer extension reaction can be to produce a starting nucleic acid chain for a subsequent amplification reaction. Such a start nucleic acid chain is used at the beginning of an amplification reaction. Its function can be seen as the initial template that allows the correct positioning of primers, the synthesis segments between the two primers, as well as the initiation of binding and extension processes. In certain embodiments, a start nucleic acid chain comprises a target sequence.

By binding primers to their respective primer binding sites (PBS 1 and PBS 2) and initiating respective primer extension reactions, generation of first primer extension products takes place. These are synthesized as specific copies of the nucleic acid chain present at the beginning of the reaction.

In certain embodiments, the nucleic acid chain to be inserted into the reaction mixture before the start of the amplification reaction (start nucleic acid chain) can be identical to the nucleic acid chain to be amplified. The amplification reaction only increases the quantity of such a nucleic acid chain.

In certain embodiments, the nucleic acid to be amplified and the start nucleic acid chain differ in that the start nucleic acid chain determines the array of individual sequence elements of the nucleic acid chain to be amplified, but the sequence composition of the start nucleic acid chain can deviate from the sequence of the nucleic acid chain to be amplified. For example, during primer binding and extension during amplification, new sequence contents (related to the start nucleic acid chain) can be integrated into the nucleic acid chain to be amplified. Furthermore, sequence elements of a nucleic acid chain to be amplified may differ from such sequence elements of a start nucleic acid chain in their sequence composition (e.g. primer binding sites or primer sequences). The start nucleic acid only serves as an initial template for the specific synthesis of the nucleic acid chain to be amplified. Said initial template can remain in the reaction mixture until the end of the amplification. However, due to the exponential character of amplification, the quantity of the nucleic acid chain to be amplified at the end of an amplification reaction outweighs the quantity of a starting nucleic acid chain added to the reaction.

In certain embodiments, the start nucleic acid chain comprises a target sequence or its subsegments, which comprise at least one expected sequence variant of a polymorphic locus of the target sequence.

A start nucleic acid further comprises at least one predominantly single-stranded sequence segment, to which at least one of the primers of the amplification system with its 3′ segment can bind predominantly complementarily, so that the polymerase used can extend such a primer, when hybridized to the start nucleic acid chain, template-specifically by incorporation of dNTPs.

A start nucleic acid, which can comprise several sequence variants in a polymorphic locus of a target sequence, preferably further comprises at least one first target sequence segment, which is characteristic and uniform for all target sequence variants. In certain embodiments, a start nucleic acid comprises at least two target sequence segments (a first target sequence segment and a second target sequence segment), which are located on both sides of a polymorphic locus of a target sequence and thus flank the sequence variants of a target sequence from both sides.

The length of a polymorphic locus of a start nucleic acid nucleus can comprise ranges from one nucleotide up to 200 nucleotides, particularly from one nucleotide up to 50 nucleotides, particularly from one nucleotide up to 20 nucleotides. The lengths of uniform sequence segments (first and second uniform sequence segment of a starting nucleic acid) can comprise the following ranges for at least one of the two uniform sequence segments: from 4 to 200 nucleotides, particularly from 6 to 100 nucleotides, particularly from 8 to 50 nucleotides.

In certain embodiments, the starting nucleic acid chain can comprise at least one sequence segment which is not amplified. Such a start nucleic acid chain is therefore not identical with the sequence to be amplified. Such segments that are not to be amplified can, for example, represent a sequence segment of a start nucleic acid chain as a sequence of sequence preparation steps or as a sequence of preceding sequence manipulation steps.

In a preferred embodiment, the start nucleic acid chain to be inserted into the reaction mixture before the start of the reaction includes at least one target sequence.

In certain embodiments, such a start nucleic acid chain includes at least one target sequence and further sequences which are not target sequences. During amplification, sequence segments comprising the target sequence are exponentially amplified and other sequence segments are either not amplified at all or only partially exponentially.

Structure of a Start Nucleic Acid Chain

An example of such a start nucleic acid chain is a nucleic acid chain which includes a target sequence or its equivalents (e.g. a sequence complementary to the target sequence) and which comprises a sequence fragment A and comprises a sequence fragment B.

The sequence fragment A of the start nucleic acid chain comprises a sequence which comprises a significant homology with the sequence of one of the two primers used in the amplification or which is essentially identical to the copyable portion of the 3′ segment of said one primer. Synthesis of a complementary strand to said segment generates a complementary sequence that represents a respective primer binding site.

Sequence fragment B of the start nucleic acid chain comprises a sequence suitable for complementary binding of a respective further primer or its 3′ segment to form an extendible primer-assembly template complex, wherein sequence fragment A and sequence fragment B are predominantly/preferably non-complementary relative to each other.

In a preferred embodiment, a start nucleic acid chain comprising a target sequence which comprises the following properties is added to the reaction mixture of an amplification method:
Sequence segment 5 (called segment 5, S5 in FIG. 34) comprising a sequence which comprises a significant homology with the sequence of the first region of the first primer or is essentially identical to the copyable portion of the first region of the first primer. Said segment 5 is located in the 5′ segment of the starting nucleic acid chain and is flanked by a non-copyable oligonucleotide tail (analogous to the first primer oligonucleotide), preferably this segment 5 forms a boundary of the copyable nucleic acid chain strand in 5′ direction.
The Sequence Fragment 7 (called segment 7, S7), which comprises a sequence suitable for complementary binding of a second primer or its 3′ segment to form an extendable primer template complex. Preferably, segment 7 is located in the 3′-direction of segment 5 of the start nucleic acid chain (FIG. 34).
The target sequence, which is located partially or completely between segment 5 and segment 7 (in FIG. 34 said portion of the target sequence is called segment 6, S6). In a preferred embodiment, the target sequence comprises segment 6 and at least one of segment 5 and/or segment 7.
The start nucleic acid chain can comprise flanking sequence segments in the 3′ segment (called segment 8 in FIG. 33), which are not amplified.

In a further embodiment, a starting nucleic acid chain comprising a sequence complementary to the target sequence (equivalent of a target sequence) is added to the reaction mixture of an amplification method, which comprises the following properties:
Sequence segment 5 (called segment 5, S5 in FIG. 34) comprising a sequence which comprises a significant homology with the sequence of the first region of the first primer or is essentially identical to the copyable portion of the first region of the first primer. Said segment 5 is located in the 5′ segment of the starting nucleic acid chain and is flanked by a non-copyable oligonucleotide tail (analogous to the first primer oligonucleotide), preferably said segment 5 forms a boundary of the copyable nucleic acid chain strand in the 5′ direction.
The sequence fragment 7 (called segment 7, S7), which comprises a sequence suitable for complementary binding of a second primer or its 3′ segment to form an extendable primer template complex. Preferably, segment 7 is located in 3′-direction of segment 5 of the start nucleic acid chain (FIG. 34).
The sequence complementary to the target sequence, which is located partially or completely between segment 5 and segment 7 (in FIG. 34 the above, this portion of the sequence is called segment 6, S6). In a preferred embodiment, the sequence comprises segment 6 and at least one of segment 5 and/or segment 7.
The start nucleic acid chain can comprise flanking sequence segments in the 3′ segment (called segment 8 in FIG. 33), which are not amplified.

Functionality of the Start Nucleic Acid Chain

At the beginning of the amplification reaction, the starting nucleic acid chain serves as a template for the initial formation of respective primer extension products. It thus represents the start template for the nucleic acid chain to be amplified. The start nucleic acid chain does not necessarily have to be identical with the nucleic acid chain to be amplified. By binding and extending both primers during the amplification reaction, the two primers essentially determine which sequences are generated at both terminal segments of the nucleic acid chain to be amplified during the amplification process.

In a preferred embodiment of the method, non-denaturing reaction conditions are maintained for a double strand during the exponential amplification process. Therefore, it is advantageous if the start nucleic acid chain comprises a limitation in its 5′ sequence segment extendable by a polymerase, which leads to a stop in the enzymatic extension of a respective primer. This limits the length of primer extension fragments generated under reaction conditions. This can have an advantageous effect on the strand displacement by the controller oligonucleotide and lead to a dissociation of the respective strand, so that primer binding sites are transformed into a single-stranded state and thus become accessible for rebinding of primers.

### Example 4

### Influence of a Controller Oligonucleotide on the Primer Extension Reaction in a Parallel Synthesis Phase and Controlling Phase:

The reactions were performed in amplification solution 2.

The following templates were used (DNA templates):

P1Ex-400-4001
(SEQ ID NO: 011)
C TTACAG TAAGTGACATTGTAG TATG AGC GATCC CTGGACAGGC
AA GGAATACAGGTA A 3′
P1EX-400-4002
(SEQ ID NO: 012)
C TTACAG TAAGTGACATTGTAG TATG AGC GATCC CTGGACAGGC
AA GAATACAGGTA A
P1EX-400-4003
(SEQ ID NO: 013)
C TTACAG TAAGTGACATTGTAG TATG AGC GATCC CTGGACAGGC
AA AATACAGGTA A
P1EX-400-4004
(SEQ ID NO: 014)
C TTACAG TAAGTGACATTGTAG TATG AGC GATCC CTGGACAGGC
AA GGAATAAAGGTA A
P1EX-400-4005
(SEQ ID NO: 015)
C TTACAG TAAGTGACATTGTAG TATG AGC GATCC CTGGACAGGC
AA AGAATACAGGTA A

DNA-Templates with Iso-dC

P1EX-400-4201 (is identical to SEQ ID NO 11; however, in the sequence shown below it comprises an iso-dC instead of the bold G at position 47:)

(SEQ ID NO: 011)
C TTACAG TAAGTGACATTGTAG TATG AGC GATCC CTGGACAGGC
AA GGAATACAGGTA A 3′
(SEQ ID NO: 016)
CTTACAGTAA GTGACATTGT AGTATGAGCG ATCCCTGGAC
AGGCAA 1GAATACAGGTA A

P1EX-400-4202 (is identical to SEQ ID NO 11, but i comprises an Iso-dC instead of the G at position 43:)

(SEO ID NO: 017)
C TTACAG TAAGTGACATTGTAG TATG AGC GATCC CTGGACAG1C
AA GGAATACAGOTA A

1=5-Me-Iso-dC (nucleotide can only form a complementary nucleobase with Iso-dG. It does not form a “typical Watson-Crick” base pairing with any of the dNTP's used, so that overcoming this position can only take place by mismatch formation)

The templates were varied in their sequence composition in such way that resulting primer extension products can interact differently with the controller oligonucleotide. Depending on the sequence composition of the template, either a perfect match or a mismatch in the primer extension product/controller complex could be generated.

To test for a polymerase error, templates were used that include an iso-dC at specific positions. Since said nucleotide does not comprise a natural correlate, a polymerase must generate a mismatch and only when said mismatch is overcome can the displacement of the probe (see below) take place. Thus, templates with iso-dC represent an example of the primer extension reactions that occur under mismatch formation (e.g., due to a polymase error). Templates with Iso-dC are only intended to illustrate the changes in the behavior of the primer extension reactions and the influence of the controller on the behavior of the polymerases/synthesis of complementary strands.

The following primers were used:

SEQP1F5 300-35X
(SEQ ID NO: 018)
5′ AC GAAG CTCGCAAGAAC TCAG AG TGTG CTCGAC AC
TACCTGTATTCC 3′

The first region of the primer is underlined. With said segment the primer can bind to perfect match templates.

A=2′-deoxy-Adenosin; C=2′-deoxy-Cytosin; G=2′-deoxy-Guanosin; T=2′-deoxy-Thymidin (Thymidin)

Said primer does not comprise a segment that can interact with the first region of the controller oligonucleotide. Thus, when said primer is extended, a controller oligonucleotide cannot separate the formed primer extension product from the template strand.

P1F5-001-1003
(SEQ ID NO: 019)
5′ TMR-[CU GAUGCUUC] 1 TACCTGTATTCC 3′

1=C3-linker

The first region of the primer is underlined. With said segment the primer can bind to perfect match templates.

The segment [CU GAUGCUUC] set in square brackets comprises 2′-O-Me modifications and represents the second region of the primer.

Modifications: A=(2′-O-Methyl-Adenosine), G=(2′-O-Methyl-Guanosine); C=(2′-O-Methyl-Cytosine); U=(2′-O-Methyl-Uridine)

TMR=Tetramethyl-Rhodamine at the 5′-end

Following controller oligonucleotide was used:

AD-F5-1001-503
(SEQ ID NO: 020)
5′[UAAUCUGUAA GAGCAGAUCC CUGGACAGGC AA GGAAUAC]
AGGTAGAAGCATC AGAG X 3′

A=2′-deoxy-Adenosin; C=2′-deoxy-Cytosin; G=2′-deoxy-Guanosin; T=2′-deoxy-Thymidin (Thymidin)

The 5′-segment of the oligonucleotide [UAAUCUGUAA GAGCAGAUCC CUGGACAGGC AA GGAAUAC] comprised 2′-O-Me nucleotide modifications:

Modifications: A=(2′-O-Methyl-Adenosine), G=(2′-O-Methyl-Guanosine); C=(2′-O-Methyl-Cytosine); U=(2′-O-Methyl-Uridine)

X=3′-phosphate group to block a possible extension by the polymerase.

In said example, the controller oligonucleotide can form a perfect match with certain primer extension products. It can form a mismatch with other primer extension products.

To monitor the progress of the primer extension reaction, a fluorescence probe was used, which can bind to the 5′ segment of the used templates. When said probe is displaced from the binding with said 5′-segment of the respective template, the signal of the probe is reduced (intramolecular quenching between dT-BHQ1 and FAM reporter)

Fluorescence probe:
P2D-LUX-1000-101
(SEQ ID NO: 021)
FAM-5′ TAGTATGAGC TTTT 1 GCTCATAC2ACAATGTCACTTA
CTGTAA GAGCAGA

1=HEG

2=dT-BHQ1

The probe was used in 0.2 μmol/L concentration. The templates were used at a concentration of 0.5 μmol/l, the primers were used at 1 μmol/l concentration and controllers (if used as indicated) were used at 2 μmol/l. Bst-2.0 Warm-Start (NEB) was used in a concentration of 4 units/10 μl.

The reactions were performed in a StepOne Plus device at a temperature of 55° C. Signal detection took place via FAM channel. The reaction was monitored continuously (sampling interval=10 sec). The signal intensity was controlled by batches without polymerase (but with all other components) or alternatively with polymerase but without template.

The Tm of PBS (perfect match) of the template with the first region of the primer was about 45° C. The Tm of controller oligonucleotide/primer (P1F5-001-1003) was about 57° C. The reaction temperature of the primer extension reaction was 55° C. The reaction ran over 100 min.

The results of individual reactions are summarized in FIG. 36.

The kinetics of individual reactions were evaluated based on the change of the fluorescence signal.

The evaluation of representative data sets is shown below:

| Reaction | Tempate | Primer | Controller | Polymerase |
|---|---|---|---|---|
| 1 | P1Ex-400-4001 | P1F5-001-1003 | No | Yes |
| 2 | P1Ex-400-4001 | P1F5-001-1003 | AD-F5- | Yes |
| | | | 1001-503 | |
| 3 | P1Ex-400-4001 | P1F5-001-1003 | No | No |

Reaction 1 (FIG. 36A): In the absence of a controller, a rapid primer extension and probe displacement took place during perfect-match binding of a primer, so that more than 70% of the probes were already detached from the 5′ segment of the templates during the first signal acquisition and the signal was strongly reduced already at the beginning of the measurement.

Reaction 2 (FIG. 36A): With perfect match binding of a primer in the presence of a controller, rapid primer extension and probe displacement also took place, but at a slower rate compared to reaction 1 without controller.

Reaction 3 (FIG. 36A): served as reference signal for probe in the absence of a polymerase.

| Reaktion | Matrize | Primer | Controller | Polymerase |
|---|---|---|---|---|
| 4 | P1Ex-400-4001 | SEQP1F5 300-35X | No | Yes |
| 5 | P1Ex-400-4001 | SEQP1F5 300-35X | AD-F5- | Yes |
| | | | 1001-503 | |
| 6 | P1Ex-400-4001 | SEQP1F5 300-35X | No | No |

Reactions 4-6 (FIG. 36B) show reactions with a primer that cannot interact with the controller in the first region (said primer has no second region that can bind to the first region of the controller in a complementary way). The presence of the controller seems to have no detectable influence on the course of this reaction. Under selected reaction conditions, the controller hardly interferes with the course of said reaction.

Introduction of a mismatch in primer binding site of the template

| Reaktion | Matrize | Primer | Controller | Polymerase |
|---|---|---|---|---|
| 7 | P1EX-400-4002 | P1F5-001-1003 | No | Yes |
| 8 | P1EX-400-4002 | P1F5-001-1003 | AD-F5- | Yes |
| | | | 1001-503 | |
| 9 | P1EX-400-4002 | P1F5-001-1003 | No | No |

Reactions 7 and 8 show that the absence of a complementary nucleotide in the template (3′-terminal mismatch) leads to a complete prevention of the primer extension reaction (FIG. 37A). Analogous reaction behavior was observed with templates P1 EX-400-4003, P1 EX-400-4004, P1 EX-400-4005, P1 EX-400-4201 (absence of 2 terminal nucleotides, a mismatch in the middle of PBS, a substitution of G on A (mismatch), a substitution of G on iso-dC). In none of the reactions the probe was displaced. This shows that the presence of a controller oligonucleotide does not have a negative effect on discrimination.

| Reaktion | Matrize | Primer | Controller | Polymerase |
|---|---|---|---|---|
| 10 | P1EX-400-4202 | P1F5-001-1003 | No | Yes |
| 11 | P1EX-400-4202 | P1F5-001-1003 | AD-F5- | Yes |
| | | | 1001-503 | |
| 12 | P1EX-400-4202 | P1F5-001-1003 | No | No |

Reaction 10 (FIG. 37B): Shows the overcoming of an Iso-dC position in the template in the absence of a controller and continuation of the synthesis until the probe is displaced using a first oligonucleotide primer (P1F5-001-1003). Although a delayed reaction kinetics is seen (displacement of the probe takes place much slower than in the perfect match template), a complementary primer extension product can be synthesized in good yields (probe has been displaced).

Reaction 11 (FIG. 37B): Shows the influence of the controller present in the reaction: overcoming the mismatch position is essentially slower. This is associated with the fact that the primer extension product formed during the reaction can be detached during the reaction and forms a complex with the controller, which has a higher Tm than the primer/controller complex. The prematurely detached, incomplete primer extension product preferably remains in the complex with the controller and is thus removed from the reaction (“trapping” of incomplete primer extension products). This prevents the polymerase from overcoming the iso-dC nucleotide. The presence of the controller thus exerts a “corrective effect” on the polymerase: although a polymerase is in principle capable of overcoming such a mismatch, in combination with the controller said property of the primer extension system is altered to such an extent that a complete synthesis of defective primer extension products is suppressed. The primer extension products generated in the meantime are withdrawn from further extension running during a primer extension reaction (on-line control).

Reaction 12: served as reference signal for the probe in the absence of a polymerase.

| Reaktion | Matrize | Primer | Controller | Polymerase |
|---|---|---|---|---|
| 13 | P1EX-400-4202 | SEQP1F5 | No | Yes |
| | | 300-35X | | |
| 14 | P1EX-400-4202 | SEQP1F5 | AD-F5- | Yes |
| | | 300-35X | 1001-503 | |
| 15 | P1EX-400-4202 | SEQP1F5 | No | No |
| | | 300-35X | | |

Reaction 13 (FIG. 38A): Shows the overcoming of an iso-dC position in the template in the absence of a controller and the continuation of the synthesis until the displacement of the probe using an oligonucleotide primer which does not comprise a second region (SEQP1 F 300-35X). Although a delayed reaction kinetics can be seen (displacement of the probe takes place much slower than in the perfect match template), a complementary primer extension product can be synthesized in good yields (probe has been displaced).

Reaction 14: However, the presence of a controller oligonucleotide seems to have no effect on the behavior of the primer extension system in such a primer design: the primer used does not have a second region, unlike the primer used in reaction 11.

Overall, this example demonstrated the ability of a primer extension system to modify/prevent the polymerase from overcoming a mismatch in the presence of a controller oligonucleotide and a first primer.

### Example 5

Use of a Synthesized Nucleic Acid Chain as the Start Nucleic Acid Chain in an Amplification Method.

The nucleic acid chain obtained by a primer extension reaction can be detached from the template strand and used in another method.

The execution of this amplification method has already been described in the PCT application PCT/EP2017/071011 and the European application 16185624.0. For details on the execution of the amplification, the skilled person is referred to this application.

For example, in a method for amplifying nucleic acid chains with a controller oligonucleotide (see examples 1 and 2). Such an amplification method comprises several steps as shown in FIGS. 40-42, which schematically show the interaction of components in an exponential amplification of a nucleic acid chain to be amplified in individual steps.

Thus, the nucleic acid chain provided by a primer extension can be used as a start nucleic acid chain in an amplification with allele discrimination as a template, for example.

FIG. 43A shows schematically a start nucleic acid chain and components of the amplification system in the batch before the start of amplification:

Start nucleic acid (start DNA) comprising a target sequence.

Primer 1 (P1.1), Primer 2 (P2.1), an activator oligonucleotide (C1.1).

Components for primer extension reaction: polymerase (Pol) and dNTPs

FIG. 43B shows schematically the result of the amplification:

The primer extension product P1.1-Ext starting from P1.1 and the primer extension product 2.1-Ext. These products (P1.1-Ext, P2.1-Ext) can form different complex forms among themselves and with activator oligonucleotide (depending on the concentration ratio and reaction conditions). In detail, said forms can comprise complexes of P1.1-Ext/C1.1 and/or P1.1-Ext and/or P1.1-Ext/C1.1/ P2.1-Ext.

The P1-Ext comprises a 3′-segment, which is not bound complementarily by the activator oligonucleotide. Said segment serves as a binding partner for the oligonucleotide probe.

FIG. 44 schematically shows potential positions of a polymorphic locus within a target sequence (positions 1 to 6) and their corresponding sequence segment positions in individual components of an amplification system. The polymorphic locus of a target sequence schematically shown here comprises 2 to 50 nucleotides, particularly 4 to 30 nucleotides which are characteristic and specific for an allelic variant of a target sequence.

In total, the arrangement of individual amplification components can be designed in such a way that several variants/combinations are possible:

Array 1 (P1): polymorphic locus (N2) of the target sequence overlays the first primer (first region) and activator oligonucleotide primer (second region). Said components of the amplification system can thus be constructed specifically and characteristically for the respective sequence variants of the target sequence. In said array, a target sequence specific second primer is used, which is not sequence variant specific.

Array 2 (P2): polymorphic locus (N2) of the target sequence overlays the first primer (first region) and activator oligonucleotide (second region). Said components of the amplification system can thus be constructed specifically and characteristically for the respective sequence variations of the said target sequence. In said array, a target sequence-specific second primer is used, which is not sequence variant-specific. Array P2 differs from P1 mainly in that N2 is located predominantly in the 3′-terminal sequence segment of the first primer. This can lead to a better specificity of the amplification.

Array 3 (P3): polymorphic locus (N2) of the target sequence overlays only the activator oligonucleotide (third region), wherein the corresponding sequence segment of the activator oligonucleotide is located in the 5′ segment of the synthesized portion of the first primer extension product. The activator oligonucleotide of the amplification system can thus be constructed specifically and characteristically for the respective sequence variation of the target sequence. In said array, a target sequence-specific first and second primer is used, which are not sequence variants specific. Due to the possible proximity of the second blocking unit, the binding of the activator oligonucleotide primer to the first primer extension product can be influenced by nucleotide modifications of the second blocking unit.

Array 4 (P4): polymorphic locus (N2) of the target sequence overlays only the activator oligonucleotide (third region). The activator oligonucleotide of the amplification system can thus be constructed specifically and characteristically for the respective sequence variations of the target sequence. In said array, a target sequence-specific first and second primer is used, which are not sequence variant-specific. Said sequence segment of the activator oligonucleotide is located in 5′ direction from the second blocking unit and may comprise several DNA nucleotide monomers, e.g. from 5 to 30.

Array 5 (P5): polymorphic locus (N2) of the target sequence overlays the second primer and the activator oligonucleotide (third region). Said components of the amplification system can thus be constructed specifically and characteristically for the respective sequence variations of the target sequence. In said array a target sequence-specific first primer is used, which is not sequence variant specific.

Array 6 (P6): polymorphic locus (N2) of the target sequence overlays the second primer and the activator oligonucleotide (third region, near the 5′-end). These components of the amplification system can thus be constructed specifically and characteristically for the respective sequence variations of the said target sequence. In said array a target sequence-specific first primer is used, which is not sequence variant specific.

The design differences of individual elements also result in differences for the primer extension products synthesized during amplification.

FIG. 45 shows schematically potential positions of a polymorphic locus with sequence variance of only one nucleotide within a target sequence (positions 1 to 6) and their corresponding sequence segment positions in individual components of an amplification system.

Overall, the arrangement of individual amplification components can be designed in such a way that several variants/combinations are possible:

Array 1 (P1): polymorphic locus (N2) of the target sequence overlays the first primer (first region) and the activator oligonucleotide (second region). Said components of the amplification system can thus be designed specifically and characteristically for the respective sequence variants of the said target sequence. In said array a target sequence-specific second primer is used, which is not sequence variant specific.

Array 2 (P2): polymorphic locus (N2) of the target sequence overlays the first primer (first region) and the activator oligonucleotide (second region). Said components of the amplification system can thus be constructed specifically and characteristically for the respective sequence variants of the said target sequence. In said array, a target sequence-specific second primer is used, which is not sequence variant specific. Array P2 differs from P1 mainly in that N2 can be located predominantly in the 3′-terminal sequence segment of the first primer or even comprises the 3′-terminal nucleotide. This may result in a further increase in the specificity of the amplification.

Array 3 (P3): polymorphic locus (N2) of the target sequence overlays only the activator oligonucleotide (third region), wherein the corresponding sequence segment of the activator oligonucleotide is located in the 5′-segment of the synthesized portion of the first region primer extension product. The activator oligonucleotide of the amplification system can thus be constructed specifically and characteristically for the respective sequence variant of the target sequence. In said array, a target sequence-specific first and second primer is used, which are not sequence variant specific. Due to the possible proximity of the second blocking unit, the binding of the activator oligonucleotide to the first primer extension product can be influenced by nucleotide modifications of the second blocking unit.

Array 4 (P4): polymorphic locus (N2) of the target sequence overlays only the activator oligonucleotide (third region). The activator oligonucleotide of the amplification system can thus be constructed specifically and characteristically for the respective sequence variant of the target sequence. In said array, a target sequence-specific first and second primer is used, which are not sequence variant specific. Said sequence segment of the activator oligonucleotide is located in 5′ direction from the second blocking unit. Said segment of the activator oligonucleotide may comprise several DNA nucleotide monomers, e.g. from 5 to 30, wherein the N2 corresponding sequence segment may be flanked by at least 3 to 15 DNA nucleotide building blocks on both sides.

Array 5 (P5): polymorphic locus (N2) of the target sequence overlays the second primer and the activator oligonucleotide (third region). Said components of the amplification system can thus be constructed specifically and characteristically for the respective sequence variant of the said target sequence. The array can be designed so that the 3′-terminal nucleotide of the second primer corresponds to the N2 locus. In said array, a target sequence-specific first primer is used, which is not sequence variant specific.

Array 6 (P6): polymorphic locus (N2) of the target sequence overlays the second primer and the activator oligonucleotide (third region, near the 5′-end). Said components of the amplification system can thus be constructed specifically and characteristically for the respective sequence variant of the said target sequence. In said arrangement a target sequence-specific first primer is used, which is not sequence variant specific.

The design differences of individual elements also result in differences for the primer extension products synthesized during amplification. FIGS. 46-54

FIG. 46 shows schematically an embodiment with topography of a specific amplification system and a starting nucleic acid chain with a polymorphic locus (N2) in an embodiment in which the segment of the activator oligonucleotide corresponding to N2 is located in the second region. Under reaction conditions, a specific P1.1 can bind preferably to the complementary position of a specific sequence variant of the start nucleic acid (SN 1.1) and initiate the synthesis of the P1.1 Ext. In the course of the amplification, P1.1-Ext and P2.1-Ext are formed and no competitor primer is used.

FIG. 47 schematically shows an embodiment with topography of a specific amplification system and a start nucleic acid chain with a polymorphic locus (N2) in an embodiment in which the segment of the activator oligonucleotide corresponding to N2 is located in the second region. A specific P1.1 can bind preferably to the complementary position of a specific sequence variant of the start nucleic acid (SN 1.1) under reaction conditions and initiate the synthesis of the P1.1 Ext. In the course of the amplification, P1.1-Ext and P2.1-Ext are formed and no competitor primer is used. Binding to another sequence variant (SN 1.2) takes place less efficiently due to the mismatch with position (N) within the primer binding site of SN 1.2. Thus, amplification is started less efficiently.

FIG. 48 shows schematically an embodiment with topography of a specific amplification system and a start nucleic acid chain with a polymorphic locus (N2) in an embodiment where the segment of the activator oligonucleotide corresponding to N2 is located in the second region (2). The N2 locus also comprises the 3′-terminal nucleotide and/or the 3′-terminal segment of the first primer. A specific P1.1, with a complementary 3′-terminal nucleotide can bind preferably specifically to the complementary position of a specific sequence variant of the start nucleic acid (SN 1.1) under reaction conditions and initiate the synthesis of the P1.1 Ext. In the course of the amplification, P1.1-Ext and P2.1-Ext are formed and no competitor primer is used. Binding and initiation of primer extension to another sequence variant (SN 1.2) takes place less efficiently due to mismatch with position (N) within the primer binding site of SN 1.2. Thus, amplification is started less efficiently.

FIG. 49 shows schematically an embodiment with topography of a specific amplification system and a start nucleic acid chain with a polymorphic locus (N2) in an embodiment where the segment of the activator oligonucleotide corresponding to N2 is located in the third region (3). A target sequence specific but not sequence variant specific first and second primer is used. A uniform P1.1 and P2.1 for all sequence variants of a target nucleic acid can bind to the start nucleic acid chain and initiate the synthesis of the P1.1-Ext or P2.1-Ext. In the course of amplification, P1.1-Ext and P2.1-Ext are formed and no competitor primer is used. The separation of the two synthesized complementary primer extension products P1.1-Ext and P2.1-Ext preferably takes place specifically by complementary binding of the P1.1-Ext to the activator oligonucleotide. The separation of strands of another sequence variant, which were synthesized using (SN 1.2), takes place less efficiently due to the mismatch with position (N) to the corresponding sequence segment of the activator oligonucleotide. The amplification of the SN 1.2 sequence variant therefore takes place less efficiently or only insignificantly or not at all. The position of the sequence segment (3) corresponding to N2 of the target sequence is located near the second blocking unit, so that the interaction between the activator oligonucleotide and the respective first primer extension product can be influenced by modifications used in the second blocking unit.

FIG. 50 shows schematically an embodiment with topography of a specific amplification system and a starting nucleic acid chain with a polymorphic locus (N2) in an embodiment where the segment of the activator oligonucleotide corresponding to N2 is located in the third region (4). A target sequence specific but not sequence variant specific first and second primer is used. A uniform P1.1 and P2.1 for all sequence variants of a target nucleic acid can bind to the start nucleic acid chain and initiate the synthesis of the P1.1-Ext or P2.1-Ext. In the course of amplification, P1.1-Ext and P2.1-Ext are formed and no competitor primer is used. The separation of the two synthesized complementary primer extension products P1.1-Ext and P2.1-Ext preferably takes place specifically by complementary binding of the P1.1-Ext to the activator oligonucleotide. The separation of strands of another sequence variant, which were synthesized using (SN 1.2), takes place less efficiently due to the mismatch with position (N) to the corresponding sequence segment of the activator oligonucleotide. The amplification of the SN 1.2 sequence variant therefore takes place less efficiently or only insignificantly or not at all. The position of the sequence segment (4) corresponding to N2 of the target sequence is located at a distance from the second blocking unit, so that the interaction between the activator oligonucleotide and the respective first primer extension product is essentially unaffected by modifications used in the second blocking unit. The segment of the activator oligonucleotide corresponding to N2 is preferably composed of DNA monomers, wherein between 4 to 20 DNA monomers are used and at least 4 to 10 DNA monomers are located around position 4 on both sides.

FIG. 51 shows schematically an embodiment with topography of a specific amplification system and a start nucleic acid chain with a polymorphic locus (N2) in an embodiment in which the segment of the activator oligonucleotide corresponding to N2 is located in the third region (5). The N2 locus also comprises the 3′-terminal nucleotide of the second primer. Under reaction conditions, a specific P2.1 with a complementary 3′-terminal nucleotide can bind preferably specifically to the complementary position of a specific sequence variant starting from strands complementary to the starting nucleic acid (SN 1.1) and preferably specifically initiate the synthesis of the P2.1 Ext. In the course of the amplification, P1.1-Ext and P2.1-Ext are formed and no competitor primer is used. Binding to another sequence variant (SN 1.2) takes place less efficiently due to the mismatch with position (N) within the primer binding site of SN 1.2. Thus, amplification is initiated less efficiently.

FIG. 52 shows schematically an embodiment with topography of a specific amplification system and a start nucleic acid chain with a polymorphic locus (N2) in an embodiment where the segment of the activator oligonucleotide corresponding to N2 is located in the second region. Under reaction conditions, a specific P1.1 can bind preferably to the complementary position of a specific sequence variant of the start nucleic acid (SN 1.1) and initiate the synthesis of the P1.1 Ext. In the course of the amplification, P1.1-Ext and P2.1-Ext are formed. Binding to another sequence variant (SN 1.2) takes place less efficiently due to the mismatch with position (N) within the primer binding site of SN 1.2. Thus, amplification is started less efficiently.

To increase the specificity, a competitor primer (P 5.1) is added to the reaction, which is capable of binding predominantly complementary to the sequence variants of the target sequence, the amplification of which must be suppressed. Due to complementary binding with such primer binding sites, a competitor primer can bind preferably and be extended by the polymerase. The resulting product blocks the single-stranded primer binding sites for an interaction of the first amplification primer. In certain embodiments, the 3′-end of a competitor primer binds within the second blocking site of the activator oligonucleotide so that no extension of said primer can take place at the activator oligonucleotide. The competitor primer does not comprise a second region and thus cannot interact with the first region of the activator oligonucleotide. This prevents the extension product of the competitor oligonucleotide from being detached from the template with the participation of an activator oligonucleotide.

FIG. 53 shows schematically an embodiment with topography of a specific amplification system and a start nucleic acid chain with a polymorphic locus (N2) in an embodiment in which the segment of activator oligonucleotides corresponding to N2 is located in the second region (2). The N2 locus also comprises the 3′-terminal nucleotide of the first primer. A specific P1.1, with a complementary 3′-terminal nucleotide can, under reaction conditions, preferably bind specifically to the complementary position of a specific sequence variant of the start nucleic acid (SN 1.1) and initiate the synthesis of the P1.1 Ext. In the course of the amplification, P1.1-Ext and P2.1-Ext are formed. Binding to another sequence variant (SN 1.2) and extension by a polymerase takes place less efficiently due to the mismatch with position (N) within the primer binding site of SN 1.2. Thus, amplification is started less efficiently.

To increase the specificity, a competitor primer (P 5.2) is added to the reaction, which is able to bind predominantly complementarily to the sequence variants of the target sequence whose amplification must be suppressed. Due to complementary binding with such primer binding sites, a competitor primer can bind preferentially to certain sequence variants (here designated N) and be extended by the polymerase. The resulting product blocks the single-stranded primer binding sites for an interaction of the first amplification primer.

In certain embodiments, the 3′-end of a competitor primer binds within the second blocking site of the activator oligonucleotide, so that no extension of this primer can take place at the activator oligonucleotide. The competitor primer does not comprise a second region and thus cannot interact with the first region of the activator oligonucleotide. This prevents the extension product of the competitor oligonucleotide from being detached from the template.

FIG. 54 shows schematically an embodiment with topography of a specific amplification system and a start nucleic acid chain with a polymorphic locus (N2) in an embodiment in which the segment of the activator oligonucleotide corresponding to N2 is located in the third region (3). A target sequence specific but not sequence variant specific first and second primer is used. A uniform P1.1 and P2.1 for all sequence variants of a target nucleic acid can bind to the start nucleic acid chain and initiate the synthesis of the P1.1-Ext or P2.1-Ext. The separation of the two synthesized complementary primer extension products P1.1-Ext and P2.1-Ext preferably takes place specifically by complementary binding of the P1.1-Ext to the activator oligonucleotide. The separation of strands of another sequence variant synthesized using (SN 1.2) takes place less efficiently due to a mismatch with position (N) to the corresponding sequence segment of the activator oligonucleotide. The amplification of the SN 1.2 sequence variant therefore takes place less efficiently or only insignificantly or not at all. The position of the sequence segment (3) corresponding to N2 of the target sequence is located near the second blocking unit, so that the interaction between the activator oligonucleotide primer and the respective first primer extension product can be influenced by modifications used in the second blocking unit.

To increase specificity, a competitor primer (P 5.3 or P5.4) is added to the reaction, which is capable of predominantly complementary binding (N) to sequence variants of the target sequence, the amplification of which must be suppressed. Due to complementary binding with such primer binding sites, a competitor primer can bind preferentially to certain sequence variants (here designated N) and be extended by the polymerase. The resulting product blocks the single-stranded primer binding sites for an interaction of the first amplification primer.

In certain embodiments, the competitor-oligonucleotide-primer (P 5.3) is longer than the first region of the first oligonucleotide primer, so that its 3′-end binds within the fourth blocking unit of the activator oligonucleotide (fourth blocking unit is composed analogously to the second blocking unit and blocks a primer extension at the activator oligonucleotide), so that no extension of said primer can take place at the activator oligonucleotide. The competitor primer can completely (P 5.3) or partially (P 5.4) cover the segment of the template that can bind P1.1 predominantly complementary. The competitor primer does not comprise a second region and thus cannot interact with the first segment of the activator oligonucleotide. This prevents the extension product of the competitor oligonucleotide from being detached from the template with the participation of an activator oligonucleotide.

Such an amplification can be performed alone or in combination, for example with a subsequent PCR.

FIGS. 55-57 shows schematically the topography of components of the first amplification system.

The starting nucleic acid 1.1 comprises (in 5′-3′ direction) the following segments: M1.Y, M1.5, M1.4, M1.3, M1.2, M1.1, M1.X

The first oligonucleotide primer comprises (in 5′-3′ direction) the following segments: the first region (P1.1.1) and the second region (P1.1.2).

The controller oligonucleotide (C1.1) comprises (in 5′-3′ direction) the following segments: the third region (C1.1.3), the second region (C1.1.2) and the first region (C.1.1.1).

The controller oligonucleotide (C1.2) comprises (in 5′-3′ direction) the following segments: the third region (C1.2.3), the second region (C1.2.2) and the first region (C.1.2.1).

The second primer P2.1 comprises (in 5′-3′ direction) the following segments: P2.1.1.

The second primer P2.2 comprises (in 5′-3′ direction) the following segments: P2.2.1.

The second primer P2.3 comprises (in 5′-3′ direction) the following segments: P2.3.1.

The first primer extension product (P1.1-Ext) comprises (in 5′-3′ direction) the following segments: P1.1E6, P1.1 E5, P1.1E4, P1.1E3, P1.1E2, P1.1E1.

The second primer extension product (P2.1-Ext) comprises (in 5′-3′ direction) the following segments: P2.1E5, P2.1E4, P2.1E3, P2.1E2, P2.1 E1.

The primer extension products P1.1-Ext and P2.1-Ext preferably obtained during the first amplification can form a complementary double strand and together represent the second amplification fragment 1.1, which can serve as starting nucleic acid 2.1 for the second amplification.

Wherein P1.1.1 can bind predominantly complementary to M1.1 and can be extended by the polymerase and P2.1.1 can bind complementary to P1.1 E1 and can be extended. P1.1-Ext and P2.1-Ext represent the nucleic acid to be amplified, which serves as start nucleic acid 2.1 for the second amplification. P2.1, P2.2 and P2.3 represent variants of second primers and lead to identical amplification fragments.

FIG. 56 shows schematically the topography of the second amplification system:

The third oligonucleotide primer comprises (in 5′-3′ direction) the following segments: P3.1.2, P3.1.1, wherein P3.1.2 is not complementary to the start nucleic acid 1.1 or P2.1-Ext. P3.1.1 is essentially complementary to P2.1 E1 of P2.1-Ext.

The fourth oligonucleotide primer comprises (in 5′-3′ direction) the following segments: P4.1.2, P4.1.1, wherein P4.1.2 is not complementary to the complementary strand of the start nucleic acid 1.1 or to P1.1-Ext. P4.1.1 is essentially complementary to P1.1 E1 of P1.1-Ext.

The primer extension products P3.1-Ext and P4.1-Ext, preferably obtained during the second amplification, can form a complementary double strand and together represent the second amplification fragment 2.1.

P3.1-Ext comprises (in 5′-3′ direction) the following segments: P3.1 E7, P3.1 E6, P3.1 E5, P3.1 E4, P3.1 E3, P3.1 E2, P3.1 E1. Segments P3.1.E5 to P3.1 E3 are identical in sequence to P1.1 E4 to P1.1E2.

P4.1-Ext comprises (in 5′-3′ direction) the following segments: P4.1 E7, P4.1 E6, P43.1 E5, P4.1 E4, P4.1 E3, P43.1 E2, P4.1 E1. The segments P4.1.E5 to P4.1 E3 are identical in sequence to P2.1 E4 to P2.1 E2.

FIGS. 57-58 show variants of the third primer (P3.1, P3.2, P3.3) and the fourth primer (P4.1, P4.2, P4.3), wherein the 3′ segment of each primer may be shifted with respect to each other.

The coupling of both amplifications using start nucleic acid chain synthesized by the method of the invention can be summarized as follows:

The method comprises the following steps:

A) Providing a nucleic acid fragment comprising a first target sequence (a start nucleic acid chain)

B) Providing an initial amplification system, comprising:
A first oligonucleotide primer
A second oligonucleotide primer
A controller oligonucleotide
A first polymerase
Substrates for the first polymerase (dNTPs) and suitable buffer solution

C) Providing a second amplification system, comprising:
A third oligonucleotide primer
A fourth oligonucleotide primer
A second polymerase comprising a thermostable polymerase
Substrates for the second polymerase (dNTPs) and suitable buffer solution
A detection system

D) Carrying out a first amplification using the start nucleic acid chain as initial template strand and the first amplification system, obtaining a first amplification fragment 1. 1 comprising the first target sequence, and comprising a first primer extension product and a second primer extension product which can form a complementary double strand, wherein the first primer extension product results from template-dependent extension of a first primer by a polymerase using the start nucleic acid chain and/or the second primer extension product as template and the second primer extension product results from template-dependent extension of a second primer by a polymerase using the first primer extension product as template, wherein the first and second primer extension products can mutually serve as templates for the respective primer extension, and

wherein both complementary strands of the first amplification product 1.1 can be at least partially converted into single-stranded form with the participation of a controller oligonucleotide, so that renewed primer binding to respectively complementary sequences of the synthesized primer extension products is possible, and

the reaction conditions used for the first amplification comprise at least one temperature step which permits hybridization and template-dependent primer extension of both primers of the first amplification system and comprise at least one temperature step in which the first primer extension product is separated from the second primer extension product with the participation of the controller oligonucleotide, wherein the reaction conditions used do not permit spontaneous separation of the first primer extension product from the second primer extension product in the absence of the controller oligonucleotide. Wherein the first amplification is carried out until the desired quantity of the first amplification product 1.1 is synthesized.

E) Performing a second amplification using at least one of the two strands of the first amplification fragment 1.1 as initial template strand and using the second amplification system to obtain a second amplification fragment 2.1 comprising a third primer extension product and a fourth primer extension product which can form a complementary double strand, wherein both strands of the amplification fragment 2. 1 can both serve as templates in a primer extension, wherein the reaction conditions used in the second amplification allow hybridization of the two primers of the second amplification system at least in one temperature step and template-dependent primer extension of the primer of the second amplification system respectively bound to complementary regions and allow separation of a double strand at least in one further temperature step, comprising a third primer extension product and a fourth primer extension product, wherein the third and fourth primer extension products are converted to single-stranded form such that another primer binding and extension can occur. Wherein the second amplification is carried out until the desired amount of the second amplification product 2.1 is synthesized.

In detail, the properties of components of the respective amplification system, the nucleic acid fragment comprising a first target sequence as well as the reaction conditions used determine the course of the two amplification reactions.

Generally, the first amplification takes place with the participation of the controller oligonucleotide. The separation of the two synthesized primer extension products takes place at least partially depending on the sequence composition of the first primer extension product and its complementarity to the composition of the controller oligonucleotide.

During the second amplification, a second amplification fragment 2.1 is amplified essentially without participation of the controller oligonucleotide.

In the course of both amplification reactions, both desired amplification products (Amplification Fragment 1.1 and Amplification Fragment 2.1) and their intermediate stages (intermediates) can be formed (FIG. 58). The composition of the intermediates depends essentially on the components of the first and second amplification system.

In the following, some molecular procedures and the resulting products and the potential intermediates are explained schematically and exemplarily.

According to a first aspect of the invention, a method for amplification is provided. The method comprises the following steps:

1) a first amplification with the steps
1.A) Hybridizing a first oligonucleotide primer to the 3′ segment of a template strand of a first nucleic acid to be amplified comprising a first target sequence, wherein the first oligonucleotide primer comprises the following regions:
   a first region which can bind sequence-specifically to the 3′ segment of a template strand of the first nucleic acid to be amplified and is complementary to at least part of the target sequence;
   a second region contiguous to the 5′ end of the first region or connected by a linker, wherein the second region can be bound by a controller oligonucleotide and remains essentially uncopied by a polymerase used for the first amplification under the selected reaction conditions;
1.B) Extension of the first oligonucleotide primer when hybridized to complementary segments of a first nucleic acid to be amplified by the first template-dependent polymerase to give a first primer extension product which comprises, in addition to the first oligonucleotide primer, a region synthesized by the polymerase, which is essentially complementary to the template strand of the first nucleic acid to be amplified, which comprises a first target sequence, wherein the first primer extension product and the template strand of the first nucleic acid to be amplified are essentially present as a double strand under reaction conditions used in the first amplification;
1.C) Binding of a controller oligonucleotide to the first primer extension product, wherein the controller oligonucleotide comprises the following regions:
   a first region that can bind to the second region of the first primer and/or the first primer extension product
   a second region which is essentially complementary or fully complementary to the first region of the first oligonucleotide primer and/or the first primer extension product, and
   a third region which is essentially complementary or fully complementary to at least part of the polymerase synthesized region of the first primer extension product;
   wherein the controller oligonucleotide does not serve as a template for primer extension of the first oligonucleotide primer, and
   the controller oligonucleotide comprises a sequence segment which is complementary or essentially complementary to a strand of the first nucleic acid to be amplified, and
   the controller oligonucleotide binds to the first region of the first segment of the primer extension product, and
   and the controller oligonucleotide binds to the second region of the first primer extension product, as well as at least partially to the synthesized region of the first primer extension product, thereby displacing complementary portions of the template strand of the first nucleic acid to be amplified; wherein the polymerase synthesized region of the first primer extension product comprising the segment of the first primer extension product complementary to the second oligonucleotide primer becomes single-stranded under reaction conditions
1.D) Hybridizing a second oligonucleotide primer to the single-stranded complementary segment of the first primer extension product, wherein the second oligonucleotide primer comprises a region which can hybridize sequence specifically to the synthesized region of the first primer extension product and comprises at least a portion of a target sequence
1.E) Extension of the second oligonucleotide primer by the first polymerase using the first primer extension product as template to obtain a second primer extension product which, in addition to the second oligonucleotide primer, comprises a region synthesized by the polymerase which comprises at least a portion of the first target sequence, wherein the first primer extension product and the second primer extension product form a first double-strand amplification product under reaction conditions of the first amplification; and;
1.F) Repeat the steps of the first amplification if necessary

2) a second amplification with the steps (FIG. 58):
2.A) Hybridizing a third oligonucleotide primer to the second primer extension product of the first amplification product, wherein the third oligonucleotide primer comprises a first region that can bind essentially sequence-specifically to a segment of the second primer extension product;
2.B) Extension of the third oligonucleotide primer by a second polymerase using the second primer extension product as template to obtain a third primer extension product (P3.1-Ext part 1), which comprises, in addition to the third oligonucleotide primer, a region synthesized by the polymerase, which comprises a sequence segment which is essentially complementary to the second primer extension product or to the first nucleic acid to be amplified or to a portion of the first target sequence, wherein the second primer extension product and the third primer extension product (P3.1-Ext part 1) are present as double strand;
2C) Hybridizing a fourth oligonucleotide primer to the first primer extension product of the first amplification product, wherein the fourth oligonucleotide primer comprises a first segment that can bind essentially sequence specifically to the polymerase synthesized region of the first primer extension product;
2D) Extension of the fourth oligonucleotide primer by the second polymerase using the first primer extension product as template to obtain a fourth primer extension product (P4.1-Ext part 1), which comprises, in addition to the fourth oligonucleotide primer, a region synthesized by the polymerase which is essentially complementary to the first primer extension product and comprises portions of the target sequence, wherein the first primer extension product and the fourth primer extension product (P4.1-Ext part 1) are present as double strands;
2E) Separation of the double strand from the first primer extension product and the fourth primer extension product (P4.1-Ext part 1) and the double strand from the second primer extension product and the third primer extension product (P3.1-Ext part 1)
2.F) Hybridizing a third oligonucleotide primer to the fourth primer extension product (obtained in step 2D, P4.1-Ext part 1), wherein the third oligonucleotide primer comprises a first region that can bind essentially sequence-specifically to a segment of the fourth primer extension product (P4.1-Ext part 1);
2.G) Extension of the third oligonucleotide primer by a second polymerase using the fourth primer extension product (P4.1-Ext part 1) as template to obtain a complete third primer extension product (P3.1-Ext), wherein the third primer extension product (P3.1-Ext) and the fourth primer extension product (P4.1-Ext part 1) are present as double strand;
2H) Hybridizing a fourth oligonucleotide primer to the third primer extension product (obtained in step 2B, P3.1-Ext part 1), wherein the fourth oligonucleotide primer comprises a first segment that can bind essentially sequence specifically to the polymerase synthesized region of the third primer extension product;
2I) Extension of the fourth oligonucleotide primer by the second polymerase using the third primer extension product (P3.1-Ext part 1) as template to obtain a complete fourth primer extension product (P4.1-Ext), wherein the third primer extension product (P3.1-Ext part 1) and the fourth primer extension product (P4.1-Ext) are present as double strand;
2J) Separation of the double strand from the third primer extension product (P3.1-Ext part 1) and the complete fourth primer extension product (P4.1-Ext) and the double strand from the fourth primer extension product (P4.1-Ext part 1) and the complete third primer extension product (P3.1-Ext);
2.K) Hybridizing a third oligonucleotide primer to the complete fourth primer extension product (obtained in step 21, P4.1-Ext), wherein the third oligonucleotide primer comprises a first region that can bind essentially sequence specifically to a segment of the fourth primer extension product (P4.1-Ext);
2.L) Extension of the third oligonucleotide primer by a second polymerase using the complete fourth primer extension product (P4.1-Ext) as template to obtain a complete third primer extension product (P3.1-Ext), wherein the complete third primer extension product (P3.1-Ext) and the complete fourth primer extension product (P4.1-Ext) are present as double strand;
2.M) Hybridizing a fourth oligonucleotide primer to the complete third primer extension product (obtained in step 2G, P3.1-Ext), wherein the fourth oligonucleotide primer comprises a first region that can bind essentially sequence specifically to the polymerase synthesized region of the complete third primer extension product;
2.N) Extension of the fourth oligonucleotide primer by the second polymerase using the complete third primer extension product (P3.1-Ext) as template to obtain a complete fourth primer extension product (P4.1-Ext), wherein the complete third primer extension product (P3.1-Ext) and the complete fourth primer extension product (P4.1-Ext) are double-stranded;
2.O) Separating the double strands formed in steps 2L and 2N from the complete third primer extension product (P3.1-Ext) and the complete fourth primer extension product (P4.1-Ext)
2.P) Repeat the steps of the second amplification, if necessary, multiplying the complete third primer extension product (P3.1-Ext) and the complete fourth primer extension product (P4.1-Ext).

wherein a detection system is added to the reaction mixture.

The extent of yields of individual products and intermediates depends on several factors. For example, higher concentrations of individual primers generally favor product/intermediate yields. Furthermore, the formation of products/intermediates can be influenced by the reaction temperature as well as by the binding/affinity of individual reactants to each other (affinity of the binding of individual primers to their complementary primer binding sites within template strands): generally, for example, longer oligonucleotides bind better than shorter oligonucleotides at higher temperatures, and the CG content can play a role as well in complementary sequence segments: CG-rich sequences also bind more strongly than AT-rich sequences at higher temperatures. Furthermore, modifications such as MGB or 2-amino-dA or LNA can increase the binding strength of primers to their respective complementary segments, which also results in preferential binding of primers at higher temperatures.

From this it can be deduced that by designing sequence segments of individual primer sequences it is possible to influence the yield of certain products/intermediates and thus to influence their enrichment during amplification.

Method for amplifying a nucleic acid (FIGS. 56-58), comprising the steps:
a) a first amplification with the steps:
   Provision of a start nucleic acid 1.1 comprising a target sequence
   Hybridizing a first oligonucleotide primer (P1.1) to a nucleic acid to be amplified with a target sequence (either start nucleic acid 1.1 or P2.1-Ext), wherein the first oligonucleotide primer (P1.1) comprises the following regions:
      a first region (P1.1.1), which can bind sequence-specifically to a region of the nucleic acid to be amplified (P2.1 E1)
      a second region (P1.1.2) which adjoins the 5′ end of the first region or is connected by a linker, wherein the second region can be bound by a controller oligonucleotide and remains essentially uncopied for the polymerase used under the selected reaction conditions;
   Extension of the first oligonucleotide primer (P1.1) by a first polymerase to obtain a first primer extension product (P1.1-Ext) which comprises, in addition to the first oligonucleotide primer (P1.1), a synthesized region (P1.1 E1 to P1.1 E4) which is essentially complementary to the nucleic acid to be amplified or to the target sequence, wherein the first primer extension product and the nucleic acid to be amplified are present as a double strand;
   Binding of a controller oligonucleotide (C1.2) to the first primer extension product (P1.1-Ext), wherein the controller oligonucleotide (C1.2) comprises the following regions:
      a first region (C1.2.1) that can bind to the second region (overhang) of the first primer extension product (P1.1 E6),
      a second region (C1.2.2) which is complementary to the first region of the first oligonucleotide primer (P1.1.1), and
      a third region (C1.2.3) essentially complementary to at least part of the synthesized region of the first segment of the primer extension product (P1.1 E4); and
      wherein the controller oligonucleotide (C1.1) does not serve as a template for primer extension of the first oligonucleotide primer (P1.1), and the first controller oligonucleotide (C1.1) binds to the first primer extension product (P1.1 E6, P1.1 E5, P1.1 E4) while displacing the region (P2.1 E1, P2.1 E2) of the nucleic acid to be amplified complementary to said region;
   Hybridizing a second oligonucleotide primer (P1.1) to the first primer extension product, wherein the second oligonucleotide primer (P2.1) comprises (P2.1.1) a region which can bind sequence-specifically to the synthesized region of the first primer extension product (P1.1 E1),
   extension of the second oligonucleotide primer (P.2 .1) by the first polymerase to give a second primer extension product (P2.1-Ext) which comprises, in addition to the second oligonucleotide primer (P2.1), a synthesized region (P2.1 E4 to P2.1 E1) which is essentially identical to the nucleic acid to be amplified or to the target sequence, wherein the first primer extension product (P1.1-Ext) and the second primer extension product (P2.1) form a first double-stranded amplification product; and
   steps of the first amplification should be repeated until the desired amount of first amplification products is synthesized.
b) a second amplification with the steps:
   Hybridizing a third oligonucleotide primer (P3.2) to the second and/or the fourth primer extension product (P1.1-Ext. or P4.1-Ext), wherein the third oligonucleotide primer (P3.2) comprises a first region (P3.1.1) which can bind sequence-specifically to a region of the second primer extension product (P2.1 E1) and the fourth primer extension product (P4.1 E2),
   extension of the third oligonucleotide primer (P3.2) by a second polymerase to obtain a third primer extension product (P3.2-Ext), which in addition to the third oligonucleotide primer (P3.2) has a synthesized region (P3.1 E5 to P3.1 E2 or P3. 1E5 to P3.1 E1), which is essentially complementary to the second primer extension product (P2.1-Ext) or to the target sequence, wherein the second primer extension product (P2.1) and the third primer extension product (P3.1) are present as double strand;
   hybridizing a fourth oligonucleotide primer (P4.2) to the first primer extension product (P1.1-Ext) and/or to the third primer extension product (P3.1-Ext), wherein the fourth oligonucleotide primer (P4. 2) comprises a first region (P4.1.1) which can bind sequence specifically to the synthesized region of the first primer extension product (P1.1 E1) and the third primer extension product (P3.1 E2),
   extension of the fourth oligonucleotide primer (P4.2) by the second polymerase to obtain a fourth primer extension product (P4.2-Ext), which in addition to the fourth oligonucleotide primer has a synthesized region (P4.1 E5 to P4.1 E2 or P4.1 E5 to P4. 1 E1) which is essentially complementary to the first primer extension product (P1.1-Ext) or identical to the target sequence, wherein the first primer extension product (P1.1-Ext) and the fourth primer extension product (P4.1-Ext) are present as double strand;
   separating the double strand from the first primer extension product (P1.1-Ext) and the fourth primer extension product (P4.2-Ext) and the double strand from the second primer extension product (P2.1-Ext) and the third primer extension product (P3.1-Ext) and the double strand from the fourth primer extension product (P4.1-Ext) and the third primer extension product (P3.1-Ext);
   hybridizing the third oligonucleotide primer (P3.2) to the fourth primer extension product (P4.2-Ext) and extending the third oligonucleotide primer (P3.2) by the second polymerase; and
   hybridization of the fourth oligonucleotide primer to the third primer extension product (P3.2-Ext) and extension of the fourth oligonucleotide primer (P4.2) by the second polymerase.

1. A method for the synthesis of a nucleic acid sequence complementary to a target sequence, wherein
a) a sample comprising a nucleic acid polymer which comprises the target sequence M, wherein the target sequence in 5′-3′ orientation comprises the sequence segments MS and MP, and MP is located immediately in the 3′ direction of MS, wherein the sample is brought into contact with the following components:
b) an oligonucleotide primer P, which in 5′-3′ orientation comprises the sequence segments PC and PM, and PM is located immediately in the 3′ direction of PC, wherein PM comprises the sequence complementary to the sequence segment MP and PC cannot bind to M or a sequence immediately following MP in the 3′ direction;
c) a controller oligonucleotide C, which in 5′-3′ orientation comprises the sequence segments CS, CP and CC, wherein CP is located immediately in 3′ direction of CS and immediately in 5′ direction of CC, and wherein CS is identical to at least the 3′ segment of MS, immediately in 5′ direction of MP, and CP and CC comprise the sequence complementary to the sequence segment PM and PC, and wherein CS comprises modified nucleotide building blocks so that CS cannot serve as a template for the activity of the template-dependent nucleic acid polymerase;
d) a template-dependent nucleic acid polymerase, particularly a DNA polymerase, as well as substrates of the template-dependent nucleic acid polymerase, and suitable cofactors,
wherein a primer extension product P′ is obtained, which in addition to the sequence regions PC and PM comprises a synthesized region PS which is essentially complementary to the target sequence MS,
and wherein
either the reaction conditions are chosen, and/or
the length and melting temperature of PC and/or MS are chosen so that P′ can form a double strand with M and P′ can form a double strand with C.

2. The method according to claim 1, wherein the sequence segment CS at its 3′ end immediately in 5′ direction of the sequence segment CP contains a sequence segment CS′ of 5 to 15 nucleotide positions in length, which consists of nucleic acid analogues, particularly of 2′O-alkyl ribonucleotides.

3. The method according to claim 1, wherein M comprises a sequence segment MB immediately in 5′ direction of MS, and further wherein a block oligonucleotide B, which can hybridize to MB, is brought into contact with the sample, and wherein
a. B comprises nucleoside analogues which are selected such that a hybrid of B with MB does not allow the template-dependent nucleic acid polymerase to react on MB, or
b. the template-dependent nucleic acid polymerase is selected such that a hybrid of B with MB does not allow the template-dependent nucleic acid polymerase to react on MB, even if B consists of natural nucleosides or deoxynucleotides.

4. The method according to claim 3, wherein B is contacted with the sample before the template-dependent nucleic acid polymerase is brought into contact with the sample.

5. The method according to claim 1, wherein CS is identical to MS and CP is identical to MP.

6. The method according to claim 1, wherein the formation of the double strand of P′ and C is preferred over the formation of the double strand of P′ and M.

7. (canceled)

8. The method according to claim 1, wherein the sample is not brought into contact with an oligonucleotide primer which can bind to a sequence segment located in PS and from which a synthesis of a counter strand to PS can take place via a template-dependent nucleic acid polymerase, and does not result in the exponential amplification of M.

9. (canceled)

10. (canceled)

11. The method according to claim 1, characterized in that PC has a length in the range of 5 nucleotides to 85 nucleotides, particularly that PC has between 50% and 300% of the length of the sequence segment PM.

12. (canceled)

13. The method according to claim 1, characterized in that the sample comprises a variant VM of the target sequence M which is ≥92%, particularly ≥95%, ≥92%, ≥94%, ≥96% or even ≥96% identical to M, wherein a segment VMS of the variant is highly identical to the segment MS and a segment VMP is highly identical to the segment MP, and VM is different from the sequence M in at least one position VMM, particularly comprising one or more substitution(s), insertion(s) and/or deletion(s).

14. (canceled)

15. The method according to claim 13, wherein VMM is located in the sequence segment comprised in VMS.

16. (canceled)

17. (canceled)

18. (canceled)

19. The method according to claim 13, further characterized in that a second controller oligonucleotide VC is brought into contact with the sample, wherein the second controller oligonucleotide VC in 5′-3′ orientation comprises the sequence segments VCS, VCP and VCC, wherein VCP is located immediately in 3′ direction of VCS and immediately in 5′ direction of VCC, and wherein VCS is identical at least to the 3′ segment of VMS, and VCP and VCC comprise the sequence complementary to the sequence segment PM and PC and wherein VCS comprises modified nucleotide building blocks so that VCS cannot serve as a template for the activity of the template-dependent nucleic acid polymerase.

20. The method according to claim 13, wherein VMM is at least partially comprised in the sequence segment comprising VMP.

21. (canceled)

22. The method according to claim 13, further characterized in that a second oligonucleotide primer VP is brought into contact with the sample, wherein the second oligonucleotide primer VP in 5′-3′ orientation comprises the sequence segments VPC and VPM, and VPM is located immediately in 3′ of VPC, wherein VPM comprises the sequence complementary to the sequence segment VMP of the target sequence VM and VPC cannot bind to VM or a sequence immediately following VMP in 3′.

23. The method according to claim 13, characterized in that VMM is at the 5′ end of VMP, in other words the 3′ end of VPM hybridizes to VMM.

24. The method according to claim 13, characterized in that a competitor oligonucleotide KP which hybridizes with VMP is further brought into contact with the sample.

25. (canceled)

26. A kit for performing a method according to claim 1, comprising:
an oligonucleotide primer P which in 5′-3′ orientation comprises the sequence segments PC and PM, and PM is located immediately in 3′ of PC, wherein PM can bind to a genomic sequence M of a eukaryotic organism or of a pathogenic bacterium, particularly of a mammal, particularly to a human target sequence comprising a primer binding site MP, and PC cannot bind to a sequence directly in the 3′ direction of MP;
a controller oligonucleotide C which in 5′-3′ orientation comprises the sequence segments CS, CP and CC, wherein CP is located immediately in 3′ of CS and immediately in 5′ of CC, and wherein CS is identical to a sequence MS located immediately in 5′ direction of MP and CP is identical to MP and CC has the sequence complementary to the sequence segment PC, and wherein CS comprises modified nucleotide building blocks such that CS cannot serve as a template for the activity of a template-dependent nucleic acid polymerase.

27. The kit according to claim 26, further comprising a block oligonucleotide B which can hybridize to a sequence segment of target sequence MB located immediately in 5′ direction of MS, wherein B comprises nucleoside analogues selected such that a hybrid of B with MB does not allow a template-dependent nucleic acid polymerase to react on MB.

28. The kit according to claim 26, further comprising
a second oligonucleotide primer VP which in 5′-3′ orientation comprises the sequence segments VPC and VPM, and VPM is located immediately in 3′ of VPC, wherein VPM can bind to a genomic sequence VM comprising a primer binding site VMP, wherein VM is identical to M to ≥85%, particularly to ≥95%, ≥92%, ≥94%, ≥96% or even to ≥92%, but is different from M in at least one position, and VPC cannot bind to a sequence located directly in 3′ direction of VMP; and/or
a second controller oligonucleotide VC, which in 5′-3′ orientation comprises the sequence segments VCS, VCP and VCC, wherein VCP is located immediately in 3′ direction of VCS and immediately in 5′ direction of VCC, and wherein VCS is identical to a genomic sequence VMS, immediately in 5′ direction of VMP, which is part of a variant VM which is ≥85%, particularly ≥95%, ≥92%, ≥94%, ≥96% or even ≥98% identical to M but different from M in at least one position, and VCP is identical to VMP and VCC comprises the complementary sequence to the sequence segment VPC, and wherein VCS comprises modified nucleotide building blocks such that VCS cannot serve as a template for the activity of a template-dependent nucleic acid polymerase,
and/or
a competitor oligonucleotide KP, which hybridizes with VMP

29. The kit according to claim 26, further comprising a template-dependent nucleic acid polymerase, particularly a DNA polymerase, as well as optionally substrates of the template-dependent nucleic acid polymerase and suitable cofactors.

30. The kit according to claim 26, wherein said kit does not comprise an oligonucleotide primer which can bind to a sequence segment located on the opposite strand of M and from which a synthesis of a sequence essentially identical to the 5′ end of MP on M can be performed by the template-dependent nucleic acid polymerase.
